SEQUENCE LISTING (2516 sequences)

SEQ ID NO: 1 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gaaacctcctcacctcctgg

SEQ ID NO: 2 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggatgccttatttccagctc

SEQ ID NO: 3 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctgagagagattgccaggag

SEQ ID NO: 4 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
caacaccaatggaagctgac

SEQ ID NO: 5 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cccacaacaccaatggaagc

SEQ ID NO: 6 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ccaactccaacctgtctctg

SEQ ID NO: 7 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gagcctcaagattggagaac

SEQ ID NO: 8 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ccaatctggatgaggagagc

SEQ ID NO: 9 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gtctccagcagtctctcctc

SEQ ID NO: 10 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cactattcatctgctctccc

SEQ ID NO: 11 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctctgactgctgcaatatcc

SEQ ID NO: 12 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gccagtggtatcttcaacag

SEQ ID NO: 13 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gaggctgcttaatccactgg

SEQ ID NO: 14 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gtctgcctcaattctgcatc

SEQ ID NO: 15 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gccaagatctctggctgcac

SEQ ID NO: 16 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctttaacaatggcatcctgg

SEQ ID NO: 17 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tgcattggttctacatagcc

SEQ ID NO: 18 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
agctgacaacagcatctagc

SEQ ID NO: 19 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gaacaggtgctccatttgac

SEQ ID NO: 20 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ttcttccttgtcctttctcc

SEQ ID NO: 21 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggccagtaatgatgtggaac

SEQ ID NO: 22 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tctgctgactcacatggcac

SEQ ID NO: 23 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cagtttccaacatttcagcc

SEQ ID NO: 24 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
agacctgaggatagaagcag

SEQ ID NO: 25 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
aggtagaagttcctcttccc

SEQ ID NO: 26 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gcccatgctaacccaaatcc

SEQ ID NO: 27 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cttcagtggtgagtgctgtg

SEQ ID NO: 28 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
agctctgcaacatgaatccc

SEQ ID NO: 29 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gaaactcctcaagttccagg

SEQ ID NO: 30 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cacttctttgctgctggttc

SEQ ID NO: 31 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tcaccttggacaggaactcc

SEQ ID NO: 32 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
acagtcctcagttacagctc

SEQ ID NO: 33 - RNA, 19 nt, synthetic construct
  source 1..19: mol_type = other RNA
agtatagagttgaagttta

SEQ ID NO: 34 - RNA, 19 nt, synthetic construct
  source 1..19: mol_type = other RNA
ggttggagttgggtatgtg

SEQ ID NO: 35 - RNA, 19 nt, synthetic construct
  source 1..19: mol_type = other RNA
gggtatgtggattccatac

SEQ ID NO: 36 - RNA, 19 nt, synthetic construct
  source 1..19: mol_type = other RNA
cactgtctgcggtaatcaa

SEQ ID NO: 37 - RNA, 19 nt, synthetic construct
  source 1..19: mol_type = other RNA
agatttacttcgtcgattc

SEQ ID NO: 38 - RNA, 19 nt, synthetic construct
  source 1..19: mol_type = other RNA
gcagcaaacttacaagatt

SEQ ID NO: 39 - RNA, 19 nt, synthetic construct
  source 1..19: mol_type = other RNA
cagggtataaatcaactac

SEQ ID NO: 40 - RNA, 19 nt, synthetic construct
  source 1..19: mol_type = other RNA
gggtataaatcaactacct

SEQ ID NO: 41 - RNA, 19 nt, synthetic construct
  source 1..19: mol_type = other RNA
ggatattactttcgtgtaa

SEQ ID NO: 42 - RNA, 19 nt, synthetic construct
  source 1..19: mol_type = other RNA
agtccttcgtaacaataaa

SEQ ID NO: 43 - RNA, 19 nt, synthetic construct
  source 1..19: mol_type = other RNA
tcgtaacaataaaaacttt

SEQ ID NO: 44 - RNA, 19 nt, synthetic construct
  source 1..19: mol_type = other RNA
ggtgttaaatttaccaaca

SEQ ID NO: 45 - RNA, 19 nt, synthetic construct
  source 1..19: mol_type = other RNA
gacactcaatgatgtgtta

SEQ ID NO: 46 - RNA, 19 nt, synthetic construct
  source 1..19: mol_type = other RNA
gcacctgttccatatgtac

SEQ ID NO: 47 - RNA, 19 nt, synthetic construct
  source 1..19: mol_type = other RNA
ccccaatatgggaggtaaa

SEQ ID NO: 48 - RNA, 19 nt, synthetic construct
  source 1..19: mol_type = other RNA
gggaggtaaatcaacatat

SEQ ID NO: 49 - RNA, 19 nt, synthetic construct
  source 1..19: mol_type = other RNA
caacatatattcgacaaac

SEQ ID NO: 50 - RNA, 19 nt, synthetic construct
  source 1..19: mol_type = other RNA
gggtgatagtactcatggc

SEQ ID NO: 51 - RNA, 19 nt, synthetic construct
  source 1..19: mol_type = other RNA
ggcccaaattgggtgtttt

SEQ ID NO: 52 - RNA, 19 nt, synthetic construct
  source 1..19: mol_type = other RNA
gggttagcatgggctatat

SEQ ID NO: 53 - RNA, 19 nt, synthetic construct
  source 1..19: mol_type = other RNA
gctatatcagaatacattg

SEQ ID NO: 54 - RNA, 19 nt, synthetic construct
  source 1..19: mol_type = other RNA
tgtctgtgatcaaagtttt

SEQ ID NO: 55 - RNA, 19 nt, synthetic construct
  source 1..19: mol_type = other RNA
ccctaagcatgtaatagag

SEQ ID NO: 56 - RNA, 19 nt, synthetic construct
  source 1..19: mol_type = other RNA
ggagaatcgcaaggatatg

SEQ ID NO: 57 - RNA, 19 nt, synthetic construct
  source 1..19: mol_type = other RNA
taaacttcaactctatact

SEQ ID NO: 58 - RNA, 19 nt, synthetic construct
  source 1..19: mol_type = other RNA
cacatacccaactccaacc

SEQ ID NO: 59 - RNA, 19 nt, synthetic construct
  source 1..19: mol_type = other RNA
gtatggaatccacataccc

SEQ ID NO: 60 - RNA, 19 nt, synthetic construct
  source 1..19: mol_type = other RNA
ttgattaccgcagacagtg

SEQ ID NO: 61 - RNA, 19 nt, synthetic construct
  source 1..19: mol_type = other RNA
gaatcgacgaagtaaatct

SEQ ID NO: 62 - RNA, 19 nt, synthetic construct
  source 1..19: mol_type = other RNA
aatcttgtaagtttgctgc

SEQ ID NO: 63 - RNA, 19 nt, synthetic construct
  source 1..19: mol_type = other RNA
gtagttgatttataccctg

SEQ ID NO: 64 - RNA, 19 nt, synthetic construct
  source 1..19: mol_type = other RNA
aggtagttgatttataccc

SEQ ID NO: 65 - RNA, 19 nt, synthetic construct
  source 1..19: mol_type = other RNA
ttacacgaaagtaatatcc

SEQ ID NO: 66 - RNA, 19 nt, synthetic construct
  source 1..19: mol_type = other RNA
tttattgttacgaaggact

SEQ ID NO: 67 - RNA, 19 nt, synthetic construct
  source 1..19: mol_type = other RNA
aaagtttttattgttacga

SEQ ID NO: 68 - RNA, 19 nt, synthetic construct
  source 1..19: mol_type = other RNA
tgttggtaaatttaacacc

SEQ ID NO: 69 - RNA, 19 nt, synthetic construct
  source 1..19: mol_type = other RNA
taacacatcattgagtgtc

SEQ ID NO: 70 - RNA, 19 nt, synthetic construct
  source 1..19: mol_type = other RNA
gtacatatggaacaggtgc

SEQ ID NO: 71 - RNA, 19 nt, synthetic construct
  source 1..19: mol_type = other RNA
tttacctcccatattgggg

SEQ ID NO: 72 - RNA, 19 nt, synthetic construct
  source 1..19: mol_type = other RNA
atatgttgatttacctccc

SEQ ID NO: 73 - RNA, 19 nt, synthetic construct
  source 1..19: mol_type = other RNA
gtttgtcgaatatatgttg

SEQ ID NO: 74 - RNA, 19 nt, synthetic construct
  source 1..19: mol_type = other RNA
gccatgagtactatcaccc

SEQ ID NO: 75 - RNA, 19 nt, synthetic construct
  source 1..19: mol_type = other RNA
aaaacacccaatttgggcc

SEQ ID NO: 76 - RNA, 19 nt, synthetic construct
  source 1..19: mol_type = other RNA
atatagcccatgctaaccc

SEQ ID NO: 77 - RNA, 19 nt, synthetic construct
  source 1..19: mol_type = other RNA
caatgtattctgatatagc

SEQ ID NO: 78 - RNA, 19 nt, synthetic construct
  source 1..19: mol_type = other RNA
aaaactttgatcacagaca

SEQ ID NO: 79 - RNA, 19 nt, synthetic construct
  source 1..19: mol_type = other RNA
ctctattacatgcttaggg

SEQ ID NO: 80 - RNA, 19 nt, synthetic construct
  source 1..19: mol_type = other RNA
catatccttgcgattctcc

SEQ ID NO: 81 - RNA, 19 nt, synthetic construct
  source 1..19: mol_type = other RNA
agtatagagttgaagttta

SEQ ID NO: 82 - RNA, 19 nt, synthetic construct
  source 1..19: mol_type = other RNA
ggttggagttgggtatgta

SEQ ID NO: 83 - RNA, 19 nt, synthetic construct
  source 1..19: mol_type = other RNA
gggtatgtggattccataa

SEQ ID NO: 84 - RNA, 19 nt, synthetic construct
  source 1..19: mol_type = other RNA
cactgtctgcggtaatcaa

SEQ ID NO: 85 - RNA, 19 nt, synthetic construct
  source 1..19: mol_type = other RNA
agatttacttcgtcgatta

SEQ ID NO: 86 - RNA, 19 nt, synthetic construct
  source 1..19: mol_type = other RNA
gcagcaaacttacaagata

SEQ ID NO: 87 - RNA, 19 nt, synthetic construct
  source 1..19: mol_type = other RNA
cagggtataaatcaactaa

SEQ ID NO: 88 - RNA, 19 nt, synthetic construct
  source 1..19: mol_type = other RNA
gggtataaatcaactacca

SEQ ID NO: 89 - RNA, 19 nt, synthetic construct
  source 1..19: mol_type = other RNA
ggatattactttcgtgtaa

SEQ ID NO: 90 - RNA, 19 nt, synthetic construct
  source 1..19: mol_type = other RNA
agtccttcgtaacaataaa

SEQ ID NO: 91 - RNA, 19 nt, synthetic construct
  source 1..19: mol_type = other RNA
tcgtaacaataaaaactta

SEQ ID NO: 92 - RNA, 19 nt, synthetic construct
  source 1..19: mol_type = other RNA
ggtgttaaatttaccaaca

SEQ ID NO: 93 - RNA, 19 nt, synthetic construct
  source 1..19: mol_type = other RNA
gacactcaatgatgtgtta

SEQ ID NO: 94 - RNA, 19 nt, synthetic construct
  source 1..19: mol_type = other RNA
gcacctgttccatatgtaa

SEQ ID NO: 95 - RNA, 19 nt, synthetic construct
  source 1..19: mol_type = other RNA
ccccaatatgggaggtaaa

SEQ ID NO: 96 - RNA, 19 nt, synthetic construct
  source 1..19: mol_type = other RNA
gggaggtaaatcaacataa

SEQ ID NO: 97 - RNA, 19 nt, synthetic construct
  source 1..19: mol_type = other RNA
caacatatattcgacaaaa

SEQ ID NO: 98 - RNA, 19 nt, synthetic construct
  source 1..19: mol_type = other RNA
gggtgatagtactcatgga

SEQ ID NO: 99 - RNA, 19 nt, synthetic construct
  source 1..19: mol_type = other RNA
ggcccaaattgggtgttta

SEQ ID NO: 100 - RNA, 19 nt, synthetic construct
  source 1..19: mol_type = other RNA
gggttagcatgggctataa

SEQ ID NO: 101 - RNA, 19 nt, synthetic construct
  source 1..19: mol_type = other RNA
gctatatcagaatacatta

SEQ ID NO: 102 - RNA, 19 nt, synthetic construct
  source 1..19: mol_type = other RNA
tgtctgtgatcaaagttta

SEQ ID NO: 103 - RNA, 19 nt, synthetic construct
  source 1..19: mol_type = other RNA
ccctaagcatgtaatagaa

SEQ ID NO: 104 - RNA, 19 nt, synthetic construct
  source 1..19: mol_type = other RNA
ggagaatcgcaaggatata

SEQ ID NO: 105 - RNA, 21 nt, synthetic construct
  source 1..21: mol_type = other RNA
taaacttcaactctatactga

SEQ ID NO: 106 - RNA, 21 nt, synthetic construct
  source 1..21: mol_type = other RNA
tacatacccaactccaacctg

SEQ ID NO: 107 - RNA, 21 nt, synthetic construct
  source 1..21: mol_type = other RNA
ttatggaatccacatacccaa

SEQ ID NO: 108 - RNA, 21 nt, synthetic construct
  source 1..21: mol_type = other RNA
ttgattaccgcagacagtgat

SEQ ID NO: 109 - RNA, 21 nt, synthetic construct
  source 1..21: mol_type = other RNA
taatcgacgaagtaaatcttc

SEQ ID NO: 110 - RNA, 21 nt, synthetic construct
  source 1..21: mol_type = other RNA
tatcttgtaagtttgctgctt

SEQ ID NO: 111 - RNA, 21 nt, synthetic construct
  source 1..21: mol_type = other RNA
ttagttgatttataccctgat

SEQ ID NO: 112 - RNA, 21 nt, synthetic construct
  source 1..21: mol_type = other RNA
tggtagttgatttataccctg

SEQ ID NO: 113 - RNA, 21 nt, synthetic construct
  source 1..21: mol_type = other RNA
ttacacgaaagtaatatccaa

SEQ ID NO: 114 - RNA, 21 nt, synthetic construct
  source 1..21: mol_type = other RNA
tttattgttacgaaggacttt

SEQ ID NO: 115 - RNA, 21 nt, synthetic construct
  source 1..21: mol_type = other RNA
taagtttttattgttacgaag

SEQ ID NO: 116 - RNA, 21 nt, synthetic construct
  source 1..21: mol_type = other RNA
tgttggtaaatttaacaccat

SEQ ID NO: 117 - RNA, 21 nt, synthetic construct
  source 1..21: mol_type = other RNA
taacacatcattgagtgtctg

SEQ ID NO: 118 - RNA, 21 nt, synthetic construct
  source 1..21: mol_type = other RNA
ttacatatggaacaggtgctc

SEQ ID NO: 119 - RNA, 21 nt, synthetic construct
  source 1..21: mol_type = other RNA
tttacctcccatattggggcc

SEQ ID NO: 120 - RNA, 21 nt, synthetic construct
  source 1..21: mol_type = other RNA
ttatgttgatttacctcccat

SEQ ID NO: 121 - RNA, 21 nt, synthetic construct
  source 1..21: mol_type = other RNA
ttttgtcgaatatatgttgat

SEQ ID NO: 122 - RNA, 21 nt, synthetic construct
  source 1..21: mol_type = other RNA
tccatgagtactatcacccca

SEQ ID NO: 123 - RNA, 21 nt, synthetic construct
  source 1..21: mol_type = other RNA
taaacacccaatttgggccat

SEQ ID NO: 124 - RNA, 21 nt, synthetic construct
  source 1..21: mol_type = other RNA
ttatagcccatgctaacccaa

SEQ ID NO: 125 - RNA, 21 nt, synthetic construct
  source 1..21: mol_type = other RNA
taatgtattctgatatagccc

SEQ ID NO: 126 - RNA, 21 nt, synthetic construct
  source 1..21: mol_type = other RNA
taaactttgatcacagacacc

SEQ ID NO: 127 - RNA, 21 nt, synthetic construct
  source 1..21: mol_type = other RNA
ttctattacatgcttagggaa

SEQ ID NO: 128 - RNA, 21 nt, synthetic construct
  source 1..21: mol_type = other RNA
tatatccttgcgattctccaa

SEQ ID NO: 129 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
agtatagagttgaagttta

SEQ ID NO: 130 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggttggagttgggtatgta

SEQ ID NO: 131 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gggtatgtggattccataa

SEQ ID NO: 132 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
cactgtctgcggtaatcaa

SEQ ID NO: 133 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
agatttacttcgtcgatta

SEQ ID NO: 134 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gcagcaaacttacaagata

SEQ ID NO: 135 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
cagggtataaatcaactaa

SEQ ID NO: 136 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gggtataaatcaactacca

SEQ ID NO: 137 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggatattactttcgtgtaa

SEQ ID NO: 138 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
agtccttcgtaacaataaa

SEQ ID NO: 139 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
tcgtaacaataaaaactta

SEQ ID NO: 140 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggtgttaaatttaccaaca

SEQ ID NO: 141 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gacactcaatgatgtgtta

SEQ ID NO: 142 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gcacctgttccatatgtaa

SEQ ID NO: 143 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ccccaatatgggaggtaaa

SEQ ID NO: 144 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gggaggtaaatcaacataa

SEQ ID NO: 145 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
caacatatattcgacaaaa

SEQ ID NO: 146 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gggtgatagtactcatgga

SEQ ID NO: 147 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggcccaaattgggtgttta

SEQ ID NO: 148 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gggttagcatgggctataa

SEQ ID NO: 149 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gctatatcagaatacatta

SEQ ID NO: 150 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
tgtctgtgatcaaagttta

SEQ ID NO: 151 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ccctaagcatgtaatagaa

SEQ ID NO: 152 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggagaatcgcaaggatata

SEQ ID NO: 153 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taaacttcaactctatactga

SEQ ID NO: 154 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tacatacccaactccaacctg

SEQ ID NO: 155 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttatggaatccacatacccaa

SEQ ID NO: 156 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttgattaccgcagacagtgat

SEQ ID NO: 157 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taatcgacgaagtaaatcttc

SEQ ID NO: 158 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tatcttgtaagtttgctgctt

SEQ ID NO: 159 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttagttgatttataccctgat

SEQ ID NO: 160 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tggtagttgatttataccctg

SEQ ID NO: 161 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttacacgaaagtaatatccaa

SEQ ID NO: 162 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tttattgttacgaaggacttt

SEQ ID NO: 163 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taagtttttattgttacgaag

SEQ ID NO: 164 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tgttggtaaatttaacaccat

SEQ ID NO: 165 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taacacatcattgagtgtctg

SEQ ID NO: 166 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttacatatggaacaggtgctc

SEQ ID NO: 167 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tttacctcccatattggggcc

SEQ ID NO: 168 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttatgttgatttacctcccat

SEQ ID NO: 169 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttttgtcgaatatatgttgat

SEQ ID NO: 170 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tccatgagtactatcacccca

SEQ ID NO: 171 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taaacacccaatttgggccat

SEQ ID NO: 172 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttatagcccatgctaacccaa

SEQ ID NO: 173 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taatgtattctgatatagccc

SEQ ID NO: 174 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taaactttgatcacagacacc

SEQ ID NO: 175 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttctattacatgcttagggaa

SEQ ID NO: 176 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tatatccttgcgattctccaa

SEQ ID NO: 177 - RNA, 23 nt, Homo sapiens
  source 1..23: mol_type = mRNA
tcagtatagagttgaagtttata

SEQ ID NO: 178 - RNA, 23 nt, Homo sapiens
  source 1..23: mol_type = mRNA
caggttggagttgggtatgtgga

SEQ ID NO: 179 - RNA, 23 nt, Homo sapiens
  source 1..23: mol_type = mRNA
ttgggtatgtggattccatacag

SEQ ID NO: 180 - RNA, 23 nt, Homo sapiens
  source 1..23: mol_type = mRNA
atcactgtctgcggtaatcaagt

SEQ ID NO: 181 - RNA, 23 nt, Homo sapiens
  source 1..23: mol_type = mRNA
gaagatttacttcgtcgattccc

SEQ ID NO: 182 - RNA, 23 nt, Homo sapiens
  source 1..23: mol_type = mRNA
aagcagcaaacttacaagattgt

SEQ ID NO: 183 - RNA, 23 nt, Homo sapiens
  source 1..23: mol_type = mRNA
atcagggtataaatcaactacct

SEQ ID NO: 184 - RNA, 23 nt, Homo sapiens
  source 1..23: mol_type = mRNA
cagggtataaatcaactacctaa

SEQ ID NO: 185 - RNA, 23 nt, Homo sapiens
  source 1..23: mol_type = mRNA
ttggatattactttcgtgtaacc

SEQ ID NO: 186 - RNA, 23 nt, Homo sapiens
  source 1..23: mol_type = mRNA
aaagtccttcgtaacaataaaaa

SEQ ID NO: 187 - RNA, 23 nt, Homo sapiens
  source 1..23: mol_type = mRNA
cttcgtaacaataaaaactttag

SEQ ID NO: 188 - RNA, 23 nt, Homo sapiens
  source 1..23: mol_type = mRNA
atggtgttaaatttaccaacagc

SEQ ID NO: 189 - RNA, 23 nt, Homo sapiens
  source 1..23: mol_type = mRNA
cagacactcaatgatgtgttagc

SEQ ID NO: 190 - RNA, 23 nt, Homo sapiens
  source 1..23: mol_type = mRNA
gagcacctgttccatatgtacga

SEQ ID NO: 191 - RNA, 23 nt, Homo sapiens
  source 1..23: mol_type = mRNA
ggccccaatatgggaggtaaatc

SEQ ID NO: 192 - RNA, 23 nt, Homo sapiens
  source 1..23: mol_type = mRNA
atgggaggtaaatcaacatatat

SEQ ID NO: 193 - RNA, 23 nt, Homo sapiens
  source 1..23: mol_type = mRNA
atcaacatatattcgacaaactg

SEQ ID NO: 194 - RNA, 23 nt, Homo sapiens
  source 1..23: mol_type = mRNA
tggggtgatagtactcatggccc

SEQ ID NO: 195 - RNA, 23 nt, Homo sapiens
  source 1..23: mol_type = mRNA
atggcccaaattgggtgttttgt

SEQ ID NO: 196 - RNA, 23 nt, Homo sapiens
  source 1..23: mol_type = mRNA
ttgggttagcatgggctatatca

SEQ ID NO: 197 - RNA, 23 nt, Homo sapiens
  source 1..23: mol_type = mRNA
gggctatatcagaatacattgca

SEQ ID NO: 198 - RNA, 23 nt, Homo sapiens
  source 1..23: mol_type = mRNA
ggtgtctgtgatcaaagttttgg

SEQ ID NO: 199 - RNA, 23 nt, Homo sapiens
  source 1..23: mol_type = mRNA
ttccctaagcatgtaatagagtg

SEQ ID NO: 200 - RNA, 23 nt, Homo sapiens
  source 1..23: mol_type = mRNA
ttggagaatcgcaaggatatgat

SEQ ID NO: 201 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ccttagcaggttctgtactc

SEQ ID NO: 202 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cttagactcagtaccacctg

SEQ ID NO: 203 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tggcaggtagaaatggtgac

SEQ ID NO: 204 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gctgactgtggtccctctgg

SEQ ID NO: 205 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggaccattttggcatactcc

SEQ ID NO: 206 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gatgcctgctgaaatgatac

SEQ ID NO: 207 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tccaagctgattggtgcaac

SEQ ID NO: 208 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tgtgcataccacaggctgtc

SEQ ID NO: 209 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctgcttctgcccaaacacag

SEQ ID NO: 210 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gaactccttccaactccatg

SEQ ID NO: 211 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gctgctgactgacatttagc

SEQ ID NO: 212 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cttcaacatccagcagtggc

SEQ ID NO: 213 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggcttgttctctgttgtgtg

SEQ ID NO: 214 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cacctgaagtgctagaagac

SEQ ID NO: 215 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gaactcactgcctctttctg

SEQ ID NO: 216 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gctttctcctgagagtccac

SEQ ID NO: 217 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cctggtttgaatggcagtcc

SEQ ID NO: 218 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gctacatcaacctgagaggc

SEQ ID NO: 219 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ccttcactttgctgtgcttc

SEQ ID NO: 220 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
aggtgctatgagcctctgcc

SEQ ID NO: 221 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gccctttcagtgactggagc

SEQ ID NO: 222 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggcaaacatctgcttgactc

SEQ ID NO: 223 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctctggaggcaaacatctgc

SEQ ID NO: 224 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gagcagtcccaatcatcacc

SEQ ID NO: 225 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tgtctcatggttggccttcc

SEQ ID NO: 226 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tctgagaaatgacacccagg

SEQ ID NO: 227 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gcaagcaatgctccatctgg

SEQ ID NO: 228 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cttgcctggacacacacaca

SEQ ID NO: 229 - RNA, 19 nt, synthetic construct
  source 1..19: mol_type = other RNA
ggaactcgactgatgtttg

SEQ ID NO: 230 - RNA, 19 nt, synthetic construct
  source 1..19: mol_type = other RNA
gaggtctaccacatgtata

SEQ ID NO: 231 - RNA, 19 nt, synthetic construct
  source 1..19: mol_type = other RNA
ggtctaccacatgtataat

SEQ ID NO: 232 - RNA, 19 nt, synthetic construct
  source 1..19: mol_type = other RNA
ctaccacatgtataatcga

SEQ ID NO: 233 - RNA, 19 nt, synthetic construct
  source 1..19: mol_type = other RNA
ccacatgtataatcgacac

SEQ ID NO: 234 - RNA, 19 nt, synthetic construct
  source 1..19: mol_type = other RNA
acagaaaaggggtatgctg

SEQ ID NO: 235 - RNA, 19 nt, synthetic construct
  source 1..19: mol_type = other RNA
ggggtatgctgtcttctag

SEQ ID NO: 236 - RNA, 19 nt, synthetic construct
  source 1..19: mol_type = other RNA
ggtatgctgtcttctagca

SEQ ID NO: 237 - RNA, 19 nt, synthetic construct
  source 1..19: mol_type = other RNA
gcgagtatgcggccagctc

SEQ ID NO: 238 - RNA, 19 nt, synthetic construct
  source 1..19: mol_type = other RNA
accggatgtaaatttcgag

SEQ ID NO: 239 - RNA, 19 nt, synthetic construct
  source 1..19: mol_type = other RNA
cggatgtaaatttcgagtt

SEQ ID NO: 240 - skipped

SEQ ID NO: 241 - RNA, 19 nt, synthetic construct
  source 1..19: mol_type = other RNA
agccaactaatctcgcaac

SEQ ID NO: 242 - RNA, 19 nt, synthetic construct
  source 1..19: mol_type = other RNA
ctcgcaaccccaaacacaa

SEQ ID NO: 243 - RNA, 19 nt, synthetic construct
  source 1..19: mol_type = other RNA
ccccaaacacaaagcgttt

SEQ ID NO: 244 - RNA, 19 nt, synthetic construct
  source 1..19: mol_type = other RNA
cccaaacacaaagcgtttt

SEQ ID NO: 245 - RNA, 19 nt, synthetic construct
  source 1..19: mol_type = other RNA
aaacacaaagcgttttaaa

SEQ ID NO: 246 - RNA, 19 nt, synthetic construct
  source 1..19: mol_type = other RNA
aacacaaagcgttttaaaa

SEQ ID NO: 247 - RNA, 19 nt, synthetic construct
  source 1..19: mol_type = other RNA
ggacatgtcagcctctcag

SEQ ID NO: 248 - RNA, 19 nt, synthetic construct
  source 1..19: mol_type = other RNA
ggctggacatagtttagtc

SEQ ID NO: 249 - RNA, 19 nt, synthetic construct
  source 1..19: mol_type = other RNA
aacccttaatgataattaa

SEQ ID NO: 250 - RNA, 19 nt, synthetic construct
  source 1..19: mol_type = other RNA
gggtcactatttgaaacat

SEQ ID NO: 251 - RNA, 19 nt, synthetic construct
  source 1..19: mol_type = other RNA
gagtttgatttcccataat

SEQ ID NO: 252 - RNA, 19 nt, synthetic construct
  source 1..19: mol_type = other RNA
gggactaatttacatactg

SEQ ID NO: 253 - RNA, 19 nt, synthetic construct
  source 1..19: mol_type = other RNA
caaacatcagtcgagttcc

SEQ ID NO: 254 - RNA, 19 nt, synthetic construct
  source 1..19: mol_type = other RNA
tatacatgtggtagacctc

SEQ ID NO: 255 - RNA, 19 nt, synthetic construct
  source 1..19: mol_type = other RNA
attatacatgtggtagacc

SEQ ID NO: 256 - RNA, 19 nt, synthetic construct
  source 1..19: mol_type = other RNA
tcgattatacatgtggtag

SEQ ID NO: 257 - RNA, 19 nt, synthetic construct
  source 1..19: mol_type = other RNA
gtgtcgattatacatgtgg

SEQ ID NO: 258 - RNA, 19 nt, synthetic construct
  source 1..19: mol_type = other RNA
cagcataccccttttctgt

SEQ ID NO: 259 - RNA, 19 nt, synthetic construct
  source 1..19: mol_type = other RNA
ctagaagacagcatacccc

SEQ ID NO: 260 - RNA, 19 nt, synthetic construct
  source 1..19: mol_type = other RNA
tgctagaagacagcatacc

SEQ ID NO: 261 - RNA, 19 nt, synthetic construct
  source 1..19: mol_type = other RNA
gagctggccgcatactcgc

SEQ ID NO: 262 - RNA, 19 nt, synthetic construct
  source 1..19: mol_type = other RNA
ctcgaaatttacatccggt

SEQ ID NO: 263 - RNA, 19 nt, synthetic construct
  source 1..19: mol_type = other RNA
aactcgaaatttacatccg

SEQ ID NO: 264 - skipped

SEQ ID NO: 265 - RNA, 19 nt, synthetic construct
  source 1..19: mol_type = other RNA
gttgcgagattagttggct

SEQ ID NO: 266 - RNA, 19 nt, synthetic construct
  source 1..19: mol_type = other RNA
ttgtgtttggggttgcgag

SEQ ID NO: 267 - RNA, 19 nt, synthetic construct
  source 1..19: mol_type = other RNA
aaacgctttgtgtttgggg

SEQ ID NO: 268 - RNA, 19 nt, synthetic construct
  source 1..19: mol_type = other RNA
aaaacgctttgtgtttggg

SEQ ID NO: 269 - RNA, 19 nt, synthetic construct
  source 1..19: mol_type = other RNA
tttaaaacgctttgtgttt

SEQ ID NO: 270 - RNA, 19 nt, synthetic construct
  source 1..19: mol_type = other RNA
ttttaaaacgctttgtgtt

SEQ ID NO: 271 - RNA, 19 nt, synthetic construct
  source 1..19: mol_type = other RNA
ctgagaggctgacatgtcc

SEQ ID NO: 272 - RNA, 19 nt, synthetic construct
  source 1..19: mol_type = other RNA
gactaaactatgtccagcc

SEQ ID NO: 273 - RNA, 19 nt, synthetic construct
  source 1..19: mol_type = other RNA
ttaattatcattaagggtt

SEQ ID NO: 274 - RNA, 19 nt, synthetic construct
  source 1..19: mol_type = other RNA
atgtttcaaatagtgaccc

SEQ ID NO: 275 - RNA, 19 nt, synthetic construct
  source 1..19: mol_type = other RNA
attatgggaaatcaaactc

SEQ ID NO: 276 - RNA, 19 nt, synthetic construct
  source 1..19: mol_type = other RNA
cagtatgtaaattagtccc

SEQ ID NO: 277 - RNA, 19 nt, synthetic construct
  source 1..19: mol_type = other RNA
ggaactcgactgatgttta

SEQ ID NO: 278 - RNA, 19 nt, synthetic construct
  source 1..19: mol_type = other RNA
gaggtctaccacatgtata

SEQ ID NO: 279 - RNA, 19 nt, synthetic construct
  source 1..19: mol_type = other RNA
ggtctaccacatgtataaa

SEQ ID NO: 280 - RNA, 19 nt, synthetic construct
  source 1..19: mol_type = other RNA
ctaccacatgtataatcga

SEQ ID NO: 281 - RNA, 19 nt, synthetic construct
  source 1..19: mol_type = other RNA
ccacatgtataatcgacaa

SEQ ID NO: 282 - RNA, 19 nt, synthetic construct
  source 1..19: mol_type = other RNA
acagaaaaggggtatgcta

SEQ ID NO: 283 - RNA, 19 nt, synthetic construct
  source 1..19: mol_type = other RNA
ggggtatgctgtcttctaa

SEQ ID NO: 284 - RNA, 19 nt, synthetic construct
  source 1..19: mol_type = other RNA
ggtatgctgtcttctagca

SEQ ID NO: 285 - RNA, 19 nt, synthetic construct
  source 1..19: mol_type = other RNA
gcgagtatgcggccagcta

SEQ ID NO: 286 - RNA, 19 nt, synthetic construct
  source 1..19: mol_type = other RNA
accggatgtaaatttcgaa

SEQ ID NO: 287 - RNA, 19 nt, synthetic construct
  source 1..19: mol_type = other RNA
cggatgtaaatttcgagta

SEQ ID NO: 288 - skipped

SEQ ID NO: 289 - RNA, 19 nt, synthetic construct
  source 1..19: mol_type = other RNA
agccaactaatctcgcaaa

SEQ ID NO: 290 - RNA, 19 nt, synthetic construct
  source 1..19: mol_type = other RNA
ctcgcaaccccaaacacaa

SEQ ID NO: 291 - RNA, 19 nt, synthetic construct
  source 1..19: mol_type = other RNA
ccccaaacacaaagcgtta

SEQ ID NO: 292 - RNA, 19 nt, synthetic construct
  source 1..19: mol_type = other RNA
cccaaacacaaagcgttta

SEQ ID NO: 293 - RNA, 19 nt, synthetic construct
  source 1..19: mol_type = other RNA
aaacacaaagcgttttaaa

SEQ ID NO: 294 - RNA, 19 nt, synthetic construct
  source 1..19: mol_type = other RNA
aacacaaagcgttttaaaa

SEQ ID NO: 295 - RNA, 19 nt, synthetic construct
  source 1..19: mol_type = other RNA
ggacatgtcagcctctcaa

SEQ ID NO: 296 - RNA, 19 nt, synthetic construct
  source 1..19: mol_type = other RNA
ggctggacatagtttagta

SEQ ID NO: 297 - RNA, 19 nt, synthetic construct
  source 1..19: mol_type = other RNA
aacccttaatgataattaa

SEQ ID NO: 298 - RNA, 19 nt, synthetic construct
  source 1..19: mol_type = other RNA
gggtcactatttgaaacaa

SEQ ID NO: 299 - RNA, 19 nt, synthetic construct
  source 1..19: mol_type = other RNA
gagtttgatttcccataaa

SEQ ID NO: 300 - RNA, 19 nt, synthetic construct
  source 1..19: mol_type = other RNA
gggactaatttacatacta

SEQ ID NO: 301 - RNA, 21 nt, synthetic construct
  source 1..21: mol_type = other RNA
taaacatcagtcgagttccaa

SEQ ID NO: 302 - RNA, 21 nt, synthetic construct
  source 1..21: mol_type = other RNA
tatacatgtggtagacctcat

SEQ ID NO: 303 - RNA, 21 nt, synthetic construct
  source 1..21: mol_type = other RNA
tttatacatgtggtagacctc

SEQ ID NO: 304 - RNA, 21 nt, synthetic construct
  source 1..21: mol_type = other RNA
tcgattatacatgtggtagac

SEQ ID NO: 305 - RNA, 21 nt, synthetic construct
  source 1..21: mol_type = other RNA
ttgtcgattatacatgtggta

SEQ ID NO: 306 - RNA, 21 nt, synthetic construct
  source 1..21: mol_type = other RNA
tagcataccccttttctgtcc

SEQ ID NO: 307 - RNA, 21 nt, synthetic construct
  source 1..21: mol_type = other RNA
ttagaagacagcatacccctt

SEQ ID NO: 308 - RNA, 21 nt, synthetic construct
  source 1..21: mol_type = other RNA
tgctagaagacagcatacccc

SEQ ID NO: 309 - RNA, 21 nt, synthetic construct
  source 1..21: mol_type = other RNA
tagctggccgcatactcgctg

SEQ ID NO: 310 - RNA, 21 nt, synthetic construct
  source 1..21: mol_type = other RNA
ttcgaaatttacatccggtat

SEQ ID NO: 311 - RNA, 21 nt, synthetic construct
  source 1..21: mol_type = other RNA
tactcgaaatttacatccggt

SEQ ID NO: 312 - skipped

SEQ ID NO: 313 - RNA, 21 nt, synthetic construct
  source 1..21: mol_type = other RNA
tttgcgagattagttggctga

SEQ ID NO: 314 - RNA, 21 nt, synthetic construct
  source 1..21: mol_type = other RNA
ttgtgtttggggttgcgagat

SEQ ID NO: 315 - RNA, 21 nt, synthetic construct
  source 1..21: mol_type = other RNA
taacgctttgtgtttggggtt

SEQ ID NO: 316 - RNA, 21 nt, synthetic construct
  source 1..21: mol_type = other RNA
taaacgctttgtgtttggggt

SEQ ID NO: 317 - RNA, 21 nt, synthetic construct
  source 1..21: mol_type = other RNA
tttaaaacgctttgtgtttgg

SEQ ID NO: 318 - RNA, 21 nt, synthetic construct
  source 1..21: mol_type = other RNA
ttttaaaacgctttgtgtttg

SEQ ID NO: 319 - RNA, 21 nt, synthetic construct
  source 1..21: mol_type = other RNA
ttgagaggctgacatgtcctg

SEQ ID NO: 320 - RNA, 21 nt, synthetic construct
  source 1..21: mol_type = other RNA
tactaaactatgtccagccag

SEQ ID NO: 321 - RNA, 21 nt, synthetic construct
  source 1..21: mol_type = other RNA
ttaattatcattaagggttga

SEQ ID NO: 322 - RNA, 21 nt, synthetic construct
  source 1..21: mol_type = other RNA
ttgtttcaaatagtgacccct

SEQ ID NO: 323 - RNA, 21 nt, synthetic construct
  source 1..21: mol_type = other RNA
tttatgggaaatcaaactctt

SEQ ID NO: 324 - RNA, 21 nt, synthetic construct
  source 1..21: mol_type = other RNA
tagtatgtaaattagtcccac

SEQ ID NO: 325 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggaactcgactgatgttta

SEQ ID NO: 326 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gaggtctaccacatgtata

SEQ ID NO: 327 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggtctaccacatgtataaa

SEQ ID NO: 328 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ctaccacatgtataatcga

SEQ ID NO: 329 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ccacatgtataatcgacaa

SEQ ID NO: 330 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
acagaaaaggggtatgcta

SEQ ID NO: 331 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggggtatgctgtcttctaa

SEQ ID NO: 332 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggtatgctgtcttctagca

SEQ ID NO: 333 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gcgagtatgcggccagcta

SEQ ID NO: 334 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
accggatgtaaatttcgaa

SEQ ID NO: 335 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
cggatgtaaatttcgagta

SEQ ID NO: 336 - skipped

SEQ ID NO: 337 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
agccaactaatctcgcaaa

SEQ ID NO: 338 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ctcgcaaccccaaacacaa

SEQ ID NO: 339 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ccccaaacacaaagcgtta

SEQ ID NO: 340 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
cccaaacacaaagcgttta

SEQ ID NO: 341 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
aaacacaaagcgttttaaa

SEQ ID NO: 342 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
aacacaaagcgttttaaaa

SEQ ID NO: 343 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggacatgtcagcctctcaa

SEQ ID NO: 344 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggctggacatagtttagta

SEQ ID NO: 345 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
aacccttaatgataattaa

SEQ ID NO: 346 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gggtcactatttgaaacaa

SEQ ID NO: 347 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gagtttgatttcccataaa

SEQ ID NO: 348 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gggactaatttacatacta

SEQ ID NO: 349 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taaacatcagtcgagttccaa

SEQ ID NO: 350 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tatacatgtggtagacctcat

SEQ ID NO: 351 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tttatacatgtggtagacctc

SEQ ID NO: 352 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tcgattatacatgtggtagac

SEQ ID NO: 353 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttgtcgattatacatgtggta

SEQ ID NO: 354 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tagcataccccttttctgtcc

SEQ ID NO: 355 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttagaagacagcatacccctt

SEQ ID NO: 356 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tgctagaagacagcatacccc

SEQ ID NO: 357 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tagctggccgcatactcgctg

SEQ ID NO: 358 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttcgaaatttacatccggtat

SEQ ID NO: 359 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tactcgaaatttacatccggt

SEQ ID NO: 360 - skipped

SEQ ID NO: 361 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tttgcgagattagttggctga

SEQ ID NO: 362 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttgtgtttggggttgcgagat

SEQ ID NO: 363 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taacgctttgtgtttggggtt

SEQ ID NO: 364 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taaacgctttgtgtttggggt

SEQ ID NO: 365 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tttaaaacgctttgtgtttgg

SEQ ID NO: 366 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttttaaaacgctttgtgtttg

SEQ ID NO: 367 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttgagaggctgacatgtcctg

SEQ ID NO: 368 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tactaaactatgtccagccag

SEQ ID NO: 369 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttaattatcattaagggttga

SEQ ID NO: 370 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttgtttcaaatagtgacccct

SEQ ID NO: 371 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tttatgggaaatcaaactctt

SEQ ID NO: 372 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tagtatgtaaattagtcccac

SEQ ID NO: 373 - RNA, 23 nt, Homo sapiens
  source 1..23: mol_type = mRNA
ttggaactcgactgatgtttgat

SEQ ID NO: 374 - RNA, 23 nt, Homo sapiens
  source 1..23: mol_type = mRNA
atgaggtctaccacatgtataat

SEQ ID NO: 375 - RNA, 23 nt, Homo sapiens
  source 1..23: mol_type = mRNA
gaggtctaccacatgtataatcg

SEQ ID NO: 376 - RNA, 23 nt, Homo sapiens
  source 1..23: mol_type = mRNA
gtctaccacatgtataatcgaca

SEQ ID NO: 377 - RNA, 23 nt, Homo sapiens
  source 1..23: mol_type = mRNA
taccacatgtataatcgacacca

SEQ ID NO: 378 - RNA, 23 nt, Homo sapiens
  source 1..23: mol_type = mRNA
ggacagaaaaggggtatgctgtc

SEQ ID NO: 379 - RNA, 23 nt, Homo sapiens
  source 1..23: mol_type = mRNA
aaggggtatgctgtcttctagca

SEQ ID NO: 380 - RNA, 23 nt, Homo sapiens
  source 1..23: mol_type = mRNA
ggggtatgctgtcttctagcact

SEQ ID NO: 381 - RNA, 23 nt, Homo sapiens
  source 1..23: mol_type = mRNA
cagcgagtatgcggccagctccc

SEQ ID NO: 382 - RNA, 23 nt, Homo sapiens
  source 1..23: mol_type = mRNA
ataccggatgtaaatttcgagtt

SEQ ID NO: 383 - RNA, 23 nt, Homo sapiens
  source 1..23: mol_type = mRNA
accggatgtaaatttcgagtttt

SEQ ID NO: 384 - RNA, 23 nt, Homo sapiens
  source 1..23: mol_type = mRNA
ccggatgtaaatttcgagttttg

SEQ ID NO: 385 - RNA, 23 nt, Homo sapiens
  source 1..23: mol_type = mRNA
tcagccaactaatctcgcaaccc

SEQ ID NO: 386 - RNA, 23 nt, Homo sapiens
  source 1..23: mol_type = mRNA
atctcgcaaccccaaacacaaag

SEQ ID NO: 387 - RNA, 23 nt, Homo sapiens
  source 1..23: mol_type = mRNA
aaccccaaacacaaagcgtttta

SEQ ID NO: 388 - RNA, 23 nt, Homo sapiens
  source 1..23: mol_type = mRNA
accccaaacacaaagcgttttaa

SEQ ID NO: 389 - RNA, 23 nt, Homo sapiens
  source 1..23: mol_type = mRNA
ccaaacacaaagcgttttaaaaa

SEQ ID NO: 390 - RNA, 23 nt, Homo sapiens
  source 1..23: mol_type = mRNA
caaacacaaagcgttttaaaaaa

SEQ ID NO: 391 - RNA, 23 nt, Homo sapiens
  source 1..23: mol_type = mRNA
caggacatgtcagcctctcaggt

SEQ ID NO: 392 - RNA, 23 nt, Homo sapiens
  source 1..23: mol_type = mRNA
ctggctggacatagtttagtcta

SEQ ID NO: 393 - RNA, 23 nt, Homo sapiens
  source 1..23: mol_type = mRNA
tcaacccttaatgataattaaat

SEQ ID NO: 394 - RNA, 23 nt, Homo sapiens
  source 1..23: mol_type = mRNA
aggggtcactatttgaaacattc

SEQ ID NO: 395 - RNA, 23 nt, Homo sapiens
  source 1..23: mol_type = mRNA
aagagtttgatttcccataattt

SEQ ID NO: 396 - RNA, 23 nt, Homo sapiens
  source 1..23: mol_type = mRNA
gtgggactaatttacatactgta

SEQ ID NO: 397 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ccttgttggcatcactcagc

SEQ ID NO: 398 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tgagaagtcacaactggtgg

SEQ ID NO: 399 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ccaaaccaaatctcctggtg

SEQ ID NO: 400 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gtaaaccagacaaggccacc

SEQ ID NO: 401 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggactgatttccctttctcg

SEQ ID NO: 402 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctcagtatttcaggctttgc

SEQ ID NO: 403 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tctcttcttcctcttctggc

SEQ ID NO: 404 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctacctccatctcttcttcc

SEQ ID NO: 405 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gctgtacttcctcttcactc

SEQ ID NO: 406 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gtgtcttaggctgtacttcc

SEQ ID NO: 407 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gccaccaatgtcactctcag

SEQ ID NO: 408 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
catcactgcttccttcctcc

SEQ ID NO: 409 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gccttcactctcactatccc

SEQ ID NO: 410 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tgcttgtttggtggctgagg

SEQ ID NO: 411 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctactgtcatcaccacctcc

SEQ ID NO: 412 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cctcctgtgctcatctcttc

SEQ ID NO: 413 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctcaggcacatagagtgtag

SEQ ID NO: 414 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
caccacttcctcatcccagg

SEQ ID NO: 415 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cccaccttgtaacagatgac

SEQ ID NO: 416 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gagcatccatgtggtacagc

SEQ ID NO: 417 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggagtctcagtctgttccac

SEQ ID NO: 418 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tccctcctcaccactctatc

SEQ ID NO: 419 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gtaagtctgtgtacccttgg

SEQ ID NO: 420 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cttccagcacactgtaagtc

SEQ ID NO: 421 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctcctggtgtcaacccaatg

SEQ ID NO: 422 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggctttggtgaagatagcac

SEQ ID NO: 423 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cactgcatctagcaccattc

SEQ ID NO: 424 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tggcaagtatcaaccctctc

SEQ ID NO: 425 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
aagccattgctttaggagcc

SEQ ID NO: 426 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggcacaaccatgaggtcttc

SEQ ID NO: 427 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggtggttctgactcttcagg

SEQ ID NO: 428 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gtcaaaggctgtatcccatc

SEQ ID NO: 429 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
aggtattccaggaggctctg

SEQ ID NO: 430 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ccttcaatgatacatccctc

SEQ ID NO: 431 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cagcagactgccagtccttg

SEQ ID NO: 432 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gacacataggaccatcaccc

SEQ ID NO: 433 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tgctcctcttcctcacagcc

SEQ ID NO: 434 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
agcacacaataggctttgcc

SEQ ID NO: 435 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctaataagccagcctgtctc

SEQ ID NO: 436 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gtctgaggcaccaagtctag

SEQ ID NO: 437 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
caccagagaatgtgctgttg

SEQ ID NO: 438 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
agccttgctgcattaaagcc

SEQ ID NO: 439 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cactagccaggcaaacttcc

SEQ ID NO: 440 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gtcagaagtcaactcaaagc

SEQ ID NO: 441 - RNA, 19 nt, synthetic construct
  source 1..19: mol_type = other RNA
cgaagtagccgccaaataa

SEQ ID NO: 442 - RNA, 19 nt, synthetic construct
  source 1..19: mol_type = other RNA
gaagtagccgccaaataaa

SEQ ID NO: 443 - RNA, 19 nt, synthetic construct
  source 1..19: mol_type = other RNA
ccgccaaataaaaaaacga

SEQ ID NO: 444 - RNA, 19 nt, synthetic construct
  source 1..19: mol_type = other RNA
ggaagtggtggcagattaa

SEQ ID NO: 445 - RNA, 19 nt, synthetic construct
  source 1..19: mol_type = other RNA
gcagaagggctataaagta

SEQ ID NO: 446 - RNA, 19 nt, synthetic construct
  source 1..19: mol_type = other RNA
gaggcacgatgtagaaaga

SEQ ID NO: 447 - RNA, 19 nt, synthetic construct
  source 1..19: mol_type = other RNA
ggcacgatgtagaaagatg

SEQ ID NO: 448 - RNA, 19 nt, synthetic construct
  source 1..19: mol_type = other RNA
ggagatctgtaggatcatt

SEQ ID NO: 449 - RNA, 19 nt, synthetic construct
  source 1..19: mol_type = other RNA
cgccattgttcgagattta

SEQ ID NO: 450 - RNA, 19 nt, synthetic construct
  source 1..19: mol_type = other RNA
gggatgcatccaaaacttt

SEQ ID NO: 451 - RNA, 19 nt, synthetic construct
  source 1..19: mol_type = other RNA
ggtgctatcttcaccaaag

SEQ ID NO: 452 - RNA, 19 nt, synthetic construct
  source 1..19: mol_type = other RNA
cagtgacattaaacaactt

SEQ ID NO: 453 - RNA, 19 nt, synthetic construct
  source 1..19: mol_type = other RNA
ggttgatacttgccatact

SEQ ID NO: 454 - RNA, 19 nt, synthetic construct
  source 1..19: mol_type = other RNA
ggttgtgcctgacaaaatc

SEQ ID NO: 455 - RNA, 19 nt, synthetic construct
  source 1..19: mol_type = other RNA
cagcagggctataatgtat

SEQ ID NO: 456 - RNA, 19 nt, synthetic construct
  source 1..19: mol_type = other RNA
gggctataatgtatgaaga

SEQ ID NO: 457 - RNA, 19 nt, synthetic construct
  source 1..19: mol_type = other RNA
ggattggtaggaaccgtta

SEQ ID NO: 458 - RNA, 19 nt, synthetic construct
  source 1..19: mol_type = other RNA
gaatttcaccactcgcaat

SEQ ID NO: 459 - RNA, 19 nt, synthetic construct
  source 1..19: mol_type = other RNA
gaatacgagttgaaatcta

SEQ ID NO: 460 - RNA, 19 nt, synthetic construct
  source 1..19: mol_type = other RNA
gaagggctgtaaacgatac

SEQ ID NO: 461 - RNA, 19 nt, synthetic construct
  source 1..19: mol_type = other RNA
ggctaatctcataaatgct

SEQ ID NO: 462 - RNA, 19 nt, synthetic construct
  source 1..19: mol_type = other RNA
ggagggatgtatcattgaa

SEQ ID NO: 463 - RNA, 19 nt, synthetic construct
  source 1..19: mol_type = other RNA
cggcgactgttctataact

SEQ ID NO: 464 - RNA, 19 nt, synthetic construct
  source 1..19: mol_type = other RNA
gcgactgttctataacttt

SEQ ID NO: 465 - RNA, 19 nt, synthetic construct
  source 1..19: mol_type = other RNA
ttatttggcggctacttcg

SEQ ID NO: 466 - RNA, 19 nt, synthetic construct
  source 1..19: mol_type = other RNA
tttatttggcggctacttc

SEQ ID NO: 467 - RNA, 19 nt, synthetic construct
  source 1..19: mol_type = other RNA
tcgtttttttatttggcgg

SEQ ID NO: 468 - RNA, 19 nt, synthetic construct
  source 1..19: mol_type = other RNA
ttaatctgccaccacttcc

SEQ ID NO: 469 - RNA, 19 nt, synthetic construct
  source 1..19: mol_type = other RNA
tactttatagcccttctgc

SEQ ID NO: 470 - RNA, 19 nt, synthetic construct
  source 1..19: mol_type = other RNA
tctttctacatcgtgcctc

SEQ ID NO: 471 - RNA, 19 nt, synthetic construct
  source 1..19: mol_type = other RNA
catctttctacatcgtgcc

SEQ ID NO: 472 - RNA, 19 nt, synthetic construct
  source 1..19: mol_type = other RNA
aatgatcctacagatctcc

SEQ ID NO: 473 - RNA, 19 nt, synthetic construct
  source 1..19: mol_type = other RNA
taaatctcgaacaatggcg

SEQ ID NO: 474 - RNA, 19 nt, synthetic construct
  source 1..19: mol_type = other RNA
aaagttttggatgcatccc

SEQ ID NO: 475 - RNA, 19 nt, synthetic construct
  source 1..19: mol_type = other RNA
ctttggtgaagatagcacc

SEQ ID NO: 476 - RNA, 19 nt, synthetic construct
  source 1..19: mol_type = other RNA
aagttgtttaatgtcactg

SEQ ID NO: 477 - RNA, 19 nt, synthetic construct
  source 1..19: mol_type = other RNA
agtatggcaagtatcaacc

SEQ ID NO: 478 - RNA, 19 nt, synthetic construct
  source 1..19: mol_type = other RNA
gattttgtcaggcacaacc

SEQ ID NO: 479 - RNA, 19 nt, synthetic construct
  source 1..19: mol_type = other RNA
atacattatagccctgctg

SEQ ID NO: 480 - RNA, 19 nt, synthetic construct
  source 1..19: mol_type = other RNA
tcttcatacattatagccc

SEQ ID NO: 481 - RNA, 19 nt, synthetic construct
  source 1..19: mol_type = other RNA
taacggttcctaccaatcc

SEQ ID NO: 482 - RNA, 19 nt, synthetic construct
  source 1..19: mol_type = other RNA
attgcgagtggtgaaattc

SEQ ID NO: 483 - RNA, 19 nt, synthetic construct
  source 1..19: mol_type = other RNA
tagatttcaactcgtattc

SEQ ID NO: 484 - RNA, 19 nt, synthetic construct
  source 1..19: mol_type = other RNA
gtatcgtttacagcccttc

SEQ ID NO: 485 - RNA, 19 nt, synthetic construct
  source 1..19: mol_type = other RNA
agcatttatgagattagcc

SEQ ID NO: 486 - RNA, 19 nt, synthetic construct
  source 1..19: mol_type = other RNA
ttcaatgatacatccctcc

SEQ ID NO: 487 - RNA, 19 nt, synthetic construct
  source 1..19: mol_type = other RNA
agttatagaacagtcgccg

SEQ ID NO: 488 - RNA, 19 nt, synthetic construct
  source 1..19: mol_type = other RNA
aaagttatagaacagtcgc

SEQ ID NO: 489 - skipped

SEQ ID NO: 490 - RNA, 19 nt, synthetic construct
  source 1..19: mol_type = other RNA
gaagtagccgccaaataaa

SEQ ID NO: 491 - RNA, 19 nt, synthetic construct
  source 1..19: mol_type = other RNA
ccgccaaataaaaaaacga

SEQ ID NO: 492 - RNA, 19 nt, synthetic construct
  source 1..19: mol_type = other RNA
ggaagtggtggcagattaa

SEQ ID NO: 493 - RNA, 19 nt, synthetic construct
  source 1..19: mol_type = other RNA
gcagaagggctataaagta

SEQ ID NO: 494 - RNA, 19 nt, synthetic construct
  source 1..19: mol_type = other RNA
gaggcacgatgtagaaaga

SEQ ID NO: 495 - RNA, 19 nt, synthetic construct
  source 1..19: mol_type = other RNA
ggcacgatgtagaaagata

SEQ ID NO: 496 - RNA, 19 nt, synthetic construct
  source 1..19: mol_type = other RNA
ggagatctgtaggatcata

SEQ ID NO: 497 - RNA, 19 nt, synthetic construct
  source 1..19: mol_type = other RNA
cgccattgttcgagattta

SEQ ID NO: 498 - RNA, 19 nt, synthetic construct
  source 1..19: mol_type = other RNA
gggatgcatccaaaactta

SEQ ID NO: 499 - RNA, 19 nt, synthetic construct
  source 1..19: mol_type = other RNA
ggtgctatcttcaccaaaa

SEQ ID NO: 500 - RNA, 19 nt, synthetic construct
  source 1..19: mol_type = other RNA
cagtgacattaaacaacta

SEQ ID NO: 501 - RNA, 19 nt, synthetic construct
  source 1..19: mol_type = other RNA
ggttgatacttgccataca

SEQ ID NO: 502 - RNA, 19 nt, synthetic construct
  source 1..19: mol_type = other RNA
ggttgtgcctgacaaaata

SEQ ID NO: 503 - RNA, 19 nt, synthetic construct
  source 1..19: mol_type = other RNA
cagcagggctataatgtaa

SEQ ID NO: 504 - RNA, 19 nt, synthetic construct
  source 1..19: mol_type = other RNA
gggctataatgtatgaaga

SEQ ID NO: 505 - RNA, 19 nt, synthetic construct
  source 1..19: mol_type = other RNA
ggattggtaggaaccgtta

SEQ ID NO: 506 - RNA, 19 nt, synthetic construct
  source 1..19: mol_type = other RNA
gaatttcaccactcgcaaa

SEQ ID NO: 507 - RNA, 19 nt, synthetic construct
  source 1..19: mol_type = other RNA
gaatacgagttgaaatcta

SEQ ID NO: 508 - RNA, 19 nt, synthetic construct
  source 1..19: mol_type = other RNA
gaagggctgtaaacgataa

SEQ ID NO: 509 - RNA, 19 nt, synthetic construct
  source 1..19: mol_type = other RNA
ggctaatctcataaatgca

SEQ ID NO: 510 - RNA, 19 nt, synthetic construct
  source 1..19: mol_type = other RNA
ggagggatgtatcattgaa

SEQ ID NO: 511 - RNA, 19 nt, synthetic construct
  source 1..19: mol_type = other RNA
cggcgactgttctataaca

SEQ ID NO: 512 - RNA, 19 nt, synthetic construct
  source 1..19: mol_type = other RNA
gcgactgttctataactta

SEQ ID NO: 513 - skipped

SEQ ID NO: 514 - RNA, 21 nt, synthetic construct
  source 1..21: mol_type = other RNA
tttatttggcggctacttcgc

SEQ ID NO: 515 - RNA, 21 nt, synthetic construct
  source 1..21: mol_type = other RNA
tcgtttttttatttggcggct

SEQ ID NO: 516 - RNA, 21 nt, synthetic construct
  source 1..21: mol_type = other RNA
ttaatctgccaccacttcctc

SEQ ID NO: 517 - RNA, 21 nt, synthetic construct
  source 1..21: mol_type = other RNA
tactttatagcccttctgcac

SEQ ID NO: 518 - RNA, 21 nt, synthetic construct
  source 1..21: mol_type = other RNA
tctttctacatcgtgcctcca

SEQ ID NO: 519 - RNA, 21 nt, synthetic construct
  source 1..21: mol_type = other RNA
tatctttctacatcgtgcctc

SEQ ID NO: 520 - RNA, 21 nt, synthetic construct
  source 1..21: mol_type = other RNA
tatgatcctacagatctccct

SEQ ID NO: 521 - RNA, 21 nt, synthetic construct
  source 1..21: mol_type = other RNA
taaatctcgaacaatggcgat

SEQ ID NO: 522 - RNA, 21 nt, synthetic construct
  source 1..21: mol_type = other RNA
taagttttggatgcatcccaa

SEQ ID NO: 523 - RNA, 21 nt, synthetic construct
  source 1..21: mol_type = other RNA
ttttggtgaagatagcaccag

SEQ ID NO: 524 - RNA, 21 nt, synthetic construct
  source 1..21: mol_type = other RNA
tagttgtttaatgtcactgca

SEQ ID NO: 525 - RNA, 21 nt, synthetic construct
  source 1..21: mol_type = other RNA
tgtatggcaagtatcaaccct

SEQ ID NO: 526 - RNA, 21 nt, synthetic construct
  source 1..21: mol_type = other RNA
tattttgtcaggcacaaccat

SEQ ID NO: 527 - RNA, 21 nt, synthetic construct
  source 1..21: mol_type = other RNA
ttacattatagccctgctgtc

SEQ ID NO: 528 - RNA, 21 nt, synthetic construct
  source 1..21: mol_type = other RNA
tcttcatacattatagccctg

SEQ ID NO: 529 - RNA, 21 nt, synthetic construct
  source 1..21: mol_type = other RNA
taacggttcctaccaatcccc

SEQ ID NO: 530 - RNA, 21 nt, synthetic construct
  source 1..21: mol_type = other RNA
tttgcgagtggtgaaattctc

SEQ ID NO: 531 - RNA, 21 nt, synthetic construct
  source 1..21: mol_type = other RNA
tagatttcaactcgtattctt

SEQ ID NO: 532 - RNA, 21 nt, synthetic construct
  source 1..21: mol_type = other RNA
ttatcgtttacagcccttctt

SEQ ID NO: 533 - RNA, 21 nt, synthetic construct
  source 1..21: mol_type = other RNA
tgcatttatgagattagccaa

SEQ ID NO: 534 - RNA, 21 nt, synthetic construct
  source 1..21: mol_type = other RNA
ttcaatgatacatccctccgt

SEQ ID NO: 535 - RNA, 21 nt, synthetic construct
  source 1..21: mol_type = other RNA
tgttatagaacagtcgccgca

SEQ ID NO: 536 - RNA, 21 nt, synthetic construct
  source 1..21: mol_type = other RNA
taagttatagaacagtcgccg

SEQ ID NO: 537 - skipped

SEQ ID NO: 538 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gaagtagccgccaaataaa

SEQ ID NO: 539 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ccgccaaataaaaaaacga

SEQ ID NO: 540 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggaagtggtggcagattaa

SEQ ID NO: 541 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gcagaagggctataaagta

SEQ ID NO: 542 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gaggcacgatgtagaaaga

SEQ ID NO: 543 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggcacgatgtagaaagata

SEQ ID NO: 544 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggagatctgtaggatcata

SEQ ID NO: 545 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
cgccattgttcgagattta

SEQ ID NO: 546 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gggatgcatccaaaactta

SEQ ID NO: 547 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggtgctatcttcaccaaaa

SEQ ID NO: 548 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
cagtgacattaaacaacta

SEQ ID NO: 549 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggttgatacttgccataca

SEQ ID NO: 550 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggttgtgcctgacaaaata

SEQ ID NO: 551 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
cagcagggctataatgtaa

SEQ ID NO: 552 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gggctataatgtatgaaga

SEQ ID NO: 553 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggattggtaggaaccgtta

SEQ ID NO: 554 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gaatttcaccactcgcaaa

SEQ ID NO: 555 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gaatacgagttgaaatcta

SEQ ID NO: 556 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gaagggctgtaaacgataa

SEQ ID NO: 557 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggctaatctcataaatgca

SEQ ID NO: 558 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggagggatgtatcattgaa

SEQ ID NO: 559 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
cggcgactgttctataaca

SEQ ID NO: 560 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gcgactgttctataactta

SEQ ID NO: 561 - skipped

SEQ ID NO: 562 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tttatttggcggctacttcgc

SEQ ID NO: 563 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tcgtttttttatttggcggct

SEQ ID NO: 564 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttaatctgccaccacttcctc

SEQ ID NO: 565 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tactttatagcccttctgcac

SEQ ID NO: 566 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tctttctacatcgtgcctcca

SEQ ID NO: 567 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tatctttctacatcgtgcctc

SEQ ID NO: 568 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tatgatcctacagatctccct

SEQ ID NO: 569 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taaatctcgaacaatggcgat

SEQ ID NO: 570 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taagttttggatgcatcccaa

SEQ ID NO: 571 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttttggtgaagatagcaccag

SEQ ID NO: 572 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tagttgtttaatgtcactgca

SEQ ID NO: 573 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tgtatggcaagtatcaaccct

SEQ ID NO: 574 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tattttgtcaggcacaaccat

SEQ ID NO: 575 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttacattatagccctgctgtc

SEQ ID NO: 576 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tcttcatacattatagccctg

SEQ ID NO: 577 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taacggttcctaccaatcccc

SEQ ID NO: 578 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tttgcgagtggtgaaattctc

SEQ ID NO: 579 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tagatttcaactcgtattctt

SEQ ID NO: 580 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttatcgtttacagcccttctt

SEQ ID NO: 581 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tgcatttatgagattagccaa

SEQ ID NO: 582 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttcaatgatacatccctccgt

SEQ ID NO: 583 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tgttatagaacagtcgccgca

SEQ ID NO: 584 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taagttatagaacagtcgccg

SEQ ID NO: 585 - RNA, 23 nt, Homo sapiens
  source 1..23: mol_type = mRNA
ggcgaagtagccgccaaataaaa

SEQ ID NO: 586 - RNA, 23 nt, Homo sapiens
  source 1..23: mol_type = mRNA
gcgaagtagccgccaaataaaaa

SEQ ID NO: 587 - RNA, 23 nt, Homo sapiens
  source 1..23: mol_type = mRNA
agccgccaaataaaaaaacgaag

SEQ ID NO: 588 - RNA, 23 nt, Homo sapiens
  source 1..23: mol_type = mRNA
gaggaagtggtggcagattaagt

SEQ ID NO: 589 - RNA, 23 nt, Homo sapiens
  source 1..23: mol_type = mRNA
gtgcagaagggctataaagtagc

SEQ ID NO: 590 - RNA, 23 nt, Homo sapiens
  source 1..23: mol_type = mRNA
tggaggcacgatgtagaaagatg

SEQ ID NO: 591 - RNA, 23 nt, Homo sapiens
  source 1..23: mol_type = mRNA
gaggcacgatgtagaaagatggc

SEQ ID NO: 592 - RNA, 23 nt, Homo sapiens
  source 1..23: mol_type = mRNA
agggagatctgtaggatcattac

SEQ ID NO: 593 - RNA, 23 nt, Homo sapiens
  source 1..23: mol_type = mRNA
atcgccattgttcgagatttagg

SEQ ID NO: 594 - RNA, 23 nt, Homo sapiens
  source 1..23: mol_type = mRNA
ttgggatgcatccaaaactttga

SEQ ID NO: 595 - RNA, 23 nt, Homo sapiens
  source 1..23: mol_type = mRNA
ctggtgctatcttcaccaaagcc

SEQ ID NO: 596 - RNA, 23 nt, Homo sapiens
  source 1..23: mol_type = mRNA
tgcagtgacattaaacaacttgg

SEQ ID NO: 597 - RNA, 23 nt, Homo sapiens
  source 1..23: mol_type = mRNA
agggttgatacttgccatactcc

SEQ ID NO: 598 - RNA, 23 nt, Homo sapiens
  source 1..23: mol_type = mRNA
atggttgtgcctgacaaaatctc

SEQ ID NO: 599 - RNA, 23 nt, Homo sapiens
  source 1..23: mol_type = mRNA
gacagcagggctataatgtatga

SEQ ID NO: 600 - RNA, 23 nt, Homo sapiens
  source 1..23: mol_type = mRNA
cagggctataatgtatgaagaaa

SEQ ID NO: 601 - RNA, 23 nt, Homo sapiens
  source 1..23: mol_type = mRNA
ggggattggtaggaaccgttacc

SEQ ID NO: 602 - RNA, 23 nt, Homo sapiens
  source 1..23: mol_type = mRNA
gagaatttcaccactcgcaattt

SEQ ID NO: 603 - RNA, 23 nt, Homo sapiens
  source 1..23: mol_type = mRNA
aagaatacgagttgaaatctacc

SEQ ID NO: 604 - RNA, 23 nt, Homo sapiens
  source 1..23: mol_type = mRNA
aagaagggctgtaaacgatactg

SEQ ID NO: 605 - RNA, 23 nt, Homo sapiens
  source 1..23: mol_type = mRNA
ttggctaatctcataaatgctga

SEQ ID NO: 606 - RNA, 23 nt, Homo sapiens
  source 1..23: mol_type = mRNA
acggagggatgtatcattgaagg

SEQ ID NO: 607 - RNA, 23 nt, Homo sapiens
  source 1..23: mol_type = mRNA
tgcggcgactgttctataacttt

SEQ ID NO: 608 - RNA, 23 nt, Homo sapiens
  source 1..23: mol_type = mRNA
cggcgactgttctataactttga

SEQ ID NO: 609 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gccagaagagccaaggaaac

SEQ ID NO: 610 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctttgatagcattagctggc

SEQ ID NO: 611 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cttggatctgaatcaacttc

SEQ ID NO: 612 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gtgccattgtcttggatctg

SEQ ID NO: 613 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggactgcagtttactagtag

SEQ ID NO: 614 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gaaatactggctaaatcctc

SEQ ID NO: 615 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gtatgcacactttccatcag

SEQ ID NO: 616 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
catctgagtaacttgctctg

SEQ ID NO: 617 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ccagcacatggtttaggagg

SEQ ID NO: 618 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ccttgattgccagcacatgg

SEQ ID NO: 619 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggtcccttgattgccagcac

SEQ ID NO: 620 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gaatacctgccaacaacttc

SEQ ID NO: 621 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctgcattgtgtactgaatac

SEQ ID NO: 622 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
catcagctactgtctctcct

SEQ ID NO: 623 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggtagtgtcctaacatcagc

SEQ ID NO: 624 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tgcacttcttcactgagtag

SEQ ID NO: 625 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggaagttgattctaccagac

SEQ ID NO: 626 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gcatacactgtttctatggc

SEQ ID NO: 627 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ggcaaataggctgcatacac

SEQ ID NO: 628 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cgctccaggatgctctcctc

SEQ ID NO: 629 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gagcccaggagcttgctctc

SEQ ID NO: 630 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
catcctggaggaattggagc

SEQ ID NO: 631 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gtcctggtagcaaagtctgg

SEQ ID NO: 632 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
catctggtgggcatagacct

SEQ ID NO: 633 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gccctgccactagaaatatc

SEQ ID NO: 634 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ctcctcatcttgctgcctag

SEQ ID NO: 635 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gaagtaggtcctctcttctc

SEQ ID NO: 636 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gagtggttatgcaacatctc

SEQ ID NO: 637 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cactgaggattcacacagcc

SEQ ID NO: 638 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gcatggcaaggtcaaagagc

SEQ ID NO: 639 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cagccactctctggactatc

SEQ ID NO: 640 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cttcctctgtccagccactc

SEQ ID NO: 641 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
ccaatcaggttcccttcctc

SEQ ID NO: 642 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gaatgaagataggcagtccc

SEQ ID NO: 643 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
cttcactctgctggcctgag

SEQ ID NO: 644 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
tcaggcaggttagcaagctg

SEQ ID NO: 645 - DNA, 20 bp, synthetic construct
  source 1..20: mol_type = other DNA
  misc_feature 1..20: note = phosphorothioate internucleotide linkage
  modified_base 1..5: mod_base = OTHER; note = 2'-O-methoxyethyl nucleotide
  modified_base 16..20: mod_base = OTHER; note = 2'-O-methoxyethyl nucleotide
  misc_feature 1..5: note = RNA
  misc_feature 6..15: note = DNA
  misc_feature 16..20: note = RNA
gaagaacacatcccacagtg

SEQ ID NO: 646 - RNA, 19 nt, synthetic construct
  source 1..19: mol_type = other RNA
ggatttagccagtatttct

SEQ ID NO: 647 - RNA, 19 nt, synthetic construct
  source 1..19: mol_type = other RNA
ggctttggccagcataagc

SEQ ID NO: 648 - RNA, 19 nt, synthetic construct
  source 1..19: mol_type = other RNA
gtggctcatgttactatta

SEQ ID NO: 649 - RNA, 19 nt, synthetic construct
  source 1..19: mol_type = other RNA
gttactattacaacgaaaa

SEQ ID NO: 650 - RNA, 19 nt, synthetic construct
  source 1..19: mol_type = other RNA
gatggaaagtgtgcataca

SEQ ID NO: 651 - RNA, 19 nt, synthetic construct
  source 1..19: mol_type = other RNA
ccatgtgctggcaatcaag

SEQ ID NO: 652 - RNA, 19 nt, synthetic construct
  source 1..19: mol_type = other RNA
cacaatgcaggcattagtt

SEQ ID NO: 653 - RNA, 19 nt, synthetic construct
  source 1..19: mol_type = other RNA
agctgatgttaggacacta

SEQ ID NO: 654 - RNA, 19 nt, synthetic construct
  source 1..19: mol_type = other RNA
gttaggacactacccaatg

SEQ ID NO: 655 - RNA, 19 nt, synthetic construct
  source 1..19: mol_type = other RNA
gcctcaaccgtggacaata

SEQ ID NO: 656 - RNA, 19 nt, synthetic construct
  source 1..19: mol_type = other RNA
ccgtggacaatattcgctc

SEQ ID NO: 657 - RNA, 19 nt, synthetic construct
  source 1..19: mol_type = other RNA
ggataagacagatatttct

SEQ ID NO: 658 - RNA, 19 nt, synthetic construct
  source 1..19: mol_type = other RNA
ggaagatgattcccgaaag

SEQ ID NO: 659 - RNA, 19 nt, synthetic construct
  source 1..19: mol_type = other RNA
ggaaatgactgcagcttgt

SEQ ID NO: 660 - RNA, 19 nt, synthetic construct
  source 1..19: mol_type = other RNA
ggagaaggatcattaacct

SEQ ID NO: 661 - RNA, 19 nt, synthetic construct
  source 1..19: mol_type = other RNA
gaaggatcattaacctcac

SEQ ID NO: 662 - RNA, 19 nt, synthetic construct
  source 1..19: mol_type = other RNA
cactagtgttttgagtctc

SEQ ID NO: 663 - RNA, 19 nt, synthetic construct
  source 1..19: mol_type = other RNA
gcagggacatgaggttctc

SEQ ID NO: 664 - RNA, 19 nt, synthetic construct
  source 1..19: mol_type = other RNA
ggttctccgggagatgttg

SEQ ID NO: 665 - RNA, 19 nt, synthetic construct
  source 1..19: mol_type = other RNA
cgggagatgttgcataacc

SEQ ID NO: 666 - RNA, 19 nt, synthetic construct
  source 1..19: mol_type = other RNA
gggagatgttgcataacca

SEQ ID NO: 667 - RNA, 19 nt, synthetic construct
  source 1..19: mol_type = other RNA
gcataaccactccttcgtg

SEQ ID NO: 668 - RNA, 19 nt, synthetic construct
  source 1..19: mol_type = other RNA
cagcatcaaaccaagttat

SEQ ID NO: 669 - RNA, 19 nt, synthetic construct
  source 1..19: mol_type = other RNA
gcatcaaaccaagttatac

SEQ ID NO: 670 - RNA, 19 nt, synthetic construct
  source 1..19: mol_type = other RNA
agaaatactggctaaatcc

SEQ ID NO: 671 - RNA, 19 nt, synthetic construct
  source 1..19: mol_type = other RNA
gcttatgctggccaaagcc

SEQ ID NO: 672 - RNA, 19 nt, synthetic construct
  source 1..19: mol_type = other RNA
taatagtaacatgagccac

SEQ ID NO: 673 - RNA, 19 nt, synthetic construct
  source 1..19: mol_type = other RNA
ttttcgttgtaatagtaac

SEQ ID NO: 674 - RNA, 19 nt, synthetic construct
  source 1..19: mol_type = other RNA
tgtatgcacactttccatc

SEQ ID NO: 675 - RNA, 19 nt, synthetic construct
  source 1..19: mol_type = other RNA
cttgattgccagcacatgg

SEQ ID NO: 676 - RNA, 19 nt, synthetic construct
  source 1..19: mol_type = other RNA
aactaatgcctgcattgtg

SEQ ID NO: 677 - RNA, 19 nt, synthetic construct
  source 1..19: mol_type = other RNA
tagtgtcctaacatcagct

SEQ ID NO: 678 - RNA, 19 nt, synthetic construct
  source 1..19: mol_type = other RNA
cattgggtagtgtcctaac

SEQ ID NO: 679 - RNA, 19 nt, synthetic construct
  source 1..19: mol_type = other RNA
tattgtccacggttgaggc

SEQ ID NO: 680 - RNA, 19 nt, synthetic construct
  source 1..19: mol_type = other RNA
gagcgaatattgtccacgg

SEQ ID NO: 681 - RNA, 19 nt, synthetic construct
  source 1..19: mol_type = other RNA
agaaatatctgtcttatcc

SEQ ID NO: 682 - RNA, 19 nt, synthetic construct
  source 1..19: mol_type = other RNA
ctttcgggaatcatcttcc

SEQ ID NO: 683 - RNA, 19 nt, synthetic construct
  source 1..19: mol_type = other RNA
acaagctgcagtcatttcc

SEQ ID NO: 684 - RNA, 19 nt, synthetic construct
  source 1..19: mol_type = other RNA
aggttaatgatccttctcc

SEQ ID NO: 685 - RNA, 19 nt, synthetic construct
  source 1..19: mol_type = other RNA
gtgaggttaatgatccttc

SEQ ID NO: 686 - RNA, 19 nt, synthetic construct
  source 1..19: mol_type = other RNA
gagactcaaaacactagtg

SEQ ID NO: 687 - RNA, 19 nt, synthetic construct
  source 1..19: mol_type = other RNA
gagaacctcatgtccctgc

SEQ ID NO: 688 - RNA, 19 nt, synthetic construct
  source 1..19: mol_type = other RNA
caacatctcccggagaacc

SEQ ID NO: 689 - RNA, 19 nt, synthetic construct
  source 1..19: mol_type = other RNA
ggttatgcaacatctcccg

SEQ ID NO: 690 - RNA, 19 nt, synthetic construct
  source 1..19: mol_type = other RNA
tggttatgcaacatctccc

SEQ ID NO: 691 - RNA, 19 nt, synthetic construct
  source 1..19: mol_type = other RNA
cacgaaggagtggttatgc

SEQ ID NO: 692 - RNA, 19 nt, synthetic construct
  source 1..19: mol_type = other RNA
ataacttggtttgatgctg

SEQ ID NO: 693 - RNA, 19 nt, synthetic construct
  source 1..19: mol_type = other RNA
gtataacttggtttgatgc

SEQ ID NO: 694 - RNA, 19 nt, synthetic construct
  source 1..19: mol_type = other RNA
ggatttagccagtatttca

SEQ ID NO: 695 - RNA, 19 nt, synthetic construct
  source 1..19: mol_type = other RNA
ggctttggccagcataaga

SEQ ID NO: 696 - RNA, 19 nt, synthetic construct
  source 1..19: mol_type = other RNA
gtggctcatgttactatta

SEQ ID NO: 697 - RNA, 19 nt, synthetic construct
  source 1..19: mol_type = other RNA
gttactattacaacgaaaa

SEQ ID NO: 698 - RNA, 19 nt, synthetic construct
  source 1..19: mol_type = other RNA
gatggaaagtgtgcataca

SEQ ID NO: 699 - RNA, 19 nt, synthetic construct
  source 1..19: mol_type = other RNA
ccatgtgctggcaatcaaa

SEQ ID NO: 700 - RNA, 19 nt, synthetic construct
  source 1..19: mol_type = other RNA
cacaatgcaggcattagta

SEQ ID NO: 701 - RNA, 19 nt, synthetic construct
  source 1..19: mol_type = other RNA
agctgatgttaggacacta

SEQ ID NO: 702 - RNA, 19 nt, synthetic construct
  source 1..19: mol_type = other RNA
gttaggacactacccaata

SEQ ID NO: 703 - RNA, 19 nt, synthetic construct
  source 1..19: mol_type = other RNA
gcctcaaccgtggacaata

SEQ ID NO: 704 - RNA, 19 nt, synthetic construct
  source 1..19: mol_type = other RNA
ccgtggacaatattcgcta

SEQ ID NO: 705 - RNA, 19 nt, synthetic construct
  source 1..19: mol_type = other RNA
ggataagacagatatttca

SEQ ID NO: 706 - RNA, 19 nt, synthetic construct
  source 1..19: mol_type = other RNA
ggaagatgattcccgaaaa

SEQ ID NO: 707 - RNA, 19 nt, synthetic construct
  source 1..19: mol_type = other RNA
ggaaatgactgcagcttga

SEQ ID NO: 708 - RNA, 19 nt, synthetic construct
  source 1..19: mol_type = other RNA
ggagaaggatcattaacca

SEQ ID NO: 709 - RNA, 19 nt, synthetic construct
  source 1..19: mol_type = other RNA
gaaggatcattaacctcaa

SEQ ID NO: 710 - RNA, 19 nt, synthetic construct
  source 1..19: mol_type = other RNA
cactagtgttttgagtcta

SEQ ID NO: 711 - RNA, 19 nt, synthetic construct
  source 1..19: mol_type = other RNA
gcagggacatgaggttcta

SEQ ID NO: 712 - RNA, 19 nt, synthetic construct
  source 1..19: mol_type = other RNA
ggttctccgggagatgtta

SEQ ID NO: 713 - RNA, 19 nt, synthetic construct
  source 1..19: mol_type = other RNA
cgggagatgttgcataaca

SEQ ID NO: 714 - RNA, 19 nt, synthetic construct
  source 1..19: mol_type = other RNA
gggagatgttgcataacca

SEQ ID NO: 715 - RNA, 19 nt, synthetic construct
  source 1..19: mol_type = other RNA
gcataaccactccttcgta

SEQ ID NO: 716 - RNA, 19 nt, synthetic construct
  source 1..19: mol_type = other RNA
cagcatcaaaccaagttaa

SEQ ID NO: 717 - RNA, 19 nt, synthetic construct
  source 1..19: mol_type = other RNA
gcatcaaaccaagttataa

SEQ ID NO: 718 - RNA, 21 nt, synthetic construct
  source 1..21: mol_type = other RNA
tgaaatactggctaaatcctc

SEQ ID NO: 719 - RNA, 21 nt, synthetic construct
  source 1..21: mol_type = other RNA
tcttatgctggccaaagcctc

SEQ ID NO: 720 - RNA, 21 nt, synthetic construct
  source 1..21: mol_type = other RNA
taatagtaacatgagccacat

SEQ ID NO: 721 - RNA, 21 nt, synthetic construct
  source 1..21: mol_type = other RNA
ttttcgttgtaatagtaacat

SEQ ID NO: 722 - RNA, 21 nt, synthetic construct
  source 1..21: mol_type = other RNA
tgtatgcacactttccatcag

SEQ ID NO: 723 - RNA, 21 nt, synthetic construct
  source 1..21: mol_type = other RNA
tttgattgccagcacatggtt

SEQ ID NO: 724 - RNA, 21 nt, synthetic construct
  source 1..21: mol_type = other RNA
tactaatgcctgcattgtgta

SEQ ID NO: 725 - RNA, 21 nt, synthetic construct
  source 1..21: mol_type = other RNA
tagtgtcctaacatcagctac

SEQ ID NO: 726 - RNA, 21 nt, synthetic construct
  source 1..21: mol_type = other RNA
tattgggtagtgtcctaacat

SEQ ID NO: 727 - RNA, 21 nt, synthetic construct
  source 1..21: mol_type = other RNA
tattgtccacggttgaggcat

SEQ ID NO: 728 - RNA, 21 nt, synthetic construct
  source 1..21: mol_type = other RNA
tagcgaatattgtccacggtt

SEQ ID NO: 729 - RNA, 21 nt, synthetic construct
  source 1..21: mol_type = other RNA
tgaaatatctgtcttatcctc

SEQ ID NO: 730 - RNA, 21 nt, synthetic construct
  source 1..21: mol_type = other RNA
ttttcgggaatcatcttccac

SEQ ID NO: 731 - RNA, 21 nt, synthetic construct
  source 1..21: mol_type = other RNA
tcaagctgcagtcatttcctt

SEQ ID NO: 732 - RNA, 21 nt, synthetic construct
  source 1..21: mol_type = other RNA
tggttaatgatccttctccgg

SEQ ID NO: 733 - RNA, 21 nt, synthetic construct
  source 1..21: mol_type = other RNA
ttgaggttaatgatccttctc

SEQ ID NO: 734 - RNA, 21 nt, synthetic construct
  source 1..21: mol_type = other RNA
tagactcaaaacactagtgag

SEQ ID NO: 735 - RNA, 21 nt, synthetic construct
  source 1..21: mol_type = other RNA
tagaacctcatgtccctgctc

SEQ ID NO: 736 - RNA, 21 nt, synthetic construct
  source 1..21: mol_type = other RNA
taacatctcccggagaacctc

SEQ ID NO: 737 - RNA, 21 nt, synthetic construct
  source 1..21: mol_type = other RNA
tgttatgcaacatctcccgga

SEQ ID NO: 738 - RNA, 21 nt, synthetic construct
  source 1..21: mol_type = other RNA
tggttatgcaacatctcccgg

SEQ ID NO: 739 - RNA, 21 nt, synthetic construct
  source 1..21: mol_type = other RNA
tacgaaggagtggttatgcaa

SEQ ID NO: 740 - RNA, 21 nt, synthetic construct
  source 1..21: mol_type = other RNA
ttaacttggtttgatgctgtg

SEQ ID NO: 741 - RNA, 21 nt, synthetic construct
  source 1..21: mol_type = other RNA
ttataacttggtttgatgctg

SEQ ID NO: 742 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggatttagccagtatttca

SEQ ID NO: 743 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggctttggccagcataaga

SEQ ID NO: 744 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gtggctcatgttactatta

SEQ ID NO: 745 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gttactattacaacgaaaa

SEQ ID NO: 746 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gatggaaagtgtgcataca

SEQ ID NO: 747 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ccatgtgctggcaatcaaa

SEQ ID NO: 748 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
cacaatgcaggcattagta

SEQ ID NO: 749 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
agctgatgttaggacacta

SEQ ID NO: 750 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gttaggacactacccaata

SEQ ID NO: 751 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gcctcaaccgtggacaata

SEQ ID NO: 752 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ccgtggacaatattcgcta

SEQ ID NO: 753 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggataagacagatatttca

SEQ ID NO: 754 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggaagatgattcccgaaaa

SEQ ID NO: 755 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggaaatgactgcagcttga

SEQ ID NO: 756 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggagaaggatcattaacca

SEQ ID NO: 757 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gaaggatcattaacctcaa

SEQ ID NO: 758 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
cactagtgttttgagtcta

SEQ ID NO: 759 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gcagggacatgaggttcta

SEQ ID NO: 760 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
ggttctccgggagatgtta

SEQ ID NO: 761 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
cgggagatgttgcataaca

SEQ ID NO: 762 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gggagatgttgcataacca

SEQ ID NO: 763 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gcataaccactccttcgta

SEQ ID NO: 764 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
cagcatcaaaccaagttaa

SEQ ID NO: 765 - RNA, 19 nt, synthetic construct
  source 1..19: mol_type = other RNA
  modified_base 1..2: mod_base = OTHER; note = 2'-O-methyl nucleotide
  modified_base 4..6: mod_base = OTHER; note = 2'-O-methyl nucleotide
  modified_base 10..11: mod_base = OTHER; note = 2'-O-methyl nucleotide
  modified_base 13..16: mod_base = OTHER; note = 2'-O-methyl nucleotide
  modified_base 18..19: mod_base = OTHER; note = 2'-O-methyl nucleotide
  misc_feature 18..19: note = phosphorothioate internucleotide linkage
gcatcaaaccaagttataa

SEQ ID NO: 766 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tgaaatactggctaaatcctc

SEQ ID NO: 767 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tcttatgctggccaaagcctc

SEQ ID NO: 768 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taatagtaacatgagccacat

SEQ ID NO: 769 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttttcgttgtaatagtaacat

SEQ ID NO: 770 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tgtatgcacactttccatcag

SEQ ID NO: 771 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tttgattgccagcacatggtt

SEQ ID NO: 772 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tactaatgcctgcattgtgta

SEQ ID NO: 773 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tagtgtcctaacatcagctac

SEQ ID NO: 774 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tattgggtagtgtcctaacat

SEQ ID NO: 775 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tattgtccacggttgaggcat

SEQ ID NO: 776 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tagcgaatattgtccacggtt

SEQ ID NO: 777 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tgaaatatctgtcttatcctc

SEQ ID NO: 778 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttttcgggaatcatcttccac

SEQ ID NO: 779 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tcaagctgcagtcatttcctt

SEQ ID NO: 780 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tggttaatgatccttctccgg

SEQ ID NO: 781 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttgaggttaatgatccttctc

SEQ ID NO: 782 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tagactcaaaacactagtgag

SEQ ID NO: 783 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tagaacctcatgtccctgctc

SEQ ID NO: 784 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
taacatctcccggagaacctc

SEQ ID NO: 785 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tgttatgcaacatctcccgga

SEQ ID NO: 786 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tggttatgcaacatctcccgg

SEQ ID NO: 787 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
tacgaaggagtggttatgcaa

SEQ ID NO: 788 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttaacttggtttgatgctgtg

SEQ ID NO: 789 - DNA, 21 bp, synthetic construct
  source 1..21: mol_type = other DNA
  modified_base 13: mod_base = OTHER; note = 2'-O-methyl nucleotide
  modified_base 20..21: mod_base = OTHER; note = 2'-O-methyl nucleotide
  misc_feature 20..21: note = phosphorothioate internucleotide linkage
  misc_feature 1: note = DNA
  misc_feature 2..21: note = RNA
ttataacttggtttgatgctg

SEQ ID NO: 790 - RNA, 23 nt, Homo sapiens
  source 1..23: mol_type = mRNA
gaggatttagccagtatttctac

SEQ ID NO: 791 - RNA, 23 nt, Homo sapiens
  source 1..23: mol_type = mRNA
gaggctttggccagcataagcca

SEQ ID NO: 792 - RNA, 23 nt, Homo sapiens
  source 1..23: mol_type = mRNA
atgtggctcatgttactattaca

SEQ ID NO: 793 - RNA, 23 nt, Homo sapiens
  source 1..23: mol_type = mRNA
atgttactattacaacgaaaaca

SEQ ID NO: 794 - RNA, 23 nt, Homo sapiens
  source 1..23: mol_type = mRNA
ctgatggaaagtgtgcatacaga

SEQ ID NO: 795 - RNA, 23 nt, Homo sapiens
  source 1..23: mol_type = mRNA
aaccatgtgctggcaatcaaggg

SEQ ID NO: 796 - RNA, 23 nt, Homo sapiens
  source 1..23: mol_type = mRNA
tacacaatgcaggcattagtttc

SEQ ID NO: 797 - RNA, 23 nt, Homo sapiens
  source 1..23: mol_type = mRNA
gtagctgatgttaggacactacc

SEQ ID NO: 798 - RNA, 23 nt, Homo sapiens
  source 1..23: mol_type = mRNA
atgttaggacactacccaatgcc

SEQ ID NO: 799 - RNA, 23 nt, Homo sapiens
  source 1..23: mol_type = mRNA
atgcctcaaccgtggacaatatt

SEQ ID NO: 800 - RNA, 23 nt, Homo sapiens
  source 1..23: mol_type = mRNA
aaccgtggacaatattcgctcca

SEQ ID NO: 801 - RNA, 23 nt, Homo sapiens
  source 1..23: mol_type = mRNA
gaggataagacagatatttctag

SEQ ID NO: 802 - RNA, 23 nt, Homo sapiens
  source 1..23: mol_type = mRNA
gtggaagatgattcccgaaagga

SEQ ID NO: 803 - RNA, 23 nt, Homo sapiens
  source 1..23: mol_type = mRNA
aaggaaatgactgcagcttgtac

SEQ ID NO: 804 - RNA, 23 nt, Homo sapiens
  source 1..23: mol_type = mRNA
ccggagaaggatcattaacctca

SEQ ID NO: 805 - RNA, 23 nt, Homo sapiens
  source 1..23: mol_type = mRNA
gagaaggatcattaacctcacta

SEQ ID NO: 806 - RNA, 23 nt, Homo sapiens
  source 1..23: mol_type = mRNA
ctcactagtgttttgagtctcca

SEQ ID NO: 807 - RNA, 23 nt, Homo sapiens
  source 1..23: mol_type = mRNA
gagcagggacatgaggttctccg

SEQ ID NO: 808 - RNA, 23 nt, Homo sapiens
  source 1..23: mol_type = mRNA
gaggttctccgggagatgttgca

SEQ ID NO: 809 - RNA, 23 nt, Homo sapiens
  source 1..23: mol_type = mRNA
tccgggagatgttgcataaccac

SEQ ID NO: 810 - RNA, 23 nt, Homo sapiens
  source 1..23: mol_type = mRNA
ccgggagatgttgcataaccact

SEQ ID NO: 811 - RNA, 23 nt, Homo sapiens
  source 1..23: mol_type = mRNA
ttgcataaccactccttcgtggg

SEQ ID NO: 812 - RNA, 23 nt, Homo sapiens
  source 1..23: mol_type = mRNA
cacagcatcaaaccaagttatac

SEQ ID NO: 813 - RNA, 23 nt, Homo sapiens
  source 1..23: mol_type = mRNA
cagcatcaaaccaagttatacct

SEQ ID NO: 814-900 - skipped (87 entries)

SEQ ID NO: 901 - protein, 677 aa, Moloney Murine Leukemia Virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 902 - protein, 1368 aa, Streptococcus pyogenes [sequence omitted]

SEQ ID NO: 903 - protein, 1052 aa, Staphylococcus lugdunensis
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKK

SEQ ID NO: 904 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 905 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 906-907 - skipped (2 entries)

SEQ ID NO: 908 - RNA, 30 nt, synthetic construct
  source 1..30: mol_type = other RNA
aacattgacgcgtctctacgtgggggcgcg

SEQ ID NO: 909-2467 - skipped (1559 entries)

SEQ ID NO: 2468 - protein, 33 aa, synthetic construct
SGGSSGGSSGSETPGTSESATPESSGGSSGGSS

SEQ ID NO: 2469 - protein, 503 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDNSRLIN

SEQ ID NO: 2470 - protein, 30 aa, synthetic construct
MDSLLMNRRKFLYQFKNVRWAKGRRETYLC

SEQ ID NO: 2471 - protein, 18 aa, synthetic construct
KRTADGSEFESPKKKRKV

SEQ ID NO: 2472 - protein, 17 aa, synthetic construct
KRTADGSEFEPKKKRKV

SEQ ID NO: 2473 - protein, 19 aa, synthetic construct
NLSKRPAAIKKAGQAKKKK

SEQ ID NO: 2474 - protein, 11 aa, synthetic construct
RQRRNELKRSF

SEQ ID NO: 2475 - protein, 38 aa, synthetic construct
NQSSNFGPMKGGNFGGRSSGPYGGGGQYFAKPRNQGGY

SEQ ID NO: 2476 - protein, 7 aa, synthetic construct
PKKKRKV

SEQ ID NO: 2477 - protein, 16 aa, synthetic construct
  VARIANT 3..12: note = X can be any amino acid
KRXXXXXXXXXXKKKL

SEQ ID NO: 2478 - protein, 16 aa, synthetic construct
KRPAATKKAGQAKKKK

SEQ ID NO: 2479 - protein, 19 aa, synthetic construct
MKRTADGSEFESPKKKRKV

SEQ ID NO: 2480 - protein, 20 aa, synthetic construct
AVKRPAATKKAGQAKKKKLD

SEQ ID NO: 2481 - protein, 25 aa, synthetic construct
MSRRRKANPTKLSENAKKLAKEVEN

SEQ ID NO: 2482 - protein, 9 aa, synthetic construct
PAAKRVKLD

SEQ ID NO: 2483 - protein, 9 aa, synthetic construct
KLKIKRPVK

SEQ ID NO: 2484 - protein, 8 aa, synthetic construct
VSRKRPRP

SEQ ID NO: 2485 - protein, 10 aa, synthetic construct
EGAPPAKRAR

SEQ ID NO: 2486 - protein, 12 aa, synthetic construct
PPQPKKKPLDGE

SEQ ID NO: 2487 - protein, 21 aa, synthetic construct
SGGSKRTADGSEFEPKKKRKV

SEQ ID NO: 2488 - protein, 16 aa, synthetic construct
KRPAATKKAGQAKKKK

SEQ ID NO: 2489 - protein, 34 aa, synthetic construct
SGGSSGGSKRTADGSEFESPKKKRKVSGGSSGGS

SEQ ID NO: 2490 - protein, 22 aa, synthetic construct
SGGSKRTADGSEFESPKKKRKV

SEQ ID NO: 2491 - protein, 12 aa, synthetic construct
GSGPAAKRVKLD

SEQ ID NO: 2492 - RNA, 33 nt, synthetic construct
  source 1..33: mol_type = other RNA
gccaacatgaggatcacccatgtctgcagggcc

SEQ ID NO: 2493 - protein, 131 aa, synthetic construct
GSASNFTQFVLVDNGGTGDVTVAPSNFANGVAEWISSNSRSQAYKVTCSVRQSSAQNRKYTIKVEVPKVATQTVGGEELPVAGWRSYLNMELTIPIFATNSDCELIVKAMQGLLKDGNPIPSAIAANSGIY

SEQ ID NO: 2494 - protein, 150 aa, synthetic construct
  SITE 1..150: note = This sequence may encompass 1-30 GGGGS repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 2495 - protein, 30 aa, synthetic construct
  SITE 1..30: note = This sequence may encompass 1-30 residues
GGGGGGGGGGGGGGGGGGGGGGGGGGGGGG

SEQ ID NO: 2496 - protein, 150 aa, synthetic construct
  SITE 1..150: note = This sequence may encompass 1-30 EAAAK repeating units
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 2497 - protein, 90 aa, synthetic construct
  SITE 1..90: note = This sequence may encompass 1-30 GGS repeating units
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 2498 - protein, 120 aa, synthetic construct
  SITE 1..120: note = This sequence may encompass 1-30 SGGS repeating units
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 2499 - protein, 60 aa, synthetic construct
  SITE 1..60: note = This sequence may encompass 1-30 XP repeating units, wherein X can be any amino acid
XPXPXPXPXPXPXPXPXPXPXPXPXPXPXPXPXPXPXPXPXPXPXPXPXPXPXPXPXPXP

SEQ ID NO: 2500 - protein, 21 aa, synthetic construct
  SITE 1..21: note = This sequence may encompass 1, 3 or 7 GGS repeating units
GGSGGSGGSGGSGGSGGSGGS

SEQ ID NO: 2501 - protein, 16 aa, synthetic construct
SGSETPGTSESATPES

SEQ ID NO: 2502 - protein, 32 aa, synthetic construct
SGGSSGGSSGSETPGTSESATPESSGGSSGGS

SEQ ID NO: 2503 - protein, 10 aa, synthetic construct
SGGSGGSGGS

SEQ ID NO: 2504 - protein, 4 aa, synthetic construct
SGGS

SEQ ID NO: 2505 - protein, 60 aa, synthetic construct
SGGSSGGSSGSETPGTSESATPESAGSYPYDVPDYAGSAAPAAKKKKLDGSGSGGSSGGS

SEQ ID NO: 2506 - protein, 6 aa, synthetic construct
GGSGGS

SEQ ID NO: 2507 - protein, 9 aa, synthetic construct
GGSGGSGGS

SEQ ID NO: 2508 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2509 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 2510 - protein, 2131 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD

SEQ ID NO: 2511 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2512 - RNA, 76 nt, synthetic construct
  source 1..76: mol_type = other RNA
gtttgagagctagaaatagcaagtttaaataaggctagtccgttatcaacttgaaaaagtgggaccgagtcggtcc

SEQ ID NO: 2513 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 2514 - protein, 16 aa, synthetic construct
RQIKIWFQNRRMKWKK

SEQ ID NO: 2515 - protein, 9 aa, synthetic construct
RRRRRRRRR

SEQ ID NO: 2516 - protein, 8 aa, synthetic construct
RRRRRRRR

(19) United States
(12) Patent Application Publication
(10) Pub. No.: US 2025/0297248 A1
(43) Pub. Date: Sep. 25, 2025

(54) METHODS AND COMPOSITIONS FOR INHIBITING MISMATCH REPAIR

(71) Applicant: Prime Medicine, Inc., Cambridge, MA (US)

(72) Inventors: David Waterman, Framingham, MA (US); Pei Ge, Arlington, MA (US); Ekambareswara Rao Kandimalla, Hopkinton, MA (US)

(21) Appl. No.: 18/712,925

(22) PCT Filed: Nov. 21, 2022
(86) PCT No.: PCT/US2022/050539

§ 371 Date: May 23, 2024

Related U.S. Application Data
(60) Provisional application No. 63/282,950, filed on Nov. 24, 2021.

(51) Int. Cl. C12N 15/113; A61K 48/00; C12N 9/12; C12N 9/22

(52) U.S. Cl. CPC C12N 15/113; A61K 48/005; C12N 9/1276; C12N 9/226; C12Y 207/07049; C12N 2310/11; C12N 2310/14; C12N 2310/3125; C12N 2310/315

(57) ABSTRACT

Disclosed herein are siRNAs and antisense oligonucleotides (ASOs) specific for an mRNA sequence of a mutS homolog 2 (MSH2) gene, PMS1 homolog 2, mismatch repair system component (PMS2) gene, mutS homolog 6 (MSH6) gene, or mutL homolog 1 (MLH1) gene. Such siRNAs and ASOs can be used in methods of inhibiting DNA mismatch repair. Also disclosed are systems and methods that combine the use of these siRNAs and ASOs with prime editing technology.

### CROSS REFERENCE TO RELATED APPLICATIONS

This application is a § 371 national-stage application based on PCT/US22/50539, filed Nov. 21, 2022, which claims the benefit of U.S. Provisional Application No. 63/282,950, filed Nov. 24, 2021, the entire contents of each are incorporated herein by reference.

### REFERENCE TO SEQUENCE LISTING

The instant application contains a Sequence Listing which has been submitted electronically in XML format via EFS-Web, and is hereby incorporated by reference in its entirety. Said XML copy, created on Jan. 9, 2023, is named PMB_00301_SeqList_ST26 and is 1,986,985 bytes in size is hereby incorporated by reference in its entirety.

### BACKGROUND

Techniques that allow manipulating DNA have numerous applications, such as in the treatment of genetic diseases. Among such techniques, prime editing can be used to introduce base pair substitutions, insertions, and deletions in the DNA. Prime editing efficiency has been shown to be improved by inhibition of the mismatch repair pathway. Thus, there is a need in the field for new methods and compositions for inhibiting the mismatch repair pathway that can be used, for example, in conjunction with prime editing technologies.

### SUMMARY

Provided herein are compositions and methods related to certain siRNA and antisense molecules that inhibit components of the mismatch repair pathway. Such siRNA and antisense molecules can be used to inhibit mismatch repair, for example, in conjunction with prime editing technology.

In some aspects, provided herein are siRNAs specific for an mRNA sequence of a mutS homolog 2 (MSH2) gene. In some embodiments, the siRNAs comprise a sequence complementarity to any one of the target nucleic acid sequences set forth in SEQ ID NO: 177 to 200. In some embodiments, the siRNAs comprise any one of the matched antisense strand and sense strand pairs set forth in Tables 2-4.

In some aspects, provided herein are siRNAs specific for an mRNA sequence of a PMS1 homolog 2, mismatch repair system component (PMS2) gene. In some embodiments, the siRNAs comprise a sequence complementarity to any one of the target nucleic acid sequences set forth in SEQ ID NO: 373 to 396. In some embodiments, the siRNAs comprise any one of the matched antisense strand and sense strand pairs set forth in Tables 7-9.

In some aspects, provided herein are siRNAs specific for an mRNA sequence of a mutS homolog 6 (MSH6) gene. In some embodiments, the siRNAs comprise a sequence complementarity to any one of the target nucleic acid sequences set forth in SEQ ID NO: 585 to 608. In some embodiments, the siRNAs comprise any one of the matched antisense strand and sense strand pairs set forth in Tables 12-14.

In some aspects, provided herein are siRNAs specific for an mRNA sequence of a mutL homolog 1 (MLH1) gene. In some embodiments, the siRNAs comprise a sequence complementarity to any one of the target nucleic acid sequences set forth in SEQ ID NO: 790 to 813. In some embodiments, the siRNAs comprise any one of the matched antisense strand and sense strand pairs set forth in Tables 17-19.

In some aspects, provided herein are antisense oligonucleotides (ASOs) specific for an mRNA sequence of the mutS homolog 2 (MSH2) gene. In some embodiments, the ASOs comprise a nucleic acid sequence set forth in SEQ ID NOs: 1-32.

In some aspects, provided herein are antisense oligonucleotides (ASOs) specific for an mRNA sequence of the PMS1 homolog 2, mismatch repair system component (PMS2) gene. In some embodiments, the ASOs comprise a nucleic acid sequence set forth in SEQ ID NOs: 201-228.

In some aspects, provided herein are antisense oligonucleotides (ASOs) specific for an mRNA sequence of the mutS homolog 6 (MSH6) gene. In some embodiments, the ASOs comprise a nucleic acid sequence set forth in SEQ ID NOs: 397-440.

In some aspects, provided herein are antisense oligonucleotides (ASOs) specific for an mRNA sequence of the mutL homolog 1 (MLH1) gene. In some embodiments, the ASOs comprise a nucleic acid sequence set forth in SEQ ID NOs: 609-645.

Numerous embodiments applicable to each of the above aspects and embodiments are described in the rest of this section and throughout this disclosure.

In some embodiments, the siRNAs provided herein comprise a phosphorothioate internucleotide bond, a methylphosphonate internucleotide bond, and/or a triazole internucleotide bond. In some embodiments, the siRNAs comprise a 2′-O-methoxyethyl oligonucleotide (2′MOE), a 2′-O-methylated nucleoside (2′OMe), a 2′-fluoro oligonucleotide (2′F), an arabino nucleotide (ANA), a 2′-F arabino nucleotide (FANA), a phosphorodiamidate morpholino oligonucleotide (PMO), a peptide nucleic acid (PNA), a phosphorothioate bond (PS), a locked nucleic acid (LNA), a hydrophobic moiety, a naphthyl modifier, or a cholesterol moiety. In some embodiments, the siRNAs are modified with a cholesterol, a dialkyl lipid, or GalNAc. In some embodiments, the siRNAs are chemically modified with poly-ethylene glycol (PEG). In some embodiments, the siRNAs comprise a 5′ end cap. In some embodiments, the siRNAs comprise a 3′ end cap. As used herein “cap” may refer to any altered nucleotide on the 5′ or 3′ end of the siRNA. In some embodiments, the siRNA comprises at least one phosphorothioate internucleotide linkage (e.g., such as a stereospecific phosphorothioate internucleotide linkage). In some embodiments, the siRNAs comprise a DNA nucleotide. In some embodiments, the DNA nucleotide is thymine.

In some embodiments, the ASOs provided herein comprise an antisense strand comprising deoxyribonucleotides and/or ribonucleotides. In some embodiments, the ASOs comprise an antisense strand comprising at least five ribonucleotides at the 5′ end of the antisense strand. In some embodiments, the ASOs comprise an antisense strand comprising at least five ribonucleotides at the 3′ end of the antisense strand. In some embodiments, the ASOs comprise an antisense strand (5′ to 3′) comprising deoxyribonucleotides from nucleotide position 6 to nucleotide position 15. In some embodiments, the ASOs comprise a chemical modification. In some embodiments, the ASOs comprise a phosphorothioate internucleotide bond, a methylphosphonate internucleotide bond, and/or a triazole internucleotide bond. In some embodiments, the ASOs comprise a 2′-O-methoxyethyl oligonucleotide (2′MOE), a 2′-O-methylated nucleoside (2′OMe), a 2′-fluoro oligonucleotide (2′F), a phosphorodiamidate morpholino oligonucleotide (PMO), an arabino nucleotide (ANA), a 2′-F arabino nucleotide (FANA), a peptide nucleic acid (PNA), a phosphorothioate bond (PS), a locked nucleic acid (LNA), a hydrophobic moiety, a naphthyl modifier, or a cholesterol moiety. In some embodiments, the ASOs are modified with a cholesterol, a dialkyl lipid, or GalNAc. In some embodiments, the ASOs are chemically modified with poly-ethylene glycol (PEG). In some embodiments, the ASOs comprise a 5′ end cap. In some embodiments, the ASO comprises a 3′ end cap. As used herein “cap” may refer to any altered nucleotide on the 5′ or 3′ end of the ASO. In some embodiments, the ASO comprises at least one phosphorothioate internucleotide linkage (e.g., such as a stereospecific phosphorothioate internucleotide linkage).

In some aspects, provided herein are systems comprising prime editing guide RNAs (PEgRNAs) and one or more siRNAs and/or ASOs (e.g., the siRNAs and/or ASOs provided herein). In some embodiments, the PEgRNA comprises: a spacer that comprises a region of complementarity to a search target sequence in target strand of a double stranded target DNA; a guide RNA (gRNA) core; an editing template that comprises an intended edit compared to the double stranded target DNA; and a primer binding site (PBS) that comprises a region of complementarity to a region upstream of a nick site in a non-target strand of the double stranded target DNA.

In some aspects, provided herein are methods of inhibiting DNA mismatch repair comprising contacting a cell with one or more siRNAs and/or one or more ASOs (e.g., the siRNAs and/or ASOs disclosed herein). In some embodiments, the one or more siRNAs and/or one or more ASOs is in a lipid nanoparticle. In some aspects, provided herein are lipid nanoparticles (LNPs) comprising one or more siRNAs and/or one or more ASOs (e.g., the siRNAs and/or ASOs disclosed herein). In some aspects, provided herein are methods for editing a gene in a cell comprising contacting the cell with a prime editing guide RNA (PEgRNA), a prime editor comprising a DNA binding domain and a DNA polymerase domain, and one or more siRNAs and/or one or more ASOs (e.g., the siRNAs and/or ASOs disclosed herein). In some embodiments, the PEgRNA comprises: a spacer that comprises a region of complementarity to a search target sequence in target strand of a double stranded target DNA; a guide RNA (gRNA) core; an editing template that comprises an intended edit compared to the double stranded target DNA; and a primer binding site (PBS) that comprises a region of complementarity to a region upstream of a nick site in a non-target strand of the double stranded target DNA. In some embodiments, the prime editor synthesizes a single stranded DNA encoded by the editing template, wherein the single stranded DNA replaces the editing target sequence and results in incorporation of the intended nucleotide edit into a region corresponding to the editing target in the gene. In some embodiments, the cell is a mammalian cell. In some embodiments, the cell is a human cell. In some embodiments, the cell is a primary cell. In some embodiments, the cell is in a subject. In some embodiments, the subject is a human. In some embodiments, the methods further comprise administering the cell to the subject after incorporation of the intended nucleotide edit.

### DETAILED DESCRIPTION

The present disclosure relates to siRNAs and antisense molecules that inhibit a component of the mismatch repair pathway, and the use of such siRNAs and antisense molecules in conjunction with prime editing.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof as used herein mean “comprising.”

Unless otherwise specified, the words “comprising,” “comprise,” “comprises,” “having,” “have,” “has,” “including,” “includes,” “include,” “containing,” “contains,” “contain” and variants thereof are inclusive or open-ended and do not exclude additional, unrecited elements or method steps.

Reference to “some embodiments,” “an embodiment,” “one embodiment,” or “other embodiments” means that a particular feature or characteristic described in connection with the embodiments is included in at least one or more embodiments, but not necessarily all embodiments, of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell).

In some embodiments, the cell is a human cell. A cell may be of or derived from different tissues, organs, and/or cell types. In some embodiments, the cell is a primary cell. In some embodiments, the term primary cell means a cell isolated from an organism, e.g., a mammal, which is grown in tissue culture (i.e., in vitro) for the first time before subdivision and transfer to a subculture. In some non-limiting examples, mammalian primary cells can be modified through introduction of one or more polynucleotides, polypeptides, and/or prime editing compositions (e.g., through transfection, transduction, electroporation and the like) and further passaged. Such modified mammalian primary cells include retinal cells (e.g., photoreceptors, retinal pigment epithelium cells), epithelial cells (e.g., mammary epithelial cells, intestinal epithelial cells, hepatocytes), endothelial cells, glial cells, neural cells, formed elements of the blood (e.g., lymphocytes, bone marrow cells), precursors of any of these somatic cell types, and stem cells. In some embodiments, the cell is a fibroblast. In some embodiments, the cell is a stem cell. In some embodiments, the cell is a pluripotent stem cell. In some embodiments, the cell is an induced pluripotent stem cell (iPSC). In some embodiments, the cell is a retinal progenitor cell. In some embodiments, the cell is a retinal precursor cell. In some embodiments, the cell is an embryonic stem cell (ESC). In some embodiments, the cell is a human stem cell. In some embodiments, the cell is a human pluripotent stem cell. In some embodiments, the cell is a human fibroblast. In some embodiments, the cell is an induced human pluripotent stem cell. In some embodiments, the cell is a human stem cell. In some embodiments, the cell is a human embryonic stem cell.

In some embodiments, a cell is not isolated from an organism but forms part of a tissue or organ of an organism, e.g., a mammal, such as a human. In some non-limiting examples, mammalian cells include muscle cells (e.g., cardiac muscle cells, smooth muscle cells, myosatellite cells), epithelial cells (e.g., mammary epithelial cells, intestinal epithelial cells, hepatocytes), endothelial cells, glial cells, neural cells, formed elements of the blood (e.g., lymphocytes, bone marrow cells), precursors of any of these somatic cell types, and stem cells. In some embodiments, the cell is a stem cell. In some embodiments, the cell is a human stem cell.

In some embodiments, the cell is a differentiated cell. In some embodiments, cell is a fibroblast. In some embodiments, the cell is differentiated from an induced pluripotent stem cell. In some embodiments, the cell is a differentiated human cell. In some embodiments, cell is a human fibroblast. In some embodiments, the cell is differentiated from an induced human pluripotent stem cell.

In some embodiments, the cell comprises a prime editor or a prime editing composition. In some embodiments, the cell is from a human subject. In some embodiments, the human subject has a disease or condition associated with a mutation to be corrected by prime editing. In some embodiments, the cell is from a human subject, and comprises a prime editor or a prime editing composition for correction of the mutation. In some embodiments, the cell is from the human subject and the mutation has been edited or corrected by prime editing. In some embodiments, the cell is in a human subject, and comprises a prime editor or a prime editing composition for correction of the mutation. In some embodiments, the cell is from the human subject and the mutation has been edited or corrected by prime editing.

The term “substantially” as used herein may refer to a value approaching 100% of a given value. In some embodiments, the term may refer to an amount that may be at least about 70%, 80%, 90%, 91%, 92%, 93%, 94%, 95%, 96%, 97%, 98%, 99%, 99.9%, or 99.99% of a total amount. In some embodiments, the term may refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein may be a full-length protein (e.g., a fully processed protein having certain biological function). In some embodiments, a protein may be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain,” “protein domain,” or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor may be a fusion protein comprising a Cas9 protein domain of S. pyogenes and a reverse transcriptase protein domain of Moloney murine leukemia virus. A protein that comprises amino acid sequences from different origins or naturally occurring proteins may be referred to as a fusion, or chimeric protein.

In some embodiments, a protein comprises a functional variant or functional fragment of a full-length wild type protein. A “functional fragment” or “functional portion,” as used herein, refers to any portion of a reference protein (e.g., a wild type protein) that encompasses less than the entire amino acid sequence of the reference protein while retaining one or more of the functions, e.g., catalytic or binding functions. For example, a functional fragment of a reverse transcriptase may encompass less than the entire amino acid sequence of a wild type reverse transcriptase, but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild type Cas9, but retains its DNA binding ability and lacks its nuclease activity partially or completely.

A “functional variant” or “functional mutant,” as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild type reverse transcriptase, but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional fragment of a Cas9 may comprise one or more amino acid substitutions in a nuclease domain, e.g., an H840A amino acid substitution, compared to the amino acid sequence of a wild type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

In some embodiments, a protein or polypeptide includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptide includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide includes both naturally occurring amino acids and non-naturally occurring amino acids. In some embodiments, a protein or polypeptide is modified.

In some embodiments, a protein or polypeptide is an isolated protein or an isolated polypeptide. The term “isolated” means free or substantially free from components which normally accompany it as found in the natural state or environment. For example, a polypeptide naturally present in a living animal is not isolated, when present in that living animal in its natural state, and the same polypeptide substantially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid or polynucleotide sequence and a corresponding reference sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, primer binding site or protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences,” Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison,” Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16: 276-277), and the GGSEARCH program https://fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998).

Amino acid (or nucleotide) positions may be determined in homologous sequences based on alignment, for example, “H840” in a reference Cas9 sequence may correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G).

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

The term “complement,” “complementary,” or “complementarity” as used herein, refers to the ability of two polynucleotide molecules to base pair with each other. Complementary polynucleotides may base pair via hydrogen bonding, which may be Watson Crick, Hoogsteen or reversed Hoogsteen hydrogen bonding. For example, an adenine on one polynucleotide molecule will base pair to a guanine on a second polynucleotide molecule and a cytosine on one polynucleotide molecule will base pair to a thymine or uracil on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. “Substantial complementary” can also refer to a 100% complementarity over a portion of two polynucleotide molecules. In some embodiments, the portion of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, translated into peptides, polypeptides, or proteins. If the polynucleotide is derived from genomic DNA, expression may include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript that is encoded by the gene after transcription the gene. In some embodiments, expression of a polynucleotide, e.g., an mRNA, is determined by the amount of the protein encoded by the mRNA after translation of the mRNA. In some embodiments, expression of a polynucleotide, e.g., an mRNA or coding RNA, is determined by the amount of a functional form of the protein encoded by the polypeptide after translation of the polynucleotide.

The term “sequencing” as used herein, may comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, or any combination thereof.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” and its grammatical equivalents as used herein may refer to a human or a non-human. A subject may be a mammal. A human subject may be male or female. A human subject may be of any age. A subject may be a human embryo. A human subject may be a newborn, an infant, a child, an adolescent, or an adult. A human subject may be up to about 100 years of age. A human subject may be in need of treatment for a genetic disease or disorder.

The terms “treatment” or “treating” and their grammatical equivalents may refer to the medical management of a subject with an intent to cure, ameliorate, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates, but does not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g., a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective amount” or “therapeutically effective amount” may refer to a quantity of a composition, for example a composition comprising a construct, that can be sufficient to result in a desired activity upon introduction into a subject as disclosed herein. An effective amount of the prime editing compositions can be provided to the target gene or cell, whether the cell is ex vivo or in vivo.

An effective amount can be the amount to induce, for example, at least about a 2-fold change (increase or decrease) or more in the amount of target nucleic acid modulation observed relative to a negative control. An effective amount or dose can induce, for example, about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in target gene modulation.

The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro or in vivo).

### Interfering Nucleic Acids

In certain embodiments, provided herein are interfering nucleic acid molecules (e.g., siRNAs and/or ASOs) that selectively target component of the mismatch repair pathway described herein (e.g., MSH2, PMS2, MSH6, MLH1). Interfering nucleic acids generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. Interfering RNA molecules include, but are not limited to, antisense molecules, and siRNA molecules.

In some embodiments, at least 17, 18, 19, 20, 21, 22 or 23 nucleotides of the complement of the target mRNA sequence are sufficient to mediate inhibition of a target transcript. In certain embodiments, perfect complementarity is not necessary. In some embodiments, the interfering nucleic acid molecule is double-stranded RNA. The double-stranded RNA molecule may have a 3′ overhang (e.g., a 2 nucleotide 3′ overhang on one or both strands).

Interfering nucleic acid molecules provided herein can contain RNA bases, non-RNA bases or a mixture of RNA bases and non-RNA bases. For example, interfering nucleic acid molecules provided herein can be primarily composed of RNA bases but also contain DNA bases or non-naturally occurring nucleotides.

The interfering nucleic acids can employ a variety of oligonucleotide chemistries. Examples of oligonucleotide chemistries include, without limitation, peptide nucleic acid (PNA), locked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone. See, e.g., PCT Publication Nos. WO/2013/112053 and WO/2009/008725, incorporated by reference in their entireties.

Interfering nucleic acids may also contain “locked nucleic acid” subunits (LNAs). “LNAs” are a member of a class of modifications called bridged nucleic acid (BNA). BNA is characterized by a covalent linkage that locks the conformation of the ribose ring in a C3-endo (northern) sugar pucker. For LNA, the bridge is composed of a methylene between the 2′-0 and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, non-phosphorous containing linkers may be employed. One embodiment is an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo- and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′O-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphorothioate oligonucleotides (PTOs) for further stabilization. 2′O-Me oligonucleotides (phosphodiester or phosphothioate) can be synthesized according to routine techniques in the art (see, e.g., Yoo et al., Nucleic Acids Res. 32:2008-16, 2004).

In some embodiments, the interfering nucleic acid molecule is a siRNA molecule. Such siRNA molecules should include a region of sufficient homology to the target region, and be of sufficient length in terms of nucleotides, such that the siRNA molecule down-regulate target RNA. The term “ribonucleotide” or “nucleotide” can, in the case of a modified RNA or nucleotide surrogate, also refer to a modified nucleotide, or surrogate replacement moiety at one or more positions. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Each strand of an siRNA molecule can be equal to or less than 35, 30, 25, 24, 23, 22, 21, or 20 nucleotides in length. In some embodiments, the strand is at least 19 nucleotides in length. For example, each strand can be between 21 and 25 nucleotides in length. In some embodiments, siRNA agents have a duplex region of 17, 18, 19, 29, 21, 22, 23, 24, or 25 nucleotide pairs, and one or more overhangs, such as one or two 3′ overhangs, of 2-3 nucleotides.

In some embodiments, antisense oligonucleotide (ASO) compounds are provided herein. In certain embodiments, the degree of complementarity between the target sequence and antisense targeting sequence is sufficient to form a stable duplex. In some embodiments, the region of complementarity of the antisense oligonucleotides with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligonucleotide of about 14-15 bases is generally long enough to have a unique complementary sequence.

In certain embodiments, antisense oligonucleotides may be 100% complementary to the target sequence, or may include mismatches. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

Interfering nucleic acid molecules can be prepared, for example, by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

Antisense Oligonucleotides and siRNAs for MSH2

In certain aspects, provided herein are various siRNAs specific for an mRNA sequence of a mutS homolog 2 (MSH2) gene (e.g., MSH2 transcript variant 1, mRNA (NCBI Reference Sequence: NM_000251.3) or any other MSH2 mRNA isoform). In some embodiments, the siRNAs comprise a sequence complementarity to any one of the target nucleic acid sequences set forth in SEQ ID NO: 177 to 200. Exemplary antisense molecules against mutS homolog 2 (MSH2) transcript variant 1, mRNA and corresponding target nucleotide ranges are described in Table 1 below. In some embodiments, the siRNAs comprise any one of the matched antisense strand and sense strand pairs set forth in Tables 2-4. Where provided, values “VEGFA fold change,” “HEK3 fold change,” and “RNF2 fold change” indicate the amount of prime editing observed at the respective locus when the given antisense molecule or siRNA pair is administered, in accordance with Example 5, below, with in an vitro-transcribed pegRNA configured to edit that locus and an in vitro-transcribed prime editor, compared to co-administration with an antisense molecule or siRNA pair having a random scrambled sequence. The VEGFA and RNF2 pegRNAs each encode a one-nucleotide substitution, and the HEK3 pegRNA encodes a 3-nucleotide insertion.

In some embodiments, these siRNAs comprise a phosphorothioate internucleotide bond, a methylphosphonate internucleotide bond, and/or a triazole internucleotide bond. Any of these siRNAs may comprise a 2′-O-methoxyethyl oligonucleotide (2′MOE), a 2′-O-methylated nucleoside (2′OMe), a 2′-fluoro oligonucleotide (2′F), an arabino nucleotide (ANA), a 2′-F arabino nucleotide (FANA), a phosphorodiamidate morpholino oligonucleotide (PMO), a peptide nucleic acid (PNA), a phosphorothioate bond (PS), a locked nucleic acid (LNA), a hydrophobic moiety, a naphthyl modifier, or a cholesterol moiety. In some embodiments, these siRNAs are modified with a cholesterol, a dialkyl lipid, or GalNAc. In certain embodiments, these siRNAs are chemically modified with poly-ethylene glycol (PEG). In some embodiments, these siRNAs comprise a 5′ end cap. In some embodiments, these siRNAs may comprise a 3′ end cap. Any of these siRNAs may comprise a DNA nucleotide (e.g., thymine). In certain embodiments, these siRNAs comprise a DNA nucleoside (e.g., thymidine).

Also provided herein are antisense oligonucleotides (ASOs) specific for an mRNA sequence of the mutS homolog 2 (MSH2) gene. In some embodiments, the ASOs comprise a nucleic acid sequence set forth in SEQ ID NOs: 1-32.

In some embodiments, these ASOs comprise an antisense strand comprising deoxyribonucleotides and/or ribonucleotides. In some embodiments, these ASOs comprise an antisense strand comprising at least five ribonucleotides at the 5′ end of the siRNA antisense strand. In some embodiments, these ASOs comprise an antisense strand comprising at least five ribonucleotides at the 3′ end of the siRNA antisense strand. In some embodiments, the ASOs comprise an antisense strand (5′ to 3′) comprising deoxyribonucleotides from nucleotide position 6 to nucleotide position 15. In some embodiments, these ASOs comprise a chemical modification. In certain embodiments, these ASOs comprise a phosphorothioate internucleotide bond, a methylphosphonate internucleotide bond, and/or a triazole internucleotide bond. In some embodiments, these ASOs comprise a 2′-O-methoxyethyl oligonucleotide (2′MOE), a 2′-O-methylated nucleoside (2′OMe), a 2′-fluoro oligonucleotide (2′F), an arabino nucleotide (ANA), a 2′-F arabino nucleotide (FANA), a phosphorodiamidate morpholino oligonucleotide (PMO), a peptide nucleic acid (PNA), a phosphorothioate bond (PS), a locked nucleic acid (LNA), a hydrophobic moiety, a naphthyl modifier, or a cholesterol moiety. In some embodiments, these ASOs are modified with a cholesterol, a dialkyl lipid, or GalNAc. In certain embodiments, these ASOs are chemically modified with poly-ethylene glycol (PEG). In some embodiments, these ASOs comprise a 5′ end cap. Any of these ASOs may comprise a 3′ end cap.

TABLE 1
Antisense molecules against mutS homolog 2 (MSH2) transcript variant 1, mRNA
(NCBI Reference Sequence: NM_000251.3)

| SEQ | Target | | | | VEGFA | HEK3 | RNF2 |
|---|---|---|---|---|---|---|---|
| ID | Nucleotide | Target | Antisense Strand | Isoform | fold | fold | fold |
| NO. | Range | location | Sequence (5′→3′) | homology | change | change | change |
| 1 | 14-33 | 5′ UTR | GAAACCTCCTCACCTCCT | v1, X1-X4 | | | |
| | | | GG | | | | |
| 2 | 353-372 | CR | GGATGCCTTATTTCCAGC | V1, 2, X1- | 2.3 | 1.2 | 6.7 |
| | | | TC | X4 | | | |
| | | | | (all) | | | |
| 3 | 407-426 | CR | CTGAGAGAGATTGCCAGG | All | | | |
| | | | AG | | | | |
| 4 | 459-478 | CR | CAACACCAATGGAAGCTG | All | 2.3 | 1.2 | 4.7 |
| | | | AC | | | | |
| 5 | 463-482 | CR | CCCACAACACCAATGGAA | All | | | |
| | | | GC | | | | |
| 6 | 508-527 | CR | CCAACTCCAACCTGTCTC | All | | | |
| | | | TG | | | | |
| 7 | 585-604 | CR | GAGCCTCAAGATTGGAGA | All | 1.1 | 0.8 | 2.0 |
| | | | AC | | | | |
| 8 | 601-620 | CR | CCAATCTGGATGAGGAGA | All | | | |
| | | | GC | | | | |
| 9 | 644-663 | CR | GTCTCCAGCAGTCTCTCC | All | 1.5 | 0.8 | 1.3 |
| | | | TC | | | | |
| 10 | 783-802 | CR | CACTATTCATCTGCTCTC | All | 2.3 | 0.9 | 2.4 |
| | | | CC | | | | |
| 11 | 942-961 | CR | CTCTGACTGCTGCAATAT | All | 1.8 | 0.8 | 1.6 |
| | | | CC | | | | |
| 12 | 983-1002 | CR | GCCAGTGGTATCTTCAAC | All | | | |
| | | | AG | | | | |
| 13 | 1065-1084 | CR | GAGGCTGCTTAATCCACT | All | 2.0 | 1.0 | 3.3 |
| | | | GG | | | | |
| 14 | 1141-1160 | CR | GTCTGCCTCAATTCTGCA | All | 2.1 | 1.1 | 6.4 |
| | | | TC | | | | |
| 15 | 1529-1548 | CR | GCCAAGATCTCTGGCTGC | All | 1.9 | 1.1 | 3.4 |
| | | | AC | | | | |
| 16 | 1755-1774 | CR | CTTTAACAATGGCATCCT | All | | | |
| | | | GG | | | | |
| 17 | 1795-1814 | CR | TGCATTGGTTCTACATAG | All | 2.9 | 1.2 | 5.1 |
| | | | CC | | | | |
| 18 | 1839-1858 | CR | AGCTGACAACAGCATCTA | All | 1.2 | 0.9 | 2.0 |
| | | | GC | | | | |
| 19 | 1869-1888 | CR | GAACAGGTGCTCCATTTG | All | 1.0 | 0.8 | 1.8 |
| | | | AC | | | | |
| 20 | 1911-1930 | CR | TTCTTCCTTGTCCTTTCT | All | 1.7 | 0.8 | 1.4 |
| | | | CC | | | | |
| 21 | 2025-2044 | CR | GGCCAGTAATGATGTGGA | All | 1.3 | 0.8 | 1.5 |
| | | | AC | | | | |
| 22 | 2119-2138 | CR | TCTGCTGACTCACATGGC | All | 2.2 | 1.0 | 2.7 |
| | | | AC | | | | |
| 23 | 2214-2233 | CR | CAGTTTCCAACATTTCAG | All | 2.1 | 0.9 | 1.9 |
| | | | CC | | | | |
| 24 | 2231-2250 | CR | AGACCTGAGGATAGAAGC | All | 2.0 | 1.0 | 2.0 |
| | | | AG | | | | |
| 25 | 2286-2305 | CR | AGGTAGAAGTTCCTCTTC | All | 1.9 | 1.0 | 3.7 |
| | | | CC | | | | |
| 26 | 2311-2330 | CR | GCCCATGCTAACCCAAAT | All | 2.1 | 1.1 | 4.7 |
| | | | CC | | | | |
| 27 | 2442-2461 | CR | CTTCAGTGGTGAGTGCTG | All | 1.5 | 0.9 | 1.8 |
| | | | TG | | | | |
| 28 | 2515-2534 | CR | AGCTCTGCAACATGAATC | All | 2.0 | 0.8 | 3.2 |
| | | | CC | | | | |
| 29 | 2580-2599 | CR | GAAACTCCTCAAGTTCCA | All | | | |
| | | | GG | | | | |
| 30 | 2635-2654 | CR | CACTTCTTTGCTGCTGGT | All | | | |
| | | | TC | | | | |
| 31 | 2691-2710 | CR | TCACCTTGGACAGGAACT | All | 2.1 | 1.0 | 3.6 |
| | | | CC | | | | |
| 32 | 3027-3046 | 3′ UTR | ACAGTCCTCAGTTACAGC | All | 2.2 | 1.1 | 3.2 |
| | | | TC | | | | |

All oligonucleotides contain phosphorothioate internucleotide linkages. Bold nucleotides indicate 2′-MOE nucleotides and plain nucleotides are deoxyribonucleotides.

TABLE 2
19/19 mer siRNAs against mutS homolog 2 (MSH2) transcript variant 1,
mRNA (NCBI Reference Sequence: NM_000251.3)

| 5′ position | | | | |
|---|---|---|---|---|
| of target | SEQ | | SEQ | |
| site in | ID | Sense Strand | ID | Antisense Strand |
| NM_000251.3 | NO. | Sequence (5′→3′) | NO. | Sequence (5′→3′) |
| 326 | 33 | AGUAUAGAGUUGAAGUUUA | 57 | UAAACUUCAACUCUAUACU |
| 516 | 34 | GGUUGGAGUUGGGUAUGUG | 58 | CACAUACCCAACUCCAACC |
| 526 | 35 | GGGUAUGUGGAUUCCAUAC | 59 | GUAUGGAAUCCACAUACCC |
| 842 | 36 | CACUGUCUGCGGUAAUCAA | 60 | UUGAUUACCGCAGACAGUG |
| 1170 | 37 | AGAUUUACUUCGUCGAUUC | 61 | GAAUCGACGAAGUAAAUCU |
| 1228 | 38 | GCAGCAAACUUACAAGAUU | 62 | AAUCUUGUAAGUUUGCUGC |
| 1261 | 39 | CAGGGUAUAAAUCAACUAC | 63 | GUAGUUGAUUUAUACCCUG |
| 1263 | 40 | GGGUAUAAAUCAACUACCU | 64 | AGGUAGUUGAUUUAUACCC |
| 1594 | 41 | GGAUAUUACUUUCGUGUAA | 65 | UUACACGAAAGUAAUAUCC |
| 1629 | 42 | AGUCCUUCGUAACAAUAAA | 66 | UUUAUUGUUACGAAGGACU |
| 1635 | 43 | UCGUAACAAUAAAAACUUU | 67 | AAAGUUUUUAUUGUUACGA |
| 1678 | 44 | GGUGUUAAAUUUACCAACA | 68 | UGUUGGUAAAUUUAACACC |
| 1815 | 45 | GACACUCAAUGAUGUGUUA | 69 | UAACACAUCAUUGAGUGUC |
| 1879 | 46 | GCACCUGUUCCAUAUGUAC | 70 | GUACAUAUGGAACAGGUGC |
| 2043 | 47 | CCCCAAUAUGGGAGGUAAA | 71 | UUUACCUCCCAUAUUGGGG |
| 2052 | 48 | GGGAGGUAAAUCAACAUAU | 72 | AUAUGUUGAUUUACCUCCC |
| 2063 | 49 | CAACAUAUAUUCGACAAAC | 73 | GUUUGUCGAAUAUAUGUUG |
| 2084 | 50 | GGGUGAUAGUACUCAUGGC | 74 | GCCAUGAGUACUAUCACCC |
| 2100 | 51 | GGCCCAAAUUGGGUGUUUU | 75 | AAAACACCCAAUUUGGGCC |
| 2317 | 52 | GGGUUAGCAUGGGCUAUAU | 76 | AUAUAGCCCAUGCUAACCC |
| 2329 | 53 | GCUAUAUCAGAAUACAUUG | 77 | CAAUGUAUUCUGAUAUAGC |
| 2496 | 54 | UGUCUGUGAUCAAAGUUUU | 78 | AAAACUUUGAUCACAGACA |
| 2544 | 55 | CCCUAAGCAUGUAAUAGAG | 79 | CUCUAUUACAUGCUUAGGG |
| 2608 | 56 | GGAGAAUCGCAAGGAUAUG | 80 | CAUAUCCUUGCGAUUCUCC |

TABLE 3
19/21 mer siRNAs against mutS homolog 2 (MSH2) transcript variant 1,
mRNA (NCBI Reference Sequence: NM_000251.3)

| 5′ position | | | | |
|---|---|---|---|---|
| of target | SEQ | | SEQ | |
| site in | ID | Sense Strand | ID | Antisense Strand |
| NM_000251.3 | NO. | Sequence (5′→3′) | NO. | Sequence (5′→3′) |
| 326 | 81 | AGUAUAGAGUUGAAGUUUA | 105 | UAAACUUCAACUCUAUACUGA |
| 516 | 82 | GGUUGGAGUUGGGUAUGUA | 106 | UACAUACCCAACUCCAACCUG |
| 526 | 83 | GGGUAUGUGGAUUCCAUAA | 107 | UUAUGGAAUCCACAUACCCAA |
| 842 | 84 | CACUGUCUGCGGUAAUCAA | 108 | UUGAUUACCGCAGACAGUGAU |
| 1170 | 85 | AGAUUUACUUCGUCGAUUA | 109 | UAAUCGACGAAGUAAAUCUUC |
| 1228 | 86 | GCAGCAAACUUACAAGAUA | 110 | UAUCUUGUAAGUUUGCUGCUU |
| 1261 | 87 | CAGGGUAUAAAUCAACUAA | 111 | UUAGUUGAUUUAUACCCUGAU |
| 1263 | 88 | GGGUAUAAAUCAACUACCA | 112 | UGGUAGUUGAUUUAUACCCUG |
| 1594 | 89 | GGAUAUUACUUUCGUGUAA | 113 | UUACACGAAAGUAAUAUCCAA |
| 1629 | 90 | AGUCCUUCGUAACAAUAAA | 114 | UUUAUUGUUACGAAGGACUUU |
| 1635 | 91 | UCGUAACAAUAAAAACUUA | 115 | UAAGUUUUUAUUGUUACGAAG |
| 1678 | 92 | GGUGUUAAAUUUACCAACA | 116 | UGUUGGUAAAUUUAACACCAU |
| 1815 | 93 | GACACUCAAUGAUGUGUUA | 117 | UAACACAUCAUUGAGUGUCUG |
| 1879 | 94 | GCACCUGUUCCAUAUGUAA | 118 | UUACAUAUGGAACAGGUGCUC |
| 2043 | 95 | CCCCAAUAUGGGAGGUAAA | 119 | UUUACCUCCCAUAUUGGGGCC |
| 2052 | 96 | GGGAGGUAAAUCAACAUAA | 120 | UUAUGUUGAUUUACCUCCCAU |
| 2063 | 97 | CAACAUAUAUUCGACAAAA | 121 | UUUUGUCGAAUAUAUGUUGAU |
| 2084 | 98 | GGGUGAUAGUACUCAUGGA | 122 | UCCAUGAGUACUAUCACCCCA |
| 2100 | 99 | GGCCCAAAUUGGGUGUUUA | 123 | UAAACACCCAAUUUGGGCCAU |
| 2317 | 100 | GGGUUAGCAUGGGCUAUAA | 124 | UUAUAGCCCAUGCUAACCCAA |
| 2329 | 101 | GCUAUAUCAGAAUACAUUA | 125 | UAAUGUAUUCUGAUAUAGCCC |
| 2496 | 102 | UGUCUGUGAUCAAAGUUUA | 126 | UAAACUUUGAUCACAGACACC |
| 2544 | 103 | CCCUAAGCAUGUAAUAGAA | 127 | UUCUAUUACAUGCUUAGGGAA |
| 2608 | 104 | GGAGAAUCGCAAGGAUAUA | 128 | UAUAUCCUUGCGAUUCUCCAA |

TABLE 4
Modified siRNAs against mutS homolog 2 (MSH2) transcript variant 1, mRNA
(NCBI Reference Sequence: NM_000251.3)

| 5′ | | | | | | |
|---|---|---|---|---|---|---|
| position | | | | | | |
| of target | SEQ | | SEQ | | VEGFA | HEK3 |
| site in | ID | Sense Strand | ID | Antisense Strand | fold | fold |
| NM_000251.3 | NO. | Sequence (5′→3′) | NO. | Sequence (5′→3′) | change | change |
| 326 | 129 | agUauaGAGuuGaaguUu | 153 | dTAAACUUCAACUcUAUACU | 15.1 | 2.2 |
| | | sa | | gsa | | |
| 516 | 130 | ggUuggAGUugGguauGu | 154 | dTACAUACCCAACuCCAACC | 9.7 | 1.6 |
| | | sa | | usg | | |
| 526 | 131 | ggGuauGUGgaUuccaUa | 155 | dTUAUGGAAUCCAcAUACCC | 3.3 | 1.6 |
| | | sa | | asa | | |
| 842 | 132 | caCuguCUGcgGuaauCa | 156 | dTUGAUUACCGCAgACAGUG | 7.2 | 2.2 |
| | | sa | | asu | | |
| 1170 | 133 | agAuuuACUucGucgaUu | 157 | dTAAUCGACGAAGuAAAUCU | 6.2 | 2.2 |
| | | sa | | usc | | |
| 1228 | 134 | gcAgcaAACuuAcaagAu | 158 | dTAUCUUGUAAGUuUGCUGC | 3.4 | 1.9 |
| | | sa | | usu | | |
| 1261 | 135 | caGgguAUAaaUcaacUa | 159 | dTUAGUUGAUUUAuACCCUG | 2.9 | 1.7 |
| | | sa | | asu | | |
| 1263 | 136 | ggGuauAAAucAacuaCc | 160 | dTGGUAGUUGAUUuAUACCC | 1.6 | 1.4 |
| | | sa | | usg | | |
| 1594 | 137 | ggAuauUACuuUcgugUa | 161 | dTUACACGAAAGUaAUAUCC | 8.6 | 2.3 |
| | | sa | | asa | | |
| 1629 | 138 | agUccuUCGuaAcaauAa | 162 | dTUUAUUGUUACGaAGGACU | 7.8 | 2.3 |
| | | sa | | usu | | |
| 1635 | 139 | ucGuaaCAAuaAaaacUu | 163 | dTAAGUUUUUAUUgUUACGA | 7.4 | 2.5 |
| | | sa | | asg | | |
| 1678 | 140 | ggUguuAAAuuUaccaAc | 164 | dTGUUGGUAAAUUuAACACC | 7.7 | 2.4 |
| | | sa | | asu | | |
| 1815 | 141 | gaCacuCAAugAugugUu | 165 | dTAACACAUCAUUgAGUGUC | 12.9 | 2.1 |
| | | sa | | usg | | |
| 1879 | 142 | gcAccuGUUccAuaugUa | 166 | dTUACAUAUGGAAcAGGUGC | 6.8 | 2.1 |
| | | sa | | usc | | |
| 2043 | 143 | ccCcaaUAUggGagguAa | 167 | dTUUACCUCCCAUaUUGGGG | 8.3 | 2.2 |
| | | sa | | csc | | |
| 2052 | 144 | ggGaggUAAauCaacaUa | 168 | dTUAUGUUGAUUUaCCUCCC | 5.1 | 2.1 |
| | | sa | | asu | | |
| 2063 | 145 | caAcauAUAuuCgacaAa | 169 | dTUUUGUCGAAUAuAUGUUG | 4.0 | 1.8 |
| | | sa | | asu | | |
| 2084 | 146 | ggGugaUAGuaCucauGg | 170 | dTCCAUGAGUACUaUCACCC | 3.9 | 1.6 |
| | | sa | | csa | | |
| 2100 | 147 | ggCccaAAUugGguguUu | 171 | dTAAACACCCAAUuUGGGCC | 4.6 | 2.0 |
| | | sa | | asu | | |
| 2317 | 148 | ggGuuaGCAugGgcuaUa | 172 | dTUAUAGCCCAUGcUAACCC | 9.2 | 2.1 |
| | | sa | | asa | | |
| 2329 | 149 | gcUauaUCAgaAuacaUu | 173 | dTAAUGUAUUCUGaUAUAGC | 4.3 | 1.7 |
| | | sa | | csc | | |
| 2496 | 150 | ugUcugUGAucAaaguUu | 174 | dTAAACUUUGAUCaCAGACA | 8.6 | 2.1 |
| | | sa | | csc | | |
| 2544 | 151 | ccCuaaGCAugUaauaGa | 175 | dTUCUAUUACAUGcUUAGGG | 6.0 | 2.2 |
| | | sa | | asa | | |
| 2608 | 152 | ggAgaaUCGcaAggauAu | 176 | dTAUAUCCUUGCGaUUCUCC | 4.8 | 1.8 |
| | | sa | | asa | | |

dN: DNA residues
N: RNA residues
n: 2′-O-methyl residues
s: phosphorothioate backbone modification

TABLE 5
Target Site Sequences of mutS homolog 2 (MSH2)
transcript variant 1, mRNA (NCBI Reference
Sequence: NM_000251.3)

| | 5′ position | |
|---|---|---|
| | of target | |
| SEQ | site in | Target Site Sequence |
| ID NO. | NM_000251.3 | (5′→3′) |
| 177 | 326 | UCAGUAUAGAGUUGAAGUUUAUA |
| 178 | 516 | CAGGUUGGAGUUGGGUAUGUGGA |
| 179 | 526 | UUGGGUAUGUGGAUUCCAUACAG |
| 180 | 842 | AUCACUGUCUGCGGUAAUCAAGU |
| 181 | 1170 | GAAGAUUUACUUCGUCGAUUCCC |
| 182 | 1228 | AAGCAGCAAACUUACAAGAUUGU |
| 183 | 1261 | AUCAGGGUAUAAAUCAACUACCU |
| 184 | 1263 | CAGGGUAUAAAUCAACUACCUAA |
| 185 | 1594 | UUGGAUAUUACUUUCGUGUAACC |
| 186 | 1629 | AAAGUCCUUCGUAACAAUAAAAA |
| 187 | 1635 | CUUCGUAACAAUAAAAACUUUAG |
| 188 | 1678 | AUGGUGUUAAAUUUACCAACAGC |
| 189 | 1815 | CAGACACUCAAUGAUGUGUUAGC |
| 190 | 1879 | GAGCACCUGUUCCAUAUGUACGA |
| 191 | 2043 | GGCCCCAAUAUGGGAGGUAAAUC |
| 192 | 2052 | AUGGGAGGUAAAUCAACAUAUAU |
| 193 | 2063 | AUCAACAUAUAUUCGACAAACUG |
| 194 | 2084 | UGGGGUGAUAGUACUCAUGGCCC |
| 195 | 2100 | AUGGCCCAAAUUGGGUGUUUUGU |
| 196 | 2317 | UUGGGUUAGCAUGGGCUAUAUCA |
| 197 | 2329 | GGGCUAUAUCAGAAUACAUUGCA |
| 198 | 2496 | GGUGUCUGUGAUCAAAGUUUUGG |
| 199 | 2544 | UUCCCUAAGCAUGUAAUAGAGUG |
| 200 | 2608 | UUGGAGAAUCGCAAGGAUAUGAU |

Antisense Oligonucleotides and siRNAs for PMS2

Provided herein are siRNAs specific for an mRNA sequence of a PMS1 homolog 2, mismatch repair system component (PMS2) gene (e.g., NCBI Reference Sequence: NM_000535.7, or any other mRNA isoform of a PMS2 gene). In some embodiments, the siRNAs comprise sequence complementarity to any one of the target nucleic acid sequences set forth in SEQ ID NO: 373 to 396. Exemplary antisense molecules against PMS1 homolog 2, mismatch repair system component (PMS2) mRNA and corresponding target nucleotide range are described in Table 6 below. In some embodiments, the siRNAs comprise any one of the matched antisense strand and sense strand pairs set forth in Tables 7-9. Where provided, values “VEGFA fold change,” “HEK3 fold change,” and “RNF2 fold change” indicate the amount of prime editing observed at the respective locus when the given antisense molecule or siRNA pair is administered, in accordance with Example 5, below, with in an vitro-transcribed pegRNA configured to edit that locus and an in vitro-transcribed prime editor, compared to co-administration with an antisense molecule or siRNA pair having a random scrambled sequence. The VEGFA and RNF2 pegRNAs each encode a one-nucleotide substitution, and the HEK3 pegRNA encodes a 3-nucleotide insertion.

In some embodiments, these siRNAs comprise a phosphorothioate internucleotide bond, a methylphosphonate internucleotide bond, and/or a triazole internucleotide bond. Any of these siRNAs may comprise a 2′-O-methoxyethyl oligonucleotide (2′MOE), a 2′-O-methylated nucleoside (2′OMe), a 2′-fluoro oligonucleotide (2′F), a arabino nucleotide (ANA), a 2′-F arabino nucleotide (FANA), a phosphorodiamidate morpholino oligonucleotide (PMO), a peptide nucleic acid (PNA), a phosphorothioate bond (PS), a locked nucleic acid (LNA), a hydrophobic moiety, a naphthyl modifier, or a cholesterol moiety. In some embodiments, these siRNAs are modified with a cholesterol, a dialkyl lipid, or GalNAc. In certain embodiments, these siRNAs are chemically modified with poly-ethylene glycol (PEG). In some embodiments, these siRNAs comprise a 5′ end cap. In some embodiments, these siRNAs may comprise a 3′ end cap. Any of these siRNAs may comprise a DNA nucleotide (e.g., thymine). In certain embodiments, these siRNAs comprise a DNA nucleoside (e.g., thymidine).

Also provided herein are antisense oligonucleotides (ASOs) specific for an mRNA sequence of the PMS1 homolog 2, mismatch repair system component (PMS2) gene. In some embodiments, the ASOs comprise a nucleic acid sequence set forth in SEQ ID NOs: 201-228.

In some embodiments, these ASOs comprise an antisense strand comprising deoxyribonucleotides and/or ribonucleotides. In some embodiments, these ASOs comprise an antisense strand comprising at least five ribonucleotides at the 5′ end of the siRNA antisense strand. In some embodiments, these ASOs comprise an antisense strand comprising at least five ribonucleotides at the 3′ end of the siRNA antisense strand. In some embodiments, the ASOs comprise an antisense strand (5′ to 3′) comprising deoxyribonucleotides from nucleotide position 6 to nucleotide position 15. In some embodiments, these ASOs comprise a chemical modification. In certain embodiments, these ASOs comprise a phosphorothioate internucleotide bond, a methylphosphonate internucleotide bond, and/or a triazole internucleotide bond. In some embodiments, these ASOs comprise a 2′-O-methoxyethyl oligonucleotide (2′MOE), a 2′-O-methylated nucleoside (2′OMe), a 2′-fluoro oligonucleotide (2′F), an arabino nucleotide (ANA), a 2′-F arabino nucleotide (FANA), a phosphorodiamidate morpholino oligonucleotide (PMO), a peptide nucleic acid (PNA), a phosphorothioate bond (PS), a locked nucleic acid (LNA), a hydrophobic moiety, a naphthyl modifier, or a cholesterol moiety. In some embodiments, these ASOs are modified with a cholesterol, a dialkyl lipid, or GalNAc. In certain embodiments, these ASOs are chemically modified with poly-ethylene glycol (PEG). In some embodiments, these ASOs comprise a 5′ end cap. Any of these ASOs may comprise a 3′ end cap.

TABLE 6
Antisense molecules against PMS1 homolog 2, mismatch repair system component
(PMS2) mRNA (NCBI Reference Sequence: NM_000535.7)

| | | | Antisense | | | | |
|---|---|---|---|---|---|---|---|
| SEQ | Target | | Strand | | VEGFA | HEK3 | RNF2 |
| ID | Nucleotide | Target | Sequence | Isoform | fold | fold | fold |
| NO. | Range | location | (5′→3′) | homology | change | change | change |
| 201 | 51-70 | CR | CCTTAGCAG | v1, 4, 5, | 1.9 | 1.2 | 1.5 |
| | | | GTTCTGTAC | 10, 13 | | | |
| | | | TC | | | | |
| 202 | 118-137 | CR | CTTAGACTC | v1, 2, 4, 5, | 1.6 | 1.4 | 1.3 |
| | | | AGTACCACC | 6, 8, 10, | | | |
| | | | TG | 11, 12, 13, | | | |
| | | | | 14, x1, x2 | | | |
| 203 | 388-407 | CR | TGGCAGGTA | v1, 2, 3, 4, | 1.7 | 1.1 | 1.1 |
| | | | GAAATGGTG | 5, 7, 8, 9, | | | |
| | | | AC | 10, 11, 12, | | | |
| | | | | 13, 14 | | | |
| 204 | 485-504 | CR | GCTGACTGT | v1, 2, 3, 4, | 0.9 | 1.1 | 1.4 |
| | | | GGTCCCTCT | 5, 7, 8, 9, | | | |
| | | | GG | 10, 11, 12, | | | |
| | | | | 13, 14, | | | |
| | | | | x1, x2 | | | |
| 205 | 567-586 | CR | GGACCATTT | v1, 2, 3, 4, | 1.6 | 1.1 | 0.7 |
| | | | TGGCATACT | 5, 6, 7, 8, | | | |
| | | | CC | 9, 13, 14 | | | |
| 206 | 605-624 | CR | GATGCCTGC | v1, 2, 3, 4, | | | |
| | | | TGAAATGAT | 5, 6, 7, 8, | | | |
| | | | AC | 9, 13, 14, | | | |
| | | | | x1 | | | |
| 207 | 632-651 | CR | TCCAAGCTG | v1, 2, 3, 4, | 1.7 | 1.0 | 1.0 |
| | | | ATTGGTGCA | 5, 6, 7, 8, | | | |
| | | | AC | 9, 10, 11, | | | |
| | | | | 13, 14, | | | |
| | | | | x1, x2 | | | |
| 208 | 662-681 | CR | TGTGCATAC | v1, 2, 3, 4, | | | |
| | | | CACAGGCTG | 5, 6, 7, 8, | | | |
| | | | TC | 9, 10, 11, | | | |
| | | | | 13, 14, | | | |
| | | | | x1, x2 | | | |
| 209 | 716-735 | CR | CTGCTTCTG | v1, 2, 3, 4, | 1.7 | 0.9 | 1.5 |
| | | | CCCAAACAC | 5, 6, 7, 8, | | | |
| | | | AG | 9, 10, 11, | | | |
| | | | | 13, 14, | | | |
| | | | | x1, x2 | | | |
| 210 | 862-881 | CR | GAACTCCTT | v1, 2, 3, 4, | 1.7 | 0.9 | 1.2 |
| | | | CCAACTCCA | 5, 6, 7, 8, | | | |
| | | | TG | 9, 10, 11, | | | |
| | | | | 12, 13, | | | |
| | | | | 14, x1 | | | |
| 211 | 1137-1156 | CR | GCTGCTGAC | v1, 2, 4, 3, | 1.4 | 1.0 | 1.4 |
| | | | TGACATTTA | 6, 7, 8, 10, | | | |
| | | | GC | 11, 12, 13, | | | |
| | | | | 14, x1, x2 | | | |
| 212 | 1155-1174 | CR | CTTCAACAT | v1, 2, 3, 4, | | | |
| | | | CCAGCAGTG | 6, 7, 8, 10, | | | |
| | | | GC | 11, 12, 13, | | | |
| | | | | 14, x1, x2 | | | |
| 213 | 1312-1331 | CR | GGCTTGTTC | v1, 2, 3, 4, | | | |
| | | | TCTGTTGTG | 5, 6, 7, 8, | | | |
| | | | TG | 9, 10, 11, | | | |
| | | | | 12, 13, | | | |
| | | | | 14, x1, x2 | | | |
| 214 | 1392-1411 | CR | CACCTGAAG | v1, 2, 3, 4, | 1.1 | 0.9 | 1.4 |
| | | | TGCTAGAAG | 5, 6, 7, 8, | | | |
| | | | AC | 9, 10, 11, | | | |
| | | | | 12, 13, | | | |
| | | | | 14, x1-x3 | | | |
| 215 | 1441-1460 | CR | GAACTCACT | v1, 2, 3, 4, | | | |
| | | | GCCTCTTTC | 5, 6, 7, 8, | | | |
| | | | TG | 9, 10, 11, | | | |
| | | | | 12, 13, 14, | | | |
| | | | | x1-x3 | | | |
| 216 | 1627-1646 | CR | GCTTTCTCC | v1, 2, 3, 4, | 1.1 | 1.0 | 0.7 |
| | | | TGAGAGTCC | 5, 6, 7, 8, | | | |
| | | | AC | 9, 10, 11, | | | |
| | | | | 12, 13, 14, | | | |
| | | | | x1-x3 | | | |
| 217 | 1677-1696 | CR | CCTGGTTTG | v1, 2, 3, 4, | | | |
| | | | AATGGCAGT | 5, 6, 7, 8, | | | |
| | | | CC | 9, 10, 11, | | | |
| | | | | 12, 13, 14, | | | |
| | | | | x1-x3 | | | |
| 218 | 1834-1853 | CR | GCTACATCA | v1, 2, 3, 4, | 0.8 | 1.0 | 0.5 |
| | | | ACCTGAGAG | 5, 6, 7, 8, | | | |
| | | | GC | 9, 10, 11, | | | |
| | | | | 12, 13, 14, | | | |
| | | | | x1-x3 | | | |
| 219 | 1933-1952 | CR | CCTTCACTT | v1, 2, 3, 4, | 1.8 | 1.1 | 0.5 |
| | | | TGCTGTGCT | 5, 6, 7, 8, | | | |
| | | | TC | 9, 10, 11, | | | |
| | | | | 12, 13, 14, | | | |
| | | | | x1-x3 | | | |
| 220 | 2189-2208 | CR | AGGTGCTAT | v1, 2, 3, 4, | 1.2 | 0.8 | 0.9 |
| | | | GAGCCTCTG | 5, 6, 7, 8, | | | |
| | | | CC | 9, 10, 11, | | | |
| | | | | 12, 13, 14, | | | |
| | | | | x1-x3 | | | |
| 221 | 2305-2324 | CR | GCCCTTTCA | v1, 2, 3, 4, | 1.2 | 1.2 | 1.5 |
| | | | GTGACTGGA | 5, 6, 7, 8, | | | |
| | | | GC | 9, 10, 11, | | | |
| | | | | 12, 13, 14, | | | |
| | | | | x1-x3 | | | |
| 222 | 2435-2454 | CR | GGCAAACAT | v1, 2, 3, 4, | 2.0 | 0.9 | 1.4 |
| | | | CTGCTTGAC | 5, 6, 7, 8, | | | |
| | | | TC | 9, 10, 11, | | | |
| | | | | 12, 13, 14, | | | |
| | | | | x1-x3 | | | |
| 223 | 2442-2461 | CR | CTCTGGAGG | v1, 2, 3, 4, | | | |
| | | | CAAACATCT | 5, 6, 7, 8, | | | |
| | | | GC | 9, 10, 11, | | | |
| | | | | 12, 13, | | | |
| | | | | 12, x1-x3 | | | |
| 224 | 2475-2494 | CR | GAGCAGTCC | v1, 2, 3, 4, | 1.5 | 1.1 | 1.8 |
| | | | CAATCATCA | 5, 6, 7, 8, | | | |
| | | | CC | 9, 10, 11, | | | |
| | | | | 12, 13, | | | |
| | | | | 14, x1-x3 | | | |
| 225 | 2566-2585 | CR | TGTCTCATG | v1, 2, 3, 4, | 1.0 | 1.1 | 0.8 |
| | | | GTTGGCCTT | 5, 6, 7, 8, | | | |
| | | | CC | 9, 10, 11, | | | |
| | | | | 12, 13, | | | |
| | | | | 14, x1-x3 | | | |
| 226 | 2595-2614 | CR | TCTGAGAAA | v1, 2, 3, 4, | 1.1 | 1.0 | 1.1 |
| | | | TGACACCCA | 5 ,6, 7, 8, | | | |
| | | | GG | 9, 10, 11, | | | |
| | | | | 12, 13, | | | |
| | | | | 14, x1-x3 | | | |
| 227 | 2775-2794 | 3′ UTR | GCAAGCAAT | v1, 2, 3, 4, | 1.9 | 0.8 | 0.7 |
| | | | GCTCCATCT | 5, 6, 7, 8, | | | |
| | | | GG | 9, 10, 11, | | | |
| | | | | 12, 13, | | | |
| | | | | 14, x1-x3 | | | |
| 228 | 2827-2846 | 3′ UTR | CTTGCCTGG | v1, 2, 3, 4, | 1.9 | 1.2 | 1.5 |
| | | | ACACACACA | 5, 6, 7, 8, | | | |
| | | | CA | 9, 10, 11, | | | |
| | | | | 12, 13, | | | |
| | | | | 14, x1-x3 | | | |

All oligonucleotides contain phosphorothioate internucleotide linkages. Bold nucleotides indicate 2′-MOE nucleotides and plain nucleotides are deoxyribonucleotides.

TABLE 7
19/19 mer siRNAs against PMS1 homolog 2, mismatch repair system component
(PMS2) mRNA (NCBI Reference Sequence: NM_000535.7)

| 5′ position | | | | |
|---|---|---|---|---|
| of target | SEQ | | SEQ | |
| site in | ID | Sense Strand | ID | Antisense Strand |
| NM_000535.7 | NO. | Sequence (5′→3′) | NO. | Sequence (5′→3′) |
| 424 | 229 | GGAACUCGACUGAUGUUUG | 253 | CAAACAUCAGUCGAGUUCC |
| 952 | 230 | GAGGUCUACCACAUGUAUA | 254 | UAUACAUGUGGUAGACCUC |
| 954 | 231 | GGUCUACCACAUGUAUAAU | 255 | AUUAUACAUGUGGUAGACC |
| 957 | 232 | CUACCACAUGUAUAAUCGA | 256 | UCGAUUAUACAUGUGGUAG |
| 960 | 233 | CCACAUGUAUAAUCGACAC | 257 | GUGUCGAUUAUACAUGUGG |
| 1374 | 234 | ACAGAAAAGGGGUAUGCUG | 258 | CAGCAUACCCCUUUUCUGU |
| 1382 | 235 | GGGGUAUGCUGUCUUCUAG | 259 | CUAGAAGACAGCAUACCCC |
| 1384 | 236 | GGUAUGCUGUCUUCUAGCA | 260 | UGCUAGAAGACAGCAUACC |
| 1580 | 237 | GCGAGUAUGCGGCCAGCUC | 261 | GAGCUGGCCGCAUACUCGC |
| 1702 | 238 | ACCGGAUGUAAAUUUCGAG | 262 | CUCGAAAUUUACAUCCGGU |
| 1704 | 239 | CGGAUGUAAAUUUCGAGUU | 263 | AACUCGAAAUUUACAUCCG |
| 1730 | 241 | AGCCAACUAAUCUCGCAAC | 265 | GUUGCGAGAUUAGUUGGCU |
| 1741 | 242 | CUCGCAACCCCAAACACAA | 266 | UUGUGUUUGGGGUUGCGAG |
| 1748 | 243 | CCCCAAACACAAAGCGUUU | 267 | AAACGCUUUGUGUUUGGGG |
| 1749 | 244 | CCCAAACACAAAGCGUUUU | 268 | AAAACGCUUUGUGUUUGGG |
| 1752 | 245 | AAACACAAAGCGUUUUAAA | 269 | UUUAAAACGCUUUGUGUUU |
| 1753 | 246 | AACACAAAGCGUUUUAAAA | 270 | UUUUAAAACGCUUUGUGUU |
| 1824 | 247 | GGACAUGUCAGCCUCUCAG | 271 | CUGAGAGGCUGACAUGUCC |
| 3230 | 248 | GGCUGGACAUAGUUUAGUC | 272 | GACUAAACUAUGUCCAGCC |
| 3259 | 249 | AACCCUUAAUGAUAAUUAA | 273 | UUAAUUAUCAUUAAGGGUU |
| 3413 | 250 | GGGUCACUAUUUGAAACAU | 274 | AUGUUUCAAAUAGUGACCC |
| 3483 | 251 | GAGUUUGAUUUCCCAUAAU | 275 | AUUAUGGGAAAUCAAACUC |
| 5039 | 252 | GGGACUAAUUUACAUACUG | 276 | CAGUAUGUAAAUUAGUCCC |

TABLE 8
19/21 mer siRNAs against PMS1 homolog 2, mismatch repair system component
(PMS2) mRNA (NCBI Reference Sequence: NM_000535.7)

| 5′ | | 5′ | | | |
|---|---|---|---|---|---|
| position | | position | | | |
| of target | SEQ | of target | | SEQ | |
| site in | ID | site in | Sense Strand | ID | Antisense Strand |
| NM_000535.7 | NO. | NM_000535.7 | Sequence (5′→3′) | NO. | Sequence (5′→3′) |
| 424 | 277 | 424 | GGAACUCGACUGAUGUUUA | 301 | UAAACAUCAGUCGAGUUCCAA |
| 952 | 278 | 952 | GAGGUCUACCACAUGUAUA | 302 | UAUACAUGUGGUAGACCUCAU |
| 954 | 279 | 954 | GGUCUACCACAUGUAUAAA | 303 | UUUAUACAUGUGGUAGACCUC |
| 957 | 280 | 957 | CUACCACAUGUAUAAUCGA | 304 | UCGAUUAUACAUGUGGUAGAC |
| 960 | 281 | 960 | CCACAUGUAUAAUCGACAA | 305 | UUGUCGAUUAUACAUGUGGUA |
| 1374 | 282 | 1374 | ACAGAAAAGGGGUAUGCUA | 306 | UAGCAUACCCCUUUUCUGUCC |
| 1382 | 283 | 1382 | GGGGUAUGCUGUCUUCUAA | 307 | UUAGAAGACAGCAUACCCCUU |
| 1384 | 284 | 1384 | GGUAUGCUGUCUUCUAGCA | 308 | UGCUAGAAGACAGCAUACCCC |
| 1580 | 285 | 1580 | GCGAGUAUGCGGCCAGCUA | 309 | UAGCUGGCCGCAUACUCGCUG |
| 1702 | 286 | 1702 | ACCGGAUGUAAAUUUCGAA | 310 | UUCGAAAUUUACAUCCGGUAU |
| 1704 | 287 | 1704 | CGGAUGUAAAUUUCGAGUA | 311 | UACUCGAAAUUUACAUCCGGU |
| 1730 | 289 | 1730 | AGCCAACUAAUCUCGCAAA | 313 | UUUGCGAGAUUAGUUGGCUGA |
| 1741 | 290 | 1741 | CUCGCAACCCCAAACACAA | 314 | UUGUGUUUGGGGUUGCGAGAU |
| 1748 | 291 | 1748 | CCCCAAACACAAAGCGUUA | 315 | UAACGCUUUGUGUUUGGGGUU |
| 1749 | 292 | 1749 | CCCAAACACAAAGCGUUUA | 316 | UAAACGCUUUGUGUUUGGGGU |
| 1752 | 293 | 1752 | AAACACAAAGCGUUUUAAA | 317 | UUUAAAACGCUUUGUGUUUGG |
| 1753 | 294 | 1753 | AACACAAAGCGUUUUAAAA | 318 | UUUUAAAACGCUUUGUGUUUG |
| 1824 | 295 | 1824 | GGACAUGUCAGCCUCUCAA | 319 | UUGAGAGGCUGACAUGUCCUG |
| 3230 | 296 | 3230 | GGCUGGACAUAGUUUAGUA | 320 | UACUAAACUAUGUCCAGCCAG |
| 3259 | 297 | 3259 | AACCCUUAAUGAUAAUUAA | 321 | UUAAUUAUCAUUAAGGGUUGA |
| 3413 | 298 | 3413 | GGGUCACUAUUUGAAACAA | 322 | UUGUUUCAAAUAGUGACCCCU |
| 3483 | 299 | 3483 | GAGUUUGAUUUCCCAUAAA | 323 | UUUAUGGGAAAUCAAACUCUU |
| 5039 | 300 | 5039 | GGGACUAAUUUACAUACUA | 324 | UAGUAUGUAAAUUAGUCCCAC |

TABLE 9
Modified siRNAs against PMS1 homolog 2, mismatch repair system component
(PMS2) mRNA (NCBI Reference Sequence: NM_000535.7)

| 5′ | | | | | | |
|---|---|---|---|---|---|---|
| position | | | | | | |
| of target | SEQ | | SEQ | | VEGFA | HEK3 |
| site in | ID | Sense Strand | ID | Antisense Strand | fold | fold |
| NM_000535.7 | NO. | Sequence (5′→3′) | NO. | Sequence (5′→3′) | change | change |
| 424 | 325 | ggAacuCGAcuGaugu | 349 | dTAAACAUCAGUCgAGUUCC | 6.3 | 2.8 |
| | | Uusa | | asa | | |
| 952 | 326 | gaGgucUACcaCaugu | 350 | dTAUACAUGUGGUaGACCUC | 4.6 | 2.5 |
| | | Ausa | | asu | | |
| 954 | 327 | ggUcuaCCAcaUguau | 351 | dTUUAUACAUGUGgUAGACC | 4.3 | 2.2 |
| | | Aasa | | usc | | |
| 957 | 328 | cuAccaCAUguAuaau | 352 | dTCGAUUAUACAUgUGGUAG | 5.0 | 2.8 |
| | | Cgsa | | asc | | |
| 960 | 329 | ccAcauGUAuaAucga | 353 | dTUGUCGAUUAUAcAUGUGG | 1.9 | 2.0 |
| | | Casa | | usa | | |
| 1374 | 330 | acAgaaAAGggGuaug | 354 | dTAGCAUACCCCUuUUCUGU | 4.6 | 2.9 |
| | | Cusa | | csc | | |
| 1382 | 331 | ggGguaUGCugUcuuc | 355 | dTUAGAAGACAGCaUACCCC | 4.9 | 2.9 |
| | | Uasa | | usu | | |
| 1384 | 332 | ggUaugCUGucUucua | 356 | dTGCUAGAAGACAgCAUACC | 4.8 | 2.6 |
| | | Gcsa | | csc | | |
| 1580 | 333 | gcGaguAUGcgGccag | 357 | dTAGCUGGCCGCAuACUCGC | 3.5 | 1.7 |
| | | Cusa | | usg | | |
| 1702 | 334 | acCggaUGUaaAuuuc | 358 | dTUCGAAAUUUACaUCCGGU | 4.6 | 1.9 |
| | | Gasa | | asu | | |
| 1704 | 335 | cgGaugUAAauUucga | 359 | dTACUCGAAAUUUaCAUCCG | 3.5 | 1.4 |
| | | Gusa | | gsu | | |
| 1730 | 337 | agCcaaCUAauCucgc | 361 | dTUUGCGAGAUUAgUUGGCU | 3.2 | 1.8 |
| | | Aasa | | gsa | | |
| 1741 | 338 | cuCgcaACCccAaaca | 362 | dTUGUGUUUGGGGuUGCGAG | 1.9 | 2.4 |
| | | Casa | | asu | | |
| 1748 | 339 | ccCcaaACAcaAagcg | 363 | dTAACGCUUUGUGuUUGGGG | 2.5 | 2.3 |
| | | Uusa | | usu | | |
| 1749 | 340 | ccCaaaCACaaAgcgu | 364 | dTAAACGCUUUGUgUUUGGG | 2.6 | 1.9 |
| | | Uusa | | gsu | | |
| 1752 | 341 | aaAcacAAAgcGuuuu | 365 | dTUUAAAACGCUUuGUGUUU | 3.5 | 2.6 |
| | | Aasa | | gsg | | |
| 1753 | 342 | aaCacaAAGcgUuuua | 366 | dTUUUAAAACGCUuUGUGUU | 4.3 | 2.8 |
| | | Aasa | | usg | | |
| 1824 | 343 | ggAcauGUCagCcucu | 367 | dTUGAGAGGCUGAcAUGUCC | 3.5 | 2.6 |
| | | Casa | | usg | | |
| 3230 | 344 | ggCuggACAuaGuuua | 368 | dTACUAAACUAUGuCCAGCC | 3.0 | 1.1 |
| | | Gusa | | asg | | |
| 3259 | 345 | aaCccuUAAugAuaau | 369 | dTUAAUUAUCAUUaAGGGUU | 2.1 | 1.1 |
| | | Uasa | | gsa | | |
| 3413 | 346 | ggGucaCUAuuUgaaa | 370 | dTUGUUUCAAAUAgUGACCC | 2.4 | 1.1 |
| | | Casa | | csu | | |
| 3483 | 347 | gaGuuuGAUuuCccau | 371 | dTUUAUGGGAAAUcAAACUC | 3.5 | 1.1 |
| | | Aasa | | usu | | |
| 5039 | 348 | ggGacuAAUuuAcaua | 372 | dTAGUAUGUAAAUuAGUCCC | 2.3 | 0.9 |
| | | Cusa | | asc | | |

dN: DNA residues
N: RNA residues
n: 2′-O-methyl residues
s: phosphorothioate backbone modification

TABLE 10
Target Site Sequences of PMS1 homolog 2, mismatch
repair system component (PMS2) mRNA (NCBI
Reference Sequence: NM_000535.7)

| | 5′ position | |
|---|---|---|
| | of target | |
| SEQ | site in | Target Site Sequence |
| ID NO. | NM_000535.7 | (5′→3′) |
| 373 | 424 | UUGGAACUCGACUGAUGUUUGAU |
| 374 | 952 | AUGAGGUCUACCACAUGUAUAAU |
| 375 | 954 | GAGGUCUACCACAUGUAUAAUCG |
| 376 | 957 | GUCUACCACAUGUAUAAUCGACA |
| 377 | 960 | UACCACAUGUAUAAUCGACACCA |
| 378 | 1374 | GGACAGAAAAGGGGUAUGCUGUC |
| 379 | 1382 | AAGGGGUAUGCUGUCUUCUAGCA |
| 380 | 1384 | GGGGUAUGCUGUCUUCUAGCACU |
| 381 | 1580 | CAGCGAGUAUGCGGCCAGCUCCC |
| 382 | 1702 | AUACCGGAUGUAAAUUUCGAGUU |
| 383 | 1704 | ACCGGAUGUAAAUUUCGAGUUUU |
| 384 | 1705 | CCGGAUGUAAAUUUCGAGUUUUG |
| 385 | 1730 | UCAGCCAACUAAUCUCGCAACCC |
| 386 | 1741 | AUCUCGCAACCCCAAACACAAAG |
| 387 | 1748 | AACCCCAAACACAAAGCGUUUUA |
| 388 | 1749 | ACCCCAAACACAAAGCGUUUUAA |
| 389 | 1752 | CCAAACACAAAGCGUUUUAAAAA |
| 390 | 1753 | CAAACACAAAGCGUUUUAAAAAA |
| 391 | 1824 | CAGGACAUGUCAGCCUCUCAGGU |
| 392 | 3230 | CUGGCUGGACAUAGUUUAGUCUA |
| 393 | 3259 | UCAACCCUUAAUGAUAAUUAAAU |
| 394 | 3413 | AGGGGUCACUAUUUGAAACAUUC |
| 395 | 3483 | AAGAGUUUGAUUUCCCAUAAUUU |
| 396 | 5039 | GUGGGACUAAUUUACAUACUGUA |

Antisense Oligonucleotides and siRNAs for MSH6

Provided herein are siRNAs specific for an mRNA sequence of a mutS homolog 6 (MSH6) gene (e.g., MSH6 transcript variant 1, (NCBI Reference Sequence: NM_000179.3)). In certain embodiments the siRNAs comprise sequence complementarity to any one of the target nucleic acid sequences set forth in SEQ ID NO: 585 to 608. Exemplary antisense molecules against mutS homolog 6 (MSH6), transcript variant 1, mRNA and corresponding target nucleotide ranges are described in Table 11 below. In certain embodiments, the siRNAs comprise any one of the matched antisense strand and sense strand pairs set forth in Tables 12-14. Where provided, values “VEGFA fold change,” “HEK3 fold change,” and “RNF2 fold change” indicate the amount of prime editing observed at the respective locus when the given antisense molecule or siRNA pair is administered, in accordance with Example 5, below, with in an vitro-transcribed pegRNA configured to edit that locus and an in vitro-transcribed prime editor, compared to co-administration with an antisense molecule or siRNA pair having a random scrambled sequence. The VEGFA and RNF2 pegRNAs each encode a one-nucleotide substitution, and the HEK3 pegRNA encodes a 3-nucleotide insertion.

In some embodiments, these siRNAs comprise a phosphorothioate internucleotide bond, a methylphosphonate internucleotide bond, and/or a triazole internucleotide bond. Any of these siRNAs may comprise a 2′-O-methoxyethyl oligonucleotide (2′MOE), a 2′-O-methylated nucleoside (2′OMe), a 2′-fluoro oligonucleotide (2′F), a arabino nucleotide (ANA), a 2′-F arabino nucleotide (FANA), a phosphorodiamidate morpholino oligonucleotide (PMO), a peptide nucleic acid (PNA), a phosphorothioate bond (PS), a locked nucleic acid (LNA), a hydrophobic moiety, a naphthyl modifier, or a cholesterol moiety. In some embodiments, these siRNAs are modified with a cholesterol, a dialkyl lipid, or GalNAc. In certain embodiments, these siRNAs are chemically modified with poly-ethylene glycol (PEG). In some embodiments, these siRNAs comprise a 5′ end cap. In some embodiments, these siRNAs may comprise a 3′ end cap. Any of these siRNAs may comprise a DNA nucleotide (e.g., thymine). In certain embodiments, these siRNAs comprise a DNA nucleoside (e.g., thymidine).

Also provided are antisense oligonucleotides (ASOs) specific for an mRNA sequence of the mutS homolog 6 (MSH6) gene. In some embodiments, the ASOs comprise a nucleic acid sequence set forth in SEQ ID NOs: 397-440.

In some embodiments, these ASOs comprise an antisense strand comprising deoxyribonucleotides and/or ribonucleotides. In some embodiments, these ASOs comprise an antisense strand comprising at least five ribonucleotides at the 5′ end of the siRNA antisense strand. In some embodiments, these ASOs comprise an antisense strand comprising at least five ribonucleotides at the 3′ end of the siRNA antisense strand. In some embodiments, the ASOs comprise an antisense strand (5′ to 3′) comprising deoxyribonucleotides from nucleotide position 6 to nucleotide position 15. In some embodiments, these ASOs comprise a chemical modification. In certain embodiments, these ASOs comprise a phosphorothioate internucleotide bond, a methylphosphonate internucleotide bond, and/or a triazole internucleotide bond. In some embodiments, these ASOs comprise a 2f-O-methoxyethyl oligonucleotide (2MOE), al methylated nucleoside (2′OMe), a 2′-fluoro oligonucleotide (2′F), an arabino nucleotide (ANA), a 2′-F arabino nucleotide (FANA), a phosphorodiamidate morpholino oligonucleotide (PMO), a peptide nucleic acid (PNA), a phosphorothioate bond (PS), a locked nucleic acid (LNA), a hydrophobic moiety, a naphthyl modifier, or a cholesterol moiety. In some embodiments, these ASOs are modified with a cholesterol, a dialkyl lipid, or GalNAc. In certain embodiments, these ASOs are chemically modified with poly-ethylene glycol (PEG). In some embodiments, these ASOs comprise a 5′ end cap. Any of these ASOs may comprise a 3′ end cap.

TABLE 11
Antisense molecules against mutS homolog 6 (MSH6), transcript variant 1,
mRNA (NCBI Reference Sequence: NM_000179.3)

| SEQ | Target | | | | VEGFA | HEK3 | RNF2 |
|---|---|---|---|---|---|---|---|
| ID | Nucleotide | Target | Antisense Strand | Isoform | fold | fold | fold |
| NO. | Range | location | Sequence (5′→3′) | homology | change | change | change |
| 397 | 137-156 | CR | CCTTGTTGGCATCACTCAG | v1-3, x1, x4 | 1.6 | 1.0 | 4.8 |
| | | | C | | | | |
| 398 | 343-362 | CR | TGAGAAGTCACAACTGGTG | v1, 3, X1, X3, X4 | | | |
| | | | G | | | | |
| 399 | 361-380 | CR | CCAAACCAAATCTCCTGGT | v1, 3, 4, x1-x4 | 2.7 | 0.9 | 9.2 |
| | | | G | | | | |
| 400 | 403-422 | CR | GTAAACCAGACAAGGCCAC | v1, 3, 4, x1-x4 | | | |
| | | | C | | | | |
| 401 | 452-471 | CR | GGACTGATTTCCCTTTCTC | v1, 3, 4, x1-x4 | 2.5 | 0.8 | 11.3 |
| | | | G | | | | |
| 402 | 591-610 | CR | CTCAGTATTTCAGGCTTTG | v1, 4, x1-x3 | 2.3 | 0.8 | 8.1 |
| | | | C | | | | |
| 403 | 692-711 | CR | TCTCTTCTTCCTCTTCTGG | v1, 4, x1-x3 | 1.4 | 0.8 | 2.1 |
| | | | C | | | | |
| 404 | 701-720 | CR | CTACCTCCATCTCTTCTTC | v1, x1-x3 | | | |
| | | | C | | | | |
| 405 | 767-786 | CR | GCTGTACTTCCTCTTCACT | v1-4, x1-x4 | 1.6 | 0.8 | 5.6 |
| | | | C | | | | |
| 406 | 776-795 | CR | GTGTCTTAGGCTGTACTTC | v1-4, x1-x4 | | | |
| | | | C | | | | |
| 407 | 850-869 | CR | GCCACCAATGTCACTCTCA | v1-4, x1-x4 | | | |
| | | | G | | | | |
| 408 | 899-918 | CR | CATCACTGCTTCCTTCCTC | v1-4, x1-x4 | 0.7 | 0.9 | 3.1 |
| | | | C | | | | |
| 409 | 937-956 | CR | GCCTTCACTCTCACTATCC | v1-4, x1-x4 | | | |
| | | | C | | | | |
| 410 | 1048-1067 | CR | TGCTTGTTTGGTGGCTGAG | v1-4, x1-x4 | | | |
| | | | G | | | | |
| 411 | 1149-1168 | CR | CTACTGTCATCACCACCTC | v1-4, x1-x4 | 1.6 | 0.8 | 2.5 |
| | | | C | | | | |
| 412 | 1222-1241 | CR | CCTCCTGTGCTCATCTCTT | v1-4, x1-x4 | 1.9 | 1.0 | 4.9 |
| | | | C | | | | |
| 413 | 1270-1289 | CR | CTCAGGCACATAGAGTGTA | v1-4, x1-x4 | | | |
| | | | G | | | | |
| 414 | 1311-1330 | CR | CACCACTTCCTCATCCCAG | v1-4, x1-x4 | | | |
| | | | G | | | | |
| 415 | 1359-1378 | CR | CCCACCTTGTAACAGATGA | v1-4, x1-x4 | 2.5 | 1.0 | 8.5 |
| | | | C | | | | |
| 416 | 1391-1410 | CR | GAGCATCCATGTGGTACAG | v1-4, x1-x4 | | | |
| | | | C | | | | |
| 417 | 1536-1555 | CR | GGAGTCTCAGTCTGTTCCA | v1-4, x1-x4, | 2.0 | 0.7 | 3.5 |
| | | | C | | | | |
| 418 | 1605-1624 | CR | TCCCTCCTCACCACTCTAT | v1-4, x1-x4 | 2.3 | 0.9 | 3.2 |
| | | | C | | | | |
| 419 | 1642-1661 | CR | GTAAGTCTGTGTACCCTTG | v1-4, x1-x4 | | | |
| | | | G | | | | |
| 420 | 1655-1674 | CR | CTTCCAGCACACTGTAAGT | v1-4, x1-x4 | 2.3 | 0.9 | 4.3 |
| | | | C | | | | |
| 421 | 2093-2112 | CR | CTCCTGGTGTCAACCCAAT | v1-4, x1-x4 | 2.9 | 0.9 | 6.5 |
| | | | G | | | | |
| 422 | 2257-2276 | CR | GGCTTTGGTGAAGATAGCA | v1-4, x1-x4 | | | |
| | | | C | | | | |
| 423 | 2284-2303 | CR | CACTGCATCTAGCACCATT | v1-4, x1-x4 | 1.2 | 0.9 | 5.4 |
| | | | C | | | | |
| 424 | 2367-2386 | CR | TGGCAAGTATCAACCCTCT | v1-4, x1-x4 | | | |
| | | | C | | | | |
| 425 | 2404-2423 | CR | AAGCCATTGCTTTAGGAGC | v1-4, x1-x4 | | | |
| | | | C | | | | |
| 426 | 2475-2494 | CR | GGCACAACCATGAGGTCTT | v1-4, x1-x4 | 2.8 | 0.9 | 5.4 |
| | | | C | | | | |
| 427 | 2582-2601 | CR | GGTGGTTCTGACTCTTCAG | v1-4, x1-x4 | 1.0 | 0.6 | 1.6 |
| | | | G | | | | |
| 428 | 2821-2840 | CR | GTCAAAGGCTGTATCCCAT | v1-4, x1-x4 | 1.9 | 1.0 | 5.1 |
| | | | C | | | | |
| 429 | 2934-2953 | CR | AGGTATTCCAGGAGGCTCT | v1-4, x1-x4 | | | |
| | | | G | | | | |
| 430 | 3161-3180 | CR | CCTTCAATGATACATCCCT | v1-4, x1-x4 | 2.9 | 1.0 | 6.2 |
| | | | C | | | | |
| 431 | 3221-3240 | CR | CAGCAGACTGCCAGTCCTT | v1-4, x1-x4 | | | |
| | | | G | | | | |
| 432 | 3296-3315 | CR | GACACATAGGACCATCACC | v1-4, x1-x4 | 2.0 | 0.9 | 5.5 |
| | | | C | | | | |
| 433 | 3435-3454 | CR | TGCTCCTCTTCCTCACAGC | v1-4, x1-x4 | 1.3 | 0.9 | 3.4 |
| | | | C | | | | |
| 434 | 3462-3481 | CR | AGCACACAATAGGCTTTGC | v1-4, x1-x4 | | | |
| | | | C | | | | |
| 435 | 3521-3540 | CR | CTAATAAGCCAGCCTGTCT | v1-4, x1-x4 | 2.3 | 1.0 | 4.3 |
| | | | C | | | | |
| 436 | 3613-3632 | CR | GTCTGAGGCACCAAGTCTA | v1-4, x1-x4 | | | |
| | | | G | | | | |
| 437 | 3700-3719 | CR | CACCAGAGAATGTGCTGTT | v1-4, x1-x4 | | | |
| | | | G | | | | |
| 438 | 3984-4003 | CR | AGCCTTGCTGCATTAAAGC | v1-4, x1-x4 | 2.0 | 0.9 | 5.5 |
| | | | C | | | | |
| 439 | 4091-4110 | CR | CACTAGCCAGGCAAACTTC | v1-4, x1-x4 | 2.7 | 1.0 | 7.0 |
| | | | C | | | | |
| 440 | 4189-4208 | 3′ UTR | GTCAGAAGTCAACTCAAAG | v1-4, x1-x4 | 2.3 | 0.9 | 8.3 |
| | | | C | | | | |

All oligonucleotides contain phosphorothioate internucleotide linkages. Bold nucleotides indicate 2′-MOE nucleotides and plain nucleotides are deoxyribonucleotides.

TABLE 12
19/19 mer siRNA against mutS homolog 6 (MSH6), transcript variant 1, mRNA
(NCBI Reference Sequence: NM_000179.3)

| 5′ position | | | | |
|---|---|---|---|---|
| of target | SEQ | | SEQ | |
| site in | ID | Sense Strand | ID | Antisense Strand |
| NM_000179.3 | NO. | Sequence (5′→3′)) | NO. | Sequence (5′→3′) |
| 807 | 441 | CGAAGUAGCCGCCAAAUAA | 465 | UUAUUUGGCGGCUACUUCG |
| 808 | 442 | GAAGUAGCCGCCAAAUAAA | 466 | UUUAUUUGGCGGCUACUUC |
| 815 | 443 | CCGCCAAAUAAAAAAACGA | 467 | UCGUUUUUUUAUUUGGCGG |
| 1321 | 444 | GGAAGUGGUGGCAGAUUAA | 468 | UUAAUCUGCCACCACUUCC |
| 1511 | 445 | GCAGAAGGGCUAUAAAGUA | 469 | UACUUUAUAGCCCUUCUGC |
| 1566 | 446 | GAGGCACGAUGUAGAAAGA | 470 | UCUUUCUACAUCGUGCCUC |
| 1568 | 447 | GGCACGAUGUAGAAAGAUG | 471 | CAUCUUUCUACAUCGUGCC |
| 1622 | 448 | GGAGAUCUGUAGGAUCAUU | 472 | AAUGAUCCUACAGAUCUCC |
| 1818 | 449 | CGCCAUUGUUCGAGAUUUA | 473 | UAAAUCUCGAACAAUGGCG |
| 1972 | 450 | GGGAUGCAUCCAAAACUUU | 474 | AAAGUUUUGGAUGCAUCCC |
| 2256 | 451 | GGUGCUAUCUUCACCAAAG | 475 | CUUUGGUGAAGAUAGCACC |
| 2299 | 452 | CAGUGACAUUAAACAACUU | 476 | AAGUUGUUUAAUGUCACUG |
| 2372 | 453 | GGUUGAUACUUGCCAUACU | 477 | AGUAUGGCAAGUAUCAACC |
| 2486 | 454 | GGUUGUGCCUGACAAAAUC | 478 | GAUUUUGUCAGGCACAACC |
| 2606 | 455 | CAGCAGGGCUAUAAUGUAU | 479 | AUACAUUAUAGCCCUGCUG |
| 2611 | 456 | GGGCUAUAAUGUAUGAAGA | 480 | UCUUCAUACAUUAUAGCCC |
| 3001 | 457 | GGAUUGGUAGGAACCGUUA | 481 | UAACGGUUCCUACCAAUCC |
| 3038 | 458 | GAAUUUCACCACUCGCAAU | 482 | AUUGCGAGUGGUGAAAUUC |
| 3066 | 459 | GAAUACGAGUUGAAAUCUA | 483 | UAGAUUUCAACUCGUAUUC |
| 3089 | 460 | GAAGGGCUGUAAACGAUAC | 484 | GUAUCGUUUACAGCCCUUC |
| 3134 | 461 | GGCUAAUCUCAUAAAUGCU | 485 | AGCAUUUAUGAGAUUAGCC |
| 3160 | 462 | GGAGGGAUGUAUCAUUGAA | 486 | UUCAAUGAUACAUCCCUCC |
| 3189 | 463 | CGGCGACUGUUCUAUAACU | 487 | AGUUAUAGAACAGUCGCCG |
| 3191 | 464 | GCGACUGUUCUAUAACUUU | 488 | AAAGUUAUAGAACAGUCGC |

TABLE 13
19/21 mer siRNA against mutS homolog 6 (MSH6), transcript variant 1, mRNA
(NCBI Reference Sequence: NM_000179.3)

| 5′ position | | | | |
|---|---|---|---|---|
| of target | SEQ | | SEQ | |
| site in | ID | Sense Strand | ID | Antisense Strand |
| NM_000179.3 | NO. | Sequence (5′→3′) | NO. | Sequence (5′→3′) |
| 808 | 490 | GAAGUAGCCGCCAAAUAAA | 514 | UUUAUUUGGCGGCUACUUCGC |
| 815 | 491 | CCGCCAAAUAAAAAAACGA | 515 | UCGUUUUUUUAUUUGGCGGCU |
| 1321 | 492 | GGAAGUGGUGGCAGAUUAA | 516 | UUAAUCUGCCACCACUUCCUC |
| 1511 | 493 | GCAGAAGGGCUAUAAAGUA | 517 | UACUUUAUAGCCCUUCUGCAC |
| 1566 | 494 | GAGGCACGAUGUAGAAAGA | 518 | UCUUUCUACAUCGUGCCUCCA |
| 1568 | 495 | GGCACGAUGUAGAAAGAUA | 519 | UAUCUUUCUACAUCGUGCCUC |
| 1622 | 496 | GGAGAUCUGUAGGAUCAUA | 520 | UAUGAUCCUACAGAUCUCCCU |
| 1818 | 497 | CGCCAUUGUUCGAGAUUUA | 521 | UAAAUCUCGAACAAUGGCGAU |
| 1972 | 498 | GGGAUGCAUCCAAAACUUA | 522 | UAAGUUUUGGAUGCAUCCCAA |
| 2256 | 499 | GGUGCUAUCUUCACCAAAA | 523 | UUUUGGUGAAGAUAGCACCAG |
| 2299 | 500 | CAGUGACAUUAAACAACUA | 524 | UAGUUGUUUAAUGUCACUGCA |
| 2372 | 501 | GGUUGAUACUUGCCAUACA | 525 | UGUAUGGCAAGUAUCAACCCU |
| 2486 | 502 | GGUUGUGCCUGACAAAAUA | 526 | UAUUUUGUCAGGCACAACCAU |
| 2606 | 503 | CAGCAGGGCUAUAAUGUAA | 527 | UUACAUUAUAGCCCUGCUGUC |
| 2611 | 504 | GGGCUAUAAUGUAUGAAGA | 528 | UCUUCAUACAUUAUAGCCCUG |
| 3001 | 505 | GGAUUGGUAGGAACCGUUA | 529 | UAACGGUUCCUACCAAUCCCC |
| 3038 | 506 | GAAUUUCACCACUCGCAAA | 530 | UUUGCGAGUGGUGAAAUUCUC |
| 3066 | 507 | GAAUACGAGUUGAAAUCUA | 531 | UAGAUUUCAACUCGUAUUCUU |
| 3089 | 508 | GAAGGGCUGUAAACGAUAA | 532 | UUAUCGUUUACAGCCCUUCUU |
| 3134 | 509 | GGCUAAUCUCAUAAAUGCA | 533 | UGCAUUUAUGAGAUUAGCCAA |
| 3160 | 510 | GGAGGGAUGUAUCAUUGAA | 534 | UUCAAUGAUACAUCCCUCCGU |
| 3189 | 511 | CGGCGACUGUUCUAUAACA | 535 | UGUUAUAGAACAGUCGCCGCA |
| 3191 | 512 | GCGACUGUUCUAUAACUUA | 536 | UAAGUUAUAGAACAGUCGCCG |

TABLE 14
Modified siRNA against mutS homolog 6 (MSH6), transcript variant 1, mRNA
(NCBI Reference Sequence: NM_000179.3)

| 5′ | | | | | | |
|---|---|---|---|---|---|---|
| position | | | | | | |
| of target | SEQ | | | | VEGFA | HEK3 |
| site in | ID | Sense Strand | | Antisense Strand | fold | fold |
| NM_000179.3 | NO. | Sequence (5′→3′) | | Sequence (5′→3′) | change | change |
| 808 | 538 | gaAguaGCCgcCaaauAa | 562 | dTUUAUUUGGCGGcUACUUC | 12.7 | 1.2 |
| | | sa | | gsc | | |
| 815 | 539 | ccGccaAAUaaAaaaaCg | 563 | dTCGUUUUUUUAUuUGGCGG | 8.8 | 1.1 |
| | | sa | | csu | | |
| 1321 | 540 | ggAaguGGUggCagauUa | 564 | dTUAAUCUGCCACcACUUCC | 8.2 | 1.3 |
| | | sa | | usc | | |
| 1511 | 541 | gcAgaaGGGcuAuaaaGu | 565 | dTACUUUAUAGCCcUUCUGC | 10.5 | 1.2 |
| | | sa | | asc | | |
| 1566 | 542 | gaGgcaCGAugUagaaAg | 566 | dTCUUUCUACAUCgUGCCUC | 10.2 | 1.0 |
| | | sa | | csa | | |
| 1568 | 543 | ggCacgAUGuaGaaagAu | 567 | dTAUCUUUCUACAuCGUGCC | 14.5 | 1.0 |
| | | sa | | usc | | |
| 1622 | 544 | ggAgauCUGuaGgaucAu | 568 | dTAUGAUCCUACAgAUCUCC | 15.4 | 1.5 |
| | | sa | | csu | | |
| 1818 | 545 | cgCcauUGUucGagauUu | 569 | dTAAAUCUCGAACaAUGGCG | 12.2 | 1.0 |
| | | sa | | asu | | |
| 1972 | 546 | ggGaugCAUccAaaacUu | 570 | dTAAGUUUUGGAUgCAUCCC | 14.3 | 1.4 |
| | | sa | | asa | | |
| 2256 | 547 | ggUgcuAUCuuCaccaAa | 571 | dTUUUGGUGAAGAuAGCACC | 21.5 | 1.3 |
| | | sa | | asg | | |
| 2299 | 548 | caGugaCAUuaAacaaCu | 572 | dTAGUUGUUUAAUgUCACUG | 12.2 | 1.1 |
| | | sa | | csa | | |
| 2372 | 549 | ggVugaUACuuGccauAc | 573 | dTGUAUGGCAAGUaUCAACC | 16.6 | 0.9 |
| | | sa | | csu | | |
| 2486 | 550 | ggUuguGCCugAcaaaAu | 574 | dTAUUUUGUCAGGcACAACC | 23.1 | 1.2 |
| | | sa | | asu | | |
| 2606 | 551 | caGcagGGCuaUaaugUa | 575 | dTUACAUUAUAGCcCUGCUG | 14.1 | 1.1 |
| | | sa | | usc | | |
| 2611 | 552 | ggGcuaUAAugUaugaAg | 576 | dTCUUCAUACAUUaUAGCCC | 17.7 | 1.0 |
| | | sa | | usg | | |
| 3001 | 553 | ggAuugGUAggAaccgUu | 577 | dTAACGGUUCCUAcCAAUCC | 11.8 | 1.0 |
| | | sa | | csc | | |
| 3038 | 554 | gaAuuuCACcaCucgcAa | 578 | dTUUGCGAGUGGUgAAAUUC | 20.9 | 1.0 |
| | | sa | | usc | | |
| 3066 | 555 | gaAuacGAGuuGaaauCu | 579 | dTAGAUUUCAACUcGUAUUC | 16.6 | 1.0 |
| | | sa | | usu | | |
| 3089 | 556 | gaAgggCUGuaAacgaUa | 580 | dTUAUCGUUUACAgCCCUUC | 15.2 | 0.9 |
| | | sa | | usu | | |
| 3134 | 557 | ggCuaaUCUcaUaaauGc | 581 | dTGCAUUUAUGAGaUUAGCC | 11.3 | 1.2 |
| | | sa | | asa | | |
| 3160 | 558 | ggAgggAUGuaUcauuGa | 582 | dTUCAAUGAUACAuCCCUCC | 18.2 | 1.1 |
| | | sa | | gsu | | |
| 3189 | 559 | cgGcgaCUGuuCuauaAc | 583 | dTGUUAUAGAACAgUCGCCG | 24.9 | 1.4 |
| | | sa | | csa | | |
| 3191 | 560 | gcGacuGUUcuAuaacUu | 584 | dTAAGUUAUAGAAcAGUCGC | 21.6 | 1.1 |
| | | sa | | csg | | |

dN: DNA residues
N: RNA residues
n: 2′-O-methyl residues
s: phosphorothioate backbone modification

TABLE 15
Target Site Sequences of mutS homolog 6 (MSH6),
transcript variant 1, mRNA (NCBI Reference
Sequence: NM_000179.3)

| | 5′ position | |
|---|---|---|
| | of target | |
| SEQ | site in | Target Site Sequence |
| ID NO. | NM_000179.3 | (5′→3′) |
| 585 | 807 | GGCGAAGUAGCCGCCAAAUAAAA |
| 586 | 808 | GCGAAGUAGCCGCCAAAUAAAAA |
| 587 | 815 | AGCCGCCAAAUAAAAAAACGAAG |
| 588 | 1321 | GAGGAAGUGGUGGCAGAUUAAGU |
| 589 | 1511 | GUGCAGAAGGGCUAUAAAGUAGC |
| 590 | 1566 | UGGAGGCACGAUGUAGAAAGAUG |
| 591 | 1568 | GAGGCACGAUGUAGAAAGAUGGC |
| 592 | 1622 | AGGGAGAUCUGUAGGAUCAUUAC |
| 593 | 1818 | AUCGCCAUUGUUCGAGAUUUAGG |
| 594 | 1972 | UUGGGAUGCAUCCAAAACUUUGA |
| 595 | 2256 | CUGGUGCUAUCUUCACCAAAGCC |
| 596 | 2299 | UGCAGUGACAUUAAACAACUUGG |
| 597 | 2372 | AGGGUUGAUACUUGCCAUACUCC |
| 598 | 2486 | AUGGUUGUGCCUGACAAAAUCUC |
| 599 | 2606 | GACAGCAGGGCUAUAAUGUAUGA |
| 600 | 2611 | CAGGGCUAUAAUGUAUGAAGAAA |
| 601 | 3001 | GGGGAUUGGUAGGAACCGUUACC |
| 602 | 3038 | GAGAAUUUCACCACUCGCAAUUU |
| 603 | 3066 | AAGAAUACGAGUUGAAAUCUACC |
| 604 | 3089 | AAGAAGGGCUGUAAACGAUACUG |
| 605 | 3134 | UUGGCUAAUCUCAUAAAUGCUGA |
| 606 | 3160 | ACGGAGGGAUGUAUCAUUGAAGG |
| 607 | 3189 | UGCGGCGACUGUUCUAUAACUUU |
| 608 | 3191 | CGGCGACUGUUCUAUAACUUUGA |

### Antisense Oligonucleotides and Silencing RNAs for MLH1

Provided herein are various siRNAs specific for an mRNA sequence of a mutL homolog 1 (MLH1) gene (e.g., MLH1 transcript variant 1 (NCBI Reference Sequence: NM_000249.4)). In some embodiments, the siRNAs comprise sequence complementarity to any one of the target nucleic acid sequences set forth in SEQ ID NO: 790 to 813. Exemplary antisense molecules against mutL homolog 1 (MLH1) transcript variant 1, mRNA and corresponding target nucleotide ranges are described in Table 16 below. In some embodiments, the siRNAs comprise any one of the matched antisense strand and sense strand pairs set forth in Tables 17-19. Where provided, values “VEGFA fold change,” “HEK3 fold change,” and “RNF2 fold change” indicate the amount of prime editing observed at the respective locus when the given antisense molecule or siRNA pair is administered, in accordance with Example 5, below, with in an vitro-transcribed pegRNA configured to edit that locus and an in vitro-transcribed prime editor, compared to co-administration with an antisense molecule or siRNA pair having a random scrambled sequence. The VEGFA and RNF2 pegRNAs each encode a one-nucleotide substitution, and the HEK3 pegRNA encodes a 3-nucleotide insertion.

In some embodiments, these siRNAs comprise a phosphorothioate internucleotide bond, a methylphosphonate internucleotide bond, and/or a triazole internucleotide bond. Any of these siRNAs may comprise a 2′-O-methoxyethyl oligonucleotide (2′MOE), a 2′-O-methylated nucleoside (2′OMe), a 2′-fluoro oligonucleotide (2′F), an arabino nucleotide (ANA), a 2′-F arabino nucleotide (FANA), a phosphorodiamidate morpholino oligonucleotide (PMO), a peptide nucleic acid (PNA), a phosphorothioate bond (PS), a locked nucleic acid (LNA), a hydrophobic moiety, a naphthyl modifier, or a cholesterol moiety. In some embodiments, these siRNAs are modified with a cholesterol, a dialkyl lipid, or GalNAc. In certain embodiments, these siRNAs are chemically modified with poly-ethylene glycol (PEG). In some embodiments, these siRNAs comprise a 5′ end cap. In some embodiments, these siRNAs may comprise a 3′ end cap. Any of these siRNAs may comprise a DNA nucleotide (e.g., thymine). In certain embodiments, these siRNAs comprise a DNA nucleoside (e.g., thymidine).

Also provided herein are antisense oligonucleotides (ASOs) specific for an mRNA sequence of the mutL homolog 1 (MLH1) gene. In some embodiments the ASOs comprise a nucleic acid sequence set forth in SEQ ID NOs: 609-645.

In some embodiments, these ASOs comprise an antisense strand comprising deoxyribonucleotides and/or ribonucleotides. In some embodiments, these ASOs comprise an antisense strand comprising at least five ribonucleotides at the 5′ end of the siRNA antisense strand. In some embodiments, these ASOs comprise an antisense strand comprising at least five ribonucleotides at the 3′ end of the siRNA antisense strand. In some embodiments, the ASOs comprise an antisense strand (5′ to 3′) comprising deoxyribonucleotides from nucleotide position 6 to nucleotide position 15. In some embodiments, these ASOs comprise a chemical modification. In certain embodiments, these ASOs comprise a phosphorothioate internucleotide bond, a methylphosphonate internucleotide bond, and/or a triazole internucleotide bond. In some embodiments, these ASOs comprise a 2′-O-methoxyethyl oligonucleotide (2′MOE), a 2′-O-methylated nucleoside (2′OMe), a 2′-fluoro oligonucleotide (2′F), an arabino nucleotide (ANA), a 2′-F arabino nucleotide (FANA), a phosphorodiamidate morpholino oligonucleotide (PMO), a peptide nucleic acid (PNA), a phosphorothioate bond (PS), a locked nucleic acid (LNA), a hydrophobic moiety, a naphthyl modifier, or a cholesterol moiety. In some embodiments, these ASOs are modified with a cholesterol, a dialkyl lipid, or GalNAc. In certain embodiments, these ASOs are chemically modified with poly-ethylene glycol (PEG). In some embodiments, these ASOs comprise a 5′ end cap. Any of these ASOs may comprise a 3′ end cap.

TABLE 16
Antisense molecules against mutL homolog 1 (MLH1) transcript variant 1,
mRNA (NCBI Reference Sequence: NM_000249.4)

| SEQ | Target | | | | VEGFA | HEK3 | RNF2 |
|---|---|---|---|---|---|---|---|
| ID | Nucleotide | Target | Antisense Strand | Isoform | fold | fold | fold |
| NO. | Range | location | Sequence (5′→3′) | homology | change | change | change |
| 609 | 5-24 | 5′ UTR | GCCAGAAGAGCCAAGGAA | v1- | 1.5 | 0.9 | |
| | | | AC | 23, X1, X2 | | | |
| | | | | (all) | | | |
| 610 | 111-130 | CR | CTTTGATAGCATTAGCTG | All | 1.2 | 0.8 | 1.0 |
| | | | GC | | | | |
| 611 | 198-217 | CR | CTTGGATCTGAATCAACT | all | 1.8 | 1.0 | 1.2 |
| | | | TC | except | | | |
| | | | | v6, 16 | | | |
| 612 | 208-227 | CR | GTGCCATTGTCTTGGATC | all | | | |
| | | | TG | except | | | |
| | | | | v6, 16 | | | |
| 613 | 272-291 | CR | GGACTGCAGTTTACTAGT | all | | | |
| | | | AG | except | | | |
| | | | | v22 | | | |
| 614 | 295-314 | CR | GAAATACTGGCTAAATCC | all | 1.5 | 0.9 | 1.0 |
| | | | TC | except | | | |
| | | | | v22 | | | |
| 615 | 389-408 | CR | GTATGCACACTTTCCATC | All | 1.5 | 0.9 | 1.4 |
| | | | AG | | | | |
| 616 | 408-427 | CR | CATCTGAGTAACTTGCTC | All | 1.6 | 1.0 | 1.3 |
| | | | TG | | | | |
| 617 | 442-461 | CR | CCAGCACATGGTTTAGGA | All | | | |
| | | | GG | | | | |
| 618 | 451-470 | CR | CCTTGATTGCCAGCACAT | All | | | |
| | | | GG | | | | |
| 619 | 455-474 | CR | GGTCCCTTGATTGCCAGC | All | 0.5 | 0.8 | 1.1 |
| | | | AC | | | | |
| 620 | 562-581 | CR | GAATACCTGCCAACAACT | all | | | |
| | | | TC | except | | | |
| | | | | v4, 8, 9, | | | |
| | | | | 18 | | | |
| 621 | 576-595 | CR | CTGCATTGTGTACTGAAT | All | 2.1 | 1.0 | 1.5 |
| | | | AC | | | | |
| 622 | 621-640 | CR | CATCAGCTACTGTCTCTC | All | | | |
| | | | CT | | | | |
| 623 | 634-653 | CR | GGTAGTGTCCTAACATCA | All | 0.9 | 1.0 | 0.7 |
| | | | GC | | | | |
| 624 | 780-799 | CR | TGCACTTCTTCACTGAGT | all | 1.4 | 0.9 | 1.1 |
| | | | AG | except | | | |
| | | | | v14, X1, | | | |
| | | | | X2 | | | |
| 625 | 824-843 | CR | GGAAGTTGATTCTACCAG | all | | | |
| | | | AC | except | | | |
| | | | | v14-20, | | | |
| | | | | X1, X2 | | | |
| 626 | 853-872 | CR | GCATACACTGTTTCTATG | all | | | |
| | | | GC | except | | | |
| | | | | v14-20, | | | |
| | | | | X1, X2 | | | |
| 627 | 865-884 | CR | GGCAAATAGGCTGCATAC | all | 1.1 | 1.0 | 1.0 |
| | | | AC | except | | | |
| | | | | v14-20, | | | |
| | | | | X1, X2 | | | |
| 628 | 985-1004 | CR | CGCTCCAGGATGCTCTCC | all | | | |
| | | | TC | except | | | |
| | | | | v17-20 | | | |
| 629 | 1021-1040 | CR | GAGCCCAGGAGCTTGCTC | all | 1.2 | 0.8 | 1.1 |
| | | | TC | except | | | |
| | | | | v17-20 | | | |
| 630 | 1037-1056 | CR | CATCCTGGAGGAATTGGA | all | | | |
| | | | GC | except | | | |
| | | | | v17-20 | | | |
| 631 | 1065-1084 | CR | GTCCTGGTAGCAAAGTCT | all | 1.3 | 0.7 | 0.8 |
| | | | GG | except | | | |
| | | | | v17-20 | | | |
| 632 | 1160-1179 | CR | CATCTGGTGGGCATAGAC | All | 0.9 | 0.9 | 0.8 |
| | | | CT | | | | |
| 633 | 1282-1301 | CR | GCCCTGCCACTAGAAATA | All | | | |
| | | | TC | | | | |
| 634 | 1301-1320 | CR | CTCCTCATCTTGCTGCCT | All | 1.4 | 0.8 | 1.5 |
| | | | AG | | | | |
| 635 | 1408-1427 | CR | GAAGTAGGTCCTCTCTTC | All | 1.3 | 0.9 | 1.0 |
| | | | TC | | | | |
| 636 | 1597-1616 | CR | GAGTGGTTATGCAACATC | All | 1.4 | 1.0 | 1.3 |
| | | | TC | | | | |
| 637 | 1624-1643 | CR | CACTGAGGATTCACACAG | All | 1.5 | 0.9 | 1.0 |
| | | | CC | | | | |
| 638 | 1773-1792 | CR | GCATGGCAAGGTCAAAGA | all | 0.2 | 0.8 | 0.6 |
| | | | GC | except | | | |
| | | | | v23 | | | |
| 639 | 1801-1820 | CR | CAGCCACTCTCTGGACTA | all | | | |
| | | | TC | except | | | |
| | | | | v23 | | | |
| 640 | 1812-1831 | CR | CTTCCTCTGTCCAGCCAC | all | 1.5 | 0.9 | 0.9 |
| | | | TC | except | | | |
| | | | | v23 | | | |
| 641 | 1924-1943 | CR | CCAATCAGGTTCCCTTCC | all | | | |
| | | | TC | except | | | |
| | | | | v5, 21, 23 | | | |
| 642 | 1983-2002 | CR | GAATGAAGATAGGCAGTC | all | 1.2 | 0.8 | 0.6 |
| | | | CC | except | | | |
| | | | | v5, 21 | | | |
| 643 | 2121-2140 | CR | CTTCACTCTGCTGGCCTG | all | 1.4 | 0.9 | 0.9 |
| | | | AG | except | | | |
| | | | | v5 | | | |
| 644 | 2254-2273 | CR | TCAGGCAGGTTAGCAAGC | All | 1.4 | 0.9 | 1.1 |
| | | | TG | | | | |
| 645 | 2317-2336 | 3′ UTR | GAAGAACACATCCCACAG | All | 1.5 | 0.9 | 1.0 |
| | | | TG | | | | |

All oligonucleotides contain phosphorothioate internucleotide linkages. Bold nucleotides indicate 2′-MOE nucleotides and plain nucleotides are deoxyribonucleotides.

TABLE 17
19/19 mer siRNAs against mutL homolog 1 (MLH1) transcript variant 1,
mRNA (NCBI Reference Sequence: NM_000249.4)

| 5′ position | | | | |
|---|---|---|---|---|
| of target | SEQ | | SEQ | |
| site in | ID | Sense Strand | ID | Antisense Strand |
| NM_000249.4 | NO. | Sequence (5′→3′) | NO. | Sequence (5′→3′) |
| 297 | 646 | GGAUUUAGCCAGUAUUUCU | 670 | AGAAAUACUGGCUAAAUCC |
| 336 | 647 | GGCUUUGGCCAGCAUAAGC | 671 | GCUUAUGCUGGCCAAAGCC |
| 358 | 648 | GUGGCUCAUGUUACUAUUA | 672 | UAAUAGUAACAUGAGCCAC |
| 367 | 649 | GUUACUAUUACAACGAAAA | 673 | UUUUCGUUGUAAUAGUAAC |
| 391 | 650 | GAUGGAAAGUGUGCAUACA | 674 | UGUAUGCACACUUUCCAUC |
| 451 | 651 | CCAUGUGCUGGCAAUCAAG | 675 | CUUGAUUGCCAGCACAUGG |
| 586 | 652 | CACAAUGCAGGCAUUAGUU | 676 | AACUAAUGCCUGCAUUGUG |
| 633 | 653 | AGCUGAUGUUAGGACACUA | 677 | UAGUGUCCUAACAUCAGCU |
| 640 | 654 | GUUAGGACACUACCCAAUG | 678 | CAUUGGGUAGUGUCCUAAC |
| 658 | 655 | GCCUCAACCGUGGACAAUA | 679 | UAUUGUCCACGGUUGAGGC |
| 665 | 656 | CCGUGGACAAUAUUCGCUC | 680 | GAGCGAAUAUUGUCCACGG |
| 1272 | 657 | GGAUAAGACAGAUAUUUCU | 681 | AGAAAUAUCUGUCUUAUCC |
| 1476 | 658 | GGAAGAUGAUUCCCGAAAG | 682 | CUUUCGGGAAUCAUCUUCC |
| 1494 | 659 | GGAAAUGACUGCAGCUUGU | 683 | ACAAGCUGCAGUCAUUUCC |
| 1520 | 660 | GGAGAAGGAUCAUUAACCU | 684 | AGGUUAAUGAUCCUUCUCC |
| 1523 | 661 | GAAGGAUCAUUAACCUCAC | 685 | GUGAGGUUAAUGAUCCUUC |
| 1539 | 662 | CACUAGUGUUUUGAGUCUC | 686 | GAGACUCAAAACACUAGUG |
| 1575 | 663 | GCAGGGACAUGAGGUUCUC | 687 | GAGAACCUCAUGUCCCUGC |
| 1587 | 664 | GGUUCUCCGGGAGAUGUUG | 688 | CAACAUCUCCCGGAGAACC |
| 1594 | 665 | CGGGAGAUGUUGCAUAACC | 689 | GGUUAUGCAACAUCUCCCG |
| 1595 | 666 | GGGAGAUGUUGCAUAACCA | 690 | UGGUUAUGCAACAUCUCCC |
| 1605 | 667 | GCAUAACCACUCCUUCGUG | 691 | CACGAAGGAGUGGUUAUGC |
| 1654 | 668 | CAGCAUCAAACCAAGUUAU | 692 | AUAACUUGGUUUGAUGCUG |
| 1656 | 669 | GCAUCAAACCAAGUUAUAC | 693 | GUAUAACUUGGUUUGAUGC |

TABLE 18
19/21 mer siRNAs against mutL homolog 1 (MLH1) transcript variant 1,
mRNA (NCBI Reference Sequence: NM_000249.4)

| 5′ position | | | | |
|---|---|---|---|---|
| of target | SEQ | | SEQ | |
| site in | ID | Sense Strand | ID | Antisense Strand |
| NM_000249.4 | NO. | Sequence (5′→3′) | NO. | Sequence (5′→3′) |
| 297 | 694 | GGAUUUAGCCAGUAUUUCA | 718 | UGAAAUACUGGCUAAAUCCUC |
| 336 | 695 | GGCUUUGGCCAGCAUAAGA | 719 | UCUUAUGCUGGCCAAAGCCUC |
| 358 | 696 | GUGGCUCAUGUUACUAUUA | 720 | UAAUAGUAACAUGAGCCACAU |
| 367 | 697 | GUUACUAUUACAACGAAAA | 721 | UUUUCGUUGUAAUAGUAACAU |
| 391 | 698 | GAUGGAAAGUGUGCAUACA | 722 | UGUAUGCACACUUUCCAUCAG |
| 451 | 699 | CCAUGUGCUGGCAAUCAAA | 723 | UUUGAUUGCCAGCACAUGGUU |
| 586 | 700 | CACAAUGCAGGCAUUAGUA | 724 | UACUAAUGCCUGCAUUGUGUA |
| 633 | 701 | AGCUGAUGUUAGGACACUA | 725 | UAGUGUCCUAACAUCAGCUAC |
| 640 | 702 | GUUAGGACACUACCCAAUA | 726 | UAUUGGGUAGUGUCCUAACAU |
| 658 | 703 | GCCUCAACCGUGGACAAUA | 727 | UAUUGUCCACGGUUGAGGCAU |
| 665 | 704 | CCGUGGACAAUAUUCGCUA | 728 | UAGCGAAUAUUGUCCACGGUU |
| 1272 | 705 | GGAUAAGACAGAUAUUUCA | 729 | UGAAAUAUCUGUCUUAUCCUC |
| 1476 | 706 | GGAAGAUGAUUCCCGAAAA | 730 | UUUUCGGGAAUCAUCUUCCAC |
| 1494 | 707 | GGAAAUGACUGCAGCUUGA | 731 | UCAAGCUGCAGUCAUUUCCUU |
| 1520 | 708 | GGAGAAGGAUCAUUAACCA | 732 | UGGUUAAUGAUCCUUCUCCGG |
| 1523 | 709 | GAAGGAUCAUUAACCUCAA | 733 | UUGAGGUUAAUGAUCCUUCUC |
| 1539 | 710 | CACUAGUGUUUUGAGUCUA | 734 | UAGACUCAAAACACUAGUGAG |
| 1575 | 711 | GCAGGGACAUGAGGUUCUA | 735 | UAGAACCUCAUGUCCCUGCUC |
| 1587 | 712 | GGUUCUCCGGGAGAUGUUA | 736 | UAACAUCUCCCGGAGAACCUC |
| 1594 | 713 | CGGGAGAUGUUGCAUAACA | 737 | UGUUAUGCAACAUCUCCCGGA |
| 1595 | 714 | GGGAGAUGUUGCAUAACCA | 738 | UGGUUAUGCAACAUCUCCCGG |
| 1605 | 715 | GCAUAACCACUCCUUCGUA | 739 | UACGAAGGAGUGGUUAUGCAA |
| 1654 | 716 | CAGCAUCAAACCAAGUUAA | 740 | UUAACUUGGUUUGAUGCUGUG |
| 1656 | 717 | GCAUCAAACCAAGUUAUAA | 741 | UUAUAACUUGGUUUGAUGCUG |

TABLE 19
Modified siRNAs against mutL homolog 1 (MLH1) transcript variant 1, mRNA
(NCBI Reference Sequence: NM_000249.4)

| 5′ | | | | | | |
|---|---|---|---|---|---|---|
| position | | | | | | |
| of target | SEQ | | SEQ | | VEGFA | HEK3 |
| site in | ID | Sense Strand | ID | Antisense Strand | fold | fold |
| NM_000249.4 | NO. | Sequence (5′→3′) | NO. | Sequence (5′→3′) | change | change |
| 297 | 742 | ggAuuuAGCcaGuauuUc | 766 | dTGAAAUACUGGCuAAAUCC | 3.8 | 1.4 |
| | | sa | | usc | | |
| 336 | 743 | ggCuuuGGCcaGcauaAg | 767 | dTCUUAUGCUGGCcAAAGCC | 3.2 | 1.5 |
| | | sa | | usc | | |
| 358 | 744 | guGgcuCAUguUacuaUu | 768 | dTAAUAGUAACAUgAGCCAC | 1.8 | 1.6 |
| | | sa | | asu | | |
| 367 | 745 | guUacuAUUacAacgaAa | 769 | dTUUUCGUUGUAAuAGUAAC | 1.3 | 1.6 |
| | | sa | | asu | | |
| 391 | 746 | gaUggaAAGugUgcauAc | 770 | dTGUAUGCACACUuUCCAUC | 1.3 | 1.1 |
| | | sa | | asg | | |
| 451 | 747 | ccAuguGCUggCaaucAa | 771 | dTUUGAUUGCCAGcACAUGG | 1.1 | 1.6 |
| | | sa | | usu | | |
| 586 | 748 | caCaauGCAggCauuaGu | 772 | dTACUAAUGCCUGcAUUGUG | 2.9 | 1.2 |
| | | sa | | usa | | |
| 633 | 749 | agCugaUGUuaGgacaCu | 773 | dTAGUGUCCUAACaUCAGCU | 3.8 | 1.7 |
| | | sa | | asc | | |
| 640 | 750 | guVaggACAcuAcccaAu | 774 | dTAUUGGGUAGUGuCCUAAC | 2.7 | 1.6 |
| | | sa | | asu | | |
| 658 | 751 | gcCucaACCguGgacaAu | 775 | dTAUUGUCCACGGuUGAGGC | 2.3 | 1.8 |
| | | sa | | asu | | |
| 665 | 752 | ccGuggACAauAuucgCu | 776 | dTAGCGAAUAUUGuCCACGG | 3.2 | 1.7 |
| | | sa | | usu | | |
| 1272 | 753 | ggAuaaGACagAuauuUc | 777 | dTGAAAUAUCUGUcUUAUCC | 3.9 | 1.4 |
| | | sa | | usc | | |
| 1476 | 754 | ggAagaUGAuuCccgaAa | 778 | dTUUUCGGGAAUCaUCUUCC | 3.8 | 1.4 |
| | | sa | | asc | | |
| 1494 | 755 | ggAaauGACugCagcuUg | 779 | dTCAAGCUGCAGUcAUUUCC | 2.1 | 1.8 |
| | | sa | | usu | | |
| 1520 | 756 | ggAgaaGGAucAuuaaCc | 780 | dTGGUUAAUGAUCcUUCUCC | 2.5 | 1.6 |
| | | sa | | gsg | | |
| 1523 | 757 | gaAggaUCAuuAaccuCa | 781 | dTUGAGGUUAAUGaUCCUUC | 1.6 | 1.3 |
| | | sa | | usc | | |
| 1539 | 758 | caCuagUGUuuUgaguCu | 782 | dTAGACUCAAAACaCUAGUG | 1.6 | 1.5 |
| | | sa | | asg | | |
| 1575 | 759 | gcAgggACAugAgguuCu | 783 | dTAGAACCUCAUGuCCCUGC | 2.5 | 1.8 |
| | | sa | | usc | | |
| 1587 | 760 | ggUucuCCGggAgaugUu | 784 | dTAACAUCUCCCGgAGAACC | 1.4 | 1.2 |
| | | sa | | usc | | |
| 1594 | 761 | cgGgagAUGuuGcauaAc | 785 | dTGUUAUGCAACAuCUCCCG | 1.1 | 1.5 |
| | | sa | | gsa | | |
| 1595 | 762 | ggGagaUGUugCauaaCc | 786 | dTGGUUAUGCAACaUCUCCC | 1.6 | 1.6 |
| | | sa | | gsg | | |
| 1605 | 763 | gcAuaaCCAcuCcuucGu | 787 | dTACGAAGGAGUGgUUAUGC | 1.6 | 1.6 |
| | | sa | | asa | | |
| 1654 | 764 | caGcauCAAacCaaguUa | 788 | dTUAACUUGGUUUgAUGCUG | 2.0 | 1.8 |
| | | sa | | usg | | |
| 1656 | 765 | gcAucaAACcaAguuaUa | 789 | dTUAUAACUUGGUuUGAUGC | 2.9 | 1.7 |
| | | sa | | usg | | |

dN: DNA residues
N: RNA residues
n: 2′-O-methyl residues
s: phosphorothioate backbone modification

TABLE 20
Target Site Sequences of mutL homolog 1 (MLH1)
transcript variant 1, mRNA (NCBI Reference
Sequence: NM_000249.4)

| | 5′ position | |
|---|---|---|
| | of target | |
| SEQ | site in | Target Site Sequence |
| ID NO. | NM_000249.4 | (5′→3′) |
| 790 | 297 | GAGGAUUUAGCCAGUAUUUCUAC |
| 791 | 336 | GAGGCUUUGGCCAGCAUAAGCCA |
| 792 | 358 | AUGUGGCUCAUGUUACUAUUACA |
| 793 | 367 | AUGUUACUAUUACAACGAAAACA |
| 794 | 391 | CUGAUGGAAAGUGUGCAUACAGA |
| 795 | 451 | AACCAUGUGCUGGCAAUCAAGGG |
| 796 | 586 | UACACAAUGCAGGCAUUAGUUUC |
| 797 | 633 | GUAGCUGAUGUUAGGACACUACC |
| 798 | 640 | AUGUUAGGACACUACCCAAUGCC |
| 799 | 658 | AUGCCUCAACCGUGGACAAUAUU |
| 800 | 665 | AACCGUGGACAAUAUUCGCUCCA |
| 801 | 1272 | GAGGAUAAGACAGAUAUUUCUAG |
| 802 | 1476 | GUGGAAGAUGAUUCCCGAAAGGA |
| 803 | 1494 | AAGGAAAUGACUGCAGCUUGUAC |
| 804 | 1520 | CCGGAGAAGGAUCAUUAACCUCA |
| 805 | 1523 | GAGAAGGAUCAUUAACCUCACUA |
| 806 | 1539 | CUCACUAGUGUUUUGAGUCUCCA |
| 807 | 1575 | GAGCAGGGACAUGAGGUUCUCCG |
| 808 | 1587 | GAGGUUCUCCGGGAGAUGUUGCA |
| 809 | 1594 | UCCGGGAGAUGUUGCAUAACCAC |
| 810 | 1595 | CCGGGAGAUGUUGCAUAACCACU |
| 811 | 1605 | UUGCAUAACCACUCCUUCGUGGG |
| 812 | 1654 | CACAGCAUCAAACCAAGUUAUAC |
| 813 | 1656 | CAGCAUCAAACCAAGUUAUACCU |

Cross-Reactivities of siRNAs for Different Transcript Variants

The cross-reactivities of siRNAs and antisense molecules provided herein to transcript variants of their target genes are provided in the tables below. In these tables “1” indicates a perfect match between the siRNA and the indicated transcript variant, whereas “0” indicates a lack of a perfect match between the siRNA and the indicated transcript variant. In certain embodiments, siRNAs and antisense molecules provided herein are selected based on the transcript variant for which inhibition is desired.

TABLE 21
Cross-Reactivities for MSH2

| MSH2 / | | | | | | |
|---|---|---|---|---|---|---|
| 1170.0-21 | | | | | | |
| 5′ | | | | | | |
| position | | | | | | |
| of 19mer | x-reactivity (detailed) | | | | | |
| target | protein_{—} | | | protein_{—} | protein_{—} | |
| site in | coding | mRNA | mRNA | coding | coding | mRNA |
| NM_000251.3 | ENST00000645506.1 | XM_005264332.4 | XM_011532867.2 | ENST00000406134.5 | ENST00000233146.7 | NM_000251.3 |
| 326 | 1 | 1 | 1 | 1 | 1 | 1 |
| 516 | 1 | 1 | 1 | 1 | 1 | 1 |
| 526 | 1 | 1 | 1 | 1 | 1 | 1 |
| 842 | 1 | 1 | 1 | 1 | 1 | 1 |
| 1170 | 1 | 1 | 1 | 1 | 1 | 1 |
| 1228 | 1 | 1 | 1 | 1 | 1 | 1 |
| 1261 | 1 | 1 | 1 | 1 | 1 | 1 |
| 1263 | 1 | 1 | 1 | 1 | 1 | 1 |
| 1594 | 1 | 1 | 1 | 1 | 1 | 1 |
| 1629 | 1 | 1 | 1 | 1 | 1 | 1 |
| 1635 | 1 | 1 | 1 | 1 | 1 | 1 |
| 1678 | 1 | 1 | 1 | 1 | 1 | 1 |
| 1815 | 1 | 1 | 1 | 1 | 1 | 1 |
| 1879 | 1 | 1 | 1 | 1 | 1 | 1 |
| 2043 | 1 | 1 | 1 | 1 | 1 | 1 |
| 2052 | 1 | 1 | 1 | 1 | 1 | 1 |
| 2063 | 1 | 1 | 1 | 1 | 1 | 1 |
| 2084 | 1 | 1 | 1 | 1 | 1 | 1 |
| 2100 | 1 | 1 | 1 | 1 | 1 | 1 |
| 2317 | 1 | 1 | 1 | 1 | 1 | 1 |
| 2329 | 1 | 1 | 1 | 1 | 1 | 1 |
| 2496 | 1 | 1 | 1 | 1 | 1 | 1 |
| 2544 | 1 | 1 | 1 | 1 | 1 | 1 |
| 2608 | 1 | 1 | 1 | 1 | 1 | 1 |

| MSH2 / | | | | | | |
|---|---|---|---|---|---|---|
| 1170.0-21 | | | | | | |
| 5′ | | | | | | |
| position | x-reactivity (detailed) | | | | | |
| of 19mer | | | nonsense_{—} | | nonsense_{—} | |
| target | | protein_{—} | mediated_{—} | misc_{—} | mediated_{—} | misc_{—} |
| site in | mRNA | coding | deca | RNA | deca | RNA |
| NM_000251.3 | NM_001258281.1 | ENST00000543555.6 | ENST00000644092.1 | XR_001738747.2 | ENST00000646415.1 | XR_939685.2 |
| 326 | 1 | 1 | 1 | 1 | 1 | 1 |
| 516 | 1 | 1 | 1 | 1 | 1 | 1 |
| 526 | 1 | 1 | 1 | 1 | 1 | 1 |
| 842 | 1 | 1 | 1 | 1 | 1 | 1 |
| 1170 | 1 | 1 | 1 | 1 | 1 | 1 |
| 1228 | 1 | 1 | 1 | 1 | 1 | 1 |
| 1261 | 1 | 1 | 1 | 1 | 1 | 1 |
| 1263 | 1 | 1 | 1 | 1 | 1 | 1 |
| 1594 | 1 | 1 | 1 | 1 | 1 | 1 |
| 1629 | 1 | 1 | 1 | 1 | 1 | 1 |
| 1635 | 1 | 1 | 1 | 1 | 1 | 1 |
| 1678 | 1 | 1 | 1 | 1 | 1 | 1 |
| 1815 | 1 | 1 | 1 | 1 | 1 | 1 |
| 1879 | 1 | 1 | 1 | 1 | 1 | 1 |
| 2043 | 1 | 1 | 1 | 1 | 1 | 1 |
| 2052 | 1 | 1 | 1 | 1 | 1 | 1 |
| 2063 | 1 | 1 | 1 | 1 | 1 | 1 |
| 2084 | 1 | 1 | 1 | 1 | 1 | 1 |
| 2100 | 1 | 1 | 1 | 1 | 1 | 1 |
| 2317 | 1 | 1 | 1 | 1 | 1 | 1 |
| 2329 | 1 | 1 | 1 | 1 | 1 | 1 |
| 2496 | 1 | 1 | 1 | 1 | 1 | 1 |
| 2544 | 1 | 1 | 1 | 1 | 1 | 1 |
| 2608 | 1 | 1 | 1 | 1 | 1 | 1 |

| MSH2 / | | | | |
|---|---|---|---|---|
| 1170.0-21 | | | | |
| 5′ | | | | |
| position | x-reactivity (detailed) | | | 19mer x- |
| of 19mer | nonsense_{—} | nonsense_{—} | | reactivity |
| target | mediated_{—} | mediated_{—} | processed_{—} | (of relevant |
| site in | deca | deca | transcript | transcripts) |
| NM_000251.3 | ENST00000645339.1 | ENST00000644900.1 | ENST00000467323.1 | HUMAN |
| 326 | 1 | 0 | 0 | 1 |
| 516 | 1 | 0 | 0 | 1 |
| 526 | 1 | 0 | 0 | 1 |
| 842 | 1 | 0 | 0 | 1 |
| 1170 | 1 | 0 | 0 | 1 |
| 1228 | 1 | 0 | 0 | 1 |
| 1261 | 1 | 0 | 0 | 1 |
| 1263 | 1 | 0 | 0 | 1 |
| 1594 | 1 | 0 | 0 | 1 |
| 1629 | 1 | 10 | 0 | 1 |
| 1635 | 1 | 0 | 0 | 1 |
| 1678 | 1 | 0 | 0 | 1 |
| 1815 | 1 | 0 | 0 | 1 |
| 1879 | 1 | 0 | 0 | 1 |
| 2043 | 1 | 0 | 0 | 1 |
| 2052 | 1 | 0 | 0 | 1 |
| 2063 | 1 | 0 | 0 | 1 |
| 2084 | 1 | 0 | 0 | 1 |
| 2100 | 1 | 0 | 0 | 1 |
| 2317 | 1 | 1 | 0 | 1 |
| 2329 | 1 | 1 | 0 | 1 |
| 2496 | 1 | 1 | 0 | 1 |
| 2544 | 1 | 1 | 0 | 1 |
| 2608 | 1 | 1 | 0 | 1 |

TABLE 22
Cross-Reactivities for PMS2

| PMS2 / | | | | | | | | | | | | |
|---|---|---|---|---|---|---|---|---|---|---|---|---|
| 1170-0.21 | | | | | | | | | | | | |
| 5′ position | x-reactivity (detailed) | | | | | | | | | | | |
| of 19mer | mRNA | mRNA | mRNA | mRNA | protein_{—} | mRNA | mRNA | mRNA | mRNA | mRNA | mRNA | mRNA |
| target | NM_{—} | NM_{—} | NM_{—} | NM_{—} | coding | NM_{—} | NM_{—} | NM_{—} | NM_{—} | NM_{—} | NM_{—} | NM_{—} |
| site in | 001322009.2 | 001322005.2 | 001322015.2 | 001322014.2 | ENST00000265849.12 | 000535.7 | 001322003.2 | 001322011.2 | 001322012.2 | 001322007.2 | 001322004.2 | 001322006.2 |
| NM_000535.7 | Hs | Hs | Hs | Hs | Hs | Hs | Hs | Hs | Hs | Hs | Hs | Hs |
| 424 | 1 | 1 | 1 | 1 | 1 | 1 | 1 | 1 | 1 | 1 | 1 | 1 |
| 952 | 1 | 1 | 1 | 1 | 1 | 1 | 1 | 1 | 1 | 1 | 1 | 1 |
| 954 | 1 | 1 | 1 | 1 | 1 | 1 | 1 | 1 | 1 | 1 | 1 | 1 |
| 957 | 1 | 1 | 1 | 1 | 1 | 1 | 1 | 1 | 1 | 1 | 1 | 1 |
| 960 | 1 | 1 | 1 | 1 | 1 | 1 | 1 | 1 | 1 | 1 | 1 | 1 |
| 1374 | 1 | 1 | 1 | 1 | 1 | 1 | 1 | 1 | 1 | 1 | 1 | 1 |
| 1382 | 1 | 1 | 1 | 1 | 1 | 1 | 1 | 1 | 1 | 1 | 1 | 1 |
| 1384 | 1 | 1 | 1 | 1 | 1 | 1 | 1 | 1 | 1 | 1 | 1 | 1 |
| 1580 | 1 | 1 | 1 | 1 | 1 | 1 | 1 | 1 | 1 | 1 | 1 | 1 |
| 1702 | 1 | 1 | 1 | 1 | 1 | 1 | 1 | 1 | 1 | 1 | 1 | 1 |
| 1704 | 1 | 1 | 1 | 1 | 1 | 1 | 1 | 1 | 1 | 1 | 1 | 1 |
| 1705 | 1 | 1 | 1 | 1 | 1 | 1 | 1 | 1 | 1 | 1 | 1 | 1 |
| 1730 | 1 | 1 | 1 | 1 | 1 | 1 | 1 | 1 | 1 | 1 | 1 | 1 |
| 1741 | 1 | 1 | 1 | 1 | 1 | 1 | 1 | 1 | 1 | 1 | 1 | 1 |
| 1748 | 1 | 1 | 1 | 1 | 1 | 1 | 1 | 1 | 1 | 1 | 1 | 1 |
| 1749 | 1 | 1 | 1 | 1 | 1 | 1 | 1 | 1 | 1 | 1 | 1 | 1 |
| 1752 | 1 | 1 | 1 | 1 | 1 | 1 | 1 | 1 | 1 | 1 | 1 | 1 |
| 1753 | 1 | 1 | 1 | 1 | 1 | 1 | 1 | 1 | 1 | 1 | 1 | 1 |
| 1824 | 1 | 1 | 1 | 1 | 1 | 1 | 1 | 1 | 1 | 1 | 1 | 1 |
| 3230 | 1 | 1 | 1 | 1 | 1 | 1 | 1 | 1 | 1 | 1 | 1 | 1 |
| 3259 | 1 | 1 | 1 | 1 | 1 | 1 | 1 | 1 | 1 | 1 | 1 | 1 |
| 3413 | 1 | 1 | 1 | 1 | 1 | 1 | 1 | 1 | 1 | 1 | 1 | 1 |
| 3483 | 1 | 1 | 1 | 1 | 1 | 1 | 1 | 1 | 1 | 1 | 1 | 1 |
| 5039 | 1 | 1 | 1 | 1 | 1 | 1 | 1 | 1 | 1 | 1 | 1 | 1 |

| PMS2 / | | | | | | | | | | |
|---|---|---|---|---|---|---|---|---|---|---|
| 1170-0.21 | | | | | | | | | | |
| 5′ position | x-reactivity (detailed) | | | | | | | | | |
| of 19mer | mRNA | mRNA | mRNA | mRNA | mRNA | mRNA | protein_{—} | protein_{—} | protein_{—} | protein_{—} |
| target | NM_{—} | NM_{—} | NM_{—} | XM_{—} | XM_{—} | XM_{—} | coding | coding | coding | coding |
| site in | 001322013.2 | 001322008.2 | 001322010.2 | 024446800.1 | 017012342.2 | 006715744.4 | ENST00000642456.1 | ENST00000441476.6 | ENST00000642292.1 | ENST00000644110.1 |
| NM_000535.7 | Hs | Hs | Hs | Hs | Hs | Hs | Hs | Hs | Hs | Hs |
| 424 | 1 | 1 | 1 | 1 | 1 | 0 | 1 | 1 | 1 | 1 |
| 952 | 1 | 1 | 1 | 1 | 1 | 1 | 1 | 1 | 1 | 1 |
| 954 | 1 | 1 | 1 | 1 | 1 | 1 | 1 | 1 | 1 | 1 |
| 957 | 1 | 1 | 1 | 1 | 1 | 1 | 1 | 1 | 1 | 1 |
| 960 | 1 | 1 | 1 | 1 | 1 | 1 | 1 | 1 | 1 | 1 |
| 1374 | 1 | 1 | 1 | 1 | 1 | 1 | 1 | 1 | 1 | 1 |
| 1382 | 1 | 1 | 1 | 1 | 1 | 1 | 1 | 1 | 1 | 1 |
| 1384 | 1 | 1 | 1 | 1 | 1 | 1 | 1 | 1 | 1 | 1 |
| 1580 | 1 | 1 | 1 | 1 | 1 | 1 | 1 | 1 | 1 | 1 |
| 1702 | 1 | 1 | 1 | 1 | 1 | 1 | 1 | 1 | 1 | 1 |
| 1704 | 1 | 1 | 1 | 1 | 1 | 1 | 1 | 1 | 1 | 1 |
| 1705 | 1 | 1 | 1 | 1 | 1 | 1 | 1 | 1 | 1 | 1 |
| 1730 | 1 | 1 | 1 | 1 | 1 | 1 | 1 | 1 | 1 | 1 |
| 1741 | 1 | 1 | 1 | 1 | 1 | 1 | 1 | 1 | 1 | 1 |
| 1748 | 1 | 1 | 1 | 1 | 1 | 1 | 1 | 1 | 1 | 1 |
| 1749 | 1 | 1 | 1 | 1 | 1 | 1 | 1 | 1 | 1 | 1 |
| 1752 | 1 | 1 | 1 | 1 | 1 | 1 | 1 | 1 | 1 | 1 |
| 1753 | 1 | 1 | 1 | 1 | 1 | 1 | 1 | 1 | 1 | 1 |
| 1824 | 1 | 1 | 1 | 1 | 1 | 1 | 1 | 1 | 1 | 1 |
| 3230 | 1 | 1 | 1 | 1 | 1 | 1 | 0 | 0 | 0 | 0 |
| 3259 | 1 | 1 | 1 | 1 | 1 | 1 | 0 | 0 | 0 | 0 |
| 3413 | 1 | 1 | 1 | 1 | 1 | 1 | 0 | 0 | 0 | 0 |
| 3483 | 1 | 1 | 1 | 1 | 1 | 1 | 0 | 0 | 0 | 0 |
| 5039 | 1 | 1 | 1 | 0 | 0 | 0 | 0 | 0 | 0 | 0 |

| PMS2 / | x-reactivity (detailed) | | | | | | | |
|---|---|---|---|---|---|---|---|---|
| 1170-0.21 | | RNA | | | | | | |
| 5′ position | | non- | nonsense_{—} | | | | | 19mer x- |
| of 19mer | protein_{—} | coding | mediated_{—} | processed_{—} | retained_{—} | retained_{—} | processed_{—} | reactivity |
| target | coding | NR_{—} | decay | transcript | intron | intron | transcript | (of relevant |
| site in | ENST00000382321.5 | 136154.1 | ENST00000643595.1 | ENST00000406569.7 | ENST00000380416.5 | ENST00000415839.2 | ENST00000469652.1 | transcripts) |
| NM_000535.7 | Hs | Hs | Hs | Hs | Hs | Hs | Hs | HUMAN |
| 424 | 1 | 1 | 1 | 1 | 1 | 0 | 0 | 0 |
| 952 | 0 | 1 | 1 | 1 | 0 | 0 | 0 | 1 |
| 954 | 0 | 1 | 1 | 1 | 0 | 0 | 0 | 1 |
| 957 | 0 | 1 | 1 | 1 | 0 | 0 | 0 | 1 |
| 960 | 0 | 1 | 1 | 1 | 0 | 0 | 0 | 1 |
| 1374 | 0 | 1 | 1 | 1 | 0 | 0 | 0 | 1 |
| 1382 | 0 | 1 | 1 | 1 | 0 | 0 | 0 | 1 |
| 1384 | 0 | 1 | 1 | 1 | 0 | 0 | 0 | 1 |
| 1580 | 0 | 1 | 1 | 0 | 0 | 0 | 0 | 1 |
| 1702 | 0 | 1 | 1 | 0 | 0 | 0 | 0 | 1 |
| 1704 | 0 | 1 | 1 | 0 | 0 | 0 | 0 | 1 |
| 1705 | 0 | 1 | 1 | 0 | 0 | 0 | 0 | 1 |
| 1730 | 0 | 1 | 1 | 0 | 0 | 0 | 0 | 1 |
| 1741 | 0 | 1 | 1 | 0 | 0 | 0 | 0 | 1 |
| 1748 | 0 | 1 | 1 | 0 | 0 | 0 | 0 | 1 |
| 1749 | 0 | 1 | 1 | 0 | 0 | 0 | 0 | 1 |
| 1752 | 0 | 1 | 1 | 0 | 0 | 0 | 0 | 1 |
| 1753 | 0 | 1 | 1 | 0 | 0 | 0 | 0 | 1 |
| 1824 | 0 | 1 | 1 | 0 | 0 | 0 | 0 | 1 |
| 3230 | 0 | 1 | 0 | 0 | 0 | 0 | 0 | 0 |
| 3259 | 0 | 1 | 0 | 0 | 0 | 0 | 0 | 0 |
| 3413 | 0 | 1 | 0 | 0 | 0 | 0 | 0 | 0 |
| 3483 | 0 | 1 | 0 | 0 | 0 | 0 | 0 | 0 |
| 5039 | 0 | 1 | 0 | 0 | 0 | 0 | 0 | 0 |

TABLE 23
Cross-Reactivities for MSH6

| MSH6 / | | | | | | | | | |
|---|---|---|---|---|---|---|---|---|---|
| 1170.0-21 | | | | | | | | | |
| 5′ position | | | | | | | | | |
| of 19mer | x-reactivity (detailed) | | | | | | | | |
| target | | mRNA | | | mRNA | mRNA | mRNA | | mRNA |
| site in | protein_coding | XM_{—} | protein_coding | protein_coding | NM_{—} | XM_{—} | XM_{—} | protein_coding | XM_{—} |
| NM_000179.3 | ENST00000652107.1 | 024452819.1 | ENST00000622629.4 | ENST00000234420.11 | 000179.3 | 024452821.1 | 024452822.1 | ENST00000614496.4 | 024452820.1 |
| 807 | 1 | 1 | 1 | 1 | 1 | 1 | 1 | 1 | 1 |
| 808 | 1 | 1 | 1 | 1 | 1 | 1 | 1 | 1 | 1 |
| 815 | 1 | 1 | 1 | 1 | 1 | 1 | 1 | 1 | 1 |
| 1321 | 1 | 1 | 1 | 1 | 1 | 1 | 1 | 1 | 1 |
| 1511 | 1 | 1 | 1 | 1 | 1 | 1 | 1 | 1 | 1 |
| 1566 | 1 | 1 | 1 | 1 | 1 | 1 | 1 | 1 | 1 |
| 1568 | 1 | 1 | 1 | 1 | 1 | 1 | 1 | 1 | 1 |
| 1622 | 1 | 1 | 1 | 1 | 1 | 1 | 1 | 1 | 1 |
| 1818 | 1 | 1 | 1 | 1 | 1 | 1 | 1 | 1 | 1 |
| 1972 | 1 | 1 | 1 | 1 | 1 | 1 | 1 | 1 | 1 |
| 2256 | 1 | 1 | 1 | 1 | 1 | 1 | 1 | 1 | 1 |
| 2299 | 1 | 1 | 1 | 1 | 1 | 1 | 1 | 1 | 1 |
| 2372 | 1 | 1 | 1 | 1 | 1 | 1 | 1 | 1 | 1 |
| 2486 | 1 | 1 | 1 | 1 | 1 | 1 | 1 | 1 | 1 |
| 2606 | 1 | 1 | 1 | 1 | 1 | 1 | 1 | 1 | 1 |
| 2611 | 1 | 1 | 1 | 1 | 1 | 1 | 1 | 1 | 1 |
| 3001 | 1 | 1 | 1 | 1 | 1 | 1 | 1 | 1 | 1 |
| 3038 | 1 | 1 | 1 | 1 | 1 | 1 | 1 | 1 | 1 |
| 3066 | 1 | 1 | 1 | 1 | 1 | 1 | 1 | 1 | 1 |
| 3089 | 1 | 1 | 1 | 1 | 1 | 1 | 1 | 1 | 1 |
| 3134 | 1 | 1 | 1 | 1 | 1 | 1 | 1 | 1 | 1 |
| 3160 | 1 | 1 | 1 | 1 | 1 | 1 | 1 | 1 | 1 |
| 3189 | 1 | 1 | 1 | 1 | 1 | 1 | 1 | 1 | 1 |
| 3191 | 1 | 1 | 1 | 1 | 1 | 1 | 1 | 1 | 1 |

| MSH6 / | |
|---|---|
| 1170.0-21 | |
| 5′ position | |
| of 19mer | x-reactivity (detailed) |

| target | mRNA | mRNA | | | mRNA | | | |
|---|---|---|---|---|---|---|---|---|
| site in | NM_{—} | NM_{—} | protein_coding | protein_coding | NM_{—} | protein_coding | protein_coding | protein_coding |
| NM_000179.3 | 001281493.2 | 001281494.2 | ENST00000538136.1 | ENST00000673637.1 | 001281492.2 | ENST00000540021.6 | ENST00000616033.4 | ENST00000455383.5 |
| 807 | 1 | 1 | 1 | 1 | 1 | 1 | 1 | 0 |
| 808 | 1 | 1 | 1 | 1 | 1 | 1 | 1 | 0 |
| 815 | 1 | 1 | 1 | 1 | 1 | 1 | 1 | 0 |
| 1321 | 1 | 1 | 1 | 1 | 1 | 1 | 1 | 0 |
| 1511 | 1 | 1 | 1 | 1 | 1 | 1 | 1 | 0 |
| 1566 | 1 | 1 | 1 | 1 | 1 | 1 | 1 | 0 |
| 1568 | 1 | 1 | 1 | 1 | 1 | 1 | 1 | 0 |
| 1622 | 1 | 1 | 1 | 1 | 1 | 1 | 1 | 0 |
| 1818 | 1 | 1 | 1 | 1 | 1 | 1 | 1 | 0 |
| 1972 | 1 | 1 | 1 | 1 | 1 | 1 | 1 | 0 |
| 2256 | 1 | 1 | 1 | 1 | 1 | 1 | 1 | 0 |
| 2299 | 1 | 1 | 1 | 1 | 1 | 1 | 1 | 0 |
| 2372 | 1 | 1 | 1 | 1 | 1 | 1 | 1 | 0 |
| 2486 | 1 | 1 | 1 | 1 | 1 | 1 | 1 | 0 |
| 2606 | 1 | 1 | 1 | 1 | 1 | 1 | 1 | 0 |
| 2611 | 1 | 1 | 1 | 1 | 1 | 1 | 1 | 0 |
| 3001 | 1 | 1 | 1 | 1 | 1 | 1 | 1 | 0 |
| 3038 | 1 | 1 | 1 | 1 | 1 | 1 | 1 | 0 |
| 3066 | 1 | 1 | 1 | 1 | 1 | 1 | 1 | 0 |
| 3089 | 1 | 1 | 1 | 1 | 1 | 1 | 1 | 0 |
| 3134 | 1 | 1 | 1 | 1 | 1 | 1 | 1 | 0 |
| 3160 | 1 | 1 | 1 | 1 | 1 | 1 | 1 | 0 |
| 3189 | 1 | 1 | 1 | 1 | 1 | 1 | 1 | 0 |
| 3191 | 1 | 1 | 1 | 1 | 1 | 1 | 1 | 0 |

| MSH6 / | | | | | |
|---|---|---|---|---|---|
| 1170.0-21 | | | | | |
| 5′ position | x-reactivity (detailed) | | | | |
| of 19mer | | | | | nonsense_{—} |
| target | | | nonsense_{—} | processed_{—} | mediated_{—} |
| site in | protein_coding | protein_coding | mediated_dec | transcript | dec |
| NM_000179.3 | ENST00000411819.1 | ENST00000420813.5 | ENST00000445503.5 | ENST00000607272.2 | ENST00000456246.1 |
| 807 | 1 | 0 | 1 | 0 | 1 |
| 808 | 1 | 0 | 1 | 0 | 1 |
| 815 | 1 | 0 | 1 | 0 | 1 |
| 1321 | 0 | 0 | 1 | 0 | 0 |
| 1511 | 0 | 0 | 1 | 0 | 0 |
| 1566 | 0 | 0 | 1 | 0 | 0 |
| 1568 | 0 | 0 | 1 | 0 | 0 |
| 1622 | 0 | 0 | 1 | 0 | 0 |
| 1818 | 0 | 0 | 1 | 0 | 0 |
| 1972 | 0 | 0 | 1 | 0 | 0 |
| 2256 | 0 | 0 | 1 | 0 | 0 |
| 2299 | 0 | 0 | 1 | 0 | 0 |
| 2372 | 0 | 0 | 1 | 0 | 0 |
| 2486 | 0 | 0 | 1 | 0 | 0 |
| 2606 | 0 | 0 | 1 | 0 | 0 |
| 2611 | 0 | 0 | 1 | 0 | 0 |
| 3001 | 0 | 0 | 1 | 0 | 0 |
| 3038 | 0 | 0 | 1 | 0 | 0 |
| 3066 | 0 | 0 | 1 | 0 | 0 |
| 3089 | 0 | 0 | 1 | 0 | 0 |
| 3134 | 0 | 0 | 1 | 0 | 0 |
| 3160 | 0 | 0 | 1 | 0 | 0 |
| 3189 | 0 | 0 | 1 | 0 | 0 |
| 3191 | 0 | 0 | 1 | 0 | 0 |

| MSH6 / | | | | |
|---|---|---|---|---|
| 1170.0-21 | | | | |
| 5′ position | | | | 19mer x- |
| of 19mer | x-reactivity (detailed) | | | reactivity |
| target | processed_{—} | processed_{—} | processed_{—} | (of relevant |
| site in | transcript | transcript | transcript | transcripts) |
| NM_000179.3 | ENST00000493177.1 | ENST00000454137.5 | ENST00000673922.1 | HUMAN |
| 807 | 0 | 0 | 1 | 1 |
| 808 | 0 | 0 | 1 | 1 |
| 815 | 0 | 0 | 1 | 1 |
| 1321 | 0 | 0 | 0 | 1 |
| 1511 | 0 | 0 | 0 | 1 |
| 1566 | 0 | 0 | 0 | 1 |
| 1568 | 0 | 0 | 0 | 1 |
| 1622 | 0 | 0 | 0 | 1 |
| 1818 | 0 | 0 | 0 | 1 |
| 1972 | 0 | 0 | 0 | 1 |
| 2256 | 0 | 0 | 0 | 1 |
| 2299 | 0 | 0 | 0 | 1 |
| 2372 | 0 | 0 | 0 | 1 |
| 2486 | 0 | 0 | 0 | 1 |
| 2606 | 0 | 0 | 0 | 1 |
| 2611 | 0 | 0 | 0 | 1 |
| 3001 | 0 | 0 | 0 | 1 |
| 3038 | 0 | 0 | 0 | 1 |
| 3066 | 0 | 0 | 0 | 1 |
| 3089 | 0 | 0 | 0 | 1 |
| 3134 | 0 | 0 | 0 | 1 |
| 3160 | 0 | 0 | 0 | 1 |
| 3189 | 0 | 0 | 0 | 1 |
| 3191 | 0 | 0 | 0 | 1 |

TABLE 24A
Cross-Reactivities for MLH1

| MLH1 / | | | | | | | | | | | |
|---|---|---|---|---|---|---|---|---|---|---|---|
| 1170.0-21 | | | | | | | | | | | |
| 5′ position | | | | | | | | | | | |
| of 19mer | x-reactivity (detailed) | | | | | | | | | | |
| target | mRNA | mRNA | mRNA | mRNA | mRNA | mRNA | mRNA | | | | mRNA |
| site in | NM_{—} | NM_{—} | NM_{—} | NM_{—} | NM_{—} | NM_{—} | NM_{—} | protein_coding | protein_coding | protein_coding | NM_{—} |
| NM_000249.4 | 001258274.3 | 001354619.2 | 001354618.2 | 001167618.3 | 001167617.3 | 001167619.3 | 001354622.2 | ENST00000458205.6 | ENST00000674019.1 | ENST00000536378.5 | 001354623.2 |
| 297 | 1 | 1 | 1 | 1 | 1 | 1 | 1 | 1 | 1 | 1 | 1 |
| 336 | 1 | 1 | 1 | 1 | 1 | 1 | 1 | 1 | 1 | 1 | 1 |
| 358 | 1 | 1 | 1 | 1 | 1 | 1 | 1 | 1 | 1 | 1 | 1 |
| 367 | 1 | 1 | 1 | 1 | 1 | 1 | 1 | 1 | 1 | 1 | 1 |
| 391 | 1 | 1 | 1 | 1 | 1 | 1 | 1 | 1 | 1 | 1 | 1 |
| 451 | 1 | 1 | 1 | 1 | 1 | 1 | 1 | 1 | 1 | 1 | 1 |
| 586 | 1 | 1 | 1 | 1 | 1 | 1 | 1 | 1 | 1 | 1 | 1 |
| 633 | 1 | 1 | 1 | 1 | 1 | 1 | 1 | 1 | 1 | 1 | 1 |
| 640 | 1 | 1 | 1 | 1 | 1 | 1 | 1 | 1 | 1 | 1 | 1 |
| 658 | 1 | 1 | 1 | 1 | 1 | 1 | 1 | 1 | 1 | 1 | 1 |
| 665 | 1 | 1 | 1 | 1 | 1 | 1 | 1 | 1 | 1 | 1 | 1 |
| 1272 | 1 | 1 | 1 | 1 | 1 | 1 | 1 | 1 | 1 | 1 | 1 |
| 1476 | 1 | 1 | 1 | 1 | 1 | 1 | 1 | 1 | 1 | 1 | 1 |
| 1494 | 1 | 1 | 1 | 1 | 1 | 1 | 1 | 1 | 1 | 1 | 1 |
| 1520 | 1 | 1 | 1 | 1 | 1 | 1 | 1 | 1 | 1 | 1 | 1 |
| 1523 | 1 | 1 | 1 | 1 | 1 | 1 | 1 | 1 | 1 | 1 | 1 |
| 1530 | 1 | 1 | 1 | 1 | 1 | 1 | 1 | 1 | 1 | 1 | 1 |
| 1575 | 1 | 1 | 1 | 1 | 1 | 1 | 1 | 1 | 1 | 1 | 1 |
| 1587 | 1 | 1 | 1 | 1 | 1 | 1 | 1 | 1 | 1 | 1 | 1 |
| 1594 | 1 | 1 | 1 | 1 | 1 | 1 | 1 | 1 | 1 | 1 | 1 |
| 1599 | 1 | 1 | 1 | 1 | 1 | 1 | 1 | 1 | 1 | 1 | 1 |
| 1605 | 1 | 1 | 1 | 1 | 1 | 1 | 1 | 1 | 1 | 1 | 1 |
| 1654 | 1 | 1 | 1 | 1 | 1 | 1 | 1 | 1 | 1 | 1 | 1 |
| 1656 | 1 | 1 | 1 | 1 | 1 | 1 | 1 | 1 | 1 | 1 | 1 |

| MLH1 / | | | | | | | | | | | |
|---|---|---|---|---|---|---|---|---|---|---|---|
| 1170.0-21 | | | | | | | | | | | |
| 5′ position | | | | | | | | | | | |
| of 19mer | x-reactivity (detailed) | | | | | | | | | | |
| target | mRNA | | mRNA | mRNA | mRNA | mRNA | | mRNA | mRNA | mRNA | mRNA |
| site in | NM_{—} | protein_coding | NM_{—} | NM_{—} | NM_{—} | NM_{—} | protein_coding | NM_{—} | NM_{—} | NM_{—} | NM_{—} |
| NM_000249.4 | 001354621.2 | ENST00000231790.8 | 000249.4 | 001354617.2 | 001354620.2 | 001354627.2 | ENST00000435176.5 | 001258273.2 | 001354628.2 | 001354616.2 | 001354615.2 |
| 297 | 1 | 1 | 1 | 1 | 1 | 1 | 1 | 1 | 1 | 1 | 1 |
| 336 | 1 | 1 | 1 | 1 | 1 | 1 | 1 | 1 | 1 | 1 | 1 |
| 358 | 1 | 1 | 1 | 1 | 1 | 1 | 1 | 1 | 1 | 1 | 1 |
| 367 | 1 | 1 | 1 | 1 | 1 | 1 | 1 | 1 | 1 | 1 | 1 |
| 391 | 1 | 1 | 1 | 1 | 1 | 1 | 1 | 1 | 1 | 1 | 1 |
| 451 | 1 | 1 | 1 | 1 | 1 | 1 | 1 | 1 | 1 | 1 | 1 |
| 586 | 1 | 1 | 1 | 1 | 1 | 1 | 1 | 1 | 1 | 1 | 1 |
| 633 | 1 | 1 | 1 | 1 | 1 | 1 | 1 | 1 | 1 | 1 | 1 |
| 640 | 1 | 1 | 1 | 1 | 1 | 1 | 1 | 1 | 1 | 1 | 1 |
| 658 | 1 | 1 | 1 | 1 | 1 | 1 | 1 | 1 | 1 | 1 | 1 |
| 665 | 1 | 1 | 1 | 1 | 1 | 1 | 1 | 1 | 1 | 1 | 1 |
| 1272 | 1 | 1 | 1 | 1 | 1 | 1 | 1 | 1 | 1 | 1 | 1 |
| 1476 | 1 | 1 | 1 | 1 | 1 | 1 | 1 | 1 | 1 | 1 | 1 |
| 1494 | 1 | 1 | 1 | 1 | 1 | 1 | 1 | 1 | 1 | 1 | 1 |
| 1520 | 1 | 1 | 1 | 1 | 1 | 1 | 1 | 1 | 1 | 1 | 1 |
| 1523 | 1 | 1 | 1 | 1 | 1 | 1 | 1 | 1 | 1 | 1 | 1 |
| 1530 | 1 | 1 | 1 | 1 | 1 | 1 | 1 | 1 | 1 | 1 | 1 |
| 1575 | 1 | 1 | 1 | 1 | 1 | 1 | 1 | 1 | 1 | 1 | 1 |
| 1587 | 1 | 1 | 1 | 1 | 1 | 1 | 1 | 1 | 1 | 1 | 1 |
| 1594 | 1 | 1 | 1 | 1 | 1 | 1 | 1 | 1 | 1 | 1 | 1 |
| 1599 | 1 | 1 | 1 | 1 | 1 | 1 | 1 | 1 | 1 | 1 | 1 |
| 1605 | 1 | 1 | 1 | 1 | 1 | 1 | 1 | 1 | 1 | 1 | 1 |
| 1654 | 1 | 1 | 1 | 1 | 1 | 1 | 1 | 1 | 1 | 1 | 1 |
| 1656 | 1 | 1 | 1 | 1 | 1 | 1 | 1 | 1 | 1 | 1 | 1 |

| MLH1 / | | | | | | | | | | |
|---|---|---|---|---|---|---|---|---|---|---|
| 1170.0-21 | | | | | | | | | | |
| 5′ position | | | | | | | | | | |
| of 19mer | x-reactivity (detailed) | | | | | | | | | |
| target | mRNA | mRNA | mRNA | | mRNA | mRNA | | mRNA | mRNA | |
| site in | NM_{—} | NM_{—} | XM_{—} | protein_coding | XM_{—} | NM_{—} | protein_coding | NM_{—} | NM_{—} | protein_coding |
| NM_000249.4 | 001354629.2 | 001354630.2 | 005265161.2 | ENST00000455445.6 | 017006450.2 | 001258271.2 | ENST00000539477.6 | 001354624.2 | 001354626.2 | ENST00000456676.6 |
| 297 | 0 | 1 | 1 | 1 | 1 | 1 | 1 | 1 | 1 | 1 |
| 336 | 1 | 1 | 1 | 1 | 1 | 1 | 1 | 1 | 1 | 1 |
| 358 | 1 | 1 | 1 | 1 | 1 | 1 | 1 | 1 | 1 | 1 |
| 367 | 1 | 1 | 1 | 1 | 1 | 1 | 1 | 1 | 1 | 1 |
| 391 | 1 | 1 | 1 | 1 | 1 | 1 | 1 | 1 | 1 | 1 |
| 451 | 1 | 1 | 1 | 1 | 1 | 1 | 1 | 1 | 1 | 1 |
| 586 | 1 | 1 | 1 | 1 | 1 | 1 | 1 | 1 | 1 | 1 |
| 633 | 1 | 1 | 1 | 1 | 1 | 1 | 1 | 1 | 1 | 1 |
| 640 | 1 | 1 | 1 | 1 | 1 | 1 | 1 | 1 | 1 | 1 |
| 658 | 1 | 1 | 1 | 1 | 1 | 1 | 1 | 1 | 1 | 1 |
| 665 | 1 | 1 | 1 | 1 | 1 | 1 | 1 | 1 | 1 | 1 |
| 1272 | 1 | 1 | 1 | 1 | 1 | 1 | 1 | 1 | 1 | 1 |
| 1476 | 1 | 1 | 1 | 1 | 1 | 1 | 1 | 1 | 1 | 1 |
| 1494 | 1 | 1 | 1 | 1 | 1 | 1 | 1 | 1 | 1 | 1 |
| 1520 | 1 | 1 | 1 | 1 | 1 | 1 | 1 | 1 | 1 | 1 |
| 1523 | 1 | 1 | 1 | 1 | 1 | 1 | 1 | 1 | 1 | 1 |
| 1530 | 1 | 1 | 1 | 1 | 1 | 1 | 1 | 1 | 1 | 1 |
| 1575 | 1 | 1 | 1 | 1 | 1 | 1 | 1 | 1 | 1 | 1 |
| 1587 | 1 | 1 | 1 | 1 | 1 | 1 | 1 | 1 | 1 | 1 |
| 1594 | 1 | 1 | 1 | 1 | 1 | 1 | 1 | 1 | 1 | 1 |
| 1599 | 1 | 1 | 1 | 1 | 1 | 1 | 1 | 1 | 1 | 1 |
| 1605 | 1 | 1 | 1 | 1 | 1 | 1 | 1 | 1 | 1 | 1 |
| 1654 | 1 | 1 | 1 | 1 | 1 | 1 | 1 | 1 | 1 | 1 |
| 1656 | 1 | 1 | 1 | 1 | 1 | 1 | 1 | 1 | 1 | 1 |

TABLE 24B
Cross-Reactivities for MLH1

| MLH1 / | | | | | | | | |
|---|---|---|---|---|---|---|---|---|
| 1170.0-21 | | | | | | | | |
| 5′ | | | | | | | | |
| position | | | | | | | | |
| of 19mer | x-reactivity (detailed) | | | | | | | |
| target | protein_{—} | mRNA | protein_{—} | protein_{—} | protein_{—} | protein_{—} | protein_{—} | protein_{—} |
| site in | coding | NM_{—} | coding | coding | coding | coding | coding | coding |
| NM_000249.4 | ENST00000673673.1 | 001354625.2 | ENST00000441265.6 | ENST00000673715.1 | ENST00000673899.1 | ENST00000616768.5 | ENST00000429117.5 | ENST00000413740.1 |
| 297 | 1 | 1 | 1 | 1 | 1 | 0 | 1 | 0 |
| 336 | 1 | 1 | 1 | 1 | 1 | 0 | 1 | 0 |
| 358 | 1 | 1 | 1 | 1 | 1 | 0 | 1 | 0 |
| 367 | 1 | 1 | 1 | 1 | 1 | 0 | 1 | 0 |
| 391 | 1 | 1 | 1 | 1 | 1 | 0 | 1 | 0 |
| 451 | 1 | 1 | 1 | 1 | 1 | 0 | 1 | 0 |
| 586 | 1 | 1 | 1 | 1 | 1 | 0 | 0 | 0 |
| 633 | 1 | 1 | 1 | 1 | 1 | 0 | 0 | 0 |
| 640 | 1 | 1 | 1 | 1 | 1 | 0 | 0 | 0 |
| 658 | 1 | 1 | 1 | 1 | 1 | 0 | 0 | 0 |
| 665 | 1 | 1 | 1 | 1 | 1 | 0 | 0 | 0 |
| 1272 | 1 | 1 | 1 | 1 | 0 | 1 | 0 | 0 |
| 1476 | 1 | 1 | 1 | 1 | 1 | 1 | 0 | 1 |
| 1494 | 1 | 1 | 1 | 1 | 1 | 1 | 0 | 1 |
| 1520 | 1 | 1 | 1 | 1 | 1 | 1 | 0 | 1 |
| 1523 | 1 | 1 | 1 | 1 | 1 | 1 | 0 | 1 |
| 1539 | 1 | 1 | 1 | 1 | 1 | 1 | 0 | 1 |
| 1575 | 1 | 1 | 1 | 1 | 1 | 1 | 0 | 1 |
| 1587 | 1 | 1 | 1 | 1 | 1 | 1 | 0 | 1 |
| 1594 | 1 | 1 | 1 | 1 | 1 | 1 | 0 | 1 |
| 1595 | 1 | 1 | 1 | 1 | 1 | 1 | 0 | 1 |
| 1605 | 1 | 1 | 1 | 1 | 1 | 1 | 0 | 1 |
| 1654 | 1 | 1 | 1 | 1 | 1 | 1 | 0 | 1 |
| 1656 | 1 | 1 | 1 | 1 | 1 | 1 | 0 | 1 |

| MLH1 / | | | | | | | |
|---|---|---|---|---|---|---|---|
| 1170.0-21 | | | | | | | |
| 5′ | | | | | | | |
| position | x-reactivity (detailed) | | | | | | |
| of 19mer | | | nonsense_{—} | nonsense_{—} | nonsense_{—} | nonsense_{—} | nonsense_{—} |
| target | protein_{—} | retained_{—} | mediated_{—} | mediated_{—} | mediated_{—} | mediated_{—} | mediated_{—} |
| site in | coding | intron | decay | decay | decay | decay | decay |
| NM_000249.4 | ENST00000450420.5 | ENST00000674107.1 | ENST00000673947.1 | ENST00000673972.1 | ENST00000432299.6 | ENST00000674111.1 | ENST00000673897.1 |
| 297 | 0 | 1 | 1 | 1 | 1 | 1 | 1 |
| 336 | 0 | 1 | 1 | 1 | 1 | 1 | 1 |
| 358 | 0 | 1 | 1 | 1 | 1 | 1 | 1 |
| 367 | 0 | 1 | 1 | 1 | 1 | 1 | 1 |
| 391 | 0 | 1 | 1 | 1 | 1 | 1 | 1 |
| 451 | 0 | 1 | 1 | 1 | 1 | 1 | 1 |
| 586 | 0 | 1 | 1 | 1 | 1 | 1 | 1 |
| 633 | 0 | 1 | 1 | 1 | 1 | 1 | 1 |
| 640 | 0 | 1 | 1 | 1 | 1 | 1 | 1 |
| 658 | 0 | 1 | 1 | 1 | 1 | 1 | 1 |
| 665 | 0 | 1 | 1 | 1 | 1 | 1 | 1 |
| 1272 | 0 | 1 | 1 | 1 | 1 | 1 | 1 |
| 1476 | 1 | 1 | 1 | 1 | 1 | 1 | 1 |
| 1494 | 1 | 1 | 1 | 1 | 1 | 1 | 1 |
| 1520 | 1 | 1 | 1 | 1 | 1 | 1 | 1 |
| 1523 | 1 | 1 | 1 | 1 | 1 | 1 | 1 |
| 1539 | 1 | 1 | 1 | 1 | 1 | 1 | 1 |
| 1575 | 0 | 0 | 1 | 1 | 1 | 1 | 1 |
| 1587 | 0 | 0 | 1 | 1 | 1 | 1 | 1 |
| 1594 | 0 | 0 | 1 | 1 | 1 | 1 | 1 |
| 1595 | 0 | 0 | 1 | 1 | 1 | 1 | 1 |
| 1605 | 0 | 0 | 1 | 1 | 1 | 1 | 1 |
| 1654 | 0 | 0 | 1 | 1 | 1 | 1 | 1 |
| 1656 | 0 | 0 | 1 | 1 | 1 | 1 | 1 |

| MLH1 / | | | | | | | |
|---|---|---|---|---|---|---|---|
| 1170.0-21 | | | | | | | |
| 5′ | | | | | | | |
| position | x-reactivity (detailed) | | | | | | |
| of 19mer | nonsense_{—} | nonsense_{—} | | | | | |
| target | mediated_{—} | mediated_{—} | retained_{—} | processed_{—} | retained_{—} | retained_{—} | retained_{—} |
| site in | decay | decay | intron | transcript | intron | intron | intron |
| NM_000249.4 | ENST00000447829.6 | ENST00000413212.2 | ENST00000673889.1 | ENST00000673990.1 | ENST00000673713.1 | ENST0000067374.1 | ENST0000044224.1 |
| 297 | 1 | 1 | 0 | 1 | 1 | 0 | 1 |
| 336 | 0 | 1 | 0 | 1 | 1 | 0 | 1 |
| 358 | 0 | 1 | 0 | 1 | 1 | 0 | 1 |
| 367 | 0 | 1 | 0 | 1 | 1 | 0 | 1 |
| 391 | 0 | 1 | 0 | 1 | 1 | 0 | 1 |
| 451 | 1 | 1 | 0 | 1 | 1 | 0 | 1 |
| 586 | 1 | 1 | 0 | 1 | 1 | 0 | 1 |
| 633 | 1 | 1 | 0 | 1 | 1 | 0 | 1 |
| 640 | 1 | 1 | 0 | 1 | 1 | 0 | 1 |
| 658 | 1 | 1 | 0 | 1 | 1 | 0 | 1 |
| 665 | 1 | 1 | 0 | 1 | 1 | 0 | 1 |
| 1272 | 1 | 1 | 1 | 1 | 1 | 0 | 1 |
| 1476 | 1 | 1 | 1 | 1 | 0 | 0 | 0 |
| 1494 | 1 | 1 | 1 | 1 | 0 | 0 | 0 |
| 1520 | 1 | 1 | 1 | 1 | 0 | 0 | 0 |
| 1523 | 1 | 1 | 1 | 1 | 0 | 0 | 0 |
| 1539 | 1 | 1 | 1 | 1 | 0 | 0 | 0 |
| 1575 | 1 | 1 | 1 | 1 | 0 | 0 | 0 |
| 1587 | 1 | 1 | 1 | 1 | 0 | 0 | 0 |
| 1594 | 1 | 1 | 1 | 1 | 0 | 0 | 0 |
| 1595 | 1 | 1 | 1 | 1 | 0 | 0 | 0 |
| 1605 | 1 | 1 | 1 | 1 | 0 | 0 | 0 |
| 1654 | 1 | 1 | 1 | 1 | 0 | 0 | 0 |
| 1656 | 1 | 1 | 1 | 1 | 0 | 0 | 0 |

| MLH1 / | |
|---|---|
| 1170.0-21 | |
| 5′ | |
| position | x-reactivity (detailed) |

| of 19mer | | | | nonsense_{—} | nonsense_{—} | |
|---|---|---|---|---|---|---|
| target | retained_{—} | retained_{—} | retained_{—} | mediated_{—} | mediated_{—} | processed_{—} |
| site in | intron | intron | intron | decay | decay | transcript |
| NM_000249.4 | ENST00000476172.6 | ENST00000674125.1 | ENST00000673686.1 | ENST00000457004.5 | ENST00000458009.5 | ENST00000492474.5 |
| 297 | 0 | 0 | 0 | 0 | 0 | 1 |
| 336 | 0 | 0 | 0 | 1 | 0 | 1 |
| 358 | 0 | 0 | 0 | 1 | 0 | 1 |
| 367 | 0 | 0 | 0 | 1 | 0 | 1 |
| 391 | 0 | 0 | 0 | 1 | 0 | 1 |
| 451 | 0 | 0 | 0 | 1 | 0 | 1 |
| 586 | 0 | 0 | 0 | 1 | 0 | 0 |
| 633 | 0 | 0 | 0 | 1 | 0 | 0 |
| 640 | 0 | 0 | 0 | 1 | 0 | 0 |
| 658 | 0 | 0 | 0 | 1 | 0 | 0 |
| 665 | 0 | 0 | 0 | 1 | 0 | 0 |
| 1272 | 0 | 0 | 0 | 0 | 1 | 0 |
| 1476 | 0 | 1 | 0 | 0 | 1 | 0 |
| 1494 | 0 | 1 | 0 | 0 | 0 | 0 |
| 1520 | 0 | 1 | 0 | 0 | 0 | 0 |
| 1523 | 0 | 1 | 0 | 0 | 0 | 0 |
| 1539 | 0 | 1 | 0 | 0 | 0 | 0 |
| 1575 | 0 | 1 | 0 | 0 | 0 | 0 |
| 1587 | 0 | 1 | 0 | 0 | 0 | 0 |
| 1594 | 0 | 1 | 0 | 0 | 0 | 0 |
| 1595 | 0 | 1 | 0 | 0 | 0 | 0 |
| 1605 | 0 | 1 | 0 | 0 | 0 | 0 |
| 1654 | 0 | 1 | 0 | 0 | 0 | 0 |
| 1656 | 0 | 1 | 0 | 0 | 0 | 0 |

| MLH1 / | | | | |
|---|---|---|---|---|
| 1170.0-21 | | | | |
| 5′ | | | | |
| position | x-reactivity (detailed) | | | 19mer x- |
| of 19mer | nonsense_{—} | | | reactivity |
| target | mediated_{—} | processed_{—} | processed_{—} | (of relevant |
| site in | decay | transcript | transcript | transcripts) |
| NM_000249.4 | ENST00000454028.5 | ENST00000485889.1 | ENST00000466900.5 | HUMAN |
| 297 | 1 | 1 | 1 | 1 |
| 336 | 1 | 1 | 1 | 1 |
| 358 | 1 | 1 | 1 | 1 |
| 367 | 1 | 1 | 1 | 1 |
| 391 | 1 | 1 | 1 | 1 |
| 451 | 1 | 1 | 1 | 1 |
| 586 | 0 | 0 | 0 | 1 |
| 633 | 0 | 0 | 0 | 1 |
| 640 | 0 | 0 | 0 | 1 |
| 658 | 0 | 0 | 0 | 1 |
| 665 | 0 | 0 | 0 | 1 |
| 1272 | 0 | 0 | 0 | 1 |
| 1476 | 0 | 0 | 0 | 1 |
| 1494 | 0 | 0 | 0 | 1 |
| 1520 | 0 | 0 | 0 | 1 |
| 1523 | 0 | 0 | 0 | 1 |
| 1539 | 0 | 0 | 0 | 1 |
| 1575 | 0 | 0 | 0 | 1 |
| 1587 | 0 | 0 | 0 | 1 |
| 1594 | 0 | 0 | 0 | 1 |
| 1595 | 0 | 0 | 0 | 1 |
| 1605 | 0 | 0 | 0 | 1 |
| 1654 | 0 | 0 | 0 | 1 |
| 1656 | 0 | 0 | 0 | 1 |

### Prime Editing

The term “prime editing” refers to programmable editing of a target DNA using a prime editor complexed with a PEgRNA to incorporate an intended nucleotide edit into the target DNA through target-primed DNA synthesis. A target polynucleotide, e.g., a target gene of prime editing may comprise a double stranded DNA molecule having two complementary strands: a first strand that may be referred to as a “target strand” or a “non-edit strand,” and a second strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence.” In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand.” In some embodiments, the PAM strand comprises a protospacer sequence and optionally a protospacer adjacent motif (PAM) sequence. In prime editing using a Cas-protein-based prime editor, a PAM sequence refers to a short DNA sequence immediately adjacent to the protospacer sequence on the PAM strand of the target gene. A PAM sequence may be specifically recognized by a programmable DNA binding protein, e.g., a Cas nickase or a Cas nuclease. In some embodiments, a specific PAM is characteristic of a specific programmable DNA binding protein, e.g., a Cas nickase or a Cas nuclease A protospacer sequence refers to a specific sequence in the PAM strand of the target gene that is complementary to the search target sequence. In a PEgRNA, a spacer sequence may have a substantially identical sequence as the protospacer sequence on the edit strand of a target gene, except that the spacer sequence may comprise Uracil (U) and the protospacer sequence may comprise Thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is 3 base pairs upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 base pairs upstream of the PAM sequence, and the PAM sequence is recognized by a Cas9 nickase, wherein the Cas9 nickase comprises a nuclease active HNH domain and a nuclease inactive RuvC domain. In some embodiments, the nick site is 2 base pairs upstream of the PAM sequence, and the PAM sequence is recognized by a S. thermophilus Cas9 nickase.

A “primer binding site” (PBS or primer binding site sequence) is a single-stranded portion of the PEgRNA that comprises a region of complementarity to the PAM strand (i.e. the non-target strand or the edit strand). The PBS is complementary or substantially complementary to a sequence on the PAM strand of the double stranded target DNA that is immediately upstream of the nick site. In some embodiments, in the process of prime editing, the PEgRNA complexes with and directs a prime editor to bind the search target sequence on the target strand of the double stranded target DNA, and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

An “editing template” of a PEgRNA is a single-stranded portion of the PEgRNA that is 5′ of the PBS and comprises a region of complementarity to the PAM strand (i.e. the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other. In some embodiments, the single stranded portion of the PEgRNA comprising both the PBS and the editing template is complementary or substantially complementary to an endogenous sequence on the PAM strand (i.e. the non-target strand or the edit strand) of the double stranded target DNA except for one or more non-complementary nucleotides at the intended nucleotide edit positions. As used herein, regardless of relative 5′-3′ positioning in other context, the relative positions as between the PBS and the editing template, and the relative positions as among elements of a PEgRNA, are determined by the 5′ to 3′ order of the PEgRNA as a single molecule regardless of the position of sequences in the double stranded target DNA that may have complementarity or identity to elements of the PEgRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence.” In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits.

In some embodiments, a PEgRNA complexes with and directs a prime editor to bind to the search target sequence of the target gene. In some embodiments, the bound prime editor generates a nick on the edit strand (PAM strand) of the target gene at the nick site. In some embodiments, a primer binding site (PBS) of the PEgRNA anneals with a free 3′ end formed at the nick site, and the prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to the endogenous target gene sequence. In some embodiments, the editing template of a PEgRNA is complementary to a sequence in the edit strand except for one or more mismatches at the intended nucleotide edit positions in the editing template partially complementary to the editing template may be referred to as an “editing target sequence.” Accordingly, in some embodiments, the newly synthesized single stranded DNA has identity or substantial identity to a sequence in the editing target sequence, except for one or more insertions, deletions, or substitutions at the intended nucleotide edit positions.

In some embodiments, the newly synthesized single-stranded DNA equilibrates with the editing target on the edit strand of the target gene for pairing with the target strand of the target gene. In some embodiments, the editing target sequence of the target gene is excised by a flap endonuclease (FEN), for example, FEN1. In some embodiments, the FEN is an endogenous FEN, for example, in a cell comprising the target gene. In some embodiments, the FEN is provided as part of the prime editor, either linked to other components of the prime editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the target gene. In some embodiments, the newly synthesized single-stranded DNA comprising the nucleotide edit is paired in the heteroduplex with the target strand of the target DNA that does not comprise the nucleotide edit, thereby creating a mismatch between the two otherwise complementary strands. In some embodiments, the mismatch is recognized by DNA repair machinery, e.g., an endogenous DNA repair machinery. In some embodiments, through DNA repair, the intended nucleotide edit is incorporated into the target gene.

### Prime Editors

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide and a reverse transcriptase polypeptide that are derived from different species. For example, a prime editor may comprise a S. pyogenes Cas9 polypeptide and a Moloney murine leukemia virus (M-MLV) reverse transcriptase polypeptide. In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

### Prime Editor Nucleotide Polymerase Domain

In some embodiments, a prime editor comprises a nucleotide polymerase domain, e.g., a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the prime editor comprises a DNA-dependent DNA polymerase. For example, a prime editor having a DNA-dependent DNA polymerase can synthesize a new single stranded DNA using a PEgRNA editing template that comprises a DNA sequence as a template. In such cases, the PEgRNA is a chimeric or hybrid PEgRNA, and comprising an extension arm comprising a DNA strand. The chimeric or hybrid PEgRNA may comprise an RNA portion (including the spacer and the gRNA core) and a DNA portion (the extension arm comprising the editing template that includes a strand of DNA).

The DNA polymerases can be wild type polymerases from eukaryotic, prokaryotic, archael, or viral organisms, and/or the polymerases may be modified by genetic engineering, mutagenesis, or directed evolution-based processes. The polymerases can be a T7 DNA polymerase, T5 DNA polymerase, T4 DNA polymerase, Klenow fragment DNA polymerase, DNA polymerase III and the like. The polymerases can be thermostable, and can include Taq, Tne, Tma, Pfu, Tfl, Tth, Stoffel fragment, VENT® and DEEPVENT® DNA polymerases, KOD, Tgo, JDF3, and mutants, variants and derivatives thereof.

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol IV family DNA polymerase. In some embodiments, the DNA polymerase is a E. coli Pol IV DNA polymerase.

In some embodiments, the DNA polymerase comprises a eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a RevI DNA polymerase. In some embodiments, the DNA polymerase is a human RevI DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcusfuriosus. In some embodiments, the DNA polymerase is a pol II type DNA polymerase. For example, in some embodiments, the DNA polymerase is a homolog of P. furiosus DP1/DP2 2-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase comprises a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol III family DNA polymerase. In some embodiments, the DNA Polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UlTma).

In some embodiments, a prime editor comprises an RNA-dependent DNA polymerase domain, for example, a reverse transcriptase (RT). A RT or an RT domain may be a wild type RT domain, a full-length RT domain, or may be a functional mutant, a functional variant, or a functional fragment thereof. An RT or an RT domain of a prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, a prime editor comprises a virus RT, for example, a retrovirus RT. Non-limiting examples of virus RT include Moloney murine leukemia virus (M-MLV or MLVRT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild type M-MLV RT. An exemplary sequence of a wild type M-MLV RT is provided in SEQ ID NO: 901. In some embodiments, the prime editor comprises a M-MLV RT comprising a H8Y amino acid substitution. Collectively, the wild type M-MLV RT and the H8Y M-MLV RT are referred to as reference M-MLV RTs.

In some embodiments, the prime editor comprises a M-MMLV RT comprising one or more of amino acid substitutions P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to the reference M-MMLV RT as set forth in SEQ ID NO: 901, where X is any amino acid other than the amino acid in the corresponding reference M-MLV. In some embodiments, the prime editor comprises a M-MMLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to the reference M-MMLV RT as set forth in SEQ ID NO: 901. In some embodiments, the prime editor comprises a M-MLV RT comprising one or more amino acid substitutions D200N, T330P, L603W, T306K, and W313F as compared to the reference-MMLV RT as set forth in SEQ ID NO: 901. In some embodiments, the prime editor comprises a M-MLV RT comprising amino acid substitutions D200N, T330P, L603W, T306K, and W313F as compared to the reference M-MMLV RT as set forth in SEQ ID NO: 901. In some embodiments, a prime editor comprising the D200N, T330P, L603W, T306K, and W313F as compared to the reference M-MMLV RT maybe referred to as a “PE2” prime editor, and the corresponding prime editing system a PE2 prime editing system.

In some embodiments, an RT variant may be a functional fragment of a reference RT that have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT, e.g., a reference RT. In some embodiments, the RT variant comprises a fragment of a reference RT, e.g., a reference RT, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of the reference RT. In some embodiments, the fragment is 30%, 35%, 40%, 45%, 50%, 55%, 60%, 65%, 70%, 75%, 80%, 85%, 90%, 95% identical, 96%, 97%, 98%, 99%, or 99.5% of the amino acid length of a corresponding reference RT (M-MLV reverse transcriptase) (e.g., SEQ ID NO: 901).

In some embodiments, the RT functional fragment is at least 100 amino acids in length. In some embodiments, the fragment is at least 100, 150, 200, 250, 300, 350, 400, 450, 500, 550, or up to 600 or more amino acids in length.

In still other embodiments, the functional RT variant is truncated at the N-terminus or the C-terminus, or both, by a certain number of amino acids which results in a truncated variant which still retains sufficient DNA polymerase function. In some embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the N-terminal end compared to a reference RT, e.g., a wild type RT. In some embodiments, the reference RT is a wild type M-MLV RT. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the C-terminal end compared to a reference RT, e.g., a wild type RT. In some embodiments, the reference RT is a wild type M-MLV RT. In still other embodiments, the RT truncated variant has a truncation at the N-terminal and the C-terminal end compared to a reference RT, e.g., a wild type RT. In some embodiments, the N-terminal truncation and the C-terminal truncation are of the same length. In some embodiments, the N-terminal truncation and the C-terminal truncation are of different lengths.

For example, the prime editors disclosed herein may include a functional variant of a wild type M-MLV reverse transcriptase. In some embodiments, the prime editor comprises a functional variant of a wild type M-MLV RT, wherein the functional variant of M-MLV RT is truncated after amino acid position 502 compared to a wild type M-MLV RT as set forth in SEQ ID NO: 901. In some embodiments, the functional variant of M-MLV RT further comprises a D200X, T306X, W313X, and/or T330X amino acid substitution compared to compared to a wild type M-MLV RT as set forth in SEQ ID NO: 901, wherein X is any amino acid other than the original amino acid. In some embodiments, the functional variant of M-MLV RT further comprises a D200N, T306K, W313F, and/or T330P amino acid substitution compared to a M-MLV RT as set forth in SEQ ID NO: 901, wherein X is any amino acid other than the original amino acid. A DNA sequence encoding a prime editor comprising this truncated RT is 522 bp smaller than PE2, and therefore makes its potentially useful for applications where delivery of the DNA sequence is challenging due to its size (i.e., adeno-associated virus and lentivirus delivery). In some embodiments, a prime editor comprises a M-MLV RT variant, wherein the M-MLV RT consists of the following amino acid sequence:

(SEQ ID NO: 2469)
TLNIEDEYRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFNEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGKAGFCRLFIPGFAEMAAPLYPLTKPGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDNSRLIN.

In some embodiments, the functional variant of M-MLV RT comprises a D200N, T306K, W313F, T330P, and L603W amino acid substitution compared to a reference M-MLV RT.

In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT.

### Programmable DNA Binding Domain

In some embodiments, the DNA-binding domain of a prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a target gene. In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprise a nuclease activity. In some embodiments, the DNA-binding domain of a prime editor comprises an endonuclease domain having single strand DNA cleavage activity. For example, the endonuclease domain may comprise a FokI nuclease domain. In some embodiments, the DNA-binding domain of a prime editor comprises a nuclease having full nuclease activity. In some embodiments, the DNA-binding domain of a prime editor comprises a nuclease having modified or reduced nuclease activity as compared to a wild type endonuclease domain. For example, the endonuclease domain may comprise one or more amino acid substitutions as compared to a wild type endonuclease domain. In some embodiments, the DNA-binding domain of a prime editor has nickase activity. In some embodiments, the DNA-binding domain of a prime editor comprises a Cas protein domain that is a nickase. In some embodiments, compared to a wild type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas9, Cas12a (Cpf1), Cas12e (CasX), Cas12d (CasY), Cas12b1 (C2c1), Cas12b2, Cas12c (C2c3), C2c4, C2c8, C2c5, C2c10, C2c9, Cas14a, Cas14b, Cas14c, Cas14d, Cas14e, Cas14f, Cas14g, Cas14h, Cas14u, Cns2, Cas Φ, and homologs, functional fragments, or modified versions thereof. A Cas protein can be a chimeric Cas protein that is fused to other proteins or polypeptides. A Cas protein can be a chimera of various Cas proteins, for example, comprising domains of Cas proteins from different organisms.

A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis.

A Cas protein, e.g., Cas9, can be a wild type or a modified form of a Cas protein. A Cas protein, e.g., Cas9, can be a nuclease active variant, nuclease inactive variant, a nickase, or a functional variant or functional fragment of a wild type Cas protein. A Cas protein, e.g., Cas9, can comprise an amino acid change such as a deletion, insertion, substitution, fusion, chimera, or any combination thereof relative to a wild-type version of the Cas protein. A Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a PEgRNA.

In some embodiments, a prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand or the non-edit strand of the target gene, but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain. In some embodiments, the Cas9 nickase comprises a D10X amino acid substitution compared to a wild type S. pyogenes Cas9, wherein X is any amino acid other than D. In some embodiments, a prime editor comprises a Cas9 nickase having an amino acid substitution in the HNH domain. In some embodiments, the Cas9 nickase comprises a H840X amino acid substitution compared to a wild type S. pyogenes Cas9, wherein X is any amino acid other than H.

In some embodiments, a prime editor comprises a Cas protein that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some aspects, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild type Cas9 protein from any organism or a homolog, ortholog, or paralog from any organisms; any functional mutants or functional variants thereof; or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild type reference Cas9 protein (e.g., Cas9 from S. pyogenes). In some embodiments, the Cas9 comprises an amino acid change such as a deletion, insertion, substitution, fusion, chimera, or any combination thereof as compared to a wild type reference Cas9 protein.

In some embodiments, a Cas9 protein may comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art. In some embodiments, a Cas9 polypeptide is a SpCas9 polypeptide. In some embodiments, a Cas9 polypeptide is a SaCas9 polypeptide. In some embodiments, a Cas9 polypeptide is a ScCas9 polypeptide. In some embodiments, a Cas9 polypeptide is a StCas9 polypeptide. In some embodiments, a Cas9 polypeptide is a SluCas9 polypeptide. In some embodiments, a Cas9 polypeptide is a NmCas9 polypeptide. In some embodiments, a Cas9 polypeptide is a CjCas9 polypeptide. In some embodiments, a Cas9 polypeptide is a FnCas9 polypeptide. In some embodiments, a Cas9 polypeptide is a TdCas9 polypeptide. In some embodiments, a Cas9 polypeptide is a chimera comprising domains from two or more of the organisms described herein or those known in the art. In some embodiments, a Cas9 polypeptide is a Cas9 polypeptide from Streptococcus macacae. In some embodiments, a Cas9 polypeptide is a Cas9 polypeptide generated by replacing a PAM interaction domain of a SpCas9 with that of a Streptococcus macacae Cas9 (Spy-mac Cas9).

An exemplary Streptococcus pyogenes Cas9 (SpCas9) amino acid sequence is provided in SEQ ID NO: 902.

In some embodiments, a prime editor comprises a Cas9 protein from Staphylococcus lugdunensis (Slu Cas9). An exemplary amino acid sequence of a Slu Cas9 is provided in SEQ ID NO: 903.

In some embodiments, a Cas9 protein comprises a variant Cas9 protein containing one or more amino acid substitutions. In some embodiments, a wildtype Cas9 protein comprises a RuvC domain and an HNH domain. In some embodiments, a prime editor comprises a nuclease active Cas9 protein that may cleave both strands of a double stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA, but can cleave only one strand of a double stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e., the PAM strand), but not the non-edit strand of a double stranded target DNA sequence. In some embodiments, a prime editor comprises a Cas9 having a non-functional RuvC domain that can cleave the target strand (i.e., the non-PAM strand), but not the edit strand of a double stranded target DNA sequence. In some embodiments, a prime editor comprises a Cas9 that has neither a functional RuvC domain nor a functional HNH domain, which may not cleave any strand of a double stranded target DNA sequence.

In some embodiments, a prime editor comprises a Cas9 having a mutation in the RuvC domain that reduces or abolishes the nuclease activity of the RuvC domain. In some embodiments, the Cas9 comprise a mutation at amino acid D10 as compared to a wild type SpCas9 as set forth in SEQ ID NO: 902, or a corresponding mutation thereof. In some embodiments, the Cas9 comprise a D10A mutation as compared to a wild type SpCas9 as set forth in SEQ ID NO: 902, or a corresponding mutation thereof. In some embodiments, the Cas9 polypeptide comprise a mutation at amino acid D10, G12, and/or G17 as compared to a wild type SpCas9 as set forth in SEQ ID NO: 902, or a corresponding mutation thereof. In some embodiments, the Cas9 polypeptide comprise a D10A mutation, a G12A mutation, and/or a G17A mutation as compared to a wild type SpCas9 as set forth in SEQ ID NO: 902, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprise a mutation at amino acid H840 as compared to a wild type SpCas9 as set forth in SEQ ID NO: 902, or a corresponding mutation thereof. In some embodiments, the Cas9 polypeptide comprise a H840A mutation as compared to a wild type SpCas9 as set forth in SEQ ID NO: 902, or a corresponding mutation thereof. In some embodiments, the Cas9 polypeptide comprise a mutation at amino acid E762, D839, H840, N854, N856, N863, H982, H983, A984, D986, and/or a A987 as compared to a wild type SpCas9 as set forth in SEQ ID NO: 902, or a corresponding mutation thereof. In some embodiments, the Cas9 polypeptide comprise a E762A, D839A, H840A, N854A, N856A, N863A, H982A, H983A, A984A, and/or a D986A mutation as compared to a wild type SpCas9 as set forth in SEQ ID NO: 902, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840X substitution and a D10X mutation compared to a wild type SpCas9 as set forth in SEQ ID NO: 902 or corresponding mutations thereof, wherein X is any amino acid other than H for the H840X substitution and any amino acid other than D for the D10X substitution. In some embodiments, the dead Cas9 comprises a H840A and a D10A mutation as compared to a wild type SpCas9 as set forth in SEQ ID NO: 902, or corresponding mutations thereof.

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto.

Besides dead Cas9 and Cas9 nickase variants, the Cas9 proteins used herein may also include other Cas9 variants having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference Cas9, e.g., a wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, a Cas9 fragment is a functional fragment that retains one or more Cas9 activities. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, a prime editor comprises a Cas protein, e.g., Cas9, containing modifications that allow altered PAM recognition. In prime editing using a Cas-protein-based prime editor, a “protospacer adjacent motif (PAM),” PAM sequence, or PAM-like motif, may be used to refer to a short DNA sequence immediately following the protospacer sequence on the PAM strand of the target gene. In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. Exemplary PAM sequences and corresponding Cas variants are described in Table A below. It should be appreciated that for each of the variants provided, the Cas protein comprises one or more of the amino acid substitutions as indicated compared to a wild type Cas protein sequence, for example, the Cas9 as set forth in SEQ ID NO: 902. The PAM motifs as shown in Table A below are in the order of 5′ to 3′.

The nucleotides listed in Table A are represented by the base codes as provided in the Handbook on Industrial Property Information and Documentation, World Intellectual Property Organization (WIPO) Standard ST.26, Version 1.4. For example, an “R” in Table A represents the nucleotide A or G, and “W” in Table A represents A or T.

TABLE A
Cas protein variants and corresponding PAM sequences

| Variant | PAM |
|---|---|
| spCas9 (wild type) | NGG, NGA, NAG, NGNGA |
| spCas9-VRVRFRR R1335V, L1111R, D1135V, | NG |
| G1218R, E1219F, A1322R, T1337R | |
| spCas9-VQR (D1135V, R1335Q, T1337R) | NGA |
| spCas9-EQR (D1135E, R1335Q, T1337R) | NGA |
| spCas9-VRER (D1135V, G1218R, R1335E, T1337R) | NGCG |
| spCas9-VRQR (D1135V, G1218R, R1335Q, T1337R) | NGA |
| Cas9-NG (L1111R, D1135V, G1218R, E1219F, | NGN |
| A1322R, T1337R, R1335V) | |
| SpG Cas9 (D1135L, S1136W, G1218K, E1219Q, | NGN |
| R1335Q, T1337R) | |
| SyRY Cas9 | NRN |
| (A61R, L1111R, N1317R, A1322R, and R1333P) | |
| xCas9 (E480K, E543D, E1219V, K294R, Q1256K, | NGN |
| A262T, S409I, M694I) | |
| SluCa9 | NNGG |
| SRGN1, sRGN2, sRGN4, sRGN3.1, sRGN3.3 | NNGG |
| saCas9 | NNGRRT NNGRRN |
| saCas9-KKH (E782K, N968K, R1015H) | NNNRRT |
| spCas9-MOKSER (D1135M, S1136Q, G1218K, | NGCG/NGCN |
| E1219S, R1335E, T1337R) | |
| spCas9-LRKIQK (D1135L, S1136R, G1218K, | NGTN |
| E1219I, R1335Q, T1337K) | |
| spCas9-LRVSQK (D1135L, S1136R, G1218V, | NGTN |
| E1219S, R1335Q, T1337K) | |
| spCas9-LRVSQL (D1135L, S1136R, G1218V, | NGTN |
| E1219S, R1335Q, T1337L) | |
| Cpf1 | TTTV |
| Spy-Mac | NAA |
| NmCas9 | NNNNGATT |
| StCas9 | NNAGAAW |
| TdCas9 | NAAAAC |

In some embodiments, a prime editor comprises a Cas9 polypeptide comprising one or mutations selected from the group consisting of: A61R, L111R, D1135V, R221K, A262T, R324L, N394K, S409I, S409I, E427G, E480K, M495V, N497A, Y515N, K526E, F539S, E543D, R654L, R661A, R661L, R691A, N692A, M694A, M694I, Q695A, H698A, R753G, M763I, K848A, K890N, Q926A, K1003A, R1060A, L1111R, R1114G, D1135E, D1135L, D1135N, S1136W, V1139A, D1180G, G1218K, G1218R, G1218S, E1219Q, E1219V, E1219V, Q1221H, P1249S, E1253K, N1317R, A1320V, P1321S, A1322R, I1322V, D1332G, R1332N, A1332R, R1333K, R1333P, R1335L, R1335Q, R1335V, T1337N, T1337R, S1338T, H1349R, and any combinations thereof as compared to a wildtype SpCas9 polypeptide as set forth in SEQ ID NO: 902.

In some embodiments, a prime editor comprises a SaCas9 polypeptide. In some embodiments, the SaCas9 polypeptide comprises one or more of mutations E782K, N968K, and R1015H as compared to a wild type SaCas9. In some embodiments, a prime editor comprises a FnCas9 polypeptide, for example, a wildtype FnCas9 polypeptide or a FnCas9 polypeptide comprising one or more of mutations E1369R, E1449H, or R1556A as compared to the wild type FnCas9. In some embodiments, a prime editor comprises a Sc Cas9, for example, a wild type ScCas9 or a ScCas9 polypeptide comprises one or more of mutations I367K, G368D, I369K, H371L, T375S, T376G, and T1227K as compared to the wild type ScCas9. In some embodiments, a prime editor comprises a St1 Cas9 polypeptide, a St3 Cas9 polypeptide, or a Slu Cas9 polypeptide.

In some embodiments, a prime editor comprises a Cas polypeptide that comprises a circular permutant Cas variant. For example, a Cas9 polypeptide of a prime editor may be engineered such that the N-terminus and the C-terminus of a Cas9 protein (e.g., a wild type Cas9 protein, or a Cas9 nickase) are topically rearranged to retain the ability to bind DNA when complexed with a guide RNA (gRNA). An exemplary circular permutant configuration may be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

In various embodiments, the circular permutants of a Cas protein, e.g., a Cas9, may have the following structure: N-terminus-[original C-terminus]-[optional linker]-[original N-terminus]-C-terminus. In some embodiments, a circular permutant Cas9 comprises any one of the following structures (amino acid positions as set forth in SEQ ID NO: 902):
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[I1-167]-C-terminus;
N-terminus-[68-1368]-[optional linker]-[1-67]-C-terminus;
N-terminus-[10-1368]-[optional linker]-[1-9]-C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 902-1368 amino acids of UniProtKB—Q99ZW2:
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 902-1368 amino acids of UniProtKB—Q99ZW2 N-terminus-[103-1368]-[optional linker]-[1-102]-C-terminus:
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment may correspond to the 95% or more of the C-terminal amino acids of a Cas9, or the 90%, 85%, 80%, 75%, 70%, 65%, 60%, 55%, 50%, 45%, 40%, 35%, 30%, 25%, 20%, 15%, 10%, or 5% or more of the C-terminal amino acids of a Cas9 (e.g., SEQ ID NO: 902 or a ortholog or a variant thereof). The N-terminal portion may correspond to 95% or more of the N-terminal amino acids of a Cas9 (e.g., amino acids about 1-1300 as set forth in SEQ ID No: 902 or corresponding amino acid positions thereof), or 90%, 85%, 80%, 75%, 70%, 65%, 60%, 55%, 50%, 45%, 40%, 35%, 30%, 25%, 20%, 15%, 10%, or 5% or more of the N terminal amino acids of a Cas9 (e.g., as set forth in SEQ ID No: 902 or corresponding amino acid positions thereof).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 as set forth in SEQ ID No: 902 or corresponding amino acid positions thereof). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., as set forth in SEQ ID No: 902 or corresponding amino acid positions thereof). In some embodiments, the C-terminal fragment that is rearranged to the N-terminus, includes or corresponds to the C-terminal 410 residues or less of a Cas9 (e.g., as set forth in SEQ ID No: 902 or corresponding amino acid positions thereof). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e/g/as set forth in SEQ ID No: 902 or corresponding amino acid positions thereof). In some embodiments, the C-terminal portion that is rearranged to the N-terminus includes or corresponds to the C-terminal 357, 341, 328, 120, or 69 residues of a Cas9 (e.g., as set forth in SEQ ID No: 902 or corresponding amino acid positions thereof).

In other embodiments, circular permutant Cas9 variants may be a topological rearrangement of a Cas9 primary structure based on the following method, which is based on S. pyogenes Cas9 of SEQ ID NO: 902: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (as set forth in SEQ ID No: 902 or corresponding amino acid positions thereof) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from any particular sequence but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

In some embodiments, a prime editor comprises a Cas9 functional variant that is of smaller molecular weight than a wild type SpCas9 protein. In some embodiments, a smaller-sized Cas9 functional variant may facilitate delivery to cells, e.g., by an expression vector, nanoparticle, or other means of delivery. In certain embodiments, a smaller-sized Cas9 functional variant is a Class 2 Type II Cas protein. In certain embodiments, a smaller-sized Cas9 functional variant is a Class 2 Type V Cas protein. In certain embodiments, a smaller-sized Cas9 functional variant is a Class 2 Type VI Cas protein.

In some embodiments, a prime editor comprises a SpCas9 that is 1368 amino acids in length and has a predicted molecular weight of 158 kilodaltons. In some embodiments, a prime editor comprises a Cas9 functional variant or functional fragment that is less than 1300 amino acids, less than 1290 amino acids, than less than 1280 amino acids, less than 1270 amino acids, less than 1260 amino acid, less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids, but at least larger than about 400 amino acids and retaining the one or more functions, e.g., DNA binding function, of the Cas9 protein.

In some embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide). In various other embodiments, the napDNAbp can be any of the following proteins: a Cas9, a Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a functional variant or fragment thereof.

Exemplary Cas proteins and nomenclature are shown in Table B below:

TABLE B
Exemplary Cas proteins and nomenclature

| | |
|---|---|
| Legacy nomenclature | Current nomenclature |

type II CRISPR-Cas enzymes

| | |
|---|---|
| Cas9 | Same |

type V CRISPR-Cas enzymes

| | |
|---|---|
| Cpf1 | Cas12a |
| CasX | Cas12e |
| C2c1 | Cas12b1 |
| Cas12b2 | Same |
| C2c3 | Cas12c |
| CasY | Cas12d |
| C2c4 | Same |
| C2c8 | Same |
| C2c5 | Same |
| C2c10 | Same |
| C2c9 | Same |

type VI CRISPR-Cas enzymes

| | |
|---|---|
| C2c2 | Cas13a |
| Cas13d | same |
| C2c7 | Cas13c |
| C2c6 | Cas13b |

In some embodiments, a prime editor as described herein may comprise a Cas12a (Cpf1) polypeptide or functional variants thereof. In some embodiments, the Cas12a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12a polypeptide.

In some embodiments, a prime editor comprises a Cas protein that is a Cas12b (C2c1) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c3) protein. In some embodiments, the Cas protein is a Cas12b nickase or a Cas12c nickase. In some embodiments, the Cas protein is a Cas12e, a Cas12d, a Cas13, Cas14a, Cas14b, Cas14c, Cas14d, Cas14e, Cas14f, Cas14g, Cas14h, Cas14u, or a CasΦ polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally-occurring Cas12e, Cas12d, Cas13, Cas14a, Cas14b, Cas14c, Cas14d, Cas14e, Cas14f, Cas14g, Cas14h, Cas14u, or Cas (protein. In some embodiments, the Cas protein is a Cas12e, Cas12d, Cas13, or Cas (nickase.

### Flap Endonuclease

In some embodiments, a prime editor further comprises additional polypeptide components, for example, a flap endonuclease (FEN, e.g., FEN1). In some embodiments, the flap endonuclease excises the 5′ single stranded DNA of the edit strand of the target gene and assists incorporation of the intended nucleotide edit into the target gene. In some embodiments, the FEN is linked or fused to another component. In some embodiments, the FEN is provided in trans, for example, as a separate polypeptide or polynucleotide encoding the FEN.

In some embodiments, a prime editor or prime editing composition comprises a flap nuclease. In some embodiments, the flap nuclease is a FEN1, or any FEN1 functional variant, functional mutant, or functional fragment thereof. In some embodiments, the flap nuclease is a TREX2, EXO1, or any other flap nuclease known in the art, or any functional variant, functional mutant, or functional fragment thereof. In some embodiments, the flap nuclease has amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the flap nucleases described herein or known in the art.

### Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase, that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing complex comprises at least one NLS. In some embodiments, a prime editor or prime editing complex comprises at least two NLSs. In embodiments with at least two NLSs, the NLSs can be the same NLS, or they can be different NLSs.

In some instances, a prime editor may further comprise at least one nuclear localization sequence (NLS). In some cases, a prime editor may further comprise 1 NLS. In some cases, a prime editor may further comprise 2 NLSs. In other cases, a prime editor may further comprise 3 NLSs. In one case, a primer editor may further comprise more than 4, 5, 6, 7, 8, 9 or 10 NLSs.

In addition, the NLSs may be expressed as part of a prime editor complex. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is fusion protein that comprises an NLS at the N terminus. In some embodiments, a prime editor is fusion protein that comprises an NLS at the C terminus. In some embodiments, a prime editor is fusion protein that comprises at least one NLS at both the N terminus and the C terminus. In some embodiments, the prime editor is a fusion protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, a nuclear localization signal (NLS) is predominantly basic. In some embodiments, the one or more NLSs of a prime editor are rich in lysine and arginine residues. In some embodiments, the one or more NLSs of a prime editor comprise proline residues. In some embodiments, a nuclear localization signal (NLS) comprises the sequence MDSLLMNRRKFLYQFKNVRWAKGRRETYLC (SEQ ID NO: 2470), KRTADGSEFESPKKKRKV (SEQ ID NO: 2471), KRTADGSEFEPKKKRKV (SEQ ID NO: 2472), NLSKRPAAIKKAGQAKKKK (SEQ ID NO: 2473), RQRRNELKRSF (SEQ ID NO: 2474), or NQSSNFGPMKGGNFGGRSSGPYGGGGQYFAKPRNQGGY (SEQ ID NO: 2475).

In some embodiments, a NLS is a monopartite NLS. For example, in some embodiments, a NLS is a SV40 large T antigen NLS PKKKRKV (SEQ ID NO: 2476). In some embodiments, a NLS is a bipartite NLS. In some embodiments, a bipartite NLS comprises two basic domains separated by a spacer sequence comprising a variable number of amino acids. In some embodiments, a NLS is a bipartite NLS. In some embodiments, a bipartite NLS consists of two basic domains separated by a spacer sequence comprising a variable number of amino acids. In some embodiments, the spacer amino acid sequence comprises the sequence KRXXXXXXXXXXKKKL (SEQ ID NO: 2477) Xenopus nucleoplasmin NLS), wherein X is any amino acid. In some embodiments, the NLS comprises a nucleoplasmin NLS sequence KRPAATKKAGQAKKKK (SEQ ID NO: 2478). In some embodiments, a NLS is a noncanonical sequences such as M9 of the hnRNP A1 protein, the influenza virus nucleoprotein NLS, and the yeast Gal4 protein NLS. In some embodiments, a NLS is a noncanonical sequences such as M9 of the hnRNP A1 protein, the influenza virus nucleoprotein NLS, and the yeast Gal4 protein NLS.

Other non-limiting examples of NLS sequences are provided in Table C below.

TABLE C
Exemplary nuclear localization sequences

| Description | SEQ ID NO: | Sequence |
|---|---|---|
| NLS of SV40 | 2476 | PKKKRKV |
| Large T-AG | | |
| NLS | 2479 | MKRTADGSEFESPKKKRKV |
| NLS | 2470 | MDSLLMNRRKFLYQFKNVRWAKGRRETYLC |
| NLS of | 2480 | AVKRPAATKKAGQAKKKKLD |
| Nucleoplasmin | | |
| NLS of EGL-13 | 2481 | MSRRRKANPTKLSENAKKLAKEVEN |
| NLS of C-Myc | 2482 | PAAKRVKLD |
| NLS of Tus-protein | 2483 | KLKIKRPVK |
| NLS of polyoma | 2484 | VSRKRPRP |
| large T-AG | | |
| NLS of Hepatitis | 2485 | EGAPPAKRAR |
| D virus antigen | | |
| NLS of murine p53 | 2486 | PPQPKKKPLDGE |
| NLS of PE1 and | 2487 | SGGSKRTADGSEFEPKKKRKV |
| PE2 | | |
| NLS | 2471 | KRTADGSEFESPKKKRKV |
| NLS | 2472 | KRTADGSEFEPKKKRKV |
| NLS | 2473 | NLSKRPAAIKKAGQAKKKK |
| NLS | 2474 | RQRRNELKRSF |
| NLS | 2475 | NQSSNFGPMKGGNFGGRSSGPYGGGGQYFAKPRNQGGY |
| NLS of Xenopus | 2477 | KRXXXXXXXXXXKKKL |
| nucleoplasmin | | |
| NLS of Xenopus | 2488 | KRPAATKKAGQAKKKK |
| nucleoplasmin | | |
| NLS | 2489 | SGGSSGGSKRTADGSEFESPKKKRKVSGGSSGGS |
| NLS | 2490 | SGGSKRTADGSEFESPKKKRKV |
| NLS | 2491 | GSGPAAKRVKLD |

### Additional Prime Editor Components

A prime editor described herein may comprise additional functional domains, for example, one or more domains that modify the folding, solubility, or charge of the prime editor. In some instances, the prime editor may comprise a solubility-enhancement (SET) domain.

In some embodiments, a split intein comprises two halves of an intein protein, which may be referred to as a N-terminal half of an intein, or intein-N, and a C-terminal half of an intein, or intein-C, respectively. In some embodiments, the intein-N and the intein-C may each be fused to a protein domain (the N-terminal and the C-terminal exteins). The exteins can be any protein or polypeptides, for example, any prime editor polypeptide component. In some embodiments, the intein-N and intein-C of a split intein can associate non-covalently to form an active intein and catalyze a-trans splicing reaction. In some embodiments, the trans splicing reaction excises the two intein sequences and links the two extein sequences with a peptide bond. As a result, the intein-N and the intein-C are spliced out, and a protein domain linked to the intein-N is fused to a protein domain linked to the intein-C. essentially in same way as a contiguous intein does. In some embodiments, a split-intein is derived from a eukaryotic intein, a bacterial intein, or an archaeal intein. Preferably, the split intein so-derived will possess only the amino acid sequences essential for catalyzing trans-splicing reactions. In some embodiments, an intein-N or an intein-C further comprise one or more amino acid substitutions as compared to a wild type intein-N or wild type intein-C, for example, amino acid substitutions that enhances the trans-splicing activity of the split intein. In some embodiments, the intein-C comprises 4 to 7 contiguous amino acid residues, wherein at least 4 amino acids of which are from the last β-strand of the intein from which it was derived. In some embodiments, the split intein is derived from a Ssp DnaE intein, e.g., Synechocytis sp. PCC6803, or any intein or split intein known in the art, or any functional variants or fragments thereof.

In some embodiments, a prime editor comprises one or more epitope tags. Non-limiting examples of epitope tags include histidine (His) tags, V5 tags, FLAG tags, influenza hemagglutinin (HA) tags, Myc tags, VSV-G tags, thioredoxin (Trx) tags, biotin carboxylase carrier protein (BCCP) tags, myc-tags, calmodulin-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, a prime editor comprises one or more polypeptide domains encoded by one or more reporter genes. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

In some embodiments, a prime editor comprises one or more polypeptide domains that binds DNA molecules or binds other cellular molecules. Examples of binding proteins or domains include, but are not limited to, maltose binding protein (MBP), S-tag, Lex A DNA binding domain (DBD) fusions, GAL4 DNA binding domain fusions, and herpes simplex virus (HSV) BP16 protein fusions.

In some embodiments, a prime editor comprises a protein domain that is capable of modifying the intracellular half-life of the prime editor.

In some embodiments, a prime editing complex comprises a fusion protein comprising a DNA binding domain (e.g., Cas9(H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 904.

Polypeptides comprising components of a prime editor may be fused via peptide linkers, or may be provided in trans relevant to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer fused or linked to a portion of the PEgRNA or ngRNA. For example, an MS2 coat protein fused or linked to the DNA polymerase and a MS2 hairpin installed on the PEgRNA for co-localization of the DNA polymerase and the RNA-guided DNA binding domain (e.g., a Cas9 nickase).

In some embodiments, a prime editor comprises a polypeptide domain, an MS2 coat protein (MCP), that recognizes an MS2 hairpin. In some embodiments, the nucleotide sequence of the MS2 hairpin (or equivalently referred to as the “MS2 aptamer”) is: GCCAACATGAGGATCACCCATGTCTGCAGGGCC (SEQ ID NO: 2492). In some embodiments, the amino acid sequence of the MCP is:

(SEQ ID NO: 2493)
GSASNFTQFVLVDNGGTGDVTVAPSNFANGVAEWISSNSRSQAYKVTCS
VRQSSAQNRKYTIKVEVPKVATQTVGGEELPVAGWRSYLNMELTIPIFA
TNSDCELIVKAMQGLLKDGNPIPSAIAANSGIY.

In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)n (SEQ ID NO: 2494), (G)n (SEQ ID NO: 2495), (EAAAK)n (SEQ ID NO: 2496), (GGS)n (SEQ ID NO: 2497), (SGGS)n (SEQ ID NO: 2498), (XP)n (SEQ ID NO: 2499), or any combination thereof, wherein n is independently an integer between 1 and 30, and wherein X is any amino acid. In some embodiments, the linker comprises the amino acid sequence (GGS)n (SEQ ID NO: 2500), wherein n is 1, 3, or 7. In some embodiments, the linker comprises the amino acid sequence SGSETPGTSESATPES (SEQ ID NO: 2501). In some embodiments, the linker comprises the amino acid sequence SGGSSGGSSGS ETPGTSESATPESSGGSSGGS (SEQ ID NO: 2502). In some embodiments, the linker comprises the amino acid sequence SGGSGGSGGS (SEQ ID NO: 2503). In some embodiments, the linker comprises the amino acid sequence SGGS (SEQ ID NO: 2504). In other embodiments, the linker comprises the amino acid sequence

(SEQ ID NO: 2505)
SGGSSGGSSGSETPGTSESATPESAGSYPYDVPDYAGSAAPAAKKKKLD
GSGSGGSSGGS.

In some embodiments, a linker comprises 1-100 amino acids. In some embodiments, the linker comprises the amino acid sequence SGSETPGTSESATPES (SEQ ID NO: 2501). In some embodiments, the linker comprises the amino acid sequence SGGSSGGSSGSETPGTSESATPESSGGSSGGS (SEQ ID NO: 2502). In some embodiments, the linker comprises the amino acid sequence SGGSGGSGGS (SEQ ID NO: 2503). In some embodiments, the linker comprises the amino acid sequence SGGS (SEQ ID NO: 2504). In some embodiments, the linker comprises the amino acid sequence GGSGGS (SEQ ID NO: 2506), GGSGGSGGS (SEQ ID NO: 2507), SGGSSGGSSGSETPGTSESATPESAGSYPYDVPDYAGSAAPAAKKKKLDGSGSGGSSGG S (SEQ ID NO: 2505), or SGGSSGGSSGSETPGTSESATPESSGGSSGGSS (SEQ ID NO: 2468).

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein, or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2-[DNA binding domain]-[polymerase]-COOH; or NH2-[polymerase]-[DNA binding domain]-COOH, wherein each instance of “]-[” indicates the presence of an optional linker sequence. In some embodiments, a prime editor comprises a fusion protein and a DNA polymerase domain provided in trans, wherein the fusion protein comprises the structure NH2-[DNA binding domain]-[RNA-protein recruitment polypeptide]-COOH. In some embodiments, a prime editor comprises a fusion protein and a DNA binding domain provided in trans, wherein the fusion protein comprises the structure NH2-[DNA polymerase domain]-[RNA-protein recruitment polypeptide]-COOH.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans splicing. In some embodiments, a prime editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete prime editor fusion protein in the target cell.

In some embodiments, a prime editor fusion protein comprises a Cas9(H840A) nickase and a wild type M-MLV RT (referred to as “PE1,” and a prime editing system or composition referred to as PE1 system or PE1 composition). In some embodiments, a prime editor fusion protein comprises one or more individual components of PE1. In some embodiments, a prime editor fusion protein comprises a Cas9(H840A) nickase and a M-MLV RT that has amino acid substitutions D200N, T330P, T306K, W313F, and L603W compared to a wild type M-MLV RT (the fusion protein referred to as “PE2,” and a prime editing system or composition referred to as PE2 system or PE2 composition). The amino acid sequence of an exemplary PE2 and its individual components in shown in Table D1. In some embodiments, a prime editor fusion protein is PE2. In some embodiments, a prime editor fusion protein comprises one or more individual components of PE2.

In some embodiments, a prime editor fusion protein comprises a Cas9 (R221K, N349K, H840A) nickase and a M-MLV RT that has amino acid substitutions D200N, T330P, T306K, W313F, and L603W compared to a wild type M-MLV RT (the fusion protein referred to as “PEmax”, and a prime editing system or composition referred to as PEmax system or PEmax composition). The amino acid sequence of an exemplary PEmax and its individual components in shown in Table D2. In some embodiments, a prime editor fusion protein is PEmax. In some embodiments, a prime editor fusion protein comprises one or more individual components of PEmax

In various embodiments, a prime editor fusion proteins comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to PE1, PE2, or any of the prime editor fusion sequences described herein or known in the art.

TABLE D1
Amino acid sequences of exemplary RT and Cas9 proteins as well as PE2 and its
individual components

| DESCRIPTION | SEQUENCE |
|---|---|
| Exemplary | TLNIEDEYRLHETSKEPDVSLGSTWLSDFPQAWAETGGMGLAVRQAPLIIPLKATSTPVSIK |
| wild type | QYPMSQEARLGIKPHIQRLLDQGILVPCQSPWNTPLLPVKKPGTNDYRPVQDLREVNKRVED |
| moloney | IHPTVPNPYNLLSGLPPSHQWYTVLDLKDAFFCLRLHPTSQPLFAFEWRDPEMGISGQLTWT |
| murine | RLPQGFKNSPTLFDEALHRDLADFRIQHPDLILLQYVDDLLLAATSELDCQQGTRALLQTLG |
| leukemia | NLGYRASAKKAQICQKQVKYLGYLLKEGQRWLTEARKETVMGQPTPKTPRQLREFLGTAGFC |
| virus | RLWIPGFAEMAAPLYPLTKTGTLFNWGPDQQKAYQEIKQALLTAPALGLPDLTKPFELFVDE |
| reverse | KQGYAKGVLTQKLGPWRRPVAYLSKKLDPVAAGWPPCLRMVAAIAVLTKDAGKLTMGQPLVI |
| transcriptase: | LAPHAVEALVKQPPDRWLSNARMTHYQALLLDTDRVQFGPVVALNPATLLPLPEEGLQHNCL |
| | DILAEAHGTRPDLTDQPLPDADHTWYTDGSSLLQEGQRKAGAAVTTETEVIWAKALPAGTSA |
| | QRAELIALTQALKMAEGKKLNVYTDSRYAFATAHIHGEIYRRRGLLTSEGKEIKNKDEILAL |
| | LKALFLPKRLSIIHCPGHQKGHSAEARGNRMADQAARKAAITETPDTSTLLIENSSP (SEQ |
| | ID NO: 901) |
| Exemplary | MDKKYSIGLDIGTNSVGWAVITDEYKVPSKKFKVLGNTDRHSIKKNLIGALLFDSGETAEAT |
| Streptococcus | RLKRTARRRYTRRKNRICYLQEIFSNEMAKVDDSFFHRLEESFLVEEDKKHERHPIFGNIVD |
| pyogenes | EVAYHEKYPTIYHLRKKLVDSTDKADLRLIYLALAHMIKFRGHFLIEGDLNPDNSDVDKLFI |
| Cas9 | QLVQTYNQLFEENPINASGVDAKAILSARLSKSRRLENLIAQLPGEKKNGLFGNLIALSLGL |
| (SpCas9) | TPNFKSNFDLAEDAKLQLSKDTYDDDLDNLLAQIGDQYADLFLAAKNLSDAILLSDILRVNT |
| amino acid | EITKAPLSASMIKRYDEHHQDLTLLKALVRQQLPEKYKEIFFDQSKNGYAGYIDGGASQEEF |
| sequence: | YKFIKPILEKMDGTEELLVKLNREDLLRKQRTFDNGSIPHQIHLGELHAILRRQEDFYPFLK |
| | DNREKIEKILTFRIPYYVGPLARGNSRFAWMTRKSEETITPWNFEEVVDKGASAQSFIERMT |
| | NFDKNLPNEKVLPKHSLLYEYFTVYNELTKVKYVTEGMRKPAFLSGEQKKAIVDLLFKTNRK |
| | VTVKQLKEDYFKKIECFDSVEISGVEDRFNASLGTYHDLLKIIKDKDFLDNEENEDILEDIV |
| | LTLTLFEDREMIEERLKTYAHLFDDKVMKQLKRRRYTGWGRLSRKLINGIRDKQSGKTILDF |
| | LKSDGFANRNFMQLIHDDSLTFKEDIQKAQVSGQGDSLHEHIANLAGSPAIKKGILQTVKVV |
| | DELVKVMGRHKPENIVIEMARENQTTQKGQKNSRERMKRIEEGIKELGSQILKEHPVENTQL |
| | QNEKLYLYYLQNGRDMYVDQELDINRLSDYDVDHIVPQSFLKDDSIDNKVLTRSDKNRGKSD |
| | NVPSEEVVKKMKNYWRQLLNAKLITQRKFDNLTKAERGGLSELDKAGFIKRQLVETRQITKH |
| | VAQILDSRMNTKYDENDKLIREVKVITLKSKLVSDFRKDFQFYKVREINNYHHAHDAYLNAV |
| | VGTALIKKYPKLESEFVYGDYKVYDVRKMIAKSEQEIGKATAKYFFYSNIMNFFKTEITLAN |
| | GEIRKRPLIETNGETGEIVWDKGRDFATVRKVLSMPQVNIVKKTEVQTGGFSKESILPKRNS |
| | DKLIARKKDWDPKKYGGFDSPTVAYSVLVVAKVEKGKSKKLKSVKELLGITIMERSSFEKNP |
| | IDFLEAKGYKEVKKDLIIKLPKYSLFELENGRKRMLASAGELQKGNELALPSKYVNFLYLAS |
| | HYEKLKGSPEDNEQKQLFVEQHKHYLDEIIEQISEFSKRVILADANLDKVLSAYNKHRDKPI |
| | REQAENIIHLFTLTNLGAPAAFKYFDTTIDRKRYTSTKEVLDATLIHQSITGLYETRIDLSQ |
| | LGGD (SEQ ID NO: 902) |
| Exemplary | MKRNYILGLDIGITSVGYGIIDYETRDVIDAGVRLFKEANVENNEGRRSKRGARRLKRRRRH |
| Staphylococcus | RIQRVKKLLFDYNLLTDHSELSGINPYEARVKGLSQKLSEEEFSAALLHLAKRRGVHNVNEV |
| lugdunensis | EEDTGNELSTKEQISRNSKALEEKYVAELQLERLKKDGEVRGSINRFKTSDYVKEAKQLLKV |
| Cas9 (Slu | QKAYHQLDQSFIDTYIDLLETRRTYYEGPGEGSPFGWKDIKEWYEMLMGHCTYFPEELRSVK |
| Cas9) amino | YAYNADLYNALNDLNNLVITRDENEKLEYYEKFQIIENVFKQKKKPTLKQIAKEILVNEEDI |
| acid | KGYRVTSTGKPEFTNLKVYHDIKDITARKEIIENAELLDQIAKILTIYQSSEDIQEELTNLN |
| sequence | SELTQEEIEQISNLKGYTGTHNLSLKAINLILDELWHTNDNQIAIFNRLKLVPKKVDLSQQK |
| WP_002460848.1: | EIPTTLVDDFILSPVVKRSFIQSIKVINAIIKKYGLPNDIIIELAREKNSKDAQKMINEMQK |
| | RNRQTNERIEEIIRTTGKENAKYLIEKIKLHDMQEGKCLYSLEAIPLEDLLNNPFNYEVDHI |
| | IPRSVSFDNSFNNKVLVKQEEASKKGNRTPFQYLSSSDSKISYETFKKHILNLAKGKGRISK |
| | TKKEYLLEERDINRFSVQKDFINRNLVDTRYATRGLMNLLRSYFRVNNLDVKVKSINGGFTS |
| | FLRRKWKFKKERNKGYKHHAEDALIIANADFIFKEWKKLDKAKKVMENQMFEEKQAESMPEI |
| | ETEQEYKEIFITPHQIKHIKDFKDYKYSHRVDKKPNRELINDTLYSTRKDDKGNTLIVNNLN |
| | GLYDKDNDKLKKLINKSPEKLLMYHHDPQTYQKLKLIMEQYGDEKNPLYKYYEETGNYLTKY |
| | SKKDNGPVIKKIKYYGNKLNAHLDITDDYPNSRNKVVKLSLKPYRFDVYLDNGVYKFVTVKN |
| | LDVIKKENYYEVNSKCYEEAKKLKKISNQAEFIASFYNNDLIKINGELYRVIGVNNDLLNRI |
| | EVNMIDITYREYLENMNDKRPPRIIKTIASKTQSIKKYSTDILGNLYEVKSKKHPQIIKK |
| | (SEQ ID NO: 903) |
| PE2 fusion | MKRTADGSEFESPKKKRKVDKKYSIGLDIGTNSVGWAVITDEYKVPSKKFKVLGNTDRHSIK |
| protein | KNLIGALLFDSGETAEATRLKRTARRRYTRRKNRICYLQEIFSNEMAKVDDSFFHRLEESFL |
| CAS9 (H840A)- | VEEDKKHERHPIFGNIVDEVAYHEKYPTIYHLRKKLVDSTDKADLRLIYLALAHMIKERGHF |
| MMLV_RT | LIEGDLNPDNSDVDKLFIQLVQTYNQLFEENPINASGVDAKAILSARLSKSRRLENLIAQLP |
| D200N T330P | GEKKNGLFGNLIALSLGLTPNFKSNFDLAEDAKLQLSKDTYDDDLDNLLAQIGDQYADLFLA |
| L603W T306K | AKNLSDAILLSDILRVNTEITKAPLSASMIKRYDEHHQDLTLLKALVRQQLPEKYKEIFFDQ |
| W313F (SEQ | SKNGYAGYIDGGASQEEFYKFIKPILEKMDGTEELLVKLNREDLLRKQRTFDNGSIPHQIHL |
| ID NO: 904) | GELHAILRRQEDFYPFLKDNREKIEKILTFRIPYYVGPLARGNSRFAWMTRKSEETITPWNF |
| | EEVVDKGASAQSFIERMTNFDKNLPNEKVLPKHSLLYEYFTVYNELTKVKYVTEGMRKPAFL |
| | SGEQKKAIVDLLFKTNRKVTVKQLKEDYFKKIECFDSVEISGVEDRFNASLGTYHDLLKIIK |
| | DKDFLDNEENEDILEDIVLTLTLFEDREMIEERLKTYAHLFDDKVMKQLKRRRYTGWGRLSR |
| | KLINGIRDKQSGKTILDFLKSDGFANRNFMQLIHDDSLTFKEDIQKAQVSGQGDSLHEHIAN |
| | LAGSPAIKKGILQTVKVVDELVKVMGRHKPENIVIEMARENQTTQKGQKNSRERMKRIEEGI |
| | KELGSQILKEHPVENTQLQNEKLYLYYLQNGRDMYVDQELDINRLSDYDVDAIVPQSFLKDD |
| | SIDNKVLTRSDKNRGKSDNVPSEEVVKKMKNYWRQLLNAKLITQRKFDNLTKAERGGLSELD |
| | KAGFIKRQLVETRQITKHVAQILDSRMNTKYDENDKLIREVKVITLKSKLVSDERKDFQFYK |
| | VREINNYHHAHDAYLNAVVGTALIKKYPKLESEFVYGDYKVYDVRKMIAKSEQEIGKATAKY |
| | FFYSNIMNFFKTEITLANGEIRKRPLIETNGETGEIVWDKGRDFATVRKVLSMPQVNIVKKT |
| | EVQTGGFSKESILPKRNSDKLIARKKDWDPKKYGGFDSPTVAYSVLVVAKVEKGKSKKLKSV |
| | KELLGITIMERSSFEKNPIDFLEAKGYKEVKKDLIIKLPKYSLFELENGRKRMLASAGELQK |
| | GNELALPSKYVNFLYLASHYEKLKGSPEDNEQKQLFVEQHKHYLDEIIEQISEFSKRVILAD |
| | ANLDKVLSAYNKHRDKPIREQAENIIHLFTLTNLGAPAAFKYFDTTIDRKRYTSTKEVLDAT |
| | LIHQSITGLYETRIDLSQLGGDSGGSSGGSSGSETPGTSESATPESSGGSSGGSSTLNIEDE |
| | YRLHETSKEPDVSLGSTWLSDFPQAWAETGGMGLAVRQAPLIIPLKATSTPVSIKQYPMSQE |
| | ARLGIKPHIQRLLDQGILVPCQSPWNTPLLPVKKPGTNDYRPVQDLREVNKRVEDIHPTVPN |
| | PYNLLSGLPPSHQWYTVLDLKDAFFCLRLHPTSQPLFAFEWRDPEMGISGQLTWTRLPQGFK |
| | NSPTLFNEALHRDLADFRIQHPDLILLQYVDDLLLAATSELDCQQGTRALLQTLGNLGYRAS |
| | AKKAQICQKQVKYLGYLLKEGQRWLTEARKETVMGQPTPKTPRQLREFLGKAGFCRLFIPGF |
| | AEMAAPLYPLTKPGTLFNWGPDQQKAYQEIKQALLTAPALGLPDLTKPFELFVDEKQGYAKG |
| | VLTQKLGPWRRPVAYLSKKLDPVAAGWPPCLRMVAAIAVLTKDAGKLTMGQPLVILAPHAVE |
| | ALVKQPPDRWLSNARMTHYQALLLDTDRVQFGPVVALNPATLLPLPEEGLQHNCLDILAEAH |
| | GTRPDLTDQPLPDADHTWYTDGSSLLQEGQRKAGAAVTTETEVIWAKALPAGTSAQRAELIA |
| | LTQALKMAEGKKLNVYTDSRYAFATAHIHGEIYRRRGWLTSEGKEIKNKDEILALLKALFLP |
| | KRLSIIHCPGHQKGHSAEARGNRMADQAARKAAITETPDTSTLLIENSSPSGGSKRTADGSE |
| | FEPKKKRKV |
| | KEY: |
| | NUCLEAR LOCALIZATION SEQUENCE (NLS) |
| | CAS9(H840A) |
| | 33-AMINO ACID LINKER |
| | M-MLV REVERSE TRANSCRIPTASE |
| PE2-N- | MKRTADGSEFESPKKKRKV (SEQ ID NO: 2479) |
| terminal | |
| NLS ( | |
| PE2-CAS9 | DKKYSIGLDIGTNSVGWAVITDEYKVPSKKFKVLGNTDRHSIKKNLIGALLFDSGETAEATR |
| (H840A) | LKRTARRRYTRRKNRICYLQEIFSNEMAKVDDSFFHRLEESFLVEEDKKHERHPIFGNIVDE |
| (MET MINUS) | VAYHEKYPTIYHLRKKLVDSTDKADLRLIYLALAHMIKFRGHFLIEGDLNPDNSDVDKLFIQ |
| | LVQTYNQLFEENPINASGVDAKAILSARLSKSRRLENLIAQLPGEKKNGLFGNLIALSLGLT |
| | PNFKSNFDLAEDAKLQLSKDTYDDDLDNLLAQIGDQYADLFLAAKNLSDAILLSDILRVNTE |
| | ITKAPLSASMIKRYDEHHQDLTLLKALVRQQLPEKYKEIFFDQSKNGYAGYIDGGASQEEFY |
| | KFIKPILEKMDGTEELLVKLNREDLLRKQRTFDNGSIPHQIHLGELHAILRRQEDFYPFLKD |
| | NREKIEKILTFRIPYYVGPLARGNSRFAWMTRKSEETITPWNFEEVVDKGASAQSFIERMTN |
| | FDKNLPNEKVLPKHSLLYEYFTVYNELTKVKYVTEGMRKPAFLSGEQKKAIVDLLEKTNRKV |
| | TVKQLKEDYFKKIECFDSVEISGVEDRFNASLGTYHDLLKIIKDKDFLDNEENEDILEDIVL |
| | TLTLFEDREMIEERLKTYAHLFDDKVMKQLKRRRYTGWGRLSRKLINGIRDKQSGKTILDEL |
| | KSDGFANRNFMQLIHDDSLTFKEDIQKAQVSGQGDSLHEHIANLAGSPAIKKGILQTVKVVD |
| | ELVKVMGRHKPENIVIEMARENQTTQKGQKNSRERMKRIEEGIKELGSQILKEHPVENTQLQ |
| | NEKLYLYYLQNGRDMYVDQELDINRLSDYDVDAIVPQSFLKDDSIDNKVLTRSDKNRGKSDN |
| | VPSEEVVKKMKNYWRQLLNAKLITQRKFDNLTKAERGGLSELDKAGFIKRQLVETRQITKHV |
| | AQILDSRMNTKYDENDKLIREVKVITLKSKLVSDFRKDFQFYKVREINNYHHAHDAYLNAVV |
| | GTALIKKYPKLESEFVYGDYKVYDVRKMIAKSEQEIGKATAKYFFYSNIMNFFKTEITLANG |
| | EIRKRPLIETNGETGEIVWDKGRDFATVRKVLSMPQVNIVKKTEVQTGGFSKESILPKRNSD |
| | KLIARKKDWDPKKYGGFDSPTVAYSVLVVAKVEKGKSKKLKSVKELLGITIMERSSFEKNPI |
| | DFLEAKGYKEVKKDLIIKLPKYSLFELENGRKRMLASAGELQKGNELALPSKYVNFLYLASH |
| | YEKLKGSPEDNEQKQLFVEQHKHYLDEIIEQISEFSKRVILADANLDKVLSAYNKHRDKPIR |
| | EQAENIIHLETLTNLGAPAAFKYFDTTIDRKRYTSTKEVLDATLIHQSITGLYETRIDLSQL |
| | GGD (SEQ ID NO: 2508) |
| PE2- | SGGSSGGSSGSETPGTSESATPESSGGSSGGSS (SEQ ID NO: 2468) |
| linker | |
| between | |
| CAS9 domain | |
| and RT | |
| domain (33 | |
| amino acids) | |
| PE2- | TLNIEDEYRLHETSKEPDVSLGSTWLSDFPQAWAETGGMGLAVRQAPLIIPLKATSTPVSIK |
| MMLV_RT | QYPMSQEARLGIKPHIQRLLDQGILVPCQSPWNTPLLPVKKPGTNDYRPVQDLREVNKRVED |
| D200N T330P | IHPTVPNPYNLLSGLPPSHQWYTVLDLKDAFFCLRLHPTSQPLFAFEWRDPEMGISGQLTWT |
| L603W T306K | RLPQGFKNSPTLFNEALHRDLADFRIQHPDLILLQYVDDLLLAATSELDCQQGTRALLQTLG |
| W313F | NLGYRASAKKAQICQKQVKYLGYLLKEGQRWLTEARKETVMGQPTPKTPRQLREFLGKAGFC |
| | RLFIPGFAEMAAPLYPLTKPGTLFNWGPDQQKAYQEIKQALLTAPALGLPDLTKPFELFVDE |
| | KQGYAKGVLTQKLGPWRRPVAYLSKKLDPVAAGWPPCLRMVAAIAVLTKDAGKLTMGQPLVI |
| | LAPHAVEALVKQPPDRWLSNARMTHYQALLLDTDRVQFGPVVALNPATLLPLPEEGLQHNCL |
| | DILAEAHGTRPDLTDQPLPDADHTWYTDGSSLLQEGQRKAGAAVTTETEVIWAKALPAGTSA |
| | QRAELIALTQALKMAEGKKLNVYTDSRYAFATAHIHGEIYRRRGWLTSEGKEIKNKDEILAL |
| | LKALFLPKRLSIIHCPGHQKGHSAEARGNRMADQAARKAAITETPDTSTLLIENSSP (SEQ |
| | ID NO: 2509) |
| PE2-C- | SGGSKRTADGSEFEPKKKRKV (SEQ ID NO: 2487) |
| terminal | |
| NLS | |

TABLE D2
Amino acid sequence of PEmax and its individual components

| DESCRIPTION | SEQUENCE |
|---|---|
| PEmax fusion | MKRTADGSEFESPKKKRKVDKKYSIGLDIGTNSVGWAVITDEYKVPSKKFKVLGNTDRHSIKKNLIGALLFD |
| protein | SGETAEATRLKRTARRRYTRRKNRICYLQEIFSNEMAKVDDSFFHRLEESFLVEEDKKHERHPIFGNIVDEVA |
| | YHEKYPTIYHLRKKLVDSTDKADLRLIYLALAHMIKFRGHFLIEGDLNPDNSDVDKLFIQLVQTYNQLFEENP |
| | INASGVDAKAILSARLSKSRKLENLIAQLPGEKKNGLFGNLIALSLGLTPNFKSNFDLAEDAKLQLSKDTYDD |
| | DLDNLLAQIGDQYADLFLAAKNLSDAILLSDILRVNTEITKAPLSASMIKRYDEHHQDLTLLKALVRQQLPEK |
| | YKEIFFDQSKNGYAGYIDGGASQEEFYKFIKPILEKMDGTEELLVKLKREDLLRKQRTFDNGSIPHQIHLGEL |
| | HAILRRQEDFYPFLKDNREKIEKILTFRIPYYVGPLARGNSRFAWMTRKSEETITPWNFEEVVDKGASAQSFI |
| | ERMTNFDKNLPNEKVLPKHSLLYEYFTVYNELTKVKYVTEGMRKPAFLSGEQKKAIVDLLFKTNRKVTVKQ |
| | LKEDYFKKIECFDSVEISGVEDRFNASLGTYHDLLKIIKDKDFLDNEENEDILEDIVLTLTLFEDREMIEERLKTY |
| | AHLFDDKVMKQLKRRRYTGWGRLSRKLINGIRDKQSGKTILDFLKSDGFANRNFMQLIHDDSLTFKEDIQK |
| | AQVSGQGDSLHEHIANLAGSPAIKKGILQTVKVVDELVKVMGRHKPENIVIEMARENQTTQKGQKNSRE |
| | RMKRIEEGIKELGSQILKEHPVENTQLQNEKLYLYYLQNGRDMYVDQELDINRLSDYDVDAIVPQSFLKDD |
| | SIDNKVLTRSDKNRGKSDNVPSEEVVKKMKNYWRQLLNAKLITQRKFDNLTKAERGGLSELDKAGFIKRQL |
| | VETRQITKHVAQILDSRMNTKYDENDKLIREVKVITLKSKLVSDFRKDFQFYKVREINNYHHAHDAYLNAV |
| | VGTALIKKYPKLESEFVYGDYKVYDVRKMIAKSEQEIGKATAKYFFYSNIMNFFKTEITLANGEIRKRPLIETN |
| | GETGEIVWDKGRDFATVRKVLSMPQVNIVKKTEVQTGGFSKESILPKRNSDKLIARKKDWDPKKYGGFDS |
| | PTVAYSVLVVAKVEKGKSKKLKSVKELLGITIMERSSFEKNPIDFLEAKGYKEVKKDLIIKLPKYSLFELENGRK |
| | RMLASAGELQKGNELALPSKYVNFLYLASHYEKLKGSPEDNEQKQLFVEQHKHYLDEIIEQISEFSKRVILAD |
| | ANLDKVLSAYNKHRDKPIREQAENIIHLFTLTNLGAPAAFKYFDTTIDRKRYTSTKEVLDATLIHQSITGLYET |
| | RIDLSQLGGDSGGSSGGSKRTADGSEFESPKKKRKVSGGSSGGSTLNIEDEYRLHETSKEPDVSLGSTWLSD |
| | FPQAWAETGGMGLAVRQAPLIIPLKATSTPVSIKQYPMSQEARLGIKPHIQRLLDQGILVPCQSPWNTPLL |
| | PVKKPGTNDYRPVQDLREVNKRVEDIHPTVPNPYNLLSGLPPSHQWYTVLDLKDAFFCLRLHPTSQPLFAF |
| | EWRDPEMGISGQLTWTRLPQGFKNSPTLFNEALHRDLADFRIQHPDLILLQYVDDLLLAATSELDCQQGT |
| | RALLQTLGNLGYRASAKKAQICQKQVKYLGYLLKEGQRWLTEARKETVMGQPTPKTPRQLREFLGKAGFC |
| | RLFIPGFAEMAAPLYPLTKPGTLFNWGPDQQKAYQEIKQALLTAPALGLPDLTKPFELFVDEKQGYAKGVL |
| | TQKLGPWRRPVAYLSKKLDPVAAGWPPCLRMVAAIAVLTKDAGKLTMGQPLVILAPHAVEALVKQPPDR |
| | WLSNARMTHYQALLLDTDRVQFGPVVALNPATLLPLPEEGLQHNCLDILAEAHGTRPDLTDQPLPDADH |
| | TWYTDGSSLLQEGQRKAGAAVTTETEVIWAKALPAGTSAQRAELIALTQALKMAEGKKLNVYTDSRYAFA |
| | TAHIHGEIYRRRGWLTSEGKEIKNKDEILALLKALFLPKRLSIIHCPGHQKGHSAEARGNRMADQAARKAAI |
| | TETPDTSTLLIENSSPSGGSKRTADGSEFESPKKKRKVGSGPAAKRVKLD (SEQ ID NO: 2510) |
| PEmax-N- | MKRTADGSEFESPKKKRKV (SEQ ID NO: 2479) |
| terminal NLS | |
| PEmax-CAS9 | DKKYSIGLDIGTNSVGWAVITDEYKVPSKKFKVLGNTDRHSIKKNLIGALLFDSGETAEATRLKRTARRRYTR |
| (R221K N394K | RKNRICYLQEIFSNEMAKVDDSFFHRLEESFLVEEDKKHERHPIFGNIVDEVAYHEKYPTIYHLRKKLVDSTD |
| H840A) (not | KADLRLIYLALAHMIKFRGHFLIEGDLNPDNSDVDKLFIQLVQTYNQLFEENPINASGVDAKAILSARLSKSR |
| including N- | KLENLIAQLPGEKKNGLFGNLIALSLGLTPNFKSNFDLAEDAKLQLSKDTYDDDLDNLLAQIGDQYADLFLA |
| terminal Met in | AKNLSDAILLSDILRVNTEITKAPLSASMIKRYDEHHQDLTLLKALVRQQLPEKYKEIFFDQSKNGYAGYIDG |
| Cas9) | GASQEEFYKFIKPILEKMDGTEELLVKLKREDLLRKQRTFDNGSIPHQIHLGELHAILRRQEDFYPFLKDNRE |
| | KIEKILTFRIPYYVGPLARGNSRFAWMTRKSEETITPWNFEEVVDKGASAQSFIERMTNFDKNLPNEKVLPK |
| | HSLLYEYFTVYNELTKVKYVTEGMRKPAFLSGEQKKAIVDLLFKTNRKVTVKQLKEDYFKKIECFDSVEISGV |
| | EDRFNASLGTYHDLLKIIKDKDFLDNEENEDILEDIVLTLTLFEDREMIEERLKTYAHLFDDKVMKOLKRRRY |
| | TGWGRLSRKLINGIRDKQSGKTILDFLKSDGFANRNFMQLIHDDSLTFKEDIQKAQVSGQGDSLHEHIANL |
| | AGSPAIKKGILQTVKVVDELVKVMGRHKPENIVIEMARENQTTQKGQKNSRERMKRIEEGIKELGSQILKE |
| | HPVENTQLQNEKLYLYYLQNGRDMYVDQELDINRLSDYDVDAIVPQSFLKDDSIDNKVLTRSDKNRGKSD |
| | NVPSEEVVKKMKNYWRQLLNAKLITQRKFDNLTKAERGGLSELDKAGFIKRQLVETRQITKHVAQILDSR |
| | MNTKYDENDKLIREVKVITLKSKLVSDFRKDFQFYKVREINNYHHAHDAYLNAVVGTALIKKYPKLESEFVY |
| | GDYKVYDVRKMIAKSEQEIGKATAKYFFYSNIMNFFKTEITLANGEIRKRPLIETNGETGEIVWDKGRDFAT |
| | VRKVLSMPQVNIVKKTEVQTGGFSKESILPKRNSDKLIARKKDWDPKKYGGFDSPTVAYSVLVVAKVEKGK |
| | SKKLKSVKELLGITIMERSSFEKNPIDFLEAKGYKEVKKDLIIKLPKYSLFELENGRKRMLASAGELQKGNELAL |
| | PSKYVNFLYLASHYEKLKGSPEDNEQKQLFVEQHKHYLDEIIEQISEFSKRVILADANLDKVLSAYNKHRDKP |
| | IREQAENIIHLFTLTNLGAPAAFKYFDTTIDRKRYTSTKEVLDATLIHQSITGLYETRIDLSQLGGD (SEQ ID |
| | NO: 2511) |
| PEmax- | SGGSSGGSKRTADGSEFESPKKKRKVSGGSSGGS (SEQ ID NO: 2489) |
| SGGSx2- | |
| bpSV40NLS- | |
| SGGSx2 linker | |
| PEmax- | TLNIEDEYRLHETSKEPDVSLGSTWLSDFPQAWAETGGMGLAVRQAPLIIPLKATSTPVSIKQYPMSQEAR |
| MMLV_RT | LGIKPHIQRLLDQGILVPCQSPWNTPLLPVKKPGTNDYRPVQDLREVNKRVEDIHPTVPNPYNLLSGLPPS |
| D200N T330P | HQWYTVLDLKDAFFCLRLHPTSQPLFAFEWRDPEMGISGQLTWTRLPQGFKNSPTLFNEALHRDLADFRI |
| L603W T306K | QHPDLILLQYVDDLLLAATSELDCQQGTRALLQTLGNLGYRASAKKAQICQKQVKYLGYLLKEGQRWLTEA |
| W313F | RKETVMGQPTPKTPRQLREFLGKAGFCRLFIPGFAEMAAPLYPLTKPGTLFNWGPDQQKAYQEIKQALLT |
| | APALGLPDLTKPFELFVDEKQGYAKGVLTQKLGPWRRPVAYLSKKLDPVAAGWPPCLRMVAAIAVLTKDA |
| | GKLTMGQPLVILAPHAVEALVKQPPDRWLSNARMTHYQALLLDTDRVQFGPVVALNPATLLPLPEEGLQ |
| | HNCLDILAEAHGTRPDLTDQPLPDADHTWYTDGSSLLQEGQRKAGAAVTTETEVIWAKALPAGTSAQRA |
| | ELIALTQALKMAEGKKLNVYTDSRYAFATAHIHGEIYRRRGWLTSEGKEIKNKDEILALLKALFLPKRLSIIHC |
| | PGHQKGHSAEARGNRMADQAARKAAITETPDTSTLLIENSSP (SEQ ID NO: 2509) |
| PEmax -C- | SGGSKRTADGSEFESPKKKRKV (SEQ ID NO: 2490) |
| terminal linker- | |
| NLS | |
| PEmax- C- | GSGPAAKRVKLD (SEQ ID NO: 2491) |
| terminal linker- | |
| NLS2 | |

### Prime Editing Guide RNAs

The term “prime editing guide RNA,” or “PEgRNA,” refers to a guide polynucleotide that comprises one or more intended nucleotide edits for incorporation into the target DNA. In some embodiments, the PEgRNA associates with and directs a prime editor to incorporate the one or more intended nucleotide edits into the target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene, or may refer to the edit encoded by the editing template on the newly synthesized single stranded DNA that replaces the editing target sequence, as compared to the editing target sequence. In some embodiments, a PEgRNA comprises a spacer sequence that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA further comprises an extended nucleotide sequence comprising one or more intended nucleotide edits compared to the endogenous sequence of the target gene, wherein the extended nucleotide sequence may be referred to as an extension arm.

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the extension arm further comprises an editing template that comprises one or more intended nucleotide edits to be incorporated in the target gene by prime editing. In some embodiments, the editing template is a template for an RNA-dependent DNA polymerase domain or polypeptide of the prime editor, for example, a reverse transcriptase domain. The reverse transcriptase editing template may also be referred to herein as an RT template, or RTT. In some embodiments, the editing template comprises partial complementarity to an editing target sequence in the target gene. In some embodiments, the editing template comprises substantial or partial complementarity to the editing target sequence except at the position of the intended nucleotide edits to be incorporated into the target gene.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer sequence, the gRNA core, or the extension arm. In some embodiments, a PEgRNA comprises DNA in the spacer sequence. In some embodiments, the entire spacer sequence of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PEgRNA, the 3′ portion of the PEgRNA, or in the middle of the gRNA core. In some embodiments, a PEgRNA comprises a PBS and an editing template sequence in 5′ to 3′ order. In some embodiments, the gRNA core of a PEgRNA of this disclosure may be located in between a spacer and an extension arm of the PEgRNA. In some embodiments, the gRNA core of a PEgRNA may be located at the 3′ end of a spacer. In some embodiments, the gRNA core of a PEgRNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, the PEgRNA comprises, from 5′ to 3′: a spacer, a gRNA core, and an extension arm. In some embodiments, the PEgRNA comprises, from 5′ to 3′: a spacer, a gRNA core, an editing template, and a PBS. In some embodiments, the PEgRNA comprises, from 5′ to 3′: an extension arm, a spacer, and a gRNA core. In some embodiments, the PEgRNA comprises, from 5′ to 3′: an editing target, a PBS, a spacer, and a gRNA core.

In some embodiments, a PEgRNA comprises a single polynucleotide molecule that comprises the spacer sequence, the gRNA core, and the extension arm. In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA may comprise a first polynucleotide comprising the spacer and a first portion of a gRNA core comprising, which may be also be referred to as a crRNA. In some embodiments, the PEgRNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem.

In some embodiments, a spacer sequence comprises a region that has substantial complementarity to a search target sequence on the target strand of a double stranded target DNA. In some embodiments, the spacer sequence of a PEgRNA is identical or substantially identical to a protospacer sequence on the edit strand of the target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the target gene. In some embodiments, the spacer comprises is substantially complementary to the search target sequence.

In some embodiments, the length of the spacer varies from at least 10 nucleotides to 100 nucleotides. For examples, a spacer may be at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, 20 to 30 nucleotides in length, 30 to 40 nucleotides in length, 40 to 50 nucleotides in length, 50 to 60 nucleotides in length, 60 to 70 nucleotides in length, 70 to 80 nucleotides in length, or 90 nucleotides to 100 nucleotides in length. In some embodiments, the spacer is 20 nucleotides in length. In some embodiments, the spacer is 17 to 18 nucleotides in length.

As used herein in a PEgRNA or a nick guide RNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PEgRNA or guide RNA or any chemically modified uracil nucleobase known in the art, such as 5-methoxyuracil.

The extension arm of a PEgRNA may comprise a primer binding site (PBS) and an editing template (e.g., an RTT). The extension arm may be partially complementary to the spacer. In some embodiments, the editing template (e.g., RTT) is partially complementary to the spacer. In some embodiments, the editing template (e.g., RTT) and the primer binding site (PBS) are each partially complementary to the spacer.

An extension arm of a PEgRNA may comprise a primer binding site sequence (PBS, or PBS sequence) that hybridizes with a free 3′ end of a single stranded DNA in the target gene generated by nicking with a prime editor. The length of the PBS sequence may vary depending on, e.g., the prime editor components, the search target sequence and other components of the PEgRNA. In some embodiments, the length of the primer binding site (PBS) varies from at least 2 nucleotides to 50 nucleotides. For examples, a primer binding site (PBS) may be at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, or at least 50 nucleotides in length. In some embodiments, the PBS is at least 6 nucleotides in length. In some embodiments, the PBS is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is about 7 to 15 nucleotides in length. In some embodiments, the PBS is 6, 7, 8, 9, 10, 11, 12, 13, 14, 15, 16, 17, 18, 19, or 20 nucleotides in length. In some embodiments, the PBS is 8, 9, 10, 11, 12, 13, or 14 nucleotides in length.

The PBS may be complementary or substantially complementary to a DNA sequence in the edit strand of the target gene. By annealing with the edit strand at a free hydroxy group, e.g., a free 3′ end generated by prime editor nicking, the PBS may initiate synthesis of a new single stranded DNA encoded by the editing template at the nick site. In some embodiments, the PBS is at least about 70%, 75%, 80%, 85%, 90%, 95%, or 100% complementary to a region of the edit strand of the target gene. In some embodiments, the PBS is perfectly complementary, or 100% complementary, to a region of the edit strand of the target gene.

An extension arm of a PEgRNA may comprise an editing template that serves as a DNA synthesis template for the DNA polymerase in a prime editor during prime editing.

The length of an editing template may vary depending on, e.g., the prime editor components, the search target sequence and other components of the PEgRNA. In some embodiments, the editing template serves as a DNA synthesis template for a reverse transcriptase, and the editing template is referred to as a reverse transcription editing template (RTT).

The editing template (e.g., RTT), in some embodiments, is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the RTT is 12, 13, 14, 15, 16, 17, 18, 19, 20, 21, 22, 23, 24, or 25 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, the editing template (e.g., RTT) sequence is about 70%, 75%, 80%, 85%, 90%, 95%, or 99% complementary to the editing target sequence on the edit strand of the target gene. In some embodiments, the editing template sequence (e.g., RTT) is substantially complementary to the editing target sequence. In some embodiments, the editing template sequence (e.g., RTT) is complementary to the editing target sequence except at positions of the intended nucleotide edits to be incorporated in the target gene. In some embodiments, the editing template comprises a nucleotide sequence comprising about 85% to about 95% complementarity to an editing target sequence in the edit strand in the target gene. In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the target gene.

An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA may comprise one or more intended nucleotide edits, compared to the gene to be edited. Position of the intended nucleotide edit(s) relevant to other components of the PEgRNA, or to particular nucleotides (e.g., mutations) in the target gene may vary. In some embodiments, the nucleotide edit is in a region of the PEgRNA corresponding to or homologous to the protospacer sequence. In some embodiments, the nucleotide edit is in a region of the PEgRNA corresponding to a region of the gene outside of the protospacer sequence.

In some embodiments, the position of a nucleotide edit incorporation in the target gene may be determined based on position of the protospacer adjacent motif (PAM). For instance, the intended nucleotide edit may be installed in a sequence corresponding to the protospacer adjacent motif (PAM) sequence. In some embodiments, a nucleotide edit in the editing template is at a position corresponding to the 5′ most nucleotide of the PAM sequence. In some embodiments, a nucleotide edit in the editing template is at a position corresponding to the 3′ most nucleotide of the PAM sequence. In some embodiments, position of an intended nucleotide edit in the editing template may be referred to by aligning the editing template with the partially complementary edit strand of the target gene, and referring to nucleotide positions on the editing strand where the intended nucleotide edit is incorporated. In some embodiments, a nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs upstream of the 5′ most nucleotide of the PAM sequence in the edit strand of the target gene. By 0 base pair upstream or downstream of a reference position, it is meant that the intended nucleotide is immediately upstream or downstream of the reference position. In some embodiments, a nucleotide edit is incorporated at a position corresponding to about 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs upstream of the 5′ most nucleotide of the PAM sequence. In some embodiments, the nucleotide edit is incorporated at a position corresponding to 3 base pairs upstream of the 5′ most nucleotide of the PAM sequence. In some embodiments, the nucleotide edit in is incorporated at a position corresponding to 4 base pairs upstream of the 5′ most nucleotide of the PAM sequence. In some embodiments, the nucleotide edit is incorporated at a position corresponding to 5 base pairs upstream of the 5′ most nucleotide of the PAM sequence. In some embodiments, the nucleotide edit in the editing template is at a position corresponding to 6 base pairs upstream of the 5′ most nucleotide of the PAM sequence.

In some embodiments, an intended nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs downstream of the 5′ most nucleotide of the PAM sequence in the edit strand of the target gene. In some embodiments, a nucleotide edit is incorporated at a position corresponding to about 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs downstream of the 5′ most nucleotide of the PAM sequence. In some embodiments, a nucleotide edit is incorporated at a position corresponding to 3 base pairs downstream of the 5′ most nucleotide of the PAM sequence. In some embodiments, a nucleotide edit is incorporated at a position corresponding to 4 base pairs downstream of the 5′ most nucleotide of the PAM sequence. In some embodiments, a nucleotide edit is incorporated at a position corresponding to 5 base pairs downstream of the 5′ most nucleotide of the PAM sequence. In some embodiments, a nucleotide edit is incorporated at a position corresponding to 6 base pairs downstream of the 5′ most nucleotide of the PAM sequence. By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

In some embodiments, the position of a nucleotide edit incorporation in the target gene may be determined based on position of the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides downstream of the nick site on the PAM strand (or the non-target strand, or the edit strand) of the double stranded target DNA. In some embodiments, position of the intended nucleotide edit in the editing template may be referred to by aligning the editing template with the partially complementary editing target sequence on the edit strand, and referring to nucleotide positions on the editing strand where the intended nucleotide edit is incorporated. Accordingly, in some embodiments, a nucleotide edit in an editing template is at a position corresponding to a position about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides apart from the nick site. In some embodiments, when referred to in the context of the PAM strand (or the non-target strand, or the edit strand), a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides downstream from the nick site. The relative positions of the intended nucleotide edit(s) and nick site may be referred to by numbers. For example, in some embodiments, the nucleotide immediately downstream of the nick site on a PAM strand (or the non-target strand, or the edit strand) may be referred to as at position 0. The nucleotide immediately upstream of the nick site on the PAM strand (or the non-target strand, or the edit strand) may be referred to as at position −1. The nucleotides downstream of position 0 on the PAM strand may be referred to as at positions +1, +2, +3, +4, . . . +n, and the nucleotides upstream of position −1 on the PAM strand may be referred to as at positions −2, −3, −4, . . . , −n. Accordingly, in some embodiments, the nucleotide in the editing template that corresponds to position 0 when the editing template is aligned with the partially complementary editing target sequence by complementarity may also be referred to as position 0 in the editing template, the nucleotides in the editing template corresponding to the nucleotides at positions +1, +2, +3, +4, . . . , +n on the PAM strand of the double stranded target DNA may also be referred to as at positions +1, +2, +3, +4, . . . , +n in the editing template, and the nucleotides in the editing template corresponding to the nucleotides at positions −1, −2, −3, −4, . . . , −n on the PAM strand on the double stranded target DNA may also be referred to as at positions −1, −2, −3, −4, . . . , −n on the editing template, even though when the PEgRNA is viewed as a standalone nucleic acid, positions +1, +2, +3, +4, . . . , +n are 5′ of position 0 and positions −1, −2, −3, −4, . . . −n are 3′ of position 0 in the editing template. In some embodiments, an intended nucleotide edit is at position +n of the editing template relative to position 0. Accordingly, the intended nucleotide edit may be incorporated at position +n of the PAM strand of the double stranded target DNA (and subsequently, the target strand of the double stranded target DNA) by prime editing. The number n may be referred to as the nick to edit distance. When referred to in the PEgRNA, positions of the one or more intended nucleotide edits may be referred to relevant to components of the PEgRNA. For example, an intended nucleotide edit may be 5′ or 3′ to the PBS. In some embodiments, a PEgRNA comprises the structure, from 5′ to 3′: a spacer, a gRNA core, an editing template, and a PBS. In some embodiments, the intended nucleotide edit is 0, 1, 2, 3, 4, 5, 6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs upstream to the 5′ most nucleotide of the PBS. In some embodiments, the intended nucleotide edit is 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs upstream to the 5′ most nucleotide of the PBS.

The corresponding positions of the intended nucleotide edit incorporated in the target gene may also be referred to based on the nicking position generated by a prime editor based on sequence homology and complementarity. For example, in embodiments, the distance between the nucleotide edit to be incorporated into the target gene and the nick generated by the prime editor may be determined when the spacer hybridizes with the search target sequence and the extension arm hybridizes with the editing target sequence. In certain embodiments, the position of the nucleotide edit can be in any position downstream of the nick site on the edit strand (or the PAM strand) generated by the prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 base pairs from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence.

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nucleobase position (e.g., for a PEgRNA following 5′-spacer-gRNA core-RTT-PBS-3′ orientation, the 5′ most nucleobase is the “first base”). In some embodiments, the editing template comprises an uracil at the first nucleobase position (e.g., for a PEgRNA following 5′-spacer-gRNA core-RTT-PBS-3′ orientation, the 5′ most nucleobase is the “first base”). In some embodiments, the editing template comprises a cytosine at the first nucleobase position (e.g., for a PEgRNA following 5′-spacer-gRNA core-RTT-PBS-3′ orientation, the 5′ most nucleobase is the “first base”). In some embodiments, the editing template does not comprise a cytosine at the first nucleobase position (e.g., for a PEgRNA following 5′-spacer-gRNA core-RTT-PBS-3′ orientation, the 5′ most nucleobase is the “first base”).

The editing template of a PEgRNA may encode a new single stranded DNA (e.g., by reverse transcription) to replace a target sequence in the target gene. In some embodiments, the editing target sequence in the edit strand of the target gene is replaced by the newly synthesized strand, and the nucleotide edit(s) are incorporated in the region of the target gene.

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a DNA nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired “lower stem” adjacent to the spacer sequence and a base paired “upper stem” following the lower stem, where the lower stem and upper stem may be connected by a “bulge” comprising unpaired RNAs. The gRNA core may further comprise a “nexus” distal from the spacer sequence, followed by a hairpin structure, e.g., at the 3′ end.

In some embodiments, the gRNA core comprises modified nucleotides as compared to a wild type gRNA core in the lower stem, upper stem, and/or the hairpin. For example, nucleotides in the lower stem, upper stem, an/or the hairpin regions may be modified, deleted, or replaced. In some embodiments, RNA nucleotides in the lower stem, upper stem, an/or the hairpin regions may be replaced with one or more DNA sequences. In some embodiments, the gRNA core comprises unmodified or wild type RNA sequences in the nexus and/or the bulge regions. In some embodiments, the gRNA core does not include long stretches of A-T pairs, for example, a GUUUU-AAAAC pairing element. In some embodiments, the gRNA core comprises the sequences (as with all RNA sequences provided herein, the T residues in the below sequences may be replaced with U residues): GTTTTAGAGCTAGAAATAGCAAGTTAAAATAAGGCTAGTCCGTTATCAACTTGAAAA AGTGGCACCGAGTCGGTGC (SEQ ID NO: 905); GTTTGAGAGCTAGAAATAGCAAGTTTAAATAAGGCTAGTCCGTTATCAACTTGAAAA AGTGGGACCGAGTCGGTCC (SEQ ID NO: 2512), or GTTTAAGAGCTATGCTGGAAACAGCATAGCAAGTTTAAATAAGGCTAGTCCGTTATC AACTTGAAAAAGTGGCACCGAGTCGGTGC (SEQ ID NO: 2513). In some embodiments, the gRNA core comprises the sequence GTTTTAGAGCTAGAAATAGCAAGTTAAAATAAGGCTAGTCCGTTATCAACTTGAAAA AGTGGCACCGAGTCGGTGC (SEQ ID NO: 905). Any gRNA core sequences known in the art are also contemplated in the prime editing compositions described herein.

A PEgRNA may also comprise optional modifiers, e.g., 3′ end modifier region and/or an 5′ end modifier region. In some embodiments, a PEgRNA comprises at least one nucleotide that is not part of a spacer, a gRNA core, or an extension arm. The optional sequence modifiers could be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

In some embodiments, a PEgRNA or a nick guide RNA (ngRNA) may be chemically synthesized, or may be assembled or cloned and transcribed from a DNA sequence, e.g., a plasmid DNA sequence, or by any RNA oligonucleotide synthesis method known in the art. In some embodiments, DNA sequence that encodes a PEgRNA (or ngRNA) may be designed to append one or more nucleotides at the 5′ end or the 3′ end of the PEgRNA (or nick guide RNA) encoding sequence to enhance PEgRNA transcription. For example, in some embodiments, a DNA sequence that encodes a PEgRNA (or nick guide RNA) (or an ngRNA) may be designed to append a nucleotide G at the 5′ end. Accordingly, in some embodiments, the PEgRNA (or nick guide RNA) may comprise an appended nucleotide G at the 5′ end. In some embodiments, a DNA sequence that encodes a PEgRNA (or nick guide RNA) may be designed to append a sequence that enhances transcription, e.g., a Kozak sequence, at the 5′ end. In some embodiments, a DNA sequence that encodes a PEgRNA (or nick guide RNA) may be designed to append the sequence CACC or CCACC at the 5′ end. Accordingly, in some embodiments, the PEgRNA (or nick guide RNA) may comprise an appended sequence CACC or CCACC at the 5′ end. In some embodiments, a DNA sequence that encodes a PEgRNA (or nick guide RNA) may be designed to append the sequence TTT, TTTT, TTTTT, TTTTTT, TTTTTTT at the 3′ end. Accordingly, in some embodiments, the PEgRNA (or nick guide RNA) may comprise an appended sequence UUU, UUUU, UUUUU, UUUUUU, or UUUUUUU at the 3′ end. In some embodiments, a PEgRNA or ngRNA may include a modifying sequence at the 3′ end having the sequence AACAUUGACGCGUCUCUACGUGGGGGCGCG (SEQ ID NO: 908).

In some embodiments, a prime editing system or composition further comprises a nick guide polynucleotide, such as a nick guide RNA (ngRNA). Without wishing to be bound by any particular theory, the non-edit strand of a double stranded target DNA in the target gene may be nicked by a CRISPR-Cas nickase directed by an ngRNA. In some embodiments, the nick on the non-edit strand directs endogenous DNA repair machinery to use the edit strand as a template for repair of the non-edit strand, which may increase efficiency of prime editing. In some embodiments, the non-edit strand is nicked by a prime editor localized to the non-edit strand by the ngRNA. Accordingly, also provided herein are PEgRNA systems comprising at least one PEgRNA and at least one ngRNA.

In some embodiments, the ngRNA is a guide RNA which contains a variable spacer sequence and a guide RNA scaffold or core region that interacts with the DNA binding domain, e.g., Cas9 of the prime editor. In some embodiments, the ngRNA comprises a spacer sequence (referred to herein as an ng spacer, or a second spacer) that is substantially complementary to a second search target sequence (or ng search target sequence), which is located on the edit strand, or the non-target strand. Thus, in some embodiments, the ng search target sequence recognized by the ng spacer and the search target sequence recognized by the spacer sequence of the PEgRNA are on opposite strands of the double stranded target DNA of target gene. A prime editing system, composition, or complex comprising a ngRNA may be referred to as a “PE3” prime editing systemPE3 prime editing composition, or PE3 prime editing complex.

In some embodiments, the ng search target sequence is located on the non-target strand, within 10 base pairs to 100 base pairs of an intended nucleotide edit incorporated by the PEgRNA on the edit strand. In some embodiments, the ng target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the PEgRNA on the edit strand. In some embodiments, the 5′ ends of the ng search target sequence and the PEgRNA search target sequence are within 1, 2, 3, 4, 5, 6, 7, 8, 9, or 10 bp apart from each other. In some embodiments, the 5′ ends of the ng search target sequence and the PEgRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp apart from each other.

In some embodiments, an ng spacer sequence is complementary to, and may hybridize with the second search target sequence only after an intended nucleotide edit has been incorporated on the edit strand, by the editing template of a PEgRNA. Such a prime editing system maybe referred to as a “PE3b” prime editing system or composition. In some embodiments, the ngRNA comprises a spacer sequence that matches only the edit strand after incorporation of the nucleotide edits, but not the endogenous target gene sequence on the edit strand. Accordingly, in some embodiments, an intended nucleotide edit is incorporated within the ng search target sequence. In some embodiments, the intended nucleotide edit is incorporated within about 1-10 nucleotides of the position corresponding to the PAM of the ng search target sequence.

A PEgRNA and/or an ngRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs and/or ngRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs and/or ngRNAs provided in this disclosure may have undergone a chemical or biological modifications. Modifications may be made at any position within a PEgRNA or ngRNA, and may include modification to a nucleobase or to a phosphate backbone of the PEgRNA or ngRNA. In some embodiments, chemical modifications can be a structure guided modifications. In some embodiments, a chemical modification is at the 5′ end and/or the 3′ end of a PEgRNA. In some embodiments, a chemical modification is at the 5′ end and/or the 3′ end of a ngRNA. In some embodiments, a chemical modification may be within the spacer sequence, the extension arm, the editing template sequence, or the primer binding site of a PEgRNA. In some embodiments, a chemical modification may be within the spacer sequence or the gRNA core of a PEgRNA or a ngRNA. In some embodiments, a chemical modification may be within the 3′ most nucleotides of a PEgRNA or ngRNA. In some embodiments, a chemical modification may be within the 3′ most end of a PEgRNA or ngRNA. In some embodiments, a chemical modification may be within the 5′ most end of a PEgRNA or ngRNA. In some embodiments, a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, or 5 or more chemically modified nucleotides at the 3′ end. In some embodiments, a PEgRNA or ngRNA comprises 1, 2, 3, 4, or 5 more chemically modified nucleotides at the 5′ end. In some embodiments, a PEgRNA or ngRNA comprises 1, 2, or 3 or more chemically modified nucleotides at the 3′ end. In some embodiments, a PEgRNA or ngRNA comprises 1, 2, or 3 more chemically modified nucleotides at the 5′ end. In some embodiments, a PEgRNA or ngRNA comprises 1, 2, 3, 4, 5, 6, 7, 8, 9, 10 or more contiguous chemically modified nucleotides at the 3′ end. In some embodiments, a PEgRNA or ngRNA comprises 1, 2, 3, 4, 5, 6, 7, 8, 9, 10 or more contiguous chemically modified nucleotides at the 5′ end. In some embodiments, a PEgRNA or ngRNA comprises 1, 2, 3, 4, or 5 contiguous chemically modified nucleotides at the 3′ end. In some embodiments, a PEgRNA or ngRNA comprises 1, 2, 3, 4, or 5 contiguous chemically modified nucleotides at the 5′ end. In some embodiments, a PEgRNA or ngRNA comprises 1, 2, or 3 contiguous chemically modified nucleotides at the 3′ end. In some embodiments, a PEgRNA or ngRNA comprises 1, 2, or 3 contiguous chemically modified nucleotides at the 5′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, or more chemically modified nucleotides near the 3′ end. In some embodiments, a PEgRNA or ngRNA comprises 3 contiguous chemically modified nucleotides at the 3′ end. In some embodiments, a PEgRNA or ngRNA comprises 3 contiguous chemically modified nucleotides at the 5′ end. In some embodiments, a PEgRNA or ngRNA comprises 1, 2, 3, 4, 5, or more chemically modified nucleotides near the 3′ end. In some embodiments, a PEgRNA or ngRNA comprises 1, 2, 3, 4, 5, or more contiguous chemically modified nucleotides near the 3′ end. In some embodiments, a PEgRNA or ngRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA or ngRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA or ngRNA comprises one or more chemical modified nucleotides in the gRNA core. The gRNA core of a PEgRNA may comprise one or more regions of a base paired lower stem, a base paired upper stem, where the lower stem and upper stem may be connected by a bulge comprising unpaired RNAs. The gRNA core may further comprise a nexus distal from the spacer sequence. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA or ngRNA can comprise a 2′-O-thionocarbamate-protected nucleoside phosphoramidite, a 2′-O-methyl (M), a 2′-O-methyl 3′phosphorothioate (MS), or a 2′-O-methyl 3′thioPACE (MSP), or any combination thereof. In some embodiments, a chemically modified PEgRNA and/or ngRNA can comprise a 2′-O-methyl (M) RNA, a 2′-O-methyl 3′phosphorothioate (MS) RNA, a 2′-O-methyl 3′thioPACE (MSP) RNA, a 2′-F RNA, a phosphorothioate bond modification, any other chemical modifications known in the art, or any combination thereof. A chemical modification may also include, for example, the incorporation of non-nucleotide linkages or modified nucleotides into the PEgRNA and/or ngRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements, such as second strand nicking ngRNAs. Components of a prime editing composition may be combined to form a complex for prime editing, or may be kept separately, e.g., for administration purposes.

In some embodiments, a prime editing composition comprises a prime editor fusion protein complexed with a PEgRNA and optionally complexed with a ngRNA. In some embodiments, the prime editing composition comprises a prime editor comprising a DNA binding domain and a DNA polymerase domain associated with each other through a PEgRNA. For example, the prime editing composition may comprise a prime editor comprising a DNA binding domain and a DNA polymerase domain linked to each other by an RNA-protein recruitment aptamer RNA sequence, which is linked to a PEgRNA. In some embodiments, a prime editing composition comprises a PEgRNA and a polynucleotide, a polynucleotide construct, or a vector that encodes a prime editor fusion protein.

In some embodiments, a prime editing composition comprises a PEgRNA, a ngRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the PEgRNA of a prime editing composition is associated with the DNA binding domain, e.g., a Cas9 nickase, of the prime editor. In some embodiments, the PEgRNA of a prime editing composition complexes with the DNA binding domain of a prime editor and directs the prime editor to the target DNA.

In some embodiments, a prime editing composition comprises one or more polynucleotides that encode prime editor components and/or PEgRNA or ngRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, and (ii) a PEgRNA or a polynucleotide encoding the PEgRNA. In some embodiments, a prime editing composition comprises (i) a polynucleotide encoding a fusion protein comprising a DNA binding domain and a DNA polymerase domain, (ii) a PEgRNA or a polynucleotide encoding the PEgRNA, and (iii) an ngRNA or a polynucleotide encoding the ngRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, and (iii) a PEgRNA or a polynucleotide encoding the PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a PEgRNA or a polynucleotide encoding the PEgRNA, and (iv) an ngRNA or a polynucleotide encoding the ngRNA.

In some embodiments, the polynucleotide encoding the DNA biding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N (ii) a polynucleotide encoding a C-terminal half of a prime editor fusion protein and an intein-C, (iii) a PEgRNA or a polynucleotide encoding the PEgRNA, and/or (iv) an ngRNA or a polynucleotide encoding the ngRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a PEgRNA or a polynucleotide encoding the PEgRNA, and/or (iv) a ngRNA or a polynucleotide encoding the ngRNA.

In some embodiments, a prime editing system comprises one or more polynucleotides encoding one or more prime editor polypeptides, wherein activity of the prime editing system may be temporally regulated by controlling the timing in which the vectors are delivered. For example, in some embodiments, a polynucleotide encoding the prime editor and a polynucleotide encoding a PEgRNA may be delivered simultaneously. For example, in some embodiments, a polynucleotide encoding the prime editor and a polynucleotide encoding a PEgRNA may be delivered sequentially.

In some embodiments, a polynucleotide encoding a component of a prime editing system may further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

Provided herein in some embodiments are example sequences for PEgRNAs, including PEgRNA spacers, PBS, RTT, and ngRNA spacers for a prime editing system comprising a nuclease that recognizes the PAM sequence “NG.” In some embodiments, a PAM motif on the edit strand comprises an “NG” motif, wherein N is any nucleotide.

### Pharmaceutical Compositions

Disclosed herein are pharmaceutical compositions comprising any of the siRNAs, antisense molecules and/or prime editing composition components provided herein (e.g., prime editors, fusion proteins, polynucleotides encoding prime editor polypeptides, PEgRNAs, ngRNAs, and/or prime editing complexes described herein).

The term “pharmaceutical composition,” as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.)

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

Methods of Using the siRNAs or the ASOs

In certain embodiments, the methods and compositions disclosed herein can be used to edit a target gene of interest by prime editing.

The siRNAs and/or the ASOs provided herein can be used to inhibit DNA mismatch repair by contacting a cell with one or more of the disclosed siRNAs and/or one or more of the disclosed ASOs. In some embodiments, the one or more siRNAs or one or more ASOs can be in a lipid nanoparticle.

Some of the disclosed methods for editing a gene comprise contacting the gene with a prime editing guide RNA (PEgRNA) (e.g., as disclosed in any of the embodiments); a prime editor comprising a DNA binding domain and a DNA polymerase domain; and one or more of the disclosed siRNAs and/or one or more of the disclosed ASOs. In these methods, the prime editor can synthesize a single stranded DNA encoded by the editing template, wherein the single stranded DNA replaces the editing target sequence and results in incorporation of the intended nucleotide edit into a region corresponding to the editing target in the gene.

In addition, as illustrated in Examples 1 through 4, which siRNA works for which alternative transcript for each of the mismatch repair genes has been mapped. Since the cells/tissues each of these transcripts is expressed by is available through public databases (e.g., those exemplified in the provided Examples), additional embodiments include using the right siRNA for the tissue of interest.

In some embodiments, the prime editing method comprises contacting a cell comprising a target gene with a PEgRNA and a prime editor (PE) polypeptide described herein and one or more of the siRNAs and/or ASOs provided herein. In some embodiments, the target gene is double stranded, and comprises two strands of DNA complementary to each other. In some embodiments, the contacting with a PEgRNA and the contacting with a prime editor are performed sequentially. In some embodiments, the contacting with a prime editor is performed after the contacting with a PEgRNA. In some embodiments, the contacting with a PEgRNA is performed after the contacting with a prime editor. In some embodiments, the contacting with a PEgRNA, and the contacting with a prime editor are performed simultaneously. In some embodiments, the PEgRNA and the prime editor are associated in a complex prior to contacting a target gene. In some embodiments, the siRNAs and/or ASOs increase the efficiency of prime editing by, e.g., at least 5%, 10%, 15%, 20%, 25%, 30%, 40%, 50%, 60%, 70%, 80%, 90%, 100%, or more as compared to the prime editing efficiency when performed in the absence of the siRNAs and/or ASOs.

In some embodiments, contacting the target gene with the prime editing composition results in binding of the PEgRNA to a target strand of the target gene. In some embodiments, contacting the target gene with the prime editing composition results in binding of the PEgRNA to a search target sequence on the target strand of the target gene upon contacting with the PEgRNA. In some embodiments, contacting the target gene with the prime editing composition results in binding of a spacer sequence of the PEgRNA to a search target sequence with the search target sequence on the target strand of the target gene upon said contacting of the PEgRNA.

In some embodiments, contacting the target gene with the prime editing composition results in binding of the prime editor to the target gene upon the contacting of the PE composition with the target gene. In some embodiments, the DNA binding domain of the PE associates with the PEgRNA. In some embodiments, the PE binds the target gene directed by the PEgRNA. Accordingly, in some embodiments, the contacting of the target gene result in binding of a DNA binding domain of a prime editor of the target gene directed by the PEgRNA.

In some embodiments, contacting the target gene with the prime editing composition results in a nick in an edit strand of the target gene by the prime editor upon contacting with the target gene, thereby generating a nicked on the edit strand of the target gene. In some embodiments, contacting the target gene with the prime editing composition results in a single-stranded DNA comprising a free 3′ end at the nick site of the edit strand of the target gene. In some embodiments, contacting the target gene with the prime editing composition results in a nick in the edit strand of the target gene by a DNA binding domain of the prime editor, thereby generating a single-stranded DNA comprising a free 3′ end at the nick site. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a part of a prime editor fusion protein or prime editing complex (in cis), or as a separate protein (in trans).

In some embodiments, contacting the target gene with the prime editing composition generates an edited single stranded DNA that is coded by the editing template of the PEgRNA by DNA polymerase mediated polymerization from the 3′ free end of the single-stranded DNA at the nick site. In some embodiments, the editing template of the PEgRNA comprises one or more intended nucleotide edits compared to endogenous sequence of the target gene. In some embodiments, the intended nucleotide edits are incorporated in the target gene, by excision of the 5′ single stranded DNA of the edit strand of the target gene generated at the nick site and DNA repair. In some embodiments, the intended nucleotide edits are incorporated in the target gene by excision of the editing target sequence and DNA repair. In some embodiments, excision of the 5′ single stranded DNA of the edit strand generated at the nick site is by a flap endonuclease. In some embodiments, the flap nuclease is FEN1. In some embodiments, the method further comprises contacting the target gene with a flap endonuclease. In some embodiments, the flap endonuclease is provided as a part of a prime editor fusion protein. In some embodiments, the flap endonuclease is provided in trans.

In some embodiments, contacting the target gene with the prime editing composition generates a mismatched heteroduplex comprising the edit strand of the target gene that comprises the edited single stranded DNA, and the unedited target strand of the target gene. Without being bound by theory, the endogenous DNA repair and replication may resolve the mismatched edited DNA to incorporate the nucleotide change(s) to form the desired edited target gene.

In some embodiments, the method further comprises contacting the target gene with a nick guide (ngRNA) disclosed herein. In some embodiments, the ngRNA comprises a spacer that binds a second search target sequence on the edit strand of the target gene. In some embodiments, the contacted ngRNA directs the PE to introduce a nick in the target strand of the target gene. In some embodiments, the nick on the target strand (non-edit strand) results in endogenous DNA repair machinery to use the edit strand to repair the non-edit strand, thereby incorporating the intended nucleotide edit in both strand of the target gene and modifying the target gene. In some embodiments, the ngRNA comprises a spacer sequence that is complementary to, and may hybridize with, the second search target sequence on the edit strand only after the intended nucleotide edit(s) are incorporated in the edit strand of the target gene.

In some embodiments, the target gene is contacted by the ngRNA, the PEgRNA, and the PE simultaneously. In some embodiments, the ngRNA, the PEgRNA, and the PE form a complex when they contact the target gene. In some embodiments, the target gene is contacted with the ngRNA, the PEgRNA, and the prime editor sequentially. In some embodiments, the target gene is contacted with the ngRNA and/or the PEgRNA after contacting the target gene with the PE. In some embodiments, the target gene is contacted with the ngRNA and/or the PEgRNA before contacting the target gene with the prime editor.

In some embodiments, the target gene is in a cell. Accordingly, also provided herein are methods of modifying a cell, such as a human cell, a human primary cell, a human iPSC-derived cell, and/or a human photoreceptor.

In some embodiments, the prime editing method comprises introducing a PEgRNA, a prime editor, a ngRNA, an siRNA provided herein and/or an ASO provided herein into the cell that has the target gene. In some embodiments, the prime editing method comprises introducing into the cell that has the target gene with a prime editing composition comprising a PEgRNA, a prime editor polypeptide, and/or a ngRNA. In some embodiments, the PEgRNA, the prime editor polypeptide, and/or the ngRNA form a complex prior to the introduction into the cell. In some embodiments, the PEgRNA, the prime editor polypeptide, and/or the ngRNA form a complex after the introduction into the cell. The prime editors, PEgRNA, ngRNAs, ASOs, siRNAs and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device.

In some embodiments, the one or more siRNAs and/or one or more ASOs is conjugated to a moiety or molecule that facilitates uptake by specific tissues and/or cells. In some embodiments, the one or more siRNAs and/or one or more ASOs is conjugated to GalNAc (e.g., mono-, di-, tri-, tetra GalNAc), which selectively binds to the asialoglycoprotein (ASGPR) receptor that is highly expressed on hepatocytes to promote selective delivery to, and endocytosis by, hepatocytes. A complete GalNAc conjugated nucleic acid can be synthesized on a solid-state oligonucleotide synthesizer. The high number, rapid turnover, and recycling of ASGPR receptors contribute to GalNAc delivery of siRNAs and ASOs. GalNAc-nucleic acid conjugates bind ASGPR and are rapidly internalized into clathrin-coated endosomes. As the endosomal pH drops, the GalNAc-siRNA is released from binding ASGPR. ASGPR is recycled back to the cell surface, while the GalNAc-siRNA remains in the lumen of the endosome.

In some embodiments, the one or more siRNAs and/or one or more ASOs is conjugated to carnitine, which promotes delivery to, and uptake by, skeletal muscle via OCTN2 transport. In some embodiments, the one or more siRNAs and/or one or more ASOs is conjugated to an antibody specific for a cell-surface marker on muscle cells to promote delivery to, and endocytosis by, muscle cells.

In some embodiments, the one or more siRNAs and/or one or more ASOs is conjugated to cholesterol to promote uptake by cells.

The prime editors, PEgRNA and/or ngRNAs, and prime editing complexes may be introduced into the cell simultaneously or sequentially.

In some embodiments, the prime editing method comprises introducing into the cell a PEgRNA or a polynucleotide encoding the PEgRNA, a prime editor polynucleotide encoding a prime editor polypeptide, an siRNA provided herein and/or an ASO provided herein and optionally an ngRNA or a polynucleotide encoding the ngRNA. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell simultaneously. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell sequentially. In some embodiments, the method comprises introducing the polynucleotide encoding the prime editor polypeptide into the cell before introduction of the PEgRNA or the polynucleotide encoding the PEgRNA and/or the ngRNA or the polynucleotide encoding the ngRNA. In some embodiments, the polynucleotide encoding the prime editor polypeptide is introduced into and expressed in the cell before introduction of the PEgRNA or the polynucleotide encoding the PEgRNA and/or the ngRNA or the polynucleotide encoding the ngRNA into the cell. In some embodiments, the polynucleotide encoding the prime editor polypeptide is introduced into the cell after the PEgRNA or the polynucleotide encoding the PEgRNA and/or the ngRNA or the polynucleotide encoding the ngRNA are introduced into the cell. The polynucleotide encoding the prime editor polypeptide, the PEgRNA or the polynucleotide encoding the PEgRNA, and/or the ngRNA or the polynucleotide encoding the ngRNA, may be introduced into the cell by any delivery approaches described herein or any delivery approach known in the art, for example, by RNPs, LNPs, viral vectors, non-viral vectors, mRNA delivery, and physical delivery.

In some embodiments, the polynucleotide encoding the prime editor polypeptide, the polynucleotide encoding the PEgRNA, and/or the polynucleotide encoding the ngRNA integrate into the genome of the cell after being introduced into the cell. In some embodiments, the polynucleotide encoding the prime editor polypeptide, the polynucleotide encoding the PEgRNA, and/or the polynucleotide encoding the ngRNA are introduced into the cell for transient expression. Accordingly, also provided herein are cells modified by prime editing.

In some embodiments, the cell is a prokaryotic cell. In some embodiments, the cell is a eukaryotic cell. In some embodiments, the cell is a mammalian cell. In some embodiments, the cell is a non-human primate cell, bovine cell, porcine cell, rodent or mouse cell. In some embodiments, the cell is a human cell.

In some embodiments, the cell is a progenitor cell. In some embodiments, the cell is a stem cell. in some embodiments, the cell is an induced pluripotent stem cell. In some embodiments, the cell is an embryonic stem cell. In some embodiments, the cell is a fibroblast.

In some embodiments, the cell is a human progenitor cell. In some embodiments, the cell is a human stem cell. In some embodiments, the cell is an induced human pluripotent stem cell. In some embodiments, the cell is a human embryonic stem cell. In some embodiments, the cell is a human fibroblast.

In some embodiments, the cell is a primary cell. In some embodiments, the cell is a human primary cell.

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, the PEgRNA or the polynucleotide encoding the PEgRNA, an siRNA provided herein and/or an ASO provided herein, and/or the ngRNA or the polynucleotide encoding the ngRNA into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the prime editor polypeptide or the polynucleotide encoding the prime editor polypeptide, the PEgRNA or the polynucleotide encoding the PEgRNA, an siRNA provided herein and/or an ASO provided herein, and/or the ngRNA or the polynucleotide encoding the ngRNA results in incorporation of one or more intended nucleotide edits in the target gene in at least one of the cells in the population of cells. In some embodiments, introduction of the prime editor polypeptide or the polynucleotide encoding the prime editor polypeptide, the PEgRNA or the polynucleotide encoding the PEgRNA, an siRNA provided herein and/or an ASO provided herein, and/or the ngRNA or the polynucleotide encoding the ngRNA results in incorporation of the one or more intended nucleotide edits in the target gene in a plurality of the population of cells. In some embodiments, introduction of the prime editor polypeptide or the polynucleotide encoding the prime editor polypeptide, the PEgRNA or the polynucleotide encoding the PEgRNA, an siRNA provided herein and/or an ASO provided herein, and/or the ngRNA or the polynucleotide encoding the ngRNA results in incorporation of the one or more intended nucleotide edits in the target gene in each cell of the population of cells. In some embodiments, introduction of the prime editor polypeptide or the polynucleotide encoding the prime editor polypeptide, the PEgRNA or the polynucleotide encoding the PEgRNA, an siRNA provided herein and/or an ASO provided herein, and/or the ngRNA or the polynucleotide encoding the ngRNA results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control. In some embodiments, the prime editing method disclosed herein has an editing efficiency of at least 30% relative to a suitable control. In some embodiments, the prime editing methods disclosed herein have an editing efficiency of at least 45% relative to a suitable control. In some embodiments, the prime editing methods disclosed herein have an editing efficiency of at least 50% relative to a suitable control.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a primary cell relative to a suitable control.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a hepatocyte relative to a corresponding control hepatocyte. In some embodiments, the hepatocyte is a human hepatocyte.

In some embodiments, the prime editing compositions provided herein are capable of incorporated one or more intended nucleotide edits without generating a significant proportion of indels. The term “indel(s),” as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37(3): 224-226 (2019), which is incorporated herein in its entirety. In some embodiments, the methods disclosed herein can have an indel frequency of less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene to a prime editing composition.

In some embodiments, the prime editing compositions provided herein are capable of incorporated one or more intended nucleotide edits efficiently without generating a significant proportion of indels. In some embodiments, the prime editing methods disclosed herein have an editing efficiency of at least about 1% and an indel frequency of less than 1% in a target cell, e.g., a human primary cell, human iPSC, human fibroblast, or human photoreceptor. In some embodiments, the prime editing methods disclosed herein have an editing efficiency of at least about 1% and an indel frequency of less than 0.5% in a target cell, e.g., a human primary cell, human iPSC, human fibroblast, or human photoreceptor. In some embodiments, the prime editing methods disclosed herein have an editing efficiency of at least about 1% and an indel frequency of less than 0.1% in a target cell, e.g., a human primary cell, human iPSC, human fibroblast, or human photoreceptor. In some embodiments, the prime editing methods disclosed herein have an editing efficiency of at least about 5% and an indel frequency of less than 1% in a target cell, e.g., a human primary cell, human iPSC, human fibroblast, or human photoreceptor. In some embodiments, the prime editing methods disclosed herein have an editing efficiency of at least about 5% and an indel frequency of less than 0.5% in a target cell, e.g., a human primary cell, human iPSC, human fibroblast, or human photoreceptor. In some embodiments, the prime editing methods disclosed herein have an editing efficiency of at least about 5% and an indel frequency of less than 0.1% in a target cell, e.g., a human primary cell, human iPSC, human fibroblast, or human photoreceptor.

In some embodiments, the prime editing methods disclosed herein have an editing efficiency of at least about 7.5% and an indel frequency of less than 1% in a target cell, e.g., a human primary cell, human iPSC, human fibroblast, or human photoreceptor. In some embodiments, the prime editing methods disclosed herein have an editing efficiency of at least about 7.5% and an indel frequency of less than 0.5% in a target cell, e.g., a human primary cell, human iPSC, human fibroblast, or human photoreceptor. In some embodiments, the prime editing methods disclosed herein have an editing efficiency of at least about 7.5% and an indel frequency of less than 0.1% in a target cell, e.g., a human primary cell, human iPSC, human fibroblast, or human photoreceptor.

In some embodiments, the prime editing methods disclosed herein have an editing efficiency of at least about 10% and an indel frequency of less than 1% in a target cell, e.g., a human primary cell, human iPSC, human fibroblast, or human photoreceptor. In some embodiments, the prime editing methods disclosed herein have an editing efficiency of at least about 10% and an indel frequency of less than 0.5% in a target cell, e.g., a human primary cell, human iPSC, human fibroblast, or human photoreceptor. In some embodiments, the prime editing methods disclosed herein have an editing efficiency of at least about 10% and an indel frequency of less than 0.1% in a target cell, e.g., a human primary cell, human iPSC, human fibroblast, or human photoreceptor.

In some embodiments, the prime editing methods disclosed herein have an editing efficiency of at least about 15% and an indel frequency of less than 1% in a target cell, e.g., a human primary cell, human iPSC, human fibroblast, or human photoreceptor. In some embodiments, the prime editing methods disclosed herein have an editing efficiency of at least about 15% and an indel frequency of less than 0.5% in a target cell, e.g., a human primary cell, human iPSC, human fibroblast, or human photoreceptor. In some embodiments, the prime editing methods disclosed herein have an editing efficiency of at least about 15% and an indel frequency of less than 0.1% in a target cell, e.g., a human primary cell, human iPSC, human fibroblast, or human photoreceptor.

In some embodiments, the prime editing methods disclosed herein have an editing efficiency of at least about 20% and an indel frequency of less than 1% in a target cell, e.g., a human primary cell, human iPSC, human fibroblast, or human photoreceptor. In some embodiments, the prime editing methods disclosed herein have an editing efficiency of at least about 20% and an indel frequency of less than 0.5% in a target cell, e.g., a human primary cell, human iPSC, human fibroblast, or human photoreceptor. In some embodiments, the prime editing methods disclosed herein have an editing efficiency of at least about 20% and an indel frequency of less than 0.1% in a target cell, e.g., a human primary cell, human iPSC, human fibroblast, or human photoreceptor.

In some embodiments, the prime editing methods disclosed herein have an editing efficiency of at least about 30% and an indel frequency of less than 1% in a target cell, e.g., a human primary cell, human iPSC, human fibroblast, or human photoreceptor. In some embodiments, the prime editing methods disclosed herein have an editing efficiency of at least about 30% and an indel frequency of less than 0.5% in a target cell, e.g., a human primary cell, human iPSC, human fibroblast, or human photoreceptor. In some embodiments, the prime editing methods disclosed herein have an editing efficiency of at least about 30% and an indel frequency of less than 0.1% in a target cell, e.g., a human primary cell, human iPSC, human fibroblast, or human photoreceptor.

In some embodiments, the prime editing methods disclosed herein have an editing efficiency of at least about 40% and an indel frequency of less than 1% in a target cell, e.g., a human primary cell, human iPSC, human fibroblast, or human photoreceptor. In some embodiments, the prime editing methods disclosed herein have an editing efficiency of at least about 40% and an indel frequency of less than 0.5% in a target cell, e.g., a human primary cell, human iPSC, human fibroblast, or human photoreceptor. In some embodiments, the prime editing methods disclosed herein have an editing efficiency of at least about 40% and an indel frequency of less than 0.1% in a target cell, e.g., a human primary cell, human iPSC, human fibroblast, or human photoreceptor.

In some embodiments, the prime editing methods disclosed herein have an editing efficiency of at least about 50% and an indel frequency of less than 1% in a target cell, e.g., a human primary cell, human iPSC, human fibroblast, or human photoreceptor. In some embodiments, the prime editing methods disclosed herein have an editing efficiency of at least about 50% and an indel frequency of less than 0.5% in a target cell, e.g., a human primary cell, human iPSC, human fibroblast, or human photoreceptor. In some embodiments, the prime editing methods disclosed herein have an editing efficiency of at least about 50% and an indel frequency of less than 0.1% in a target cell, e.g., a human primary cell, human iPSC, human fibroblast, or human photoreceptor.

In some embodiments, the prime editing methods disclosed herein have an editing efficiency of at least about 60% and an indel frequency of less than 1% in a target cell, e.g., a human primary cell, human iPSC, human fibroblast, or human photoreceptor. In some embodiments, the prime editing methods disclosed herein have an editing efficiency of at least about 60% and an indel frequency of less than 0.5% in a target cell, e.g., a human primary cell, human iPSC, human fibroblast, or human photoreceptor. In some embodiments, the prime editing methods disclosed herein have an editing efficiency of at least about 60% and an indel frequency of less than 0.1% in a target cell, e.g., a human primary cell, human iPSC, human fibroblast, or human photoreceptor.

In some embodiments, the prime editing methods disclosed herein have an editing efficiency of at least about 70% and an indel frequency of less than 1% in a target cell, e.g., a human primary cell, human iPSC, human fibroblast, or human photoreceptor. In some embodiments, the prime editing methods disclosed herein have an editing efficiency of at least about 70% and an indel frequency of less than 0.5% in a target cell, e.g., a human primary cell, human iPSC, human fibroblast, or human photoreceptor. In some embodiments, the prime editing methods disclosed herein have an editing efficiency of at least about 70% and an indel frequency of less than 0.1% in a target cell, e.g., a human primary cell, human iPSC, human fibroblast, or human photoreceptor.

In some embodiments, the prime editing methods disclosed herein have an editing efficiency of at least about 80% and an indel frequency of less than 1% in a target cell, e.g., a human primary cell, human iPSC, human fibroblast, or human photoreceptor. In some embodiments, the prime editing methods disclosed herein have an editing efficiency of at least about 80% and an indel frequency of less than 0.5% in a target cell, e.g., a human primary cell, human iPSC, human fibroblast, or human photoreceptor. In some embodiments, the prime editing methods disclosed herein have an editing efficiency of at least about 80% and an indel frequency of less than 0.1% in a target cell, e.g., a human primary cell, human iPSC, human fibroblast, or human photoreceptor.

In some embodiments, the prime editing methods disclosed herein have an editing efficiency of at least about 90% and an indel frequency of less than 1% in a target cell, e.g., a human primary cell, human iPSC, human fibroblast, or human photoreceptor. In some embodiments, the prime editing methods disclosed herein have an editing efficiency of at least about 90% and an indel frequency of less than 0.5% in a target cell, e.g., a human primary cell, human iPSC, human fibroblast, or human photoreceptor. In some embodiments, the prime editing methods disclosed herein have an editing efficiency of at least about 90% and an indel frequency of less than 0.1% in a target cell, e.g., a human primary cell, human iPSC, human fibroblast, or human photoreceptor.

In some embodiments, the prime editing methods disclosed herein have an editing efficiency of at least about 95% and an indel frequency of less than 1% in a target cell, e.g., a human primary cell, human iPSC, human fibroblast, or human photoreceptor. In some embodiments, the prime editing methods disclosed herein have an editing efficiency of at least about 95% and an indel frequency of less than 0.5% in a target cell, e.g., a human primary cell, human iPSC, human fibroblast, or human photoreceptor. In some embodiments, the prime editing methods disclosed herein have an editing efficiency of at least about 95% and an indel frequency of less than 0.1% in a target cell, e.g., a human primary cell, human iPSC, human fibroblast, or human photoreceptor. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene to a prime editing composition.

In some embodiments, the prime editing composition described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

In some embodiments, the siRNAs and/or ASOs provided herein, the prime editing compositions (e.g., PEgRNAs and prime editors as described herein) and prime editing methods disclosed herein can be used to edit a target gene. In some embodiments, the target gene comprises a mutation compared to a wild type gene. In some embodiments, the mutation is in a coding region of the target gene. In some embodiments, the mutation is in an exon of the target gene. In some embodiments, the prime editing method comprises contacting a target gene with a prime editing composition comprising a prime editor, a PEgRNA, and/or a ngRNA. In some embodiments, contacting the target gene with the prime editing composition results in incorporation of one or more intended nucleotide edits in the target gene. In some embodiments, the incorporation is in a region of the target gene that corresponds to an editing target sequence in the gene. In some embodiments, the one or more intended nucleotide edits comprises a single nucleotide substitution, an insertion, a deletion, or any combination thereof, compared to the endogenous sequence of the target gene. In some embodiments, incorporation of the one or more intended nucleotide edits results in replacement of the one or more mutations with the corresponding sequence in a wild type gene. In some embodiments, incorporation of the one more intended nucleotide edits results in correction of a mutation in the target gene. In some embodiments, the target gene comprises an editing target sequence that contains the mutation. In some embodiments, contacting the target gene with the prime editing composition results in incorporation of one or more intended nucleotide edits in the target gene, which corrects the mutation in the editing target sequence (or a double stranded region comprising the editing target sequence and the complementary sequence to the editing target sequence on a target strand) in the target gene.

In some embodiments, the target gene is in a target cell. Accordingly, in one aspect provided herein is a method of editing a target cell comprising a target gene that encodes a polypeptide that comprises one or more mutations relative to a wild type gene. In some embodiments, the methods of the present disclosure comprise introducing a prime editing composition comprising the siRNAs and/or ASOs provided herein, a PEgRNA, a prime editor polypeptide, and/or a ngRNA into the target cell that has the target gene to edit the target gene, thereby generating an edited cell. In some embodiments, the target cell is a mammalian cell. In some embodiments, the target cell is a human cell. In some embodiments, the target cell is a progenitor cell. In some embodiments, the target cell is a stem cell. in some embodiments, the target cell is an induced pluripotent stem cell. In some embodiments, the target cell is an embryonic stem cell. In some embodiments, the target cell is a fibroblast. In some embodiments, the target cell is a human progenitor cell. In some embodiments, the target cell is a human stem cell. in some embodiments, the target cell is an induced human pluripotent stem cell. In some embodiments, the target cell is a human embryonic stem cell. In some embodiments, the target cell is a human fibroblast. In some embodiments, the target cell is a primary cell. In some embodiments, the target cell is a human primary cell.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, protein expression can be measured using a protein assay. In some embodiments, protein expression can be measured using antibody testing. In some embodiments, protein expression can be measured using ELISA, mass spectrometry, Western blot, sodium dodecyl sulfate polyacrylamide gel electrophoresis (SDS-PAGE), high performance liquid chromatography (HPLC), electrophoresis, or any combination thereof. In some embodiments, a protein assay can comprise SDS-PAGE and densitometric analysis of a Coomassie Blue-stained gel.

### Delivery

The siRNAs and/or ASOs provided herein and/or prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, an siRNA, ASO, and/or PEgRNA can be delivered directly as an RNA or as a DNA encoding the siRNA, ASO, and/or PEgRNA.

In some embodiments, an siRNA, ASOs, and/or prime editing composition component is encoded by a polynucleotide, a vector, or a construct. In some embodiments, an siRNA, ASO, a prime editor polypeptide, a PEgRNA and/or a ngRNA is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA polymerase domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes an siRNA, an ASO, a PEgRNA and/or a ngRNA. In some embodiments, the polypeptide encodes two or more components of a prime editing composition, for example, a prime editor fusion protein and a PEgRNA.

In some embodiments, the polynucleotide encoding an siRNA, ASO, and/or one or more prime editing composition components is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding an siRNA, ASO, and/or one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, H1 promoter).

In some embodiments, the polynucleotide encoding an siRNA, ASO, and/or one or more prime editing composition components is a part of, or is encoded by, a vector. In some embodiments, the vector is a viral vector. In some embodiments, the vector is a non-viral vector.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA, e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, a mRNA that encodes a prime editor polypeptide is generated using in vitro transcription. Guide polynucleotides (e.g., PEgRNA or ngRNA) can also be transcribed using in vitro transcription from a cassette containing a T7 promoter, followed by the sequence “GG,” and guide polynucleotide sequence. In some embodiments, the prime editor encoding mRNA, PEgRNA, and/or ngRNA are synthesized in vitro using an RNA polymerase enzyme (e.g., T7 polymerase, T3 polymerase, SP6 polymerase, etc.). Once synthesized, the RNA can directly contact a target gene or can be introduced into a cell using any suitable technique for introducing nucleic acids into cells (e.g., microinjection, electroporation, transfection). In some embodiments, the prime editor-coding sequences, the PEgRNAs, and/or the ngRNAs are modified to include one or more modified nucleoside e.g., using pseudo-U or 5-Methyl-C.

Methods of non-viral delivery of nucleic acids can include lipofection, nucleofection, microinjection, biolistics, virosomes, liposomes, exosomes, immunoliposomes, polycation or lipid:nucleic acid conjugates, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid:nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), simian immunodeficiency virus (SIV), human immunodeficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector.

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and ψ2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g., a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the full-length prime editor fusion protein is reassembled upon co-infection of the same cell by both dual AAV vectors, expression of both halves of the prime editor fusion protein, and self-excision of the inteins.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a including peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 2514). As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine (SEQ ID NO: 2515), and octa-arginine (SEQ ID NO: 2516). The nona-arginine (R9) (SEQ ID NO: 2515) sequence can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, an siRNA and/or ASO, prime editor polypeptide components and PEgRNA/ngRNA are introduced to a target cell by nanoparticles. In some embodiments, the prime editor polypeptide components and the PEgRNA and/or ngRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a hepatocyte, in an organic nanoparticle, e.g., a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids used to produce LNPs are provided in Table E below.

In some embodiments, components of a prime editing composition form a complex prior to delivery to a target cell. For example, an siRNA and/or ASO, a prime editor fusion protein, a PEgRNA, and/or a ngRNA can form a complex prior to delivery to the target cell. In some embodiments, a prime editing polypeptide (e.g., a prime editor fusion protein) and a guide polynucleotide (e.g., a PEgRNA or ngRNA) form a ribonucleoprotein (RNP) for delivery to a target cell. In some embodiments, the RNP comprises a prime editor fusion protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary lipids for use in nanoparticle formulations and/or gene transfer are shown in Table E below.

TABLE E
Exemplary lipids for nanoparticle formulation or gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N41-(2,3-Dioleyloxy)prophyliN,N,N-trimethylammonium | DOTMA | Cationic |
| chloride | | |
| 1,2-Dioleoyloxy-3-trimethylammonium-propane | DOGS | Cationic |
| Dioctadecylamidoglycylspermine | | |
| N-(3-Aminopropyl)-N,N-dimethyl-2,3-bis(dodecyloxy)- | GAP-DLRIE | Cationic |
| 1- | | |
| propanaminium bromide | | |
| Cetyltrimethylammonium bromide | CTAB | Cationic |
| 6-Lauroxyhexyl omithinate | LHON | Cationic |
| 1-(2,3-Dioleoyloxypropy1)-2,4,6-trimethylpyridinium | 20c | Cationic |
| 2,3-Dioleyloxy-N-P(spenninecarboxamido-ethy1J- | DOSPA | Cationic |
| N,Ndimethyl- | | |
| l-propanatninium trifluoroacetate | | |
| 1,2-Dioley1-3-trimethylamtnonium-propane | DOPA | Cationic |
| N-(2-Hydroxyethyl)-N,N-dimethyl-2,3- | MDRIE | Cationic |
| bis(tetradecyloxy)-1- | | |
| propanaminium bromide | | |
| Dimyristooxypropyl dimethyl hydroxyethyl ammonium | DMRI | Cationic |
| bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermy1)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyloxymethyloxy) | CLIP-6 | Cationic |
| ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spenmine | CCS | Cationic |
| N-t-Butyl-N0-tetradecyl-3- | diC14- | Cationic |
| tetradecylaminopropionamidine | amidine | |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9- | CDAN | Cationic |
| diamine | | |
| 2-(3-Bis(3-amino-propy1)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

Exemplary polymers for use in nanoparticle formulations and/or gene transfer are shown in Table F below.

TABLE F
Exemplary lipids for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for polynucleotides encoding prime editing composition components are shown in Table G below.

TABLE G
Exemplary polynucleotide delivery methods

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic |
| | electroporation, | | | | Acids and |
| | particle gun, | | | | Proteins |
| | Calcium | | | | |
| | phosphate | | | | |
| | transfection) | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic |
| | | | | what is | acids and |
| | | | | delivered | Proteins |
| | Polymeric | YES | Transient | NO | Nucleic |
| | Nanoparticles | | | | Acids |
| Biological | Attenuated | YES | Transient | NO | Nucleic |
| | Bacteria | | | | Acids |
| Non-Viral | Engineered | YES | Transient | NO | Nucleic |
| Delivery | Bacteriophages | | | | Acids |
| Vehicles | Mammalian Virus- | YES | Transient | NO | Nucleic |
| | like Particles | | | | Acids |
| | Biological | YES | Transient | NO | Nucleic |
| | liposomes: | | | | Acids |
| | Erythrocyte | | | | |
| | Ghosts and | | | | |
| | Exosomes | | | | |

The siRNAs, ASOs, and prime editing compositions of the disclosure, whether introduced as polynucleotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g., two different polynucleotide constructs are provided to the cell (e.g., different components of the same prime editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The siRNAs, ASOs, and prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g., two different polynucleotide constructs are administered to the subject (e.g., different components of the same prim editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

### EXAMPLES

The disclosure will be further illustrated with reference to the following specific examples. These examples are given by way of illustration and are not meant to limit the disclosure or the claims that follow.

### Example 1: Identification of Potential Screening Candidate siRNAs Targeting Human MSH2 mRNA

Identification of potential screening candidate siRNAs targeting the following mRNA: MSH2 (mutShomolog 2, Gene ID: 4436) included a bioinformatical approach that assumes a canonical siRNA structure. Positions 2-18 (5′-3′) of the sense and antisense strand are used for the specificity calculations. Positions 1-19 (5′-3′) of the antisense strand are used for cross-reactivity and human SNP analysis. The following parameters are assessed (1) Species cross-reactivity for human: Analysis is based on a canonical siRNA design using 19 bases for cross-reactivity; (2) Predicted specificity in human: Analysis of sense and antisense strand separately; (3) Identicalness of siRNA seed region and seed region of known miRNAs; (4) Analysis of human SNP database (NCBI-DB-SNP) to identify siRNAs targeting regions with known SNPs Information to include positions of SNPs within the target sequence as well as minor allele frequency (MAF) in case data are available; and (5) siRNA activity prediction based on canonical siRNA design.

In the bioinformatics procedure, all possible siRNAs were created from human MSH2 mRNA sequence (NM_000251.3); siRNA off-target genes were predicted for human; a specificity score was assigned to each siRNA strand; siRNA strands were analyzed for presence of human, rhesus monkey, dog, pig, mouse, rat and rabbit miRNA seed regions; specificity categories were assigned to siRNAs (combined specificity score+miRNA seed analysis); siRNA cross-reactivity was calculated: for transcript variants and with 19mers; human SNPs were mapped to siRNA target sites in MSH2 transcript NM_000251.3; siRNA activity was predicted and a score was assigned to each siRNA.

The siRNA selection considered cross-reactivity with 19mer in human MSH2 mRNA transcripts; predicted as specific in human; siRNA target sites do not harbor SNPs with a MAF ≥1% (pos. 2-18); and having a base composition with no stretches of more than 4 G's in a row. For cross-reactivity, “1” means siRNA is perfectly cross-reactive with the relevant target transcripts, and “0” means relevant transcripts are not perfectly matched. Results are presented in Table 21.

The databases used included NCBI RefSeqDB release 208 (September 2021) and EnsemblDB release 104 (May 2021) for RNA sequences; NCBI RefSeqDB release 208 (September 2021) for Specificity prediction; miRBaseR22 (human, rhesus, mouse, rat, dog, pig) (March 2018) for miRNA seed analysis; and NCBI dbSNPBuild 2.0 155 (April 2021) for SNPs.

In silico evaluation of siRNA candidates considered identifying siRNAs with lowest sequence complementarity to any non-target transcript and siRNAs whose seed region (pos. 2-7) is ideally not identical to a seed region (pos. 2-7) of known miRNAs; selecting siRNAs that target at least all protein-coding transcripts of the target gene and for each species; perfect match (19mer) with target sequences in primary species; 17mer (pos. 2-18 of 19mer) perfect match and possibly single mismatch hits (within pos. 2-18) with target sequences in the secondary species; SNPs (single nucleotide polymorphism) to exclude target sites with abundant SNPs (NCBI, dbSNP) and single mismatch to target site can weaken siRNA activity; and siRNA activity prediction by in-house and published algorithms.

Off target prediction by identification of near perfect matched genes considers the likelihood of unintended downregulation of any other transcript by full or partial complementarity of a siRNA strand (up to 4 mismatches within positions 2-18); is based on number and position of mismatches; describes the predicted most likely off-target(s) for antisense and sense strand of each siRNA; prefers siRNAs with low number of predicted off-targets; and is used for refined ranking within siRNA candidate sets.

Seed dependent, microRNA like off target effects considers that siRNAs can function in a miRNA like manner via base-pairing with complementary sequences within the 3′-UTR of mRNA molecules; the complementarity typically encompasses the 5′-bases 2-7 of the miRNA (seed region); in order to circumvent siRNAs to act via functional miRNA binding sites, we avoid siRNA strands, that contain natural miRNA seed regions; and seed regions identified in miRNAs from human, mouse, rat, rhesus monkey, dog, pig and rabbit were referred to as “conserved.”

### Example 2: Identification of Potential Screening Candidate siRNAs Targeting Human PMS2 mRNA

Identification of potential screening candidate siRNAs targeting the following mRNA: PMS2 (PMS1 homolog 2, mismatch repair system component, Gene ID: 5395) included a bioinformatical approach that assumes a canonical siRNA structure. Positions 2-18 (5′-3′) of the sense and antisense strand are used for the specificity calculations. Positions 1-19 (5′-3′) of the antisense strand are used for cross-reactivity and human SNP analysis. The following parameters are assessed (1) Species cross-reactivity for human: Analysis is based on a canonical siRNA design using 19 bases for cross-reactivity; (2) Predicted specificity in human: Analysis of sense and antisense strand separately; (3) Identicalness of siRNA seed region and seed region of known miRNAs; (4) Analysis of human SNP database (NCBI-DB-SNP) to identify siRNAs targeting regions with known SNPs-Information to include positions of SNPs within the target sequence as well as minor allele frequency (MAF) in case data are available; and (5) siRNA activity prediction based on canonical siRNA design.

In the bioinformatics procedure, all possible siRNAs were created from human PMS2 mRNA sequence (NM_000535.7); siRNA off-target genes were predicted for human; a specificity score was assigned to each siRNA strand; siRNA strands were analyzed for presence of human, rhesus monkey, dog, pig, mouse, rat and rabbit miRNA seed regions; specificity categories were assigned to siRNAs (combined specificity score+miRNA seed analysis); siRNA cross-reactivity was calculated: for transcript variants and with 19mers; human SNPs were mapped to siRNA target sites in PMS2 transcript NM_000535.7; siRNA activity was predicted and a score was assigned to each siRNA.

The siRNA selection considered cross-reactivity with 19mer in human PMS2 mRNA transcripts; predicted as specific in human; siRNA target sites do not harbor SNPs with a MAF ≥1% (pos. 2-18); and having a base composition with no stretches of more than 4 G's in a row. For cross-reactivity, “1” means siRNA is perfectly cross-reactive with the relevant target transcripts, and “0” means relevant transcripts are not perfectly matched. Results are presented in Table 22.

The databases used included NCBI RefSeqDB release 208 (September 2021) and EnsemblDB release 104 (May 2021) for RNA sequences; NCBI RefSeqDB release 208 (September 2021) for Specificity prediction; miRBaseR22 (human, rhesus, mouse, rat, dog, pig) (March 2018) for miRNA seed analysis; and NCBI dbSNPBuild 2.0 155 (April 2021) for SNPs.

In silico evaluation of siRNA candidates considered identifying siRNAs with lowest sequence complementarity to any non-target transcript and siRNAs whose seed region (pos. 2-7) is ideally not identical to a seed region (pos. 2-7) of known miRNAs; selecting siRNAs that target at least all protein-coding transcripts of the target gene and for each species; perfect match (19mer) with target sequences in primary species; 17mer (pos. 2-18 of 19mer) perfect match and possibly single mismatch hits (within pos. 2-18) with target sequences in the secondary species; SNPs (single nucleotide polymorphism) to exclude target sites with abundant SNPs (NCBI, dbSNP) and single mismatch to target site can weaken siRNA activity; and siRNA activity prediction by in-house and published algorithms.

Off target prediction by identification of near perfect matched genes considers the likelihood of unintended downregulation of any other transcript by full or partial complementarity of a siRNA strand (up to 4 mismatches within positions 2-18); is based on number and position of mismatches; describes the predicted most likely off-target(s) for antisense and sense strand of each siRNA; prefers siRNAs with low number of predicted off-targets; and is used for refined ranking within siRNA candidate sets.

Seed dependent, microRNA like off target effects considers that siRNAs can function in a miRNA like manner via base-pairing with complementary sequences within the 3′-UTR of mRNA molecules; the complementarity typically encompasses the 5′-bases 2-7 of the miRNA (seed region); in order to circumvent siRNAs to act via functional miRNA binding sites, we avoid siRNA strands, that contain natural miRNA seed regions; and seed regions identified in miRNAs from human, mouse, rat, rhesus monkey, dog, pig and rabbit were referred to as “conserved.”

### Example 3: Identification of Potential Screening Candidate siRNAs Targeting Human MSH6 mRNA

Identification of potential screening candidate siRNAs targeting the following mRNA: MSH6 (mutS homolog 6, Gene ID: 2956) included a bioinformatical approach that assumes a canonical siRNA structure. Positions 2-18 (5′-3′) of the sense and antisense strand are used for the specificity calculations. Positions 1-19 (5′-3′) of the antisense strand are used for cross-reactivity and human SNP analysis. The following parameters are assessed (1) Species cross-reactivity for human: Analysis is based on a canonical siRNA design using 19 bases for cross-reactivity; (2) Predicted specificity in human: Analysis of sense and antisense strand separately; (3) Identicalness of siRNA seed region and seed region of known miRNAs; (4) Analysis of human SNP database (NCBI-DB-SNP) to identify siRNAs targeting regions with known SNPs Information to include positions of SNPs within the target sequence as well as minor allele frequency (MAF) in case data are available; and (5) siRNA activity prediction based on canonical siRNA design.

In the bioinformatics procedure, all possible siRNAs were created from human MSH6 mRNA sequence (NM_000179.3); siRNA off-target genes were predicted for human; a specificity score was assigned to each siRNA strand; siRNA strands were analyzed for presence of human, rhesus monkey, dog, pig, mouse, rat and rabbit miRNA seed regions; specificity categories were assigned to siRNAs (combined specificity score+miRNA seed analysis); siRNA cross-reactivity was calculated: for transcript variants and with 19mers; human SNPs were mapped to siRNA target sites in MSH6 transcript NM_000179.3; siRNA activity was predicted and a score was assigned to each siRNA.

The siRNA selection considered cross-reactivity with 19mer in human MSH6 mRNA transcripts; predicted as specific in human; siRNA target sites do not harbor SNPs with a MAF ≥1% (pos. 2-18); and having a base composition with no stretches of more than 4 G's in a row. For cross-reactivity, “1” means siRNA is perfectly cross-reactive with the relevant target transcripts, and “0” means relevant transcripts are not perfectly matched. Results are presented in Table 23.

The databases used included NCBI RefSeqDB release 208 (September 2021) and EnsemblDB release 104 (May 2021) for RNA sequences; NCBI RefSeqDB release 208 (September 2021) for Specificity prediction; miRBaseR22 (human, rhesus, mouse, rat, dog, pig) (March 2018) for miRNA seed analysis; and NCBI dbSNPBuild 2.0 155 (April 2021) for SNPs.

In silico evaluation of siRNA candidates considered identifying siRNAs with lowest sequence complementarity to any non-target transcript and siRNAs whose seed region (pos. 2-7) is ideally not identical to a seed region (pos. 2-7) of known miRNAs; selecting siRNAs that target at least all protein-coding transcripts of the target gene and for each species; perfect match (19mer) with target sequences in primary species; 17mer (pos. 2-18 of 19mer) perfect match and possibly single mismatch hits (within pos. 2-18) with target sequences in the secondary species; SNPs (single nucleotide polymorphism) to exclude target sites with abundant SNPs (NCBI, dbSNP) and single mismatch to target site can weaken siRNA activity; and siRNA activity prediction by in-house and published algorithms.

Off target prediction by identification of near perfect matched genes considers the likelihood of unintended downregulation of any other transcript by full or partial complementarity of a siRNA strand (up to 4 mismatches within positions 2-18); is based on number and position of mismatches; describes the predicted most likely off-target(s) for antisense and sense strand of each siRNA; prefers siRNAs with low number of predicted off-targets; and is used for refined ranking within siRNA candidate sets.

Seed dependent, microRNA like off target effects considers that siRNAs can function in a miRNA like manner via base-pairing with complementary sequences within the 3′-UTR of mRNA molecules; the complementarity typically encompasses the 5′-bases 2-7 of the miRNA (seed region); in order to circumvent siRNAs to act via functional miRNA binding sites, we avoid siRNA strands, that contain natural miRNA seed regions; and seed regions identified in miRNAs from human, mouse, rat, rhesus monkey, dog, pig and rabbit were referred to as “conserved.”

### Example 4: Identification of Potential Screening Candidate siRNAs Targeting Human MLH1 mRNA

Identification of potential screening candidate siRNAs targeting the following mRNA: MLH1 (mutL homolog 1, Gene ID: 4292) included a bioinformatical approach that assumes a canonical siRNA structure. Positions 2-18 (5′-3′) of the sense and antisense strand are used for the specificity calculations. Positions 1-19 (5′-3′) of the antisense strand are used for cross-reactivity and human SNP analysis. The following parameters are assessed (1) Species cross-reactivity for human: Analysis is based on a canonical siRNA design using 19 bases for cross-reactivity; (2) Predicted specificity in human: Analysis of sense and antisense strand separately; (3) Identicalness of siRNA seed region and seed region of known miRNAs; (4) Analysis of human SNP database (NCBI-DB-SNP) to identify siRNAs targeting regions with known SNPs Information to include positions of SNPs within the target sequence as well as minor allele frequency (MAF) in case data are available; and (5) siRNA activity prediction based on canonical siRNA design.

In the bioinformatics procedure, all possible siRNAs were created from human MLH1 mRNA sequence (NM_000249.4); siRNA off-target genes were predicted for human; a specificity score was assigned to each siRNA strand; siRNA strands were analyzed for presence of human, rhesus monkey, dog, pig, mouse, rat and rabbit miRNA seed regions; specificity categories were assigned to siRNAs (combined specificity score+miRNA seed analysis); siRNA cross-reactivity was calculated: for transcript variants and with 19mers; human SNPs were mapped to siRNA target sites in MLH1 transcript NM_000249.4; siRNA activity was predicted and a score was assigned to each siRNA.

The siRNA selection considered cross-reactivity with 19mer in human MLH1 mRNA transcripts; predicted as specific in human; siRNA target sites do not harbor SNPs with a MAF ≥1% (pos. 2-18); and having a base composition with no stretches of more than 4 G's in a row. For cross-reactivity, “1” means siRNA is perfectly cross-reactive with the relevant target transcripts, and “0” means relevant transcripts are not perfectly matched. Results are presented in Table 24 (which includes Table 24A and Table 24B).

The databases used included NCBI RefSeqDB release 208 (September 2021) and EnsemblDB release 104 (May 2021) for RNA sequences; NCBI RefSeqDB release 208 (September 2021) for Specificity prediction; miRBaseR22 (human, rhesus, mouse, rat, dog, pig) (March 2018) for miRNA seed analysis; and NCBI dbSNPBuild 2.0 155 (April 2021) for SNPs.

In silico evaluation of siRNA candidates considered identifying siRNAs with lowest sequence complementarity to any non-target transcript and siRNAs whose seed region (pos. 2-7) is ideally not identical to a seed region (pos. 2-7) of known miRNAs; selecting siRNAs that target at least all protein-coding transcripts of the target gene and for each species; perfect match (19mer) with target sequences in primary species; 17mer (pos. 2-18 of 19mer) perfect match and possibly single mismatch hits (within pos. 2-18) with target sequences in the secondary species; SNPs (single nucleotide polymorphism) to exclude target sites with abundant SNPs (NCBI, dbSNP) and single mismatch to target site can weaken siRNA activity; and siRNA activity prediction by in-house and published algorithms.

Off target prediction by identification of near perfect matched genes considers the likelihood of unintended downregulation of any other transcript by full or partial complementarity of a siRNA strand (up to 4 mismatches within positions 2-18); is based on number and position of mismatches; describes the predicted most likely off-target(s) for antisense and sense strand of each siRNA; prefers siRNAs with low number of predicted off-targets; and is used for refined ranking within siRNA candidate sets.

Seed dependent, microRNA like off target effects considers that siRNAs can function in a miRNA like manner via base-pairing with complementary sequences within the 3′-UTR of mRNA molecules; the complementarity typically encompasses the 5′-bases 2-7 of the miRNA (seed region); in order to circumvent siRNAs to act via functional miRNA binding sites, we avoid siRNA strands, that contain natural miRNA seed regions; and seed regions identified in miRNAs from human, mouse, rat, rhesus monkey, dog, pig and rabbit were referred to as “conserved.”

### Example 5: Testing the Activity of siRNAs on Prime Editing Outcomes

To test the activity of siRNAs on prime editing, siRNAs either individually or combined in a pool are mixed with a lipid-based transfection reagent and added to cells seeded in cell culture plates. Twenty-four, forty-eight or seven two hours later, plasmid DNA encoding U6 promoter-driven pegRNA expression cassettes and plasmid encoding the prime editor PE2 are co-transfected into HEK293T cells. Alternatively, pegRNA and/or a prime editor may be administered to the cell in mRNA form, such as in vitro transcribed RNA. Seventy-two hours after prime editor and pegRNA transfection, gDNA is extracted from cells and the prime editing-targeted region of the genome is amplified by PCR. siRNA activity on prime editing outcomes is evaluated by amplicon sequencing and scored for the percentage of sequence reads with the intended prime edit.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

### INCORPORATION BY REFERENCE

All U.S. patents, and U.S. and PCT patent application publications mentioned herein are hereby incorporated by reference in their entirety as if each individual patent or patent application publication was specifically and individually indicated to be incorporated by reference. In case of conflict, the present application, including any definitions herein, will control.

### EQUIVALENTS

Those skilled in the art will recognize or be able to ascertain using no more than routine experimentation many equivalents to the specific embodiments of the present disclosure described herein. Such equivalents are intended to be encompassed by the following claims.

1.-46. (canceled)

47. A system comprising:
a) prime editing guide RNA (PEgRNA) comprising:
i) a spacer that comprises a region of complementarity to a search target sequence in target strand of a double stranded target DNA;
ii) a guide RNA (gRNA) core;
iii) an editing template that comprises an intended edit compared to the double stranded target DNA; and
iv) a primer binding site (PBS) that comprises a region of complementarity to a region upstream of a nick site in a non-target strand of the double stranded target DNA, and
b) an siRNA or an antisense oligo nucleotide (ASO),
wherein the siRNA is
i) specific for an mRNA sequence of a mutS homolog 2 (MSH2) gene and comprises sequence complementarity to any one of the target nucleic acid sequences set forth in SEQ ID NO: 177 to 200;
ii) specific for an mRNA sequence of a PMS1 homolog 2 mismatch repair system component (PMS2) gene and comprises sequence complementarity to any one of the target nucleic acid sequences set forth in SEQ ID NO: 373 to 396;
iii) specific for an mRNA sequence of a mutS homolog 6 (MSH6) gene and comprises sequence complementarity to any one of the target nucleic acid sequences set forth in SEQ ID NO: 585 to 608(iii); or
iv) specific for an mRNA sequence of a mutL homolog 1 (MLH1) gene and comprises sequence complementarity to any one of the target nucleic acid sequences set forth in SEQ ID NO: 790 to 813,
wherein the ASO is
i) specific for an mRNA sequence of the mutS homolog 2 (MSH2) gene and comprises a nucleic acid sequence set forth in SEQ ID NOs: 1-32;
ii) specific for an mRNA sequence of the PMS1 homolog 2 mismatch repair system component (PMS2) gene and comprises a nucleic acid sequence set forth in SEQ ID NOs: 201-228;
iii) specific for an mRNA sequence of the mutS homolog 6 (MSH6) gene and comprises a nucleic acid sequence set forth in SEQ ID NOs: 397-440;
or
iv) specific for an mRNA sequence of the mutL homolog 1 (MLH1) gene and comprises a nucleic acid sequence set forth in SEQ ID NOs: 609-645.

48. The system of claim 47, wherein the siRNA is specific for an mRNA sequence of a mutS homolog 2 (MSH2) gene and comprises any one of the matched antisense strand and sense strand pairs set forth in Tables 2-4.

49. The system of claim 47, wherein the siRNA is specific for an mRNA sequence of a PMS1 homolog 2 mismatch repair system component (PMS2) gene and comprises any one of the matched antisense strand and sense strand pairs set forth in Tables 7-9.

50. The system of claim 47, wherein the siRNA is specific for an mRNA sequence of a mutS homolog 6 (MSH6) gene and comprises any one of the matched antisense strand and sense strand pairs set forth in Tables 12-14.

51. The system of claim 47, wherein the siRNA is specific for an mRNA sequence of the mutL homolog 1 (MLH1) gene and comprises any one of the matched antisense strand and sense strand pairs set forth in Tables 17-19.

52. The system of claim 47, wherein the siRNA comprises
a) a phosphorothioate internucleotide bond, a methylphosphonate internucleotide bond, and/or a triazole internucleotide bond, optionally wherein siRNA comprises a 2′-O-methoxyethyl oligonucleotide (2′MOE), a 2′-O-methylated nucleoside (2′OMe), a 2′-fluoro oligonucleotide (2′F), an arabino nucleotide (ANA), a 2′-F arabino nucleotide (FANA), a phosphorodiamidate morpholino oligonucleotide (PMO), a peptide nucleic acid (PNA), a phosphorothioate bond (PS), a locked nucleic acid (LNA), a hydrophobic moiety, a naphthyl modifier, or a cholesterol moiety;
b) a cholesterol, a dialkyl lipid, GalNAc, or a poly-ethylene glycol (PEG);
or
c) a 5′ end cap or a 3′ end cap.

53. The system of claim 45, wherein the siRNA comprises a DNA nucleotide or a DNA nucleoside, optionally wherein the DNA nucleotide or the DNA nucleoside is thymine.

54. The system of claim 47, wherein the ASO comprises an antisense strand comprising deoxyribonucleotides and/or ribonucleotides.

55. The system of claim 47, wherein the ASO comprises an antisense strand, wherein the antisense strand comprises at least five ribonucleotides at the 5′ end of the antisense strand or at least five ribonucleotides at the 3′ end of the antisense strand.

56. The system of claim 54, wherein the ASO comprises an antisense strand (5′ to 3′) comprising deoxyribonucleotides from nucleotide position 6 to nucleotide position 15.

57. The system of claim 54, wherein the ASO comprises
a) a chemical modification, optionally wherein the ASO comprises a phosphorothioate internucleotide bond, a methylphosphonate internucleotide bond, and/or a triazole internucleotide bond, optionally wherein ASO comprises a 2′-O-methoxyethyl oligonucleotide (2′MOE), a 2′-O-methylated nucleoside (2′OMe), a 2′-fluoro oligonucleotide (2′F), an arabino nucleotide (ANA), a 2′-F arabino nucleotide (FANA), a phosphorodiamidate morpholino oligonucleotide (PMO), a peptide nucleic acid (PNA), a phosphorothioate bond (PS), a locked nucleic acid (LNA), a hydrophobic moiety, a naphthyl modifier, or a cholesterol moiety;
b) a cholesterol, a dialkyl lipid, GalNAc, or poly-ethylene glycol (PEG);
or
c) a 5′ end cap or a 3′ end cap.

58. The system of claim 47, further comprising a prime editor or one or more polynucleotides encoding the prime editor, wherein the prime editor comprises a DNA binding domain and a DNA polymerase domain.

59. The system of claim 58, wherein the DNA binding domain comprises a Cas9 nickase comprising a mutation in an HNH domain, and wherein the DNA polymerase domain comprises a reverse transcriptase.

60. A lipid nanoparticle (LNP) or polymer nanoparticle comprising the system of claim 47.

61. A lipid nanoparticle (LNP) or polymer nanoparticle comprising the system of claim 58.

62. A method for editing a gene, the method comprising contacting the gene with the system of claim 58.

63. A method for editing a gene, the method comprising contacting the gene with the LNP or polymer nanoparticle of claim 60.

64. A method for editing a gene in a subject in need thereof, the method comprising contacting the gene with the system of claim 47.

65. The method of claim 64, wherein the system further comprises a prime editor or one or more polynucleotides encoding the prime editor, wherein the prime editor comprises a DNA binding domain and a DNA polymerase domain.

66. A method for editing a gene in a subject in need thereof, the method comprising contacting the gene with LNP or polymer nanoparticle of claim 60.
